(19) United States
(12) Patent Application Publication
(10) Pub. No.: US 2022/0154282 A1
(43) Pub. Date: May 19, 2022

(54) DETECTION MEANS, COMPOSITIONS AND METHODS FOR MODULATING SYNOVIAL SARCOMA CELLS

(71) Applicants: The Broad Institute, Inc., Cambridge, MA (US); Massachusetts Institute of Technology, Boston, MA (US); The General Hospital Corporation, Boston, MA (US)

(72) Inventors: Aviv Regev, Cambridge, MA (US); Livnat Jerby-Arnon, Cambridge, MA (US); Mario Suva, Boston, MA (US); Nicolo Riggi, Boston, MA (US)

(21) Appl. No.: 17/438,051

(22) PCT Filed: Mar. 12, 2020
(86) PCT No.: PCT/US2020/022466

§ 371 Date: Sep. 10, 2021

Related U.S. Application Data
(60) Provisional application No. 62/817,545, filed on Mar. 12, 2019.
(60) Provisional application No. 62/880,438, filed on Jul. 30, 2019.

(51) Int. Cl. C12Q 1/6886

(52) U.S. Cl. CPC C12Q 1/6886; A61K 35/17

(57) ABSTRACT

The present invention provides novel compositions and methods based on the discovery of the mechanisms and gene expression programs associated with synovial sarcoma. In particular, core oncogenic programs were expressed by a distinct subpopulation of malignant cells and associated with poor clinical outcome, a cell cycle program distinguished cycling from non-cycling cells, with cycling cells having a tendency to be poorly differentiated and indicative of increased risk of metastatic disease, and a (de)differentiation program that can identify poorly differentiated cells, the absence of which was prognostic of metastasis free survival. Methods of treatment include use of HDAC and CDK4/6 inhibitors to block oncogenic program to selectively target synovial sarcoma cells. Finally, macrophages and T cells can mimic the effect of SS18-SSX inhibition by secreting TNFa and IFNg, which allows for adoptive cell therapy to provide cells with increased expression of TNFa and IFNg.

### CROSS-REFERENCE TO RELATED APPLICATIONS

This application claims the benefit of U.S. Provisional Application No. 62/817,545 filed Mar. 12, 2019 and U.S. Provisional Application 62/880,438 filed Jul. 30, 2019. The entire contents of the above-identified applications are fully incorporated herein by reference.

### STATEMENT REGARDING FEDERALLY SPONSORED RESEARCH

This invention was made with government support under grant numbers CA180922, CA202820, CA14051 granted by the National Institutes of Health. The government has certain rights in the invention.

### REFERENCE TO AN ELECTRONIC SEQUENCE LISTING

The contents of the electronic sequence listing (BROD-4110WP_ST25.txt”; Size is 12 Kilobytes and it was created on Mar. 12, 2020) is herein incorporated by reference in its entirety.

### TECHNICAL FIELD

The subject matter disclosed herein is generally directed to compositions and methods for modulating synovial sarcoma cells and responses by targeting SS18-SSX oncoprotein/core oncogenic program.

### BACKGROUND

Synovial sarcoma (SyS) is a highly aggressive mesenchymal neoplasm that accounts for 10-20% of all soft-tissue sarcomas in young adults (1). It is invariably driven by the SS18-SSX oncoprotein, where the BAF subunit SS18 is fused to the repressive domain of SSX1, SSX2 or, rarely, SSX4. The BAF complex, the mammalian ortholog of SWI/SNF, is a major chromatin regulator involved in gene activation, whereas the SSX genes represent a family of highly immunogenic cancer-testis antigens involved in transcriptional repression. SS18-SSX promotes gene activation by changing the BAF complex configuration and chromatin targeting, while it also mediates gene silencing by forming a complex with ATF2 and TLE1.

Despite the relatively low number of secondary mutations, SyS tumors display different degrees of cellular differentiation and plasticity, and are classified accordingly as monophasic (mesenchymal cells), biphasic (mesenchymal and epithelial cells), or poorly differentiated (undifferentiated cells). The co-existence of distinct cellular phenotypes and morphologies in a single SyS tumor provides a unique opportunity to explore intratumor heterogeneity and cell state transitions. However, since human SyS has been studied primarily in established cellular models and through bulk profiling of tumor tissues, the molecular features of the different SyS subpopulations have so far remained elusive. In particular, because it remains unclear how this malignant cellular diversity comes about, which malignant cell states drive tumor progression, and how to selectively target aggressive synovial sarcoma cells to blunt tumor growth and dissemination, identification of cellular states, genetic drivers and bases for therapeutic strategies for this aggressive malignancy are needed.

Citation or identification of any document in this application is not an admission that such document is available as prior art to the present invention.

### SUMMARY

In certain example embodiments, methods of detecting an expression signature in synovial sarcoma (Sys) tumor are provided, comprising detecting in tumor cells obtained from a subject the expression or activity of a malignant cell gene signature comprising one or more genes or polypeptides selected from Table 6. In embodiments, the one or more genes or polypeptides are selected from the epithelial malignant signature of Table 1E, the mesenchymal malignant cell signature of Table 1D, the core oncogenic expression signature of Table 1A.1, and/or the cell cycle malignant signature of Table 1C. In certain example embodiments the core oncogenic signature may comprise the core oncogenic upregulated signature of Table 1A.2 or the core oncogenic downregulated signature of Table 1A.3.

In some embodiments, the methods comprise detecting a cell cycle malignant signature, which is indicative of increased risk of metastatic disease, an increased number of cycling cells and/or the presence of an increase of poorly differentiated cells.

In some embodiments, the methods comprise detecting core oncogenic upregulated malignant signatures, core oncogenic downregulated signature, or a combination thereof are detected, wherein detecting is indicative of increased metastatic Sys disease.

In certain embodiments, the method comprises detecting the epithelial malignant signature, the mesenchymal malignant signature or a combination thereof. In embodiments, the absence of the mesenchymal or epithelial malignant signature is indicative of higher progression free survival.

Methods for diagnosing a subject with Sys are also provided, and comprise detecting one or more signatures from Tables 1A-E. Methods of diagnosing a subject with increased risk of metastatic disease are also provided and can comprise detecting one or more signatures of Table 1A-1E.

In certain embodiments, methods of treating SyS in a subject in need thereof are provided, comprising administering an inhibitor of HDAC, CDK4/6, or a combination thereof to selectively target synovial sarcoma cells. In some embodiments, methods of treating may further comprise administering immune checkpoint inhibitors.

In embodiments, methods of distinguishing Sys from other cancer types and sarcomas are provided and comprise detecting a signature comprising a fusion program signature comprising one or more genes or polypeptides of Table 8.

In embodiments, methods of detecting a subject at high risk for metastatic disease comprising detecting core oncogenic program gene signatures. Methods of monitoring therapy are also provided and can comprise detecting the expression or activity of one or more gene signatures of Tables 1A-1E in tumor samples obtained from the subject for at least two time points. In embodiments, at least one sample is obtained before treatment, on some embodiments, at least one sample is obtained after treatment.

Methods of treatment can comprise in some embodiments targeting one or more genes or polypeptides of one or more signatures of Tables 1A-1E. Methods of treatment can also comprise treating a subject with SyS comprising administration of an isolated or engineered CD8+ T cell characterized by expression of an expansion program as defined in Table 1F, or a CD8+ T cell characterized by increased expression of IFN gamma or macrophage with increased expression of TNF alpha. Isolated or engineered CD8+ T cells characterized by increased expression of IFN gamma and/or macrophages with increased expression of TNF alpha are also provided. Methods of treatment for Synovial Sarcoma can comprise treatment with TNF and IFN-gamma, in some embodiments, the treatment providing a synergistic effect. Methods of treatment comprising administration of a modulator of one or more genes of cell cycle signature as defined in Table 1C, a SS18-SSX signature as defined in Table 8, or a combination thereof are also provided. In embodiments, administration of both modulators provides a synergistic effect.

In certain embodiments, the one or more agents comprise an antibody, small molecule, small molecule degrader, genetic modifying agent, antibody-like protein scaffold, aptamer, protein, or any combination thereof. In certain embodiments, the genetic modifying agent comprises a CRISPR system, RNAi system, a zinc finger nuclease system, a TALE, or a meganuclease. In certain embodiments, the CRISPR system comprises Cas9, Cas12, or Cas14. In certain embodiments, the CRISPR system comprises a dCas fused or otherwise linked to a nucleotide deaminase. In certain embodiments, the nucleotide deaminase is a cytidine deaminase or an adenosine deaminase. In certain embodiments, the dCas is a dCas9, dCas12, dCas13, or dCas14.

Methods of treating Synovial Sarcoma (Sys) in a subject are provided comprising: i) detecting the expression or activity of a malignant cell gene signature is a sample from a subject, the signature comprising one or more biomarkers selected from the group consisting of: a) epithelial malignant signature as defined in Table 1E; b) mesenchymal malignant cell signature as defined in Table 1D; c) cell cycle signature as defined in Table 1C; d) core oncogenic signature as defined in Table 1A.1; e) a fusion signature as defined in Table 8; or f) a combination thereof and ii) administering an effective amount of a modulating agent of the signature. In an aspect, the modulating agent is inhibitor of HDAC, CDK4/6, or a combination thereof, to selectively target synovial sarcoma cells.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1C—Mapping the cellular ecosystem of SyS tumors with single-cell transcriptomics. (1A) Study workflow. (1B) Converging assignments of cell identity. t-SNE plots of single cells (dots), shaded according to (1) tumor sample, (2) inferred cell type, (3) SS18-SSX1/2 fusion detection, (4) CNV detection, and (5) differential similarity to SyS compared to other sarcomas (see Methods). (1C) Inferred large-scale CNVs distinguish malignant (top) from non-malignant (bottom) cells, and are concordant with WES data. The inferred CNVs (amplifications in gray, and deletions in black) are shown along the chromosomes (x axis) for each cell (y axis).

FIG. 2A-2D—Consistent classification of cells based on transcriptomic and genetic features. (2A) Converging assignments of cell identity. tSNE plots of single cells (dots), colored according to (1) tumor sample, (2) inferred cell type, (3) SS18-SSX1/2 and MEOX2-AGMO fusion detection, (4) SSX1/2 gene detection (mRNA level >0), (5) MEOX2 and AGMO gene detection (mRNA level >0), (6-12) overall expression of well-established cell type markers (provided in Table 4). (2B) tSNE plots of single cells (dots), sequenced with a droplet-based approach (Zheng et al. Nat. Commun. 8, 14049 (2017)), colored according to (1) tumor sample, (2) inferred cell type, (3) SSX1/2 gene detection (mRNA level >0). (2C) tSNE plots of malignant cells (dots), sequenced with a droplet-based approach (Zheng et al. Nat. Commun. 8, 14049 (2017)), shaded according to the different malignant programs. (2D) Differential similarity to SyS compared to other sarcomas (Methods) is distinguishing malignant from non-malignant cells.

FIG. 3A-3C—Identifying the unique characteristics of SyS cells. (3A) The SyS program includes genes which are overexpressed by malignant cells compared to all types of non-malignant cells in the cohort; the expression of this program distinguishes between SyS and non-SyS cancer types, including those with hallmark BAF complex genomic aberrations: malignant rhabdoid tumor (MRT), epitheloid sarcoma (EpS), renal medullary carcinoma (RMC), small-cell carcinoma of the ovary, hypercalcemic type (SCCOHT), and SMARCA4-deficient thoracic sarcomas (SA4DTS). (3B) MEOX2 expression is highest in SyS tumors compared to other cancer types. (3C) MEOX2, and the cancer testis antigens CTAG1A, CTAG1B (encoding for NY-ESO-1), and PRAME are included in the SyS program; the expression of these genes across the malignant and non-malignant cells is shown.

FIG. 4A-4F—Intratumor heterogeneity couples between de-differentiation, cell cycle, and the core oncogenic program. (4A) t-SNE plots of malignant cells (dots), shaded by: (1) sample, (2) the epithelial vs. mesenchymal differentiation scores, (3) cycling status, and (4) the expression of the core oncogenic state. In (1), the mesenchymal and epithelial subpopulations of the biphasic tumors (BP), and the poorly differentiated (PD) tumor are marked with dashed circles. The other tumors are monophasic. (4B) Top core oncogenic genes (rows) across the malignant cells (columns), sorted according to the overall expression of the core oncogenic program (bottom bar). Top bar: biphasic tumor and sample. (4C) Left: Differentiation trajectories. A spectrum of malignant cell states along the mesenchymal to epithelial x axis and the stem-like to differentiated y axis; right: The expression of a G2/M phase signature (y axis) vs. the expression of a G1/S phase signature (x axis) across the malignant cells; in both plots the cells are shaded according to the expression of the cell cycle program, uncovering a strong association between cell cycle and poor differentiation (see also FIGS. 12B-12F). (4D) The percentage of cycling and poorly differentiated cells, among malignant cells with a high (above median) and low (below median) overall expression of the core oncogenic program. (4E-4F) In situ detection of core oncogenic, epithelial and mesenchymal programs. (4E) Immunofluorescence (t-CyCIF) and (4F) immunohistochemical stains of differentiation and core oncogenic markers.

FIG. 5A, 5B—The core oncogenic program and de-differentiation are associated with aggressive and metastatic disease. (5A) The expression of the different malignant programs across 34 SyS tumors (McBride et al. Cancer Cell (2018) doi:10.1016/j.ccell.2018.05.002), stratified according to tumor type: biphasic (BP), monophasic (MP), and poorly differentiated (PD). (5B) The programs are predictive of metastatic disease in an independent cohort obtained from 58 SyS patients (Banito et al. Cancer Cell 33:527-541.e8 (2018)). Kaplan-Meier (KM) curves of metastasis free survival, when stratifying the patients by high (top 25%), low (bottom 25%), or intermediate (remainder) expression of the respective program. Number of subjects at risk indicated at the bottom. P: COX regression p-value; Pc: COX regression p-value when controlling for fusion type and patient age group.

FIG. 6A, 6B—The core oncogenic program captures inter-patient variation. The inter-patient variation of the program was evaluated based on an independent RNA-Seq cohort from 64 SyS tumors (McBride et al. Cancer Cell (2018), doi:10.1016/j.ccell.2018.05.002), which were previously classified into two transcriptionally distinct clusters (McBride et al. Cancer Cell (2018), doi:10.1016/j.ccell.2018.05.002), denoted here as MYC-high and MYC-low. (6A) The overall expression of the program is correlated with the second Principle Component (PC2) of the data, and is significantly higher in the MYC-high cluster (P=1.66*10−7, t-test). (6B) The core oncogenic genes (columns) mostly correlated with PC2 are shown across the tumors (columns). Tumors are sorted according to PC2 (bottom bar).

FIG. 7A-7F—The SS18-SSX oncoprotein sustains the core oncogenic program, cell cycle, and dedifferentiation. (7A) Co-embedding (using PCA and canonical correlation analyses (Butler et al. Nat Biotechnol 36:411 (2018)) of ASKA and SYO1 cells (dots), shaded by: (1) condition, the overall expression of the (2) the cell cycle, (3) core oncogenic, and (4) mesenchymal differentiation (Taube et al. PNAS 107:15449-15454 (2010); Gröger et al. PLOS ONE 7:e51136 (2012)) programs. (7B) The overall expression of the cell cycle and core oncogenic programs is repressed in cells with the SSX shRNA (shSSX), while mesenchymal differentiation (Taube et al. PNAS 107:15449-15454 (2010); Gröger et al. PLOS ONE 7:e51136 (2012)) is induced; the shSSX impact on the core oncogenic and mesenchymal programs are observed both in the cycling and non-cycling cells. (7C) The expression of the overlapping fusion and core oncogenic program genes (columns) across the ASKA and SYO1 cells (rows), with a control (shCt) or SSX (shSSX) shRNA. The cells are sorted according to the overall expression of the fusion program (rightmost bar). (7D-7E) The fusion program distinguishes SyS from (7D) other cancer types and (7E) other sarcomas. (7F) The most overrepresented gene sets in the fusion program, when considering the induced (left) and repressed (right) genes, stratified to direct (black) and indirect (grey) target genes.

FIGS. 8A-8C—Cancer-immune interactions. (8A) The fusion KD is inducing multiple immune responses. The topmost differentially expressed pathways in SyS cells with SS18-SSX (shSSX) vs. control (shCt) shRNA. The overall expression of each pathway is shown, when stratifying the cells according to their cycling status. (8B) Inferred level of various immune cell types is associated with the malignant programs in bulk SyS tumors, when controlling for tumor purity. (8C) Short-term (4-6 hours) TNF treatment repressed the core oncogenic and fusion programs, but the effect was not observed after 24 h.

FIGS. 9A-9F—Immune cells and their association with malignant cell states. (9A) TNF and IFNγ are detected primarily in macrophages and T cells, respectively. (9B) TNF and IFNγ synergistically repress the core oncogenic and fusion programs (see also FIG. 8C). (9C) t-SNE plots of immune and stroma cells (dots), colored according to inferred cell type (left) and sample (right). (9D) T cell exhaustion is correlated with T cell cytotoxicity. The cytotoxicity (x axis) and exhaustion (y axis) scores of CD8 T cells, colored according to the T cell expansion program (see Methods). (9E) The effector vs. exhaustion scores of CD8 T cells in SyS and melanoma (top; Methods), and their predicted responsiveness to immune checkpoint blockade (Sade-Feldman et al. Cell 175:998-1013.e20 (2018)) (bottom; Methods). (9F) SyS tumors manifest a cold phenotype. The inferred level of intratumoral immune cells is exceptionally low in SyS tumors compared to (left) other cancer types and (right) other sarcomas.

FIGS. 10A-10D—Exploring the cancer-immune interplay in SyS. (10A) tSNE plots of macrophages, shaded according to inferred cell subtype, and the M1/M2 polarization scores (expression of the M1 minus M2 program), according to previously defined gene signatures (Janky et al. PLOS Comput. Biol. 10, e1003731 (2014)), and new signatures defined here by comparing between the two macrophage clusters (Table 12). (10B) The M1/M2 polarization scores of the M1-like and M2-like macrophages, according to previously defined gene signatures (Janky et al. PLOS Comput. Biol. 10, e1003731 (2014)). (10C) Gene-gene correlations across macrophages in SyS (top) and melanoma (Jerby-Arnon et al. Cell. 175, 984-997.e24 (2018)), when considering genes from M1 and M2 signatures (10C) as previously defined (Martinez et al. J. Immunol. Baltim. Md. 1950. 177, 7303-7311 (2006)), and as defined here (Table 12). (10D) The prognostic value of T cell infiltration levels (Methods) in (left) melanoma, (middle) sarcoma and (right) SyS (Li et al. BMC Bioinformatics. 12, 323 (2011)). Kaplan-Meier (KM) curves stratified by high (top 25%), low (bottom 25%), or intermediate (remainder) T cell infiltration levels. Number of subjects at risk indicated at the bottom. P: COX regression p-value.

FIG. 11—Blocking the core oncogenic program as a therapeutic strategy. Here Applicants show the results of the pharmacological single/combinatorial interventions of cell viability and single-cell transcriptome (in two synovial sarcoma cell lines and mesenchymal stems cells). Applicants' findings demonstrate that the SS18-SSX oncoprotein sustains de-differentiation, proliferation and the core oncogenic program, while immune cells in the tumor microenvironment can repress the core oncogenic and fusion programs through TNF and IFNγ secretion; inhibition of HDAC and CDK4/6 inhibitors mimic these effects.

FIGS. 12A-12F—Associations between poor differentiation, cell cycle and the core oncogenic program. (12A) The expression of the top epithelial and mesenchymal program genes (rows) across the malignant cells (columns), sorted according to their epithelial vs. mesenchymal differentiation scores (topmost bar). Top bar: biphasic tumor, cell cycling status, epithelial vs. non-epithelial cell status, and tumor. (12B) The expression of the G2/M phase signatures (y axis) vs. the expression of the G1/S phase signature (x axis) across the malignant cells, shaded according to their cycling states. (12C) The differentiation scores of cycling and non-cycling malignant cells, shown across all tumors together and when stratifying the cells according to their tumor sample (only tumors with at least 10 cycling cells are shown). (12D-12F) Left: A spectrum of malignant cell states along the mesenchymal to epithelial x axis and the stem-like to differentiated y axis; middle: The expression of a G2/M phase signatures (y axis) vs. the expression of a G1/S phase signature (x axis) across the malignant cells; right: The percentage of cycling and poorly differentiated cells, among malignant cells with a high (above median) and low (below median) overall expression of the core oncogenic program. In (12D) only the malignant cells which were sequenced with a droplet-based approach are shown, in (12E) only malignant cells from treatment naïve tumors and (12F) post-treatment tumors are shown.

FIG. 13A-13F A single-cell map of the cellular ecosystem of synovial sarcoma tumors (13A-D) Consistent assignment of cell identity. t-SNE plots of scRNA-Seq profiles (dots), shaded by either (13A) tumor sample, (13B) inferred cell type, (13C) SS18-SSX1/2 fusion detection, (13D) CNA detection, and (13E) differential similarity to SyS compared to other sarcomas (Methods). Dashed ovals (13A): mesenchymal and epithelial malignant subpopulations of biphasic (BP) tumors or poorly differentiated (PD) tumor. (13F) Inferred large-scale CNAs distinguish malignant (top) from non-malignant (bottom) cells, and are concordant with WES data (bold). The CNAs (gray: amplifications, black: deletions) are shown along the chromosomes (x axis) for each cell (y axis).

FIG. 14A-14D SyS tumors manifest antitumor immunity with limited immune infiltration. FIG. 14A Immune and stroma cells in SyS tumors. t-SNE of immune and stroma cell profiles (dots), shaded by inferred cell type (left) or sample (right). (14B) The CD8 T cell expansion program is associated with particularly high cytotoxicity and lower than expected exhaustion. The cytotoxicity (x axis) and exhaustion (y axis) scores of SyS CD8 T cells, colored by the score of the T cell expansion program (METHODS). (14C) CD8 T cells in SyS (light gray) have higher effector programs than in melanoma (dark gray). Distribution of effector vs. exhaustion scores (x axis, top, METHODS) or an immune checkpoint blockade responsiveness program (x axis, bottom, METHODS) in CD8 T cells from each cancer type. (14D) SyS tumors manifest a particularly cold phenotype. Overall Expression of the immune cell signatures (y axis, METHODS) in SyS tumors (dark gray) and other cancer types (left panel) or other sarcomas (right panel).

FIG. 15A-15C—(15A) Distinct differentiation pattern in biphasic tumors. Single cell profiles dots arranged by the first two diffusion-map components (DCs) for representative examples of a biphasic (SyS12, left) and monophasic (SyS11, right) tumors, and shadred by the Overall Expression of the epithelial vs. mesenchymal programs (bar). (15B) Core oncogenic program genes. Normalized expression (centered TPM values, bar) of the top 100 genes in the core oncogenic program (columns) across the malignant cells (rows), sorted according to the Overall Expression of the program (bar plot, right). Leftmost bars: biphasic tumor and sample ID. (15C) The program is expressed in a higher proportion of cycling and poorly differentiated cells. Fraction of malignant cells (y axis) with a high (above median, black) and low (below median, gray) Overall Expression of the core oncogenic program, in cells stratified by cycling and differentiation status (x axis).

FIG. 16 The core oncogenic program and de-differentiation co-vary within and across tumors and are associated with aggressive and cold tumors. Inferred level of immune cell types is associated with the malignant programs in bulk SyS tumors, when controlling for tumor purity. Partial correlation (bar) between the inferred level of each immune subset (rows) and the core oncogenic and differentiation levels (columns).

FIG. 17 The genetic driver and immune cells form two opposing forces in shaping SyS malignant cell states. Overlap of SS18-SSX and core oncogenic programs. Expression (centered TPM) of genes (rows) shared between the fusion and core oncogenic programs across the Aska and SYO1 cells (columns), with a control (shCt) or SSX (shSSX) shRNA. Cells are ordered by the Overall Expression of the SS18-SSX program (bottom plot) and labeled by type and condition (bar, top).

FIG. 18A-18I The core oncogenic program can be selectively blocked in SyS cells by combined HDAC and CDK4/6 inhibitors. (18A) Gene regulatory model of control of the core oncogenic program by SS18-SSX. Light gray/gray: genes that are induced/repressed in the core oncogenic program. Banded light Gray: genes that are repressed in the core oncogenic program and directly repressed by HDAC1-SS18-SSX. Blunt arrows: repression; pointy arrows: activation. Thick edges represent paths from SS18-SSX to p21. (18B) Model of regulation and intervention in the core oncogenic program. SS18-SSX activates the core oncogenic program in an HDAC-dependent manner and promotes cell cycle through direct activation of CDK6 and CCND2 (CycD) transcription. The core program suppresses p21 and inhibits immunogenic features. HDAC and CDK6 inhibitors target SyS dependencies. (18C-18F) TNF, HDAC and CDK6 inhibitors suppress the core oncogenic program. Overall Expression of the core oncogenic program (18B), SS18-SSX program (18C), an immune resistance program identified in melanoma (18D), and MHC-1 genes (18E) in SyS cells and MSCs (x axis). (18C-18F) *P<0.1, **P<0.01, ***P<1*10⁻³, ****P<1*10⁻⁴, t-test. (18F,18G) Selective toxicity for SyS cell lines. (18G) Viability (y axis) of SyS cell lines and MSCs (x axis) under different drugs (x axis, *P<5*10⁻², **P<5*10⁻³, ***P<5*10⁻⁴, ANOVA test). (18H) Selective toxicity to SyS lines vs. MSC (y axis, −log₁₀(P-value), ANOVA) in each treatment (x axis). In (18C-18G) middle line: median; box edges: 25^{th} and 75^{th} percentiles, whiskers: most extreme points that do not exceed IQR*1.5; further outliers are marked individually. (18I) Model of intrinsic and microenvironment determinants of SyS cell states. Left: The SS18-SSX oncoprotein sustains de-differentiation, proliferation and the core oncogenic program. Right: immune cells in the tumor microenvironment can repress the core oncogenic and SS18-SSX programs through TNF and IFNγ secretion. Combined inhibition of HDAC and CDK4/6 mimics these effects selectively in SyS cells.

FIG. 19—The SyS program distinguishes between SyS and non-SyS cancer types. Distribution of the SyS program Overall Expression (y axis) across BAF driven tumors (left, x axis) and in TCGA (right, x axis). Middle line: median; box edges: 25th and 75th percentiles, whiskers: most extreme points that do not exceed ±IQR*1.5; further outliers are marked individually; P-value: Wilcoxon-rank sum test; AUC: Area Under the receiver operating characteristic Curve.

FIG. 20A-20C Characterizing mesenchymal, epithelial and poorly differentiated malignant cells. FIG. 20A Epithelial and mesenchymal program genes. The expression of the top epithelial and mesenchymal program genes (rows) across the malignant cells (columns), with cells sorted according to the difference in epithelial vs. mesenchymal OE scores (bottom plot). Topmost bar: epithelial vs. non-epithelial cell status, and sample. Canonical markers include HLA-B, HLA-C, IFITM2, IRF7, XAF1, and immune-related genes are CDH1, EPCAM, MUC1, SNAI2, TCF4, ZEB1 and ZEB 2). FIG. 20B RNA velocities are visualized on top of the two first principle components (PCs), showing the state and velocity of the malignant cells obtained from patient SyS12 using the droplet-based approach. FIG. 20C t-SNE plots of malignant cells obtained from patient SyS12 before and after treatment, revealing a subpopulation of mesenchymal cells without copy number amplifications in chromosomes 15, 18 and 19 (FIG. 1G).

FIG. 21A-21C The core oncogenic program is detected using different approaches and datasets. FIG. 21A Agreement between the core oncogenic program detected by a PCA and an iNMF approach. Overall Expression (OE) of the core oncogenic program across malignant SyS cells, as identified in the PCA-based approach (x axis) and in the integrative-NMF approach (y axis) (METHODS). FIG. 21B-FIG. 21C Program Overall Expression captures inter-tumor variation and the MYC-high cluster in 64 SyS tumors from an independent RNA-Seq cohort. The tumors were previously classified into two transcriptionally distinct clusters, denoted here as MYC-high and MYC-low. FIG. 21B For each tumor (dots), shown is the Overall Expression (OE) of the core oncogenic program (y axis) vs. the projection on the second Principle Component (PC2) of the data. FIG. 21C Normalized expression (centered log-transformed RPKM) of the core oncogenic program genes (columns) most correlated with PC2 across the tumors (columns). Tumors are sorted by their PC2 projection (bottom bar).

FIG. 22A-22C Characterizing the transcriptional impact of SS18-SSX inhibition and tumor microenvironment cytokines on synovial sarcoma cells. FIG. 22A Biological processes regulated in the SS18-SSX program. Gene sets (rows) most enriched (−log₁₀(P-value), hypergeometric test, x axis) in induced (left) and repressed (right) SS18-SSX program genes, which are either direct (black bars) or indirect (grey bars) targets of SS18-SSX based on ChIP-Seq data (35, 36) and genetic perturbation. Vertical line denotes statistical significance following multiple hypotheses correction. FIG. 22B The SS18-SSX program distinguishes SyS from other cancer types and other sarcomas. Overall Expression of the SS18-SSX program (y axis) in either TCGA samples (n=9,391, top), stratified by cancer types (x axis), or in another independent cohort of sarcoma tumors (n=164, bottom) (48). Middle line: median; box edges: 25th and 75th percentiles, whiskers: most extreme points that do not exceed ±IQR*1.5; further outliers are marked individually. **P<0.01, ***P<1*10⁻³, ****P<1*10⁻⁴, t-test. FIG. 22C Repression of the core oncogenic and SS18-SSX programs by short term TNF treatment is not sustained long term. Distribution of Overall Expression scores (y axis) of the core oncogenic program and the direct and indirect SS18-SSX programs (x axis) in control cells (light gray) and cells treated with TNF for 4-6 hours (right) or more than 24 hours (left).

FIG. 23A-23C. HDAC and CDK4/6 inhibitors synergistically repress the core oncogenic program and induce cell autonomous immune responses. Distribution of the expression (y axis) of core oncogenic genes (FIG. 23A), as well as the Overall Expression of TNF (FIG. 23B) and IFN (FIG. 23C) signaling pathways in SyS cells and MSCs (x axis) under different treatments (legend). Middle line: median; box edges: 25th and 75th percentiles, whiskers: most extreme points that do not exceed ±IQR*1.5; further outliers are marked individually. **P<0.01, ***P<1*10⁻³, ****P<1*10⁻⁴, t-test.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to International Application No. PCT/US2018/024082, published as WO2018175924A1 on Sep. 27, 2018.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide methods and compositions for modulating an innate immune response, in particular an innate lymphoid cell class 2 innate immune response by modulating activity of SS18-SSX oncoprotein. Embodiments disclosed herein also provide for methods of monitoring an innate immune response in response to disease or treatment.

Oncogenic program comprises dedifferentiations, cell cycle and new cellular modality.

Differentiation trajectory includes mesenchymal and epithelial lineage programs, with mesenchymal program overlapping signatures of epithelial to mesenchymal transition (s1 and s4) and comprises markers of ZEB1, ZEB2, PDGFRA and SNAI2).

Applicants disclose herein methods and systems used to comprehensively map and interrogate cell states in Synovial Sarcoma (SyS), along with their regulatory circuits and clinical implications. Applicants demonstrate that the SS18-SSX oncoprotein and the tumor microenvironment coordinately shape cell states in SyS, with the present invention providing modulating, regulating and/or targeting of the programs to result in more effective treatment strategies. In particular, Applicants leverage scRNA-Seq data to map cell states in human SyS tumors to reveal the core oncogenic program associated with aggressive disease. Applicants further identified that TNF and IFNγ repress the program, and counteract the transcriptional alterations induced by the oncoprotein. Advantageously, Applicants discovered that targeting the program with HDAC and CDK4/6 inhibitors repressed the program and was detrimental to SyS cells, while sparing nonmalignant cells. Accordingly, the discovery provides a basis for the development of specific therapeutic strategies of Sys.

The discovery presented herein identifies programs tightly linked to clinical outcomes. The overall expression of the programs in bulk tumors can be used for synovial sarcoma patient stratification. The methods and compositions described herein may be used to shift the balance of cellular responses in Synovial Sarcoma patients in order to treat inflammatory allergic diseases and cancer.

### Expression Signatures

In certain example embodiments, the therapeutic, diagnostic, and screening methods disclosed herein target, detect, or otherwise make use of one or more biomarkers of an expression signature. As used herein, the term “biomarker” can refer to a gene, an mRNA, cDNA, an antisense transcript, a miRNA, a polypeptide, a protein, a protein fragment, or any other nucleic acid sequence or polypeptide sequence that indicates either gene expression levels or protein production levels. Accordingly, it should be understood that reference to a “signature” in the context of those embodiments may encompass any biomarker or biomarkers whose expression profile or whose occurrence is associated with a specific cell type, subtype, or cell state of a specific cell type or subtype within a population of cells (e.g., Synovial Sarcoma cells) or a specific biological program. As used herein the term “module” or “biological program” can be used interchangeably with “expression program” and refers to a set of biomarkers that share a role in a biological function (e.g., an activation program, cell differentiation program, proliferation program). Biological programs can include a pattern of biomarker expression that result in a corresponding physiological event or phenotypic trait. Biological programs can include up to several hundred biomarkers that are expressed in a spatially and temporally controlled fashion. Expression of individual biomarkers can be shared between biological programs. Expression of individual biomarkers can be shared among different single cell types; however, expression of a biological program may be cell type specific or temporally specific (e.g., the biological program is expressed in a cell type at a specific time). Expression of a biological program may be regulated by a master switch, such as a nuclear receptor or transcription factor. As used herein, the term “topic” refers to a biological program. Topics are described further herein. The biological program (topic) can be modeled as a distribution over expressed biomarkers.

In certain embodiments, the expression of the signatures disclosed herein (e.g., core oncogenic signature) is dependent on epigenetic modification of the biomarkers or regulatory elements associated with the signatures (e.g., chromatin modifications or chromatin accessibility). Thus, in certain embodiments, use of signature biomarkers includes epigenetic modifications of the biomarkers that may be detected or modulated. As used herein, the terms “signature”, “expression profile”, or “expression program” may be used interchangeably (e.g., expression of genes, expression of gene products or polypeptides). It is to be understood that also when referring to proteins (e.g. differentially expressed proteins), such may fall within the definition of “gene” signature. Levels of expression or activity may be compared between different cells in order to characterize or identify for instance signatures specific for cell (sub)populations. Increased or decreased expression or activity or prevalence of signature biomarkers may be compared between different cells in order to characterize or identify for instance specific cell (sub)populations. The detection of a signature in single cells may be used to identify and quantitate, for instance, specific cell (sub)populations. A signature may include a biomarker whose expression or occurrence is specific to a cell (sub)population, such that expression or occurrence is exclusive to the cell (sub)population. An expression signature as used herein, may thus refer to any set of up- and/or down-regulated biomarkers that are representative of a cell type or subtype. An expression signature as used herein, may also refer to any set of up- and/or down-regulated biomarkers between different cells or cell (sub)populations derived from a gene-expression profile. For example, an expression signature may comprise a list of biomarkers differentially expressed in a distinction of interest.

The signature according to certain embodiments of the present invention may comprise or consist of one or more biomarkers, such as for instance 1, 2, 3, 4, 5, 6, 7, 8, 9, 10 or more. In certain embodiments, the signature may comprise or consist of two or more biomarkers, such as for instance 2, 3, 4, 5, 6, 7, 8, 9, 10 or more. In certain embodiments, the signature may comprise or consist of three or more biomarkers, such as for instance 3, 4, 5, 6, 7, 8, 9, 10 or more. In certain embodiments, the signature may comprise or consist of four or more biomarkers, such as for instance 4, 5, 6, 7, 8, 9, 10 or more. In certain embodiments, the signature may comprise or consist of five or more biomarkers, such as for instance 5, 6, 7, 8, 9, 10 or more. In certain embodiments, the signature may comprise or consist of six or more biomarkers for instance 6, 7, 8, 9, 10 or more. In certain embodiments, the signature may comprise or consist of seven or more biomarkers, such as for instance 7, 8, 9, 10 or more. In certain embodiments, the signature may comprise or consist of eight or more biomarkers, such as for instance 8, 9, 10 or more. In certain embodiments, the signature may comprise or consist of nine or more biomarkers, such as for instance 9, 10 or more. In certain embodiments, the signature may comprise or consist of ten or more biomarkers, such as for instance 10, 11, 12, 13, 14, 15, or more. It is to be understood that a signature according to the invention may for instance also include different types of biomarkers combined (e.g. genes and proteins).

In certain embodiments, a signature is characterized as being specific for a particular cell or cell (sub)population if it is upregulated or only present, detected or detectable in that particular cell or cell (sub)population, or alternatively is downregulated or only absent, or undetectable in that particular cell or cell (sub)population. In this context, a signature consists of one or more differentially expressed genes/proteins or differential epigenetic elements when comparing different cells or cell (sub)populations, including comparing different cells or cell (sub)populations (e.g., synovial sarcoma cells), as well as comparing malignant cells or malignant cell (sub)populations with other non-malignant cells or non-malignant cell (sub)populations. It is to be understood that “differentially expressed” biomarkers include biomarkers which are up- or down-regulated as well as biomarkers which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art. Differential expression of biomarkers may also be determined by comparing expression of biomarkers in a population of cells or in a single cell. In certain embodiments, expression of one or more biomarkers is mutually exclusive in cells having a different cell state or subtype (e.g., two genes are not expressed at the same time). In certain embodiments, a specific signature may have one or more biomarkers upregulated or downregulated as compared to other biomarkers in the signature within a single cell (see, e.g., Table 4). Thus a cell type or subtype can be determined by determining the pattern of expression in a single cell.

As discussed herein, differentially expressed biomarkers may be differentially expressed on a single cell level, or may be differentially expressed on a cell population level. Preferably, the differentially expressed biomarkers as discussed herein, such as constituting the expression signatures as discussed herein, when as to the cell population level, refer to biomarkers that are differentially expressed in all or substantially all cells of the population (such as at least 80%, preferably at least 90%, such as at least 95% of the individual cells). This allows one to define a particular subpopulation of cells. As referred to herein, a “subpopulation” of cells preferably refers to a particular subset of cells of a particular cell type (e.g., Synovial Sarcoma) which can be distinguished or are uniquely identifiable and set apart from other cells of this cell type. The cell subpopulation may be phenotypically characterized, and is preferably characterized by the signature as discussed herein. A cell (sub)population as referred to herein may constitute of a (sub)population of cells of a particular cell type characterized by a specific cell state.

When referring to induction, or alternatively suppression of a particular signature, preferable is meant induction or alternatively suppression (or upregulation or downregulation) of at least one biomarker of the signature, such as for instance at least two, at least three, at least four, at least five, at least six, or all biomarkers of the signature.

Example gene signatures and topics are further described below.

### Malignant Programs

In certain embodiments, a malignant signature (e.g., signature of differentially expressed genes between malignant cells and non-malignant cells, e.g. epithelial cells, CAFs, CD8 and CD4 T cells, B cells, NK cells, macrophages, or mastocytes; or genes that can be modulated by HDAC and CDK4/6 inhibitors) comprises one or more biomarkers selected from one of Tables 1A-1E. In particular embodiments when core oncogenic program gene signatures of Table 1A is upregulated, or the core oncogenic gene signatures of Table 1B is downregulated, or a combination thereof are detected, the detected signature is indicative of increased metastatic disease.

TABLE 1A.1
Core Oncogenic Program

| | | | | | | |
|---|---|---|---|---|---|---|
| AFG3L1P | CD63 | EIF4EBP1 | LARP1 | NDUFA4 | PRKDC | SULT1A1 |
| AGPAT2 | CD7 | ELAC2 | LDHB | NDUFA7 | PSMA5 | SUMF2 |
| AGPAT5 | CDK2AP1 | ELOVL1 | LECT1 | NDUFA8 | PSMA7 | SYNPR |
| AHCY | CECR5 | EML3 | LGALS1 | NDUFAB1 | PSMB7 | TBCD |
| AKR1B1 | CHCHD1 | ENO1 | LINC00115 | NDUFB10 | PSMD4 | TCEB2 |
| AKR1C3 | CHCHD2 | EPRS | LINC00116 | NDUFB11 | PSMG3 | TELO2 |
| AKT1 | CIAPIN1 | ERGIC3 | LINC00516 | NDUFB2 | PTPRF | TFAP2A |
| ALDH1A1 | CKAP5 | ETAA1 | LINC00665 | NDUFB3 | PTPRS | THY1 |
| ALG3 | CLDN4 | EXOSC4 | LOC100131234 | NDUFB4 | PUS7 | TIGD1 |
| ALX4 | CLNS1A | EXOSC7 | LOC100272216 | NDUFB7 | PXDN | TIMM13 |
| ANAPC7 | CNPY2 | FADD | LOC101101776 | NDUFB9 | PYCR1 | TIMM8B |
| ANKRD26P1 | COA5 | FADS2 | LOC202781 | NDUFS6 | RABAC1 | TKT |
| APEH | COL18A1 | FAM178A | LOC375295 | NDUFS8 | RABL6 | TMA7 |
| APEX1 | COL5A1 | FAM19A5 | LOC441081 | NEDD8 | RANBP1 | TMC6 |
| APP | COL6A2 | FAM213B | LOC654433 | NEFL | RBM26 | TMEM101 |
| APRT | COL9A3 | FAM50B | LOXL1 | NHP2 | RBM6 | TMEM147 |
| ARF5 | COX4I1 | FARSA | LSM4 | NIPSNAP3A | RBX1 | TMEM177 |
| ARL6IP4 | COX5A | FARSB | LSM7 | NKAIN4 | REST | TMSB10 |
| ARL6IP5 | COX5B | FBN3 | LUC7L3 | NME1 | RGMA | TMTC2 |
| ASB13 | COX6A1 | FGF19 | LY6E | NME2 | RGS10 | TOMM40 |
| ATF7IP | COX6B1 | FGF9 | MAB21L1 | NNT | RHOBTB3 | TOMM6 |
| ATIC | COX6C | FLAD1 | MAGEA4 | NOMO1 | RNASEK | TOMM7 |
| ATP5A1 | COX7C | FMO1 | MAGEA9 | NOMO2 | RNPC3 | TRAPPC1 |
| ATP5C1 | CRIP1 | FRG1B | MAGEC2 | NPEPL1 | RNPEP | TSPAN3 |
| ATP5E | CRLF1 | FSD1 | MAP1B | NRBP2 | ROMO1 | TSR3 |
| ATP5G2 | CRMP1 | G6PC3 | MATN3 | NREP | RUVBL1 | TSTA3 |
| ATP5I | CSAG3 | GABPB1-AS1 | MBD6 | NSMF | RUVBL2 | TTYH3 |
| ATP5J | CSE1L | GADD45GIP1 | MDH2 | NSUN5 | SARS2 | TUBG1 |
| ATP5J2 | CSRP2BP | GAPDH | MDK | NSUN5P1 | SELENBP1 | TUFM |
| ATP5O | CST3 | GCN1L1 | METTL3 | NSUN5P2 | SEMA3A | TUSC3 |
| ATR | CSTB | GDI2 | MFSD3 | NT5DC2 | SERF2 | TWIST2 |
| ATRAID | CSTF3 | GEMIN7 | MGC21881 | NUBP2 | SERTAD4 | TXN |
| AUP1 | CTAG1A | GGH | MGST1 | NUDT5 | SETD4 | TXNDC17 |
| AURKAIP1 | CTAG1B | GLB1L | MGST3 | NUTF2 | SFN | TXNDC5 |
| BCAP31 | CYC1 | GLB1L2 | MIF | OBSL1 | SGK196 | TXNDC9 |
| BCL7C | CYHR1 | GLI1 | MIS18A | OGG1 | SH2D4A | UBA52 |
| BMP1 | DAD1 | GNAS | MKKS | OST4 | SH3PXD2B | UBE2T |
| BOP1 | DANCR | GNB2L1 | MMP14 | OXLD1 | SHMT2 | UBE3B |
| BRK1 | DBNDD1 | GNPTAB | MRPL12 | PAFAH1B3 | SIGIRR | UCK2 |
| BSG | DCHS1 | GOLM1 | MRPL15 | PARK7 | SIM2 | UCP2 |
| BTF3 | DCP1B | GPR124 | MRPL17 | PATZ1 | SIX1 | UPK3B |
| C11orf48 | DCTPP1 | GPR126 | MRPL28 | PAX3 | SLC25A23 | UQCR10 |
| C14orf2 | DCXR | GPRC5B | MRPL35 | PAX9 | SLC25A6 | UQCR11 |
| C16orf88 | DGCR6L | GSTO2 | MRPL4 | PCDHA3 | SLC35B4 | UQCRB |
| C17orf76-AS1 | DHFR | GUSB | MRPL52 | PDCD11 | SLC6A15 | UQCRC1 |
| C1QBP | DNMT3A | H19 | MRPS17 | PDCD5 | SMARCA4 | UQCRQ |
| C2orf68 | DPEP3 | HERC2 | MRPS21 | PDIA4 | SMC2 | USMG5 |
| C4orf48 | DPYSL2 | HERC2P7 | MRPS26 | PEBP1 | SMC3 | USP5 |
| C7orf73 | DYNLRB1 | HIGD2A | MRPS34 | PET100 | SNHG6 | VARS |
| C9orf16 | DYNLT1 | HINT1 | MTG1 | PFKL | SNRPD2 | VCAN |
| CAD | EDF1 | HMG20B | MTRNR2L1 | PFKP | SNRPD3 | VKORC1 |
| CALML3 | EEF1B2 | HN1L | MTRNR2L10 | PFN1 | SNRPF | VPS28 |
| CAPNS1 | EEF1D | HNRNPD | MTRNR2L2 | PFN1P2 | SOX11 | VPS72 |
| CBX6 | EEF1G | HOXD11 | MTRNR2L6 | PGD | SPCS1 | VSNL1 |
| CCDC137 | EIF2AK1 | HOXD9 | MTRNR2L8 | PGLS | SPDYE8P | WDR12 |
| CCDC140 | EIF3C | HSD17B10 | MYBBP1A | PHF14 | SRI | YWHAB |
| CCT3 | EIF3H | HYAL2 | MZT2B | PIGM | SRM | ZNF212 |
| CD320 | EIF3K | HYLS1 | NACA | PIGQ | SRSF9 | ZNF605 |
| IFT81 | IMP3 | ICT1 | NAT14 | PIGT | SSNA1 | ING4 |
| IRS4 | ITM2C | ITPA | NDUFA1 | PKD2 | SSR4 | JMJD8 |
| KDM1A | KIAA0020 | KIF1A | NDUFA11 | PLP2 | SSX2 | KRT14 |
| KRT15 | KRT8 | KRTCAP2 | NDUFA13 | PMS2P5 | SSX2B | LAMA2 |
| POLR1B | POLR2F | PPIA | NDUFA3 | POLD2 | STAG3L1 | PPIB |
| PPIP5K2 | PPP1R16A | PRDX2 | PRDX4 | PRELID1 | STAG3L2 | STAG3L3 |
| STAG3L4 | STARD4-AS1 | SULF2 | DDX3Y | IFRD1 | NFKBIZ | SRSF3 |
| AKIRIN1 | DDX5 | FOSL2 | IRF1 | NR4A1 | TNFAIP3 | CDKN1A |
| AMD1 | DLX2 | GADD45B | JUN | NR4A2 | TNFRSF12A | CKS2 |
| ARC | DNAJA1 | GEM | JUNB | NR4A3 | TOB1 | CLK1 |
| ATF3 | DNAJA4 | GTF2B | JUND | PAFAH1B2 | TRIB1 | COQ10B |
| ATF4 | DNAJB1 | H3F3B | KLF10 | PER1 | TSPYL1 | CSRNP1 |
| BHLHE40 | DNAJB9 | HBP1 | KLF4 | PER2 | TSPYL2 | CYCS |
| BRD2 | DUSP1 | HERPUD1 | KLF6 | PPP1R15A | TUBA1A | DDIT3 |
| BTG1 | DUSP2 | HES1 | KLHL15 | RGS16 | TUBA1B | DDX3X |
| BTG2 | EGR1 | HSP90AA1 | LMNA | RHOB | TUBB2A | EIF4A3 |
| C12orf44 | EGR2 | HSP90AB1 | LOC284454 | RIPK4 | TUBB4B | EIF5 |
| C6orf62 | EGR3 | HSPA1A | MAFF | RRP12 | UBB | ERF |
| CCNL1 | EIF1 | HSPA1B | MCL1 | SAT1 | UBC | ETF1 |
| FOSL1 | IER3 | HSPA8 | MIR22HG | SELK | XBP1 | NFATC1 |
| FAM53C | ID2 | HSPH1 | MLF1 | SERTAD1 | YWHAG | NFATC2 |
| FOS | ID3 | ICAM1 | MXD1 | SF1 | ZBTB21 | NFKBIA |
| FOSB | IER2 | ID1 | MYADM | SIK1 | ZFAND5 | SLC25A44 |
| SOCS3 | SLC25A25 | ZFP36 | | | | |

TABLE 1A.2
Core Oncogenic Program Upregulated

| | | | | | | |
|---|---|---|---|---|---|---|
| AFG3L1P | CD63 | EIF4EBP1 | LARP1 | NDUFA4 | PRKDC | SULT1A1 |
| AGPAT2 | CD7 | ELAC2 | LDHB | NDUFA7 | PSMA5 | SUMF2 |
| AGPAT5 | CDK2AP1 | ELOVL1 | LECT1 | NDUFA8 | PSMA7 | SYNPR |
| AHCY | CECR5 | EML3 | LGALS1 | NDUFAB1 | PSMB7 | TBCD |
| AKR1B1 | CHCHD1 | ENO1 | LINC00115 | NDUFB10 | PSMD4 | TCEB2 |
| AKR1C3 | CHCHD2 | EPRS | LINC00116 | NDUFB11 | PSMG3 | TELO2 |
| AKT1 | CIAPIN1 | ERGIC3 | LINC00516 | NDUFB2 | PTPRF | TFAP2A |
| ALDH1A1 | CKAP5 | ETAA1 | LINC00665 | NDUFB3 | PTPRS | THY1 |
| ALG3 | CLDN4 | EXOSC4 | LOC100131234 | NDUFB4 | PUS7 | TIGD1 |
| ALX4 | CLNS1A | EXOSC7 | LOC100272216 | NDUFB7 | PXDN | TIMM13 |
| ANAPC7 | CNPY2 | FADD | LOC101101776 | NDUFB9 | PYCR1 | TIMM8B |
| ANKRD26P1 | COA5 | FADS2 | LOC202781 | NDUFS6 | RABAC1 | TKT |
| APEH | COL18A1 | FAM178A | LOC375295 | NDUFS8 | RABL6 | TMA7 |
| APEX1 | COL5A1 | FAM19A5 | LOC441081 | NEDD8 | RANBP1 | TMC6 |
| APP | COL6A2 | FAM213B | LOC654433 | NEFL | RBM26 | TMEM101 |
| APRT | COL9A3 | FAM50B | LOXL1 | NHP2 | RBM6 | TMEM147 |
| ARF5 | COX4I1 | FARSA | LSM4 | NIPSNAP3A | RBX1 | TMEM177 |
| ARL6IP4 | COX5A | FARSB | LSM7 | NKAIN4 | REST | TMSB10 |
| ARL6IP5 | COX5B | FBN3 | LUC7L3 | NME1 | RGMA | TMTC2 |
| ASB13 | COX6A1 | FGF19 | LY6E | NME2 | RGS10 | TOMM40 |
| ATF7IP | COX6B1 | FGF9 | MAB21L1 | NNT | RHOBTB3 | TOMM6 |
| ATIC | COX6C | FLAD1 | MAGEA4 | NOMO1 | RNASEK | TOMM7 |
| ATP5A1 | COX7C | FMO1 | MAGEA9 | NOMO2 | RNPC3 | TRAPPC1 |
| ATP5C1 | CRIP1 | FRG1B | MAGEC2 | NPEPL1 | RNPEP | TSPAN3 |
| ATP5E | CRLF1 | FSD1 | MAP1B | NRBP2 | ROMO1 | TSR3 |
| ATP5G2 | CRMP1 | G6PC3 | MATN3 | NREP | RUVBL1 | TSTA3 |
| ATP5I | CSAG3 | GABPB1-AS1 | MBD6 | NSMF | RUVBL2 | TTYH3 |
| ATP5J | CSE1L | GADD45GIP1 | MDH2 | NSUN5 | SARS2 | TUBG1 |
| ATP5J2 | CSRP2BP | GAPDH | MDK | NSUN5P1 | SELENBP1 | TUFM |
| ATP5O | CST3 | GCN1L1 | METTL3 | NSUN5P2 | SEMA3A | TUSC3 |
| ATR | CSTB | GDI2 | MFSD3 | NT5DC2 | SERF2 | TWIST2 |
| ATRAID | CSTF3 | GEMIN7 | MGC21881 | NUBP2 | SERTAD4 | TXN |
| AUP1 | CTAG1A | GGH | MGST1 | NUDT5 | SETD4 | TXNDC17 |
| AURKAIP1 | CTAG1B | GLB1L | MGST3 | NUTF2 | SFN | TXNDC5 |
| BCAP31 | CYC1 | GLB1L2 | MIF | OBSL1 | SGK196 | TXNDC9 |
| BCL7C | CYHR1 | GLI1 | MIS18A | OGG1 | SH2D4A | UBA52 |
| BMP1 | DAD1 | GNAS | MKKS | OST4 | SH3PXD2B | UBE2T |
| BOP1 | DANCR | GNB2L1 | MMP14 | OXLD1 | SHMT2 | UBE3B |
| BRK1 | DBNDD1 | GNPTAB | MRPL12 | PAFAH1B3 | SIGIRR | UCK2 |
| BSG | DCHS1 | GOLM1 | MRPL15 | PARK7 | SIM2 | UCP2 |
| BTF3 | DCP1B | GPR124 | MRPL17 | PATZ1 | SIX1 | UPK3B |
| C11orf48 | DCTPP1 | GPR126 | MRPL28 | PAX3 | SLC25A23 | UQCR10 |
| C14orf2 | DCXR | GPRC5B | MRPL35 | PAX9 | SLC25A6 | UQCR11 |
| C16orf88 | DGCR6L | GSTO2 | MRPL4 | PCDHA3 | SLC35B4 | UQCRB |
| C17orf76-AS1 | DHFR | GUSB | MRPL52 | PDCD11 | SLC6A15 | UQCRC1 |
| C1QBP | DNMT3A | H19 | MRPS17 | PDCD5 | SMARCA4 | UQCRQ |
| C2orf68 | DPEP3 | HERC2 | MRPS21 | PDIA4 | SMC2 | USMG5 |
| C4orf48 | DPYSL2 | HERC2P7 | MRPS26 | PEBP1 | SMC3 | USP5 |
| C7orf73 | DYNLRB1 | HIGD2A | MRPS34 | PET100 | SNHG6 | VARS |
| C9orf16 | DYNLT1 | HINT1 | MTG1 | PFKL | SNRPD2 | VCAN |
| CAD | EDF1 | HMG20B | MTRNR2L1 | PFKP | SNRPD3 | VKORC1 |
| CALML3 | EEF1B2 | HN1L | MTRNR2L10 | PFN1 | SNRPF | VPS28 |
| CAPNS1 | EEF1D | HNRNPD | MTRNR2L2 | PFN1P2 | SOX11 | VPS72 |
| CBX6 | EEF1G | HOXD11 | MTRNR2L6 | PGD | SPCS1 | VSNL1 |
| CCDC137 | EIF2AK1 | HOXD9 | MTRNR2L8 | PGLS | SPDYE8P | WDR12 |
| CCDC140 | EIF3C | HSD17B10 | MYBBP1A | PHF14 | SRI | YWHAB |
| CCT3 | EIF3H | HYAL2 | MZT2B | PIGM | SRM | ZNF212 |
| CD320 | EIF3K | HYLS1 | NACA | PIGQ | SRSF9 | ZNF605 |
| | | ICT1 | NAT14 | PIGT | SSNA1 | |
| | | IFT81 | NDUFA1 | PKD2 | SSR4 | |
| | | IMP3 | NDUFA11 | PLP2 | SSX2 | |
| | | ING4 | NDUFA13 | PMS2P5 | SSX2B | |
| | | IRS4 | NDUFA3 | POLD2 | STAG3L1 | |
| | | ITM2C | | POLR1B | STAG3L2 | |
| | | ITPA | | POLR2F | STAG3L3 | |
| | | JMJD8 | | PPIA | STAG3L4 | |
| | | KDM1A | | PPIB | STARD4-AS1 | |
| | | KIAA0020 | | PPIP5K2 | SULF2 | |
| | | KIF1A | | PPP1R16A | | |
| | | KRT14 | | PRDX2 | | |
| | | KRT15 | | PRDX4 | | |
| | | KRT8 | | PRELID1 | | |
| | | KRTCAP2 | | | | |
| | | LAMA2 | | | | |

TABLE 1A.3
Core Oncogenic Program Downregulated

| | | | | | |
|---|---|---|---|---|---|
| AKIRIN1 | DDX5 | FOSL2 | IRF1 | NR4A1 | TNFAIP3 |
| AMD1 | DLX2 | GADD45B | JUN | NR4A2 | TNFRSF12A |
| ARC | DNAJA1 | GEM | JUNB | NR4A3 | TOB1 |
| ATF3 | DNAJA4 | GTF2B | JUND | PAFAH1B2 | TRIB1 |
| ATF4 | DNAJB1 | H3F3B | KLF10 | PER1 | TSPYL1 |
| BHLHE40 | DNAJB9 | HBP1 | KLF4 | PER2 | TSPYL2 |
| BRD2 | DUSP1 | HERPUD1 | KLF6 | PPP1R15A | TUBA1A |
| BTG1 | DUSP2 | HES1 | KLHL15 | RGS16 | TUBA1B |
| BTG2 | EGR1 | HSP90AA1 | LMNA | RHOB | TUBB2A |
| C12orf44 | EGR2 | HSP90AB1 | LOC284454 | RIPK4 | TUBB4B |
| C6orf62 | EGR3 | HSPA1A | MAFF | RRP12 | UBB |
| CCNL1 | EIF1 | HSPA1B | MCL1 | SAT1 | UBC |
| CDKN1A | EIF4A3 | HSPA8 | MIR22HG | SELK | XBP1 |
| CKS2 | EIF5 | HSPH1 | MLF1 | SERTAD1 | YWHAG |
| CLK1 | ERF | ICAM1 | MXD1 | SF1 | ZBTB21 |
| COQ10B | ETF1 | ID1 | MYADM | SIK1 | ZFAND5 |
| CSRNP1 | FAM53C | ID2 | NFATC1 | SLC25A25 | ZFP36 |
| CYCS | FOS | ID3 | NFATC2 | SLC25A44 | |
| DDIT3 | FOSB | IER2 | NFKBIA | SOCS3 | |
| DDX3X | FOSL1 | IER3 | NFKBIZ | SRSF3 | |
| DDX3Y | | IFRD1 | | | |

TABLE 1C
Malignant Cell Cycle Program

ANLN
ARHGAP11A
ATAD5
BIRC5
BRCA2
BUB1B
C21orf58
CASC5
CCNA2
CCNB2
CCNE2
CDC6
CDKN3
CENPE
CENPF
CENPH
CENPK
CENPW
CHAF1B
CLSPN
DHFR
DNA2
DTL
EZH2
FANCA
FANCD2
FANCI
FOXM1
GINS2
HELLS
KIAA0101
KIF11
KIF14
KIF18A
KIF20B
KIF2C
KNSTRN
KNTC1
MAD2L1
MCM2
MCM3
MCM4
MCM5
MKI67
MLF1IP
NCAPD2
NCAPG2
NUSAP1
OAS3
OIP5
ORC6
PRC1
PSMC3IP
PTTG1
RACGAP1
RFC4
RNASEH2A
RRM2
SGOL2
SMC4
SPAG5
SPDL1
STIL
TCF19
TIMELESS
TK1
TOP2A
TPX2
TYMS
UBE2C
UBE2T
UHRF1
WDHD1
ZWINT

In particular embodiments, cell cycle program genes are detected, in particular embodiments, detecting is indicative of increased risk of metastatic disease, with absence i.e. detection of high differentiations is prognostic of metastasis free survival.

TABLE 1D
Mesenchymal Cell Malignant Program

AASS
ADAM33
AKAP13
ANKRD44
ARMCX3
ATP1B2
BMP5
C14orf37
C14orf39
C16orf45
C1orf151-NBL1
CACNB2
CADM1
CALD1
CCBE1
CCDC88A
CD302
CLIP3
CNRIP1
CNTLN
COL1A2
COL21A1
COL4A1
COL4A2
COL5A1
COL5A2
COL6A3
COL8A1
CPXM1
CRTAP
CXCL12
CYGB
DAB2
DCN
DEGS1
DNAJA4
DNAJC12
DNM3OS
DZIP1
EDNRA
EGFR
EMP1
F2R
FBXO32
FERMT2
FGF10
FHL1
FKBP7
FLJ42709
FLNB
FN1
FOSL2
FRZB
FSTL1
GALNT18
GEM
GFPT2
GFRA1
GPM6B
GPX7
GSTA4
GSTM5
GYPC
HAAO
HCG11
HENMT1
HMGCLL1
HOXC10
HOXC9
HSD17B11
IFFO1
IL17RD
IL1R1
INHBA
INPP4B
ITPRIPL2
KIF26B
LAMA2
LAMB1
LEF1
LEPRE1
LOXL2
LRP1
LUM
MEF2A
MEOX2
MFAP4
MLF1
MMP2
MSN
MSRB3
MXRA5
MYL9
NCAM1
NDNF
NDOR1
NEDD4
NEFH
NID1
NID2
NR4A2
NUDT11
OXER1
PALLD
PDGFRA
PDIA5
PDLIM4
PDZRN3
PLIN2
PLK1S1
PLSCR4
PMP22
PPP1R15B
PROS1
QKI
QPRT
RAB31
RAI14
RASL11B
RBMS3
RCBTB2
RCN3
RGL1
RGS3
RHOJ
RUNX1T1
SEMA6A
SERTAD1
SESN1
SH3PXD2A
SIX1
SLC2A10
SNAI2
SPARC
ST3GAL3
STARD13
TCF12
TCF4
TGFB1I1
TMEM30B
TMEM45A
TNFRSF19
TSC22D3
UBE2E2
UBL3
UNC5B
WIF1
WNT16
ZEB1
ZEB2
ZFHX4
ZNF302

TABLE 1E
Epithelial Cell Malignant Program

ABCG1
ABHD11
ABRACL
ACOT7
ACP5
ADAMTSL2
AES
AGPAT2
AGRN
AGTRAP
AHNAK2
AIG1
AKR1C3
ALDH1A3
ALDH3A2
ALDH4A1
ALOX15
ANK3
ANO9
ANXA11
ANXA3
AP1M2
APOE
APP
ARHGAP8
ARID5A
ARRDC1
ASS1
ATHL1
ATP6V0E2
BAIAP2L1
BARX2
BCAM
BSCL2
C14orf1
C19orf21
C19orf33
C1GALT1C1
C1orf210
CAP2
CAPN6
CARD16
CARNS1
CBLC
CCDC153
CCDC24
CCND1
CD151
CD55
CD59
CD7
CD74
CD9
CDCP1
CDH1
CDH3
CDH4
CDK2AP2
CHST9
CKB
CLDN3
CLDN4
CLDN7
CLIC3
CLU
COL12A1
CRB3
CRIP1
CRIP2
CXADR
CXCL1
CYB561
CYBA
CYFIP2
CYHR1
CYP39A1
CYP4X1
CYSTM1
DBNDD2
DCXR
DDR1
DDX58
DHCR7
DMKN
DRD1
DSP
EFCAB4A
EFNA5
ELOVL1
ELOVL7
EMB
ENO2
ENPP5
ENTPD3
EPB41L5
EPCAM
EPHA2
EPS8L2
ERBB2
ERBB3
ESRP1
ESRP2
EZR
F11R
F2RL1
FAAH
FAAH2
FAM111A
FAM167A
FAM213A
FAM221A
FAM65C
FAM84B
FBXO2
FBXO44
FGF19
FGFRL1
FMO2
FXYD3
FXYD5
FZD6
GALNT3
GAS6
GCHFR
GPR56
GPRC5A
GPRC5C
GRB7
GSDMD
HERC6
HIGD2A
HLA-B
HMGA1
HOOK2
HPN
HSPB2
IFITM1
IFITM2
IFITM5
IGFBP6
IGSF9
INADL
INF2
IQGAP1
IRF6
IRF7
ISLR
ITGA3
ITGB4
ITGB8
ITPR2
ITPR3
JUP
KIAA1522
KIAA1598
KIF1A
KLF5
KLK1
KLK10
KLK11
KLK7
KLK8
KRT18
KRT19
KRT7
KRT8
KRTCAP3
LBH
LECT1
LGALS3BP
LIME1
LLGL2
LOC100505761
LOC541471
LOC646329
LPAR2
LPIN3
LRRC16A
LSR
LY6E
LYPD6B
MAGI1
MAL2
MAP7
MBOAT1
MCAM
MDK
MFSD3
MGAT4B
MIF4GD
MLXIPL
MPZL2
MSLN
MSMO1
MSX2
MUC1
MX1
MYH9
MYO6
NCOA7
NDUFA4L2
NDUFS8
NET1
NPNT
NSMF
NT5DC1
NT5E
NUDT14
OAS1
OCIAD2
OCLN
ORMDL2
P4HTM
PARD6B
PARP8
PARP9
PARVG
PCBD1
PDGFB
PDHX
PDLIM1
PDLIM2
PERP
PHYHD1
PIGV
PIM1
PKP3
PKP4
PLEKHB1
PLEKHG1
PLEKHN1
PLLP
PLXDC2
PLXNA2
PLXNB1
PNOC
PNP
PPL
PPP1CA
PPP1R16A
PPP1R1B
PPP1R9A
PRKCG
PRPH
PRR15
PRR15L
PRSS8
PSME1
PSME2
PTGER4
PTGES
PTN
PTPRF
PTRH1
RAB3IP
RALGPS1
RASSF7
RBM47
REC8
REEP2
RGL3
RHBDF2
RHBDL1
RIPK4
ROBO3
RTN3
S100A16
S100A4
S100A6
SAMD12
SCG5
SCNN1A
SCRN2
SEC11C
SECTM1
SELENBP1
SEMA3B
SGPL1
SH3YL1
SHANK2
SHANK2-AS3
SIM2
SLC11A2
SLC12A2
SLC16A5
SLC25A25
SLC25A29
SLC29A1
SLC35F2
SLC50A1
SLC6A9
SLC7A5
SLC7A8
SLFN5
SLPI
SMAD1
SMPDL3B
SORT1
SOX14
SPINT1
SPINT2
ST14
ST3GAL5
STAP2
STRA13
STRA6
STXBP2
SULF1
SULF2
SUMF1
SVIP
SYNGR2
SYTL1
TACSTD2
TAPBPL
TCF7L2
TENM1
TFAP2B
TFAP2C
TLE2
TLE6
TM4SF1
TM7SF2
TMC4
TMCC3
TMEM125
TMEM176B
TNFAIP2
TNFRSF12A
TNFRSF14
TNFRSF21
TNFSF13
TNKS1BP1
TNNI3
TNNT1
TOM1L1
TPD52
TSPO
TUBB2B
TUBB3
UCP2
VAMP8
WDR34
WDR54
WFDC2
XAF1
ZDHHC12
ZMAT1
ZNF165
ZNF423
ZNF664

TABLE 1F
Expansion Program
T cell expansion

| | |
|---|---|
| UP | DOWN |

| | | |
|---|---|---|
| ANO6 | ABHD3 | MAFF |
| B2M | ALDH6A1 | MAP4K4 |
| BCL10 | ALG13 | MASTL |
| BHLHE40 | AMICA1 | MFNG |
| C6orf25 | ARF5 | MVD |
| CADM1 | ARHGAP25 | NAGPA |
| CHSY1 | ARID3B | NBPF10 |
| CMAHP | ARL5B | NDUFA1 |
| CREB3 | ARSD | NDUFB9 |
| CRIP2 | ATP5O | NFATC1 |
| CX3CR1 | ATXN10 | NUDCD1 |
| DDX20 | ATXN7L1 | OAS2 |
| DHRS7 | BANF1 | OMA1 |
| DSCR3 | C14orf1 | ORAOV1 |
| EIF4A2 | C9orf72 | PECAM1 |
| FAM83D | CBLL1 | PECR |
| FCGR3A | CCR7 | PFN1 |
| FCRL6 | CD27 | PNPLA6 |
| FGFBP2 | CD28 | PSMD3 |
| GNLY | CD83 | PTPRB |
| GPR114 | CD84 | REST |
| GPR56 | CDK2 | RGPD6 |
| GRIPAP1 | CETN2 | SEC13 |
| GSR | CLDND1 | SLC25A32 |
| GZMB | CLIC5 | SNAP47 |
| GZMH | CMBL | SOCS3 |
| HOPX | CORO1A | TAGAP |
| HSDL1 | COTL1 | TBC1D7 |
| KAT2B | CRTAM | THAP5 |
| KIR2DS4 | DCTN6 | TIMMDC1 |
| KLRD1 | ECHDC1 | TMPPE |
| KRR1 | EDEM1 | TOMM22 |
| LTBP4 | EIF3G | TOX |
| LZTR1 | EXOSC2 | TRAPPC5 |
| MALAT1 | FAIM3 | TRIT1 |
| METTL13 | FCGRT | TRPT1 |
| MGAT4A | GALM | TSPAN3 |
| MMD | GGA2 | URGCP |
| MRPL33 | GPR183 | USP16 |
| N4BP2L2 | GSKIP | VPS33B |
| NKG7 | GUK1 | VPS52 |
| NSFP1 | GUSBP3 | YIPF5 |
| PDCD7 | GUSBP9 | |
| PFKM | GZMK | |
| PLEKHA5 | GZMM | |
| PLEKHG3 | HGS | |
| PRF1 | HIF1AN | |
| RAD52 | HIST1H1E | |
| RASSF1 | HNRPLL | |
| RPP38 | HPCAL1 | |
| RPS10 | ISG20L2 | |
| SEC23B | JUNB | |
| SERPINB9 | KIAA0226 | |
| TM4SF19 | KIAA1551 | |
| TSFM | KREMEN1 | |
| TTC21B | LDHB | |
| ZDHHC7 | LIMS1 | |
| ZNF41 | LYST | |

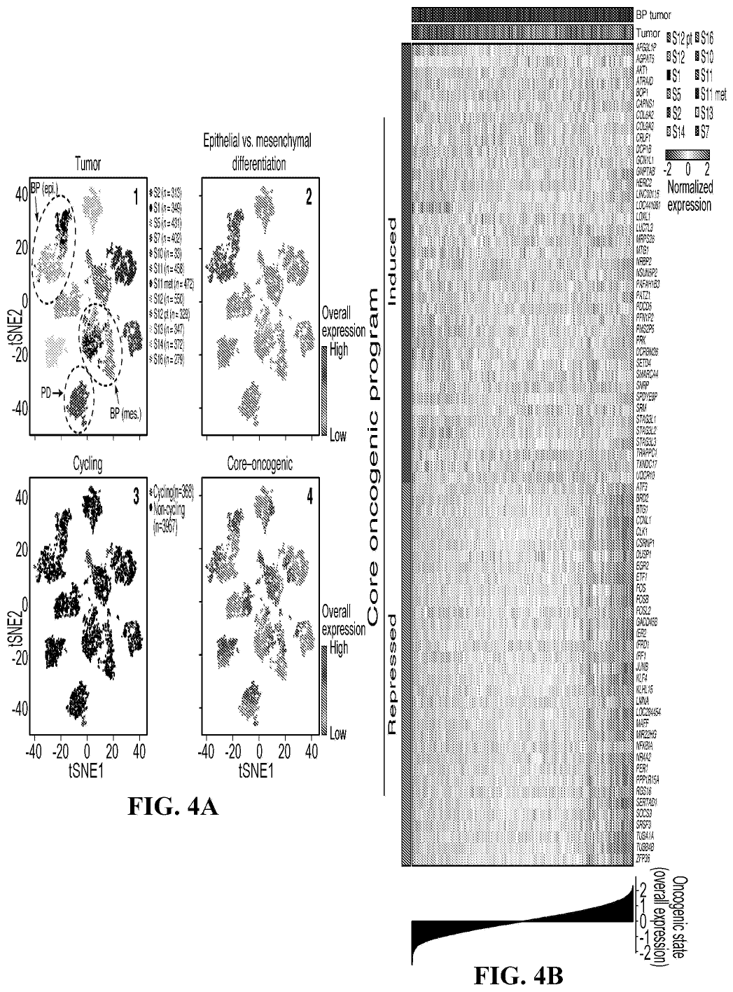

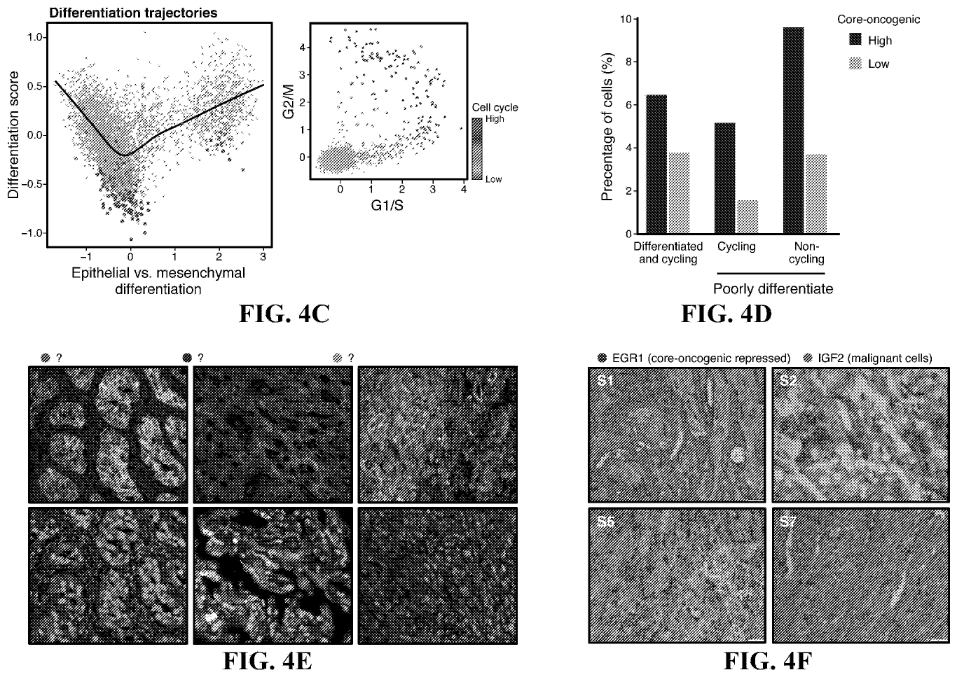

In one example embodiment, the expression signature consists of overrepresented gene sets when considering induced and repressed genes, with both direct and indirect genes, as provided in FIG. 4. In some embodiments, the up-regulated targets are selected from E2F targets, RNA splicing, RNA processing, RNA binding, Ribonucleoprotein complex, Poly-a RNA binding, mRNA metabolic process, G2M checkpoint, Myc targets, Oxidative phosphorylation, Single-cell cell cycle, Single-cell oncogenic, Embryo development, Neurogenesis, Organ morphogenesis, Pattern specification process, Tissue development, Hedgehog signaling, Wnt beta catenin signaling, Single-cell synovial sarcoma. In one example embodiment, the down-regulated targets are selected from Cell junction, Extracellular matrix, Positive regulation of cell death, Regulation of cell differentiation, Regulation of cell proliferation, Regulation of organismal development, Response to lipid, Tissue development, Apoptosis, Coagulation, Epithelial mesenchymal transition, Hypoxia, Tnfa signaling via NFKb, Single-cell anti-oncogenic, Single-cell mesenchymal, Embryonic morphogenesis, Epithelium development, and Regulation of cell death.

In certain embodiments, Sys induces the malignant gene signature in synovial sarcoma cells and the Sys cells can be selectively targeted and this signature can be modulated by treatment with an inhibitor of HDAC or an inhibitor of CDK4/6.

In one example embodiment the malignant gene signature comprises ALDH1A1 and at least N additional biomarker from Tables 1A-1E, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

### Malignant Epithelial Cell Signature

In one example embodiment, the Malignant Epithelial Program signature consists of one or more of ABCG1, ABHD11, ABRACL, ACOT7, ACP5, ADAMTSL2, AES, AGPAT2, AGRN, AGTRAP, AHNAK2, AIG1, AKR1C3, ALDH1A3, ALDH3A2, ALDH4A1, ALOX15, ANK3, ANO9, ANXA11, ANXA3, AP1M2, APOE, APP, ARHGAP8, ARID5A, ARRDC1, ASS1, ATHL1, ATP6VOE2, BAIAP2L1, BARX2, BCAM, BSCL2, C14orf1, C19orf21, C19orf33, C1GALT1C1, C1orf210, CAP2, CAPN6, CARD16, CARNS1, CBLC, CCDC153, CCDC24, CCND1, CD151, CD55, CD59, CD7, CD74, CD9, CDCP1, CDH1, CDH3, CDH4, CDK2AP2, CHST9, CKB, CLDN3, CLDN4, CLDN7, CLIC3, CLU, COL12A1, CRB3, CRIP1, CRIP2, CXADR, CXCL1, CYB561, CYBA, CYFIP2, CYHR1, CYP39A1, CYP4X1, CYSTM1, DBNDD2, DCXR, DDR1, DDX58, DHCR7, DMKN, DRD1, DSP, EFCAB4A, EFNA5, ELOVL1, ELOVL7, EMB, ENO2, ENPP5, ENTPD3, EPB41L5, EPCAM, EPHA2, EPS8L2, ERBB2, ERBB3, ESRP1, ESRP2, EZR, F11R, F2RL1, FAAH, FAAH2, FAM111A, FAM167A, FAM213A, FAM221A, FAM65C, FAM84B, FBXO2, FBXO44, FGF19, FGFRL1, FMO2, FXYD3, FXYD5, FZD6, GALNT3, GAS6, GCHFR, GPR56, GPRC5A, GPRC5C, GRB7, GSDMD, HERC6, HIGD2A, HLA-B, HMGA1, HOOK2, HPN, HSPB2, IFITM1, IFITM2, IFITM5, IGFBP6, IGSF9, INADL, INF2, IQGAP1, IRF6, IRF7, ISLR, ITGA3, ITGB4, ITGB8, ITPR2, ITPR3, JUP, KIAA1522, KIAA1598, KIF1A, KLF5, KLK1, KLK10, KLK11, KLK7, KLK8, KRT18, KRT19, KRT7, KRT8, KRTCAP3, LBH, LECT1, LGALS3BP, LIME1, LLGL2, LOC100505761, L00541471, LOC646329, LPAR2, LPIN3, LRRC16A, LSR, LY6E, LYPD6B, MAGI1, MAL2, MAP7, MBOAT1, MCAM, MDK, MFSD3, MGAT4B, MIF4GD, MLXIPL, MPZL2, MSLN, MSMO1, MSX2, MUC1, MX1, MYH9, MYO6, NCOA7, NDUFA4L2, NDUFS8, NET1, NPNT, NSMF, NT5DC1, NTSE, NUDT14, OAS1, OCIAD2, OCLN, ORMDL2, P4HTM, PARD6B, PARP8, PARP9, PARVG, PCBD1, PDGFB, PDHX, PDLIM1, PDLIM2, PERP, PHYHD1, PIGV, PIM1, PKP3, PKP4, PLEKHB1, PLEKHG1, PLEKHN1, PLLP, PLXDC2, PLXNA2, PLXNB1, PNOC, PNP, PPL, PPP1CA, PPP1R16A, PPP1R1B, PPP1R9A, PRKCG, PRPH, PRR15, PRR15L, PRSS8, PSME1, PSME2, PTGER4, PTGES, PTN, PTPRF, PTRH1, RAB3IP, RALGPS1, RASSF7, RBM47, REC8, REEP2, RGL3, RHBDF2, RHBDL1, RIPK4, ROBO3, RTN3, S100A16, S100A4, S100A6, SAMD12, SCG5, SCNN1A, SCRN2, SEC11C, SECTM1, SELENBP1, SEMA3B, SGPL1, SH3YL1, SHANK2, SHANK2-AS3, SIM2, SLC11A2, SLC12A2, SLC16A5, SLC25A25, SLC25A29, SLC29A1, SLC35F2, SLC50A1, SLC6A9, SLC7A5, SLC7A8, SLFN5, SLPI, SMAD1, SMPDL3B, SORT1, SOX14, SPINT1, SPINT2, ST14, ST3GAL5, STAP2, STRA13, STRA6, STXBP2, SULF1, SULF2, SUMF1, SVIP, SYNGR2, SYTL1, TACSTD2, TAPBPL, TCF7L2, TENM1, TFAP2B, TFAP2C, TLE2, TLE6, TM4SF1, TM7SF2, TMC4, TMCC3, TMEM125, TMEM176B, TNFAIP2, TNFRSF12A, TNFRSF14, TNFRSF21, TNFSF13, TNKS1BP1, TNNI3, TNNT1, TOM1L1, TPD52, TSPO, TUBB2B, TUBB3, UCP2, VAMP8, WDR34, WDR54, WFDC2, XAF1, ZDHHC12, ZMAT1, ZNF165, ZNF423, and ZNF664.

### Malignant Mesenchymal Cell Signature

In one example embodiment, a malignant mesenchymal cell signature comprises one or more genes or polypeptides selected from the group consisting of: ANLN, CLSPN, KNSTRN, RFC4, ARHGAP11A, DHFR, KNTC1, RNASEH2A, ATAD5, DNA2, MAD2L1, RRM2, BIRC5, DTL, MCM2, SGOL2, BRCA2, EZH2, MCM3, SMC4, BUB1B, FANCA, MCM4, SPAG5, C21orf58, FANCD2, MCM5, SPDL1, CASC5, FANCI, MKI67, STIL, CCNA2, FOXM1, MLF1IP, TCF19, CCNB2, GINS2, NCAPD2, TIMELESS, CCNE2, HELLS, NCAPG2, TK1, CDC6, KIAA0101, NUSAP1, TOP2A, CDKN3, KIF11, OAS3, TPX2, CENPE, KIF14, OIP5, TYMS, CENPF, KIF18A, ORC6, UBE2C, CENPH, KIF20B, PRC1, UBE2T, CENPK, KIF2C, PSMC3IP, UHRF1, CENPW, PTTG1, WDHD1, CHAF1B, RACGAP1, ZWINT.

### Modulation Using a HDAC Inhibitor, CDK4/6 Inhibitor, or a Combination Thereof.

The following section provides multiple example embodiments for modulating one or more malignant signatures associated with Sys. Methods may include administration to subjects at risk for or having Sys, including metastatic or at risk for having metastatic Sys. Thus, the embodiments may be used to prevent and/or treat Sys or metastatic Sys.

In another aspect, methods of treatment may comprise administering a HDAC inhibitor, a CDK4/6 inhibitor or a combination thereof, to a subject in need thereof. In certain example embodiments, a subject in need thereof may be a subject at risk for or having synovial sarcoma.

### HDAC Inhibitor

In certain embodiments, the agent capable of modulating a signature as described herein is an HDAC inhibitor. Examples of HDAC inhibitors include hydroxamic acid derivatives, Short Chain Fatty Acids (SCFAs), cyclic tetrapeptides, benzamide derivatives, or electrophilic ketone derivatives, as defined herein. Specific non-limiting examples of HDAC inhibitors include: A) Hydroxamic acid derivatives selected from m-carboxycinnamic acid bishydroxamide (CBHA), Trichostatin A (TSA), Trichostatin C, Salicylhydroxamic Acid, Azelaic Bishydroxamic Acid (ABHA), Azelaic-1-Hydroxamate-9-Anilide (AAHA), 6-(3-Chlorophenylureido) carpoic Hydroxamic Acid (3C1-UCHA), Oxamflatin, A-161906, Scriptaid, PXD-101, LAQ-824, CHAP, MW2796, and MW2996; B) Cyclic tetrapeptides selected from Trapoxin A, FR901228 (FK 228 or Depsipeptide), FR225497, Apicidin, CHAP, HC-Toxin, WF27082, and Chlamydocin; C) Short Chain Fatty Acids (SCFAs) selected from Sodium Butyrate, Isovalerate, Valerate, 4 Phenylbutyrate (4-PBA), Phenylbutyrate (PB), Propionate, Butyramide, Isobutyramide, Phenylacetate, 3-Bromopropionate, Tributyrin, Valproic Acid and Valproate; D) Benzamide Derivatives selected from C 1-994, MS-27-275 (MS-275) and a 3′-amino derivative of MS-27-275; E) Electrophilic Ketone Derivatives selected from a trifluoromethyl ketone and an α-keto amide such as an N-methyl-α-ketoamide; and F) Miscellaneous HDAC inhibitors including natural products, psammaplins and Depudecin.

Additional examples of HDAC inhibitors include vorinostat, romidepsin, chidamide, panobinostat, belinostat, mocetinostat, abexinostat, entinostat, resminostat, givinostat, quisinostat, CI-994, BML-210, M344, NVP-LAQ824, suberoylanilide hydroxamic acid (SAHA), MS-275, TSA, LAQ-824, trapoxin, depsipeptide, and tacedinaline.

Further examples of HDAC inhibitors include trichostatin A (TSA) ((R,2E,4E)-7-(4-(dimethylamino)phenyl)-N-hydroxy-4,6-dimethyl-7-oxohepta-2,4-dienamide); sulfonamides such as oxamflatin ((E)-N-hydroxy-5-(3-(phenylsulfonamido)phenyl)pent-2-en-4-ynamide). Other hydroxamic-acid-sulfonamide inhibitors of histone deacetylase are described in: Lavoie et al. (2001) Bioorg. Med. Chem. Lett. 11:2847-50; Bouchain et al. (2003) J. Med. Chem. 846:820-830; Bouchain et al. (2003) Curr. Med. Chem. 10:2359-2372; Marson et al. (2004) Bioorg. Med. Chem. Lett. 14:2477-2481; Finn et al. (2005) Helv. Chim. Acta 88:1630-1657; International Patent Publication Nos. WO 2002/030879, WO 2003/082288, WO 2005/0011661, WO 2005/108367, WO 2006123121, WO 2006/017214, WO 2006/017215, and US Patent Publication No. 2005/0234033. Other structural classes of histone deacetylase inhibitors include short chain fatty acids, cyclic peptides, and benzamides. Acharya et al. (2005) Mol. Pharmacol. 68:917-932.

Other examples of HDAC inhibitors include those disclosed in, e.g., Dokmanovic et al. (2007) Mol. Cancer. Res. 5:981; U.S. Pat. Nos. 7,642,275; 7,683,185; 7,732,475; 7,737,184; 7,741,494; 7,772,245; 7,795,304; 7,799,825; 7,803,800; 7,842,727; 7,842,835; U.S. Patent Publication No. 2010/0317739; U.S. Patent Publication No. 2010/0311794; U.S. Patent Publication No. 2010/0310500; U.S. Patent Publication No. 2010/0292320; and U.S. Patent Publication No. 2010/0291003.

### CDK4/6 Inhibitor

In certain embodiments, the agent capable of modulating a signature as described herein is a cell cycle inhibitor (see e.g., Dickson and Schwartz, Development of cell-cycle inhibitors for cancer therapy, Curr Oncol. 2009 March; 16(2): 36-43). In one embodiment, the agent capable of modulating a signature as described herein is a CDK4/6 inhibitor, such as LEE011, palbociclib (PD-0332991), and Abemaciclib (LY2835219) (see, e.g., U.S. Pat. No. 9,259,399B2; International Patent Publication No. WO 2016/025650A1; US Patent Publication No. 2014/0031325; US Patent Publication No. 2014/0080838; US Patent Publication No. 2013/0303543; US Patent Publication No. 2007/0027147; US Patent Publication No. 2003/0229026; US Patent Publication No 2004/0048915; US Patent Publication No. 2004/0006074; and US Patent Publication No. 2007/0179118, each of which is incorporated herein by reference in its entirety). Currently there are three CDK4/6 inhibitors that are either approved or in late-stage development: palbociclib (PD-0332991; Pfizer), ribociclib (LEE011; Novartis), and abemaciclib (LY2835219; Lilly) (see e.g., Hamilton and Infante, Targeting CDK4/6 in patients with cancer, Cancer Treatment Reviews, Volume 45, April 2016, Pages 129-138).

### Checkpoint Inhibitors

Because immune checkpoint inhibitors target the interactions between different cells in the tumor, their impact depends on multicellular circuits between malignant and non-malignant cells (Tirosh et al., 2016a). In principle, resistance can stem from different compartment of the tumor's ecosystem, for example, the proportion of different cell types (e.g., T cells, macrophages, fibroblasts), the intrinsic state of each cell (e.g., memory or dysfunctional T cell), and the impact of one cell on the proportions and states of other cells in the tumor (e.g., malignant cells inducing T cell dysfunction by expressing PD-L1 or promoting T cell memory formation by presenting neoantigens). These different facets are interconnected through the cellular ecosystem: intrinsic cellular states control the expression of secreted factors and cell surface receptors that in turn affect the presence and state of other cells, and vice versa. In particular, brisk tumor infiltration with T cell has been associated with patient survival and improved immunotherapy responses (Fridman et al., 2012), but the determinants that dictate if a tumor will have high (“hot”) or low (“cold”) levels of T cell infiltration are only partially understood. Among multiple factors, malignant cells may play an important role in determining this phenotype (Spranger et al., 2015). Resolving this relationship with bulk genomics approaches has been challenging; single-cell RNA-seq (scRNA-seq) of tumors (Li et al., 2017; Patel et al., 2014; Tirosh et al., 2016a, 2016b; Venteicher et al., 2017) has the potential to shed light on a wide range of immune evasion mechanisms and immune suppression programs.

### Phased Combination

In certain embodiments, a subject in need thereof is treated with a combination therapy, which may be a phased combination therapy. The phased combination therapy may be a treatment regimen comprising checkpoint inhibition followed by a CDK4/6 inhibitor, an HDAC inhibitor, an/or checkpoint inhibitor combination. Checkpoint inhibitors may be administered at regular intervals, for example, daily, weekly, every two weeks, every month. The combination therapy may be administered when a signature disclosed herein is detected. This may be after two weeks to six months after the initial checkpoint inhibition. The immunotherapy may be adoptive cell transfer therapy, as described herein or may be an inhibitor of any check point protein described herein. The checkpoint blockade therapy may comprise anti-TIM3, anti-CTLA4, anti-PD-L1, anti-PD1, anti-TIGIT, anti-LAG3, or combinations thereof. Specific check point inhibitors include, but are not limited to anti-CTLA4 antibodies (e.g., Ipilimumab), anti-PD-1 antibodies (e.g., Nivolumab, Pembrolizumab), and anti-PD-L1 antibodies (e.g., Atezolizumab). Dosages for the immunotherapy and/or CDK4/6 inhibitors may be determined according to the standard of care for each therapy and may be incorporated into the standard of care (see, e.g., Rivalland et al., Standard of care in immunotherapy trials: Challenges and considerations, Hum Vaccin Immunother. 2017 July; 13(9): 2164-2178; and Pernas et al., CDK4/6 inhibition in breast cancer: current practice and future directions, Ther Adv Med Oncol. 2018). The standard of care is the current treatment that is accepted by medical experts as a proper treatment for a certain type of disease and that is widely used by healthcare professionals. Standard or care is also called best practice, standard medical care, and standard therapy.

### Methods of Treatment

Treatment with Adoptive Cell Transfer

In embodiments, methods of treatment of Sys may comprise treatment with adoptive cell therapy via CD8 T cells, CAR T and/or macrophages. In embodiments, macrophages are edited to provide increased IFNgamma, CD8 T cells are edited to provide increased TNF expression, or a combination thereof. In embodiments, methods of treatment include adoptive cell therapy utilizing CD8 and/or CAR T cells edited to have the expansion program phenotype as provided herein. As described further in the examples, IFNg and TNF was strongly associated with the repression of the core oncogenic program in malignant cells. Further, the T cells in SyS tumors have been found to have a cytotoxic potential which might be unleashed by immune checkpoint blockade. Accordingly, the methods of treatment using these adoptive cell therapies have potential to modulate, reduce and/or repress the oncogenic program in malignant cells and/or increase cytotoxicity.

As used herein, “ACT”, “adoptive cell therapy” and “adoptive cell transfer” may be used interchangeably. In certain embodiments, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8(1):424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 June; 24(6):724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the invention involve the adoptive transfer of immune system cells, such as T cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3):453-62).

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostate; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); κ-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDG1cp(1-1)Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDG1cp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukaemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; and any combination thereof.

In certain embodiments, an antigen to be targeted in adoptive cell therapy (such as particularly CAR or TCR T-cell therapy) of a disease (such as particularly of tumor or cancer) is a tumor-specific antigen (TSA).

In certain embodiments, an antigen to be targeted in adoptive cell therapy (such as particularly CAR or TCR T-cell therapy) of a disease (such as particularly of tumor or cancer) is a neoantigen.

In certain embodiments, an antigen to be targeted in adoptive cell therapy (such as particularly CAR or TCR T-cell therapy) of a disease (such as particularly of tumor or cancer) is a tumor-associated antigen (TAA).

In certain embodiments, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (Dl), and any combinations thereof.

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CD70, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CML), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+ malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR a and β chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8α hinge domain and a CD8α transmembrane domain, to the transmembrane and intracellular signaling domains of either CD3ζ or FcRγ (scFv-CD3ζ or scFv-FcRγ; see U.S. Pat. Nos. 7,741,465; 5,912,172; and 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; and 9,102,761). Third-generation CARs include a combination of costimulatory endodomains, such a CD3ζ-chain, CD97, GDI 1a-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO 2014/134165; PCT Publication No. WO 2012/079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon Rib), CD79a, CD79b, Fc gamma RIM, DAP10, and DAP12. In certain preferred embodiments, the primary signaling domain comprises a functional signaling domain of CD3ζ or FcRγ. In certain embodiments, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of 4-1BB, CD27, and CD28. In certain embodiments, a chimeric antigen receptor may have the design as described in U.S. Pat. No. 7,446,190, comprising an intracellular domain of CD3ζ chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139 (sequence version 1, 2 or 3): IEVMYPPPYLDNEKSNGTIIHVKGKHLCPSPLFPGPSKPFWVLVVVGGVLACYSLLVT VAFIIFWVRSKRSRLLHSDYMNMTPRRPGPTRKHYQPYAPPRDFAAYRS)) (SEQ ID NO:1). Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3 ζ chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-ζ molecule. The exact sequence of the CD28 molecule included in the FMC63-28Z CAR corresponded to Genbank identifier NM_006139; the sequence included all amino acids starting with the amino acid sequence IEVMYPPPY (SEQ. I.D. No. 2) and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and Nothdigested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3 ζ chain, and a costimulatory signaling region comprising a signaling domain of CD28. Preferably, the CD28 amino acid sequence is as set forth in Genbank identifier NM 006139 (sequence version 1, 2 or 3) starting with the amino acid sequence IEVMYPPPY (SEQ ID NO: 2) and continuing all the way to the carboxy-terminus of the protein. The sequence is reproduced herein: IEVMYPPPYLDNEKSNGTIIHVKGKHLCPSPLFPGPSKPFWVLVVVGGVLACYSLLVT VAFIIFWVRSKRSRLLHSDYMNMTPRRPGPTRKHYQPYAPPRDFAAYRS (SEQ ID NO: 1). Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in International Patent Publication No. WO 2015/187528. More particularly, Example 1 and Table 1 of WO 2015/187528, incorporated by reference herein, demonstrate the generation of anti-CD19 CARs based on a fully human anti-CD19 monoclonal antibody (47G4, as described in US Patent Publication No. 2010/0104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signalling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-CD27-FcεRI gamma chain; or CD28-FcεRI gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO 2015/187528 and an intracellular T-cell signalling domain as set forth in Table 1 of WO 2015/187528. Preferably, the antigen is CD19, more preferably the antigen-binding element is an anti-CD19 scFv, even more preferably the mouse or human anti-CD19 scFv as described in Example 1 of WO 2015/187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO 2015/187528.

By means of an example and without limitation, chimeric antigen receptor that recognizes the CD70 antigen is described in International Patent Publication No. WO 2012/058460A2 (see also, Park et al., CD70 as a target for chimeric antigen receptor T cells in head and neck squamous cell carcinoma, Oral Oncol. 2018 March; 78:145-150; and Jin et al., CD70, a novel target of CAR T-cell therapy for gliomas, Neuro Oncol. 2018 Jan. 10; 20(1):55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am. J. Pathol. 1995; 147: 1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US Patent Publication No. 2016/0046724 A1; International Patent Publication Nos. WO 2016/014789 A2, WO 2017/211900 A1, WO 2015/158671 A1, WO2018028647A1, and WO 2013/154760 A1; and US Patent Publication Nos. 2018/0085444 A1 and 2017/0283504 A1).

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an MHC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its WIC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., International Patent Publication Nos. WO 2013/044225, WO 2016/000304, WO 2015/057834, WO 2015/057852, WO 2016/070061, U.S. Pat. No. 9,233,125, and US Patent Publication No. 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US Patent Publication Nos. 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLoS One, 2013) or to downregulate expression of the CAR following binding to the target antigen (WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CART cells of this kind may for example be used in animal models, for example to treat tumor xenografts.

In certain embodiments, ACT includes co-transferring CD4+Th1 cells and CD8+ CTLs to induce a synergistic antitumour response (see, e.g., Li et al., Adoptive cell therapy with CD4+T helper 1 cells and CD8+ cytotoxic T cells enhances complete rejection of an established tumour, leading to generation of endogenous memory responses to non-targeted tumour epitopes. Clin Transl Immunology. 2017 October; 6(10): e160).

In certain embodiments, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112(2):362-73; and Martin-Orozco N, et al., T helper 17 cells promote cytotoxic T cell activation in tumor immunity. Immunity. 2009 Nov. 20; 31(5):787-98). Those studies involved an adoptive T cell transfer (ACT) therapy approach, which takes advantage of CD4⁺ T cells that express a TCR recognizing tyrosinase tumor antigen. Exploitation of the TCR leads to rapid expansion of Th17 populations to large numbers ex vivo for reinfusion into the autologous tumor-bearing hosts.

In certain embodiments, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257(1):56-71. doi:10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In certain embodiments, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10):1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells or population of cells, may be made resistant to at least one immunosuppressive agent due to the inactivation of a gene encoding a receptor for such immunosuppressive agent. In certain embodiments, the immunosuppressive treatment provides for the selection and expansion of the immunoresponsive T cells within the patient.

In certain embodiments, the treatment can be administered before primary treatment (e.g., surgery or radiation therapy) to shrink a tumor before the primary treatment. In another embodiment, the treatment can be administered after primary treatment to remove any remaining cancer cells.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017. 00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e. intracavity delivery) or directly into a tumor prior to resection (i.e. intratumoral delivery). In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount (e.g. number) of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be done directly by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 2013/0071414; PCT Patent Publication Nos. WO 2011/146862, WO 2014/011987, WO 2013/040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23(9):2255-2266. doi: 10.1158/1078-0432.CCR-16-1300. Epub 2016 Nov. 4; Qasim et al., 2017, Molecular remission of infant B-ALL after infusion of universal TALEN gene-edited CAR T cells, Sci Transl Med. 2017 Jan. 25; 9(374); Legut, et al., 2018, CRISPR-mediated TCR replacement generates superior anticancer transgenic T cells. Blood, 131(3), 311-322; and Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018). Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more WIC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see PCT Patent Publications: WO2013176915, WO2014059173, WO2014172606, WO2014184744, and WO2014191128).

In certain embodiments, editing may result in inactivation of a gene. By inactivating a gene, it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In certain embodiments, homology directed repair (HDR) is used to concurrently inactivate a gene (e.g., TRAC) and insert an endogenous TCR or CAR into the inactivated locus.

Hence, in certain embodiments, editing of cells (such as by CRISPR/Cas), particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing of cells (such as by CRISPR/Cas), particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In certain embodiments, editing of cells (such as by CRISPR/Cas), particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, WO2016196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as CRISPR, TALEN or ZFN) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CART cells deficient of TCR, HLA class I molecule and PD1.

In certain embodiments, cells may be engineered to express a CAR, wherein expression and/or function of methylcytosine dioxygenase genes (TET1, TET2 and/or TET3) in the cells has been reduced or eliminated, such as by CRISPR, ZNF or TALEN (for example, as described in WO201704916).

In certain embodiments, editing of cells (such as by CRISPR/Cas), particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in WO2016011210 and WO2017011804).

In certain embodiments, editing of cells (such as by CRISPR/Cas), particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In certain embodiments, a cell may be multiply edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells (PBMC), bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3X28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (i.e., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶/ml. In other embodiments, the concentration used can be from about 1×10⁵/ml to 1×10⁶/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-MHC tetramers (Altman, et al., Science. 1996 Oct. 4; 274(5284):94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MEW molecule of interest and any antigen of interest as described herein. Specific epitopes to be used in this context can be identified using numerous assays known in the art. For example, the ability of a polypeptide to bind to MEW class I may be evaluated indirectly by monitoring the ability to promote incorporation of ¹²⁵I labeled β2-microglobulin (β2m) into MEW class I/β2m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003057171, U.S. Pat. No. 8,034,334, and U.S. Patent Application Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In certain embodiments, T cells comprising a CAR or an exogenous TCR, may be manufactured as described in WO2015120096, by a method comprising: enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in WO2015120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In certain embodiments, T cell maturation or differentiation in vitro may be delayed or inhibited by the method as described in WO2017070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In certain embodiments, a patient in need of a T cell therapy may be conditioned by a method as described in WO2016191756 comprising administering to the patient a dose of cyclophosphamide between 200 mg/m2/day and 2000 mg/m2/day and a dose of fludarabine between 20 mg/m2/day and 900 mg/m²/day.

### Modulation of One or More Biomarkers of a Malignant Expression Signature

In certain embodiments, a method of treating Sys cells comprises administering or more agents capable of modulating expression, activity, or function of one or more biomarkers of the malignant gene signatures defined in Tables 1A-1E.

### Modulation of an Expansion Signature

In certain embodiments, a method of selectively treating Sys cells or reducing or repressing metastasis comprises administering one or more agents capable of modulating expression, activity, or function of one or more biomarkers of the malignant signatures in Tables 1A-1E. In another example embodiment, method of selectively targeting synovial sarcoma cells comprises administering one or more agents capable of modulating expression, activity, or function of one or more biomarkers of the malignant signatures defined at any one of Tables 1A-1E.

### Modulation of Cell-Type Specific Biological Programs

In another aspect, embodiments disclosed herein provide a method of modulating an malignant signature comprising administering to a population of cells comprising Sys cells, one or more agents capable of modulating expression, activity of one or more signatures as defined in Tables 1A to 1E.

In one example embodiment, the method comprises administering to a population of cells comprising Sys cells one or more agents capable of modulating expression, activity of one or more biological programs characterized by one or more of Tables 1A-1E.

In one example embodiment, the method comprises administering to a population of cells comprising Sys cells one or more agents capable of modulating expression, activity of one or more biological programs characterized by the one or more of the signatures of Tables 1A-1E.

In certain example embodiments, the agent suppresses one of the above biological programs, whereby Sys cells are selectively targeted while sparing non-malignant cells. The one or more agents may comprise agent(s) that modulate the expression, activity or function of one or more genes of or polypeptides in Tables 1A-1E.

In certain example embodiments, the population of cells is in vivo. In certain embodiments, the in vivo population is present in the gut of a subject. In other example embodiments, the population of cell is an in vitro or ex vivo population of cells. In certain other example embodiments, the population of cells is an intestinal organoid.

### Modulation and Modulating Agents

As used herein, “modulating” or “to modulate” generally means either reducing or inhibiting the expression or activity of, or alternatively increasing the expression or activity of a target or antigen. In particular, “modulating” or “to modulate” can mean either reducing or inhibiting the activity of, or alternatively increasing a (relevant or intended) biological activity of, a target or antigen as measured using a suitable in vitro, cellular or in vivo assay (which will usually depend on the target involved), by at least 5%, at least 10%, at least 25%, at least 50%, at least 60%, at least 70%, at least 80%, at least 90%, or more, compared to activity of the target in the same assay under the same conditions but without the presence of an agent. An “increase” or “decrease” refers to a statistically significant increase or decrease respectively. For the avoidance of doubt, an increase or decrease will be at least 10% relative to a reference, such as at least 10%, at least 20%, at least 30%, at least 40%, at least 50%, at least 60%, at least 70%, at least 80%, at least 90%, at least 95%, at least 97%, at least 98%, or more, up to and including at least 100% or more, in the case of an increase, for example, at least 2-fold, at least 3-fold, at least 4-fold, at least 5-fold, at least 6-fold, at least 7-fold, at least 8-fold, at least 9-fold, at least 10-fold, at least 50-fold, at least 100-fold, or more. “Modulating” can also involve effecting a change (which can either be an increase or a decrease) in affinity, avidity, specificity and/or selectivity of a target or antigen. “Modulating” can also mean effecting a change with respect to one or more biological or physiological mechanisms, effects, responses, functions, pathways or activities in which the target or antigen (or in which its substrate(s), ligand(s) or pathway(s) are involved, such as its signaling pathway or metabolic pathway and their associated biological or physiological effects) is involved. Again, as will be clear to the skilled person, such an action as an agonist or an antagonist can be determined in any suitable manner and/or using any suitable assay known or described herein (e.g., in vitro or cellular assay), depending on the target or antigen involved.

Modulating can, for example, also involve allosteric modulation of the target and/or reducing or inhibiting the binding of the target to one of its substrates or ligands and/or competing with a natural ligand, substrate for binding to the target. Modulating can also involve activating the target or the mechanism or pathway in which it is involved. Modulating can for example also involve effecting a change in respect of the folding or confirmation of the target, or in respect of the ability of the target to fold, to change its conformation (for example, upon binding of a ligand), to associate with other (sub)units, or to disassociate. Modulating can for example also involve effecting a change in the ability of the target to signal, phosphorylate, dephosphorylate, and the like.

As used herein, an “agent” can refer to a protein-binding agent that permits modulation of activity of proteins or disrupts interactions of proteins and other biomolecules, such as but not limited to disrupting protein-protein interaction, ligand-receptor interaction, or protein-nucleic acid interaction. Agents can also refer to DNA targeting or RNA targeting agents. Agents can also refer to a protein. Agents may include a fragment, derivative and analog of an active agent. The terms “fragment,” “derivative” and “analog” when referring to polypeptides as used herein refers to polypeptides which either retain substantially the same biological function or activity as such polypeptides. An analog includes a proprotein which can be activated by cleavage of the proprotein portion to produce an active mature polypeptide. Such agents include, but are not limited to, antibodies (“antibodies” includes antigen-binding portions of antibodies such as epitope- or antigen-binding peptides, paratopes, functional CDRs; recombinant antibodies; chimeric antibodies; humanized antibodies; nanobodies; tribodies; midibodies; or antigen-binding derivatives, analogs, variants, portions, or fragments thereof), protein-binding agents, nucleic acid molecules, small molecules, recombinant protein, peptides, aptamers, avimers and protein-binding derivatives, portions or fragments thereof. An “agent” as used herein, may also refer to an agent that inhibits expression of a gene, such as but not limited to a DNA targeting agent (e.g., CRISPR system, TALE, Zinc finger protein) or RNA targeting agent (e.g., inhibitory nucleic acid molecules such as RNAi, miRNA, ribozyme).

In certain embodiments, the agent modulates Sys malignant signature. In certain embodiments, the agent is an inhibitor of HDAC and/or CDK4/6.

The composition of the invention can also advantageously be formulated in order to release inhibitor of HDAC and/or CDK4/6 in the subject in a timely controlled fashion. In a particular embodiment, the composition of the invention is formulated for controlled release of inhibitor of HDAC and/or CDK4/6.

In some embodiments, the modulating agent modulated one or more biomarkers of a) epithelial malignant signature as defined in Table 1E; b) mesenchymal malignant cell signature as defined in Table 1D; c) cell cycle signature as defined in Table 1C; d) core oncogenic signature as defined in Table 1A.1; e) a fusion signature as defined in Table 8; or f) a combination thereof. In certain embodiments, an effective amount of the modulating agent is administered.

In certain embodiments, the agent is capable of inhibitor of HDAC and/or CDK4/6. In certain embodiments, HDAC and/or CDK4/6 expression is inhibited, e.g., by a DNA targeting agent (e.g., CRISPR system, TALE, Zinc finger protein) or a RNA targeting agent (e.g., inhibitory nucleic acid molecules). In certain embodiments, the antagonist is an antibody or fragment thereof. In certain embodiments, the antibody is specific for HDAC and/or CDK4/6.

The agents of the present invention may be modified, such that they acquire advantageous properties for therapeutic use (e.g., stability and specificity), but maintain their biological activity.

It is well known that the properties of certain proteins can be modulated by attachment of polyethylene glycol (PEG) polymers, which increases the hydrodynamic volume of the protein and thereby slows its clearance by kidney filtration. (See, e.g., Clark et al., J. Biol. Chem. 271: 21969-21977 (1996)). Therefore, it is envisioned that certain agents can be PEGylated (e.g., on peptide residues) to provide enhanced therapeutic benefits such as, for example, increased efficacy by extending half-life in vivo. In certain embodiments, PEGylation of the agents may be used to extend the serum half-life of the agents and allow for particular agents to be capable of crossing the blood-brain barrier. Thus, in one embodiment, PEGylating inhibitor of HDAC and/or CDK4/6 improve the pharmacokinetics and pharmacodynamics of the inhibitors.

In regards to peptide PEGylation methods, reference is made to Lu et al., Int. J. Pept. Protein Res. 43: 127-38 (1994); Lu et al., Pept. Res. 6: 140-6 (1993); Felix et al., Int. J. Pept. Protein Res. 46: 253-64 (1995); Gaertner et al., Bioconjug. Chem. 7: 38-44 (1996); Tsutsumi et al., Thromb. Haemost. 77: 168-73 (1997); Francis et al., hit. J. Hematol. 68: 1-18 (1998); Roberts et al., J. Pharm. Sci. 87: 1440-45 (1998); and Tan et al., Protein Expr. Purif. 12: 45-52 (1998). Polyethylene glycol or PEG is meant to encompass any of the forms of PEG that have been used to derivatize other proteins, including, but not limited to, mono-(C1-10) alkoxy or aryloxy-polyethylene glycol. Suitable PEG moieties include, for example, 40 kDa methoxy poly(ethylene glycol) propionaldehyde (Dow, Midland, Mich.); 60 kDa methoxy poly(ethylene glycol) propionaldehyde (Dow, Midland, Mich.); 40 kDa methoxy poly(ethylene glycol) maleimido-propionamide (Dow, Midland, Mich.); 31 kDa alpha-methyl-w-(3-oxopropoxy), polyoxyethylene (NOF Corporation, Tokyo); mPEG2-NHS-40k (Nektar); mPEG2-MAL-40k (Nektar), SUNBRIGHT GL2-400MA ((PEG)240 kDa) (NOF Corporation, Tokyo), SUNBRIGHT ME-200MA (PEG20 kDa) (NOF Corporation, Tokyo). The PEG groups are generally attached to the peptide via acylation or alkylation through a reactive group on the PEG moiety (for example, a maleimide, an aldehyde, amino, thiol, or ester group) to a reactive group on the peptide (for example, an aldehyde, amino, thiol, a maleimide, or ester group).

The PEG molecule(s) may be covalently attached to any Lys, Cys, or K(CO(CH2)2SH) residues at any position in a peptide. In certain embodiments, the peptides described herein can be PEGylated directly to any amino acid at the N-terminus by way of the N-terminal amino group. A “linker arm” may be added to a peptide to facilitate PEGylation. PEGylation at the thiol side-chain of cysteine has been widely reported (see, e.g., Caliceti & Veronese, Adv. Drug Deliv. Rev. 55: 1261-77 (2003)). If there is no cysteine residue in the peptide, a cysteine residue can be introduced through substitution or by adding a cysteine to the N-terminal amino acid. PEGylaeion can be effected through the side chains of a cysteine residue added to the N-terminal amino acid.

In exemplary embodiments, the PEG molecule(s) may be covalently attached to an amide group in the C-terminus of a peptide. In preferred embodiments, there is at least one PEG molecule covalently attached to the peptide. In certain embodiments, the PEG molecule used in modifying an agent of the present invention is branched while in other embodiments, the PEG molecule may be linear. In particular aspects, the PEG molecule is between 1 kDa and 100 kDa in molecular weight. In further aspects, the PEG molecule is selected from 10, 20, 30, 40, 50, 60, and 80 kDa. In further still aspects, it is selected from 20, 40, or 60 kDa. Where there are two PEG molecules covalently attached to the agent of the present invention, each is 1 to 40 kDa and in particular aspects, they have molecular weights of 20 and 20 kDa, 10 and 30 kDa, 30 and 30 kDa, 20 and 40 kDa, or 40 and 40 kDa. In particular aspects, the agent (e.g., neuromedin U receptor agonists or antagonists) contain mPEG-cysteine. The mPEG in mPEG-cysteine can have various molecular weights. The range of the molecular weight is preferably 5 kDa to 200 kDa, more preferably 5 kDa to 100 kDa, and further preferably 20 kDa to 60 kDA. The mPEG can be linear or branched.

In particular embodiments, the agents (include a protecting group covalently joined to the N-terminal amino group. In exemplary embodiments, a protecting group covalently joined to the N-terminal amino group of the agent reduces the reactivity of the amino terminus under in vivo conditions. Amino protecting groups include —C1-10 alkyl, —C1-10 substituted alkyl, —C2-10 alkenyl, —C2-10 substituted alkenyl, aryl, —C1-6 alkyl aryl, —C(O)—(CH2)1-6-COOH, —C(O)—C1-6 alkyl, —C(O)-aryl, —C(O)—O—C1-6 alkyl, or C(O)—O-aryl. In particular embodiments, the amino terminus protecting group is selected from the group consisting of acetyl, propyl, succinyl, benzyl, benzyloxycarbonyl, and t-butyloxycarbonyl. In other embodiments, deamination of the N-terminal amino acid is another modification that may be used for reducing the reactivity of the amino terminus under in vivo conditions.

Chemically modified compositions of the agents wherein the agent is linked to a polymer are also included within the scope of the present invention. The polymer selected is usually modified to have a single reactive group, such as an active ester for acylation or an aldehyde for alkylation, so that the degree of polymerization may be controlled. Included within the scope of polymers is a mixture of polymers. Preferably, for therapeutic use of the end-product preparation, the polymer will be pharmaceutically acceptable. The polymer or mixture thereof may include but is not limited to polyethylene glycol (PEG), monomethoxy-polyethylene glycol, dextran, cellulose, or other carbohydrate based polymers, poly-(N-vinyl pyrrolidone) polyethylene glycol, propylene glycol homopolymers, a polypropylene oxide/ethylene oxide co-polymer, polyoxyethylated polyols (for example, glycerol), and polyvinyl alcohol.

In other embodiments, the agents are modified by PEGylation, cholesterylation, or palmitoylation. The modification can be to any amino acid residue. In preferred embodiments, the modification is to the N-terminal amino acid of the agent, either directly to the N-terminal amino acid or by way coupling to the thiol group of a cysteine residue added to the N-terminus or a linker added to the N-terminus such as trimesoyl tris(3,5-dibromosalicylate (Ttds). In certain embodiments, the N-terminus of the agent comprises a cysteine residue to which a protecting group is coupled to the N-terminal amino group of the cysteine residue and the cysteine thiolate group is derivatized with N-ethylmaleimide, PEG group, cholesterol group, or palmitoyl group. In other embodiments, an acetylated cysteine residue is added to the N-terminus of the agents, and the thiol group of the cysteine is derivatized with N-ethylmaleimide, PEG group, cholesterol group, or palmitoyl group. In certain embodiments, the agent of the present invention is a conjugate. In certain embodiments, the agent of the present invention is a polypeptide consisting of an amino acid sequence which is bound with a methoxypolyethylene glycol(s) via a linker.

Substitutions of amino acids may be used to modify an agent of the present invention. The phrase “substitution of amino acids” as used herein encompasses substitution of amino acids that are the result of both conservative and non-conservative substitutions. Conservative substitutions are the replacement of an amino acid residue by another similar residue in a polypeptide. Typical but not limiting conservative substitutions are the replacements, for one another, among the aliphatic amino acids Ala, Val, Leu and Ile; interchange of Ser and Thr containing hydroxy residues, interchange of the acidic residues Asp and Glu, interchange between the amide-containing residues Asn and Gln, interchange of the basic residues Lys and Arg, interchange of the aromatic residues Phe and Tyr, and interchange of the small-sized amino acids Ala, Ser, Thr, Met, and Gly. Non-conservative substitutions are the replacement, in a polypeptide, of an amino acid residue by another residue which is not biologically similar. For example, the replacement of an amino acid residue with another residue that has a substantially different charge, a substantially different hydrophobicity, or a substantially different spatial configuration.

In certain embodiments, the present invention provides for one or more therapeutic agents. In certain embodiments, the one or more agents comprises a small molecule inhibitor, small molecule degrader (e.g., PROTAC), genetic modifying agent, antibody, antibody fragment, antibody-like protein scaffold, aptamer, protein, or any combination thereof.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse). In certain embodiments, the present invention provides for one or more therapeutic agents against combinations of targets identified. Targeting the identified combinations may provide for enhanced or otherwise previously unknown activity in the treatment of disease.

In certain embodiments, the one or more agents is a small molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking a binding site or activating a receptor by binding to a ligand binding site).

One type of small molecule applicable to the present invention is a degrader molecule. Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481; Bondeson and Crews, Targeted Protein Degradation by Small Molecules, Annu Rev Pharmacol Toxicol. 2017 Jan. 6; 57: 107-123; and Lai et al., Modular PROTAC Design for the Degradation of Oncogenic BCR-ABL Angew Chem Int Ed Engl. 2016 Jan. 11; 55(2): 807-810).

In certain embodiments, combinations of targets are modulated (e.g., ALDH1A1 and one or more targets related to a gene signature gene). In certain embodiments, an agent against one of the targets in a combination may already be known or used clinically. In certain embodiments, targeting the combination may require less of the agent as compared to the current standard of care and provide for less toxicity and improved treatment.

### Immune Checkpoint

Immune checkpoints are regulators of the immune system. These pathways are crucial for self-tolerance, which prevents the immune system from attacking cells indiscriminately. Modulating immune checkpoint activity may reduce a Sys phenotype or signature. In certain embodiments, a combination treatment may include inhibitors of HDAC and/or CDK4/6 and a checkpoint agonist. Immune checkpoint agonists may activate checkpoint signaling, for example, by binding to the checkpoint protein. The agonists may include a ligand (e.g., PD-L1). PD-1 agonist antibodies that mimic PD-1 ligand (PD-L1) have been described (see, e.g., US Patent Publication No. 2017/0088618A1; International Patent Publication No. WO 2018/053405 A1). Such agonist antibodies against any receptor described herein are applicable to the present invention.

### Antibodies

The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, V_{HH} and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g. the IgG1, IgG2, IgG3, and IgG4 subclassess of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, lgM antibodies exist in pentameric form, and IgA antibodies exist in monomeric, dimeric or multimeric form.

The term “IgG subclass” refers to the four subclasses of immunoglobulin class IgG—IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins—harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CHI domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii) isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}-Cₕ1-V_{H}-Cₕ1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. For example, an antagonist antibody may bind an antigen or antigen receptor and inhibit the ability to suppress a response. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

The disclosure also encompasses nucleic acid molecules, in particular those that inhibit HDAC and/or CDK4/6. Exemplary nucleic acid molecules include aptamers, siRNA, artificial microRNA, interfering RNA or RNAi, dsRNA, ribozymes, antisense oligonucleotides, and DNA expression cassettes encoding said nucleic acid molecules. Preferably, the nucleic acid molecule is an antisense oligonucleotide. Antisense oligonucleotides (ASO) generally inhibit their target by binding target mRNA and sterically blocking expression by obstructing the ribosome. ASOs can also inhibit their target by binding target mRNA thus forming a DNA-RNA hybrid that can be a substance for RNase H. Preferred ASOs include Locked Nucleic Acid (LNA), Peptide Nucleic Acid (PNA), and morpholinos Preferably, the nucleic acid molecule is an RNAi molecule, i.e., RNA interference molecule. Preferred RNAi molecules include siRNA, shRNA, and artificial miRNA. The design and production of siRNA molecules is well known to one of skill in the art (e.g., Hajeri P B, Singh S K. Drug Discov Today. 2009 14(17-18):851-8). The nucleic acid molecule inhibitors may be chemically synthesized and provided directly to cells of interest. The nucleic acid compound may be provided to a cell as part of a gene delivery vehicle. Such a vehicle is preferably a liposome or a viral gene delivery vehicle.

### Genetic Modifying Agents

In certain embodiments, the one or more modulating agents may be a genetic modifying agent. The genetic modifying agent may comprise a CRISPR system, a zinc finger nuclease system, a TALEN, a meganuclease or RNAi system.

### CRISPR-Cas Modification

In some embodiments, a polynucleotide of the present invention described elsewhere herein can be modified using a CRISPR-Cas and/or Cas-based system.

In general, a CRISPR-Cas or CRISPR system as used herein and in other documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g., CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10. 1016/j.molcel.2015.10.008.

CRISPR-Cas systems can generally fall into two classes based on their architectures of their effector molecules, which are each further subdivided by type and subtype. The two class are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

In some embodiments, the CRISPR-Cas system that can be used to modify a polynucleotide of the present invention described herein can be a Class 1 CRISPR-Cas system. In some embodiments, the CRISPR-Cas system that can be used to modify a polynucleotide of the present invention described herein can be a Class 2 CRISPR-Cas system.

### Class 1 CRISPR-Cas Systems

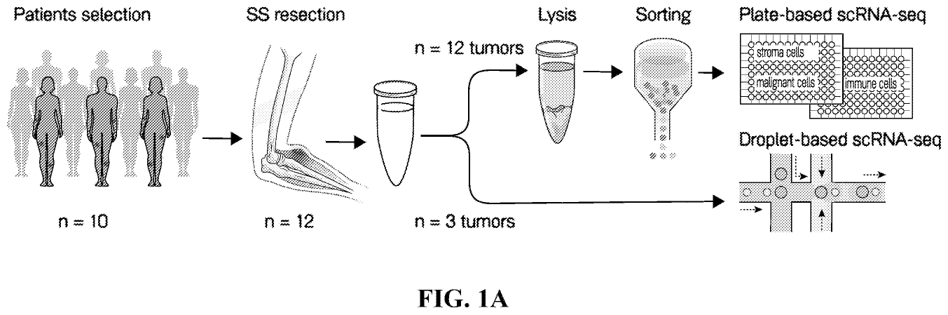

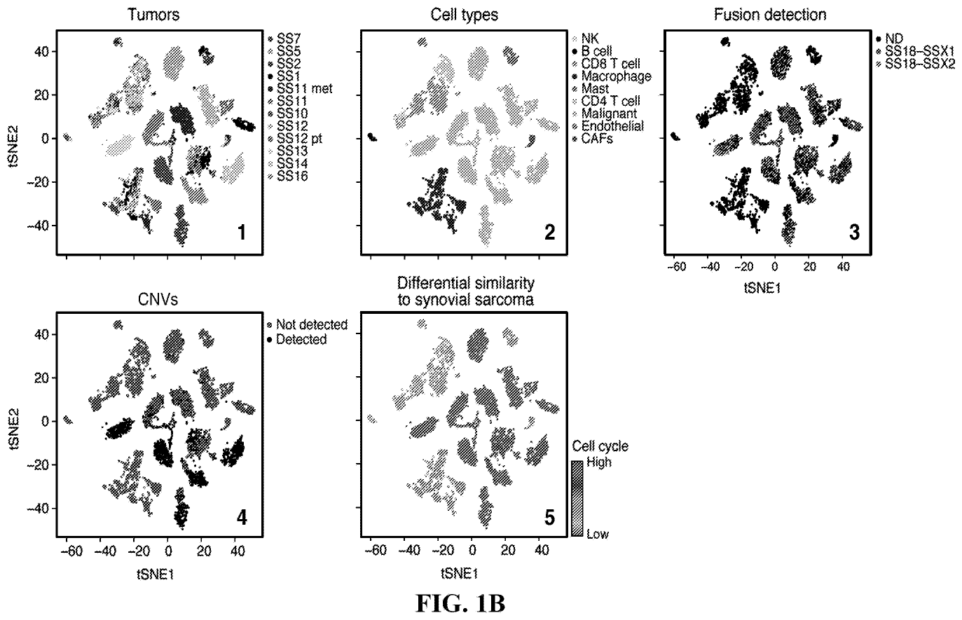

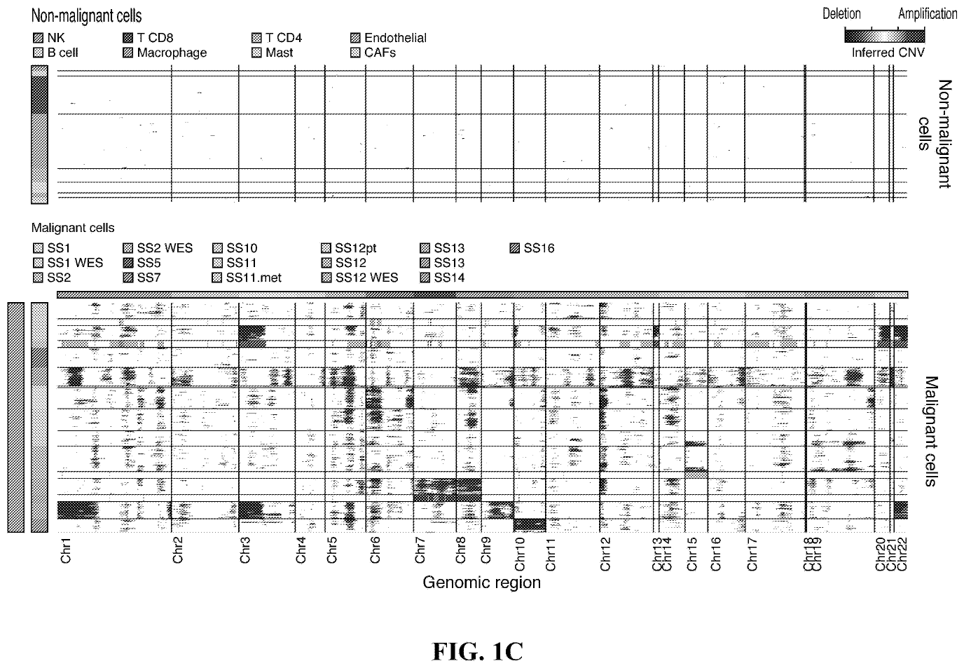

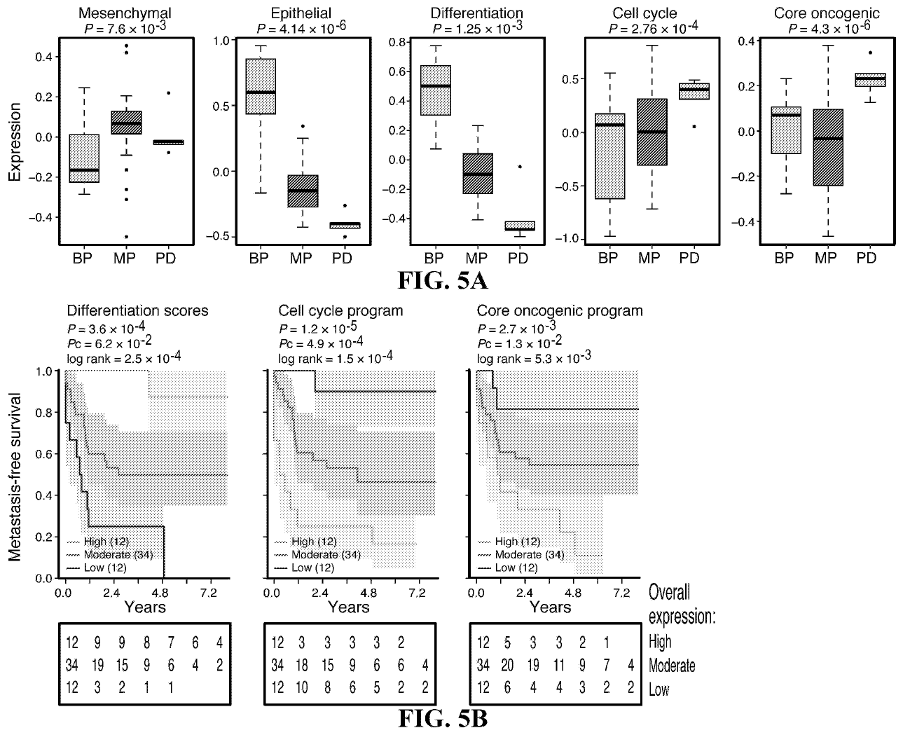

In some embodiments, the CRISPR-Cas system that can be used to modify a polynucleotide of the present invention described herein can be a Class 1 CRISPR-Cas system. Class 1 CRISPR-Cas systems are divided into types I, II, and IV. Makarova et al. 2020. Nat. Rev. 18: 67-83., particularly as described in FIG. 1. Type I CRISPR-Cas systems are divided into 9 subtypes (I-A, I-B, I-C, I-D, I-E, I-F1, I-F2, I-F3, and IG). Makarova et al., 2020. Class 1, Type I CRISPR-Cas systems can contain a Cas3 protein that can have helicase activity. Type III CRISPR-Cas systems are divided into 6 subtypes (III-A, III-B, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes. (IV-A, IV-B, and IV-C). Makarova et al., 2020. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al. 2018. The CRISPR Journal, v. 1, n5, FIG. 5.

The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits (e.g., Cas 5, Cas6, and/or Cas7). RAMP proteins are characterized by having one or more RNA recognition motif domains. In some embodiments, multiple copies of RAMPS can be present. In some embodiments, the Class I CRISPR-Cas system can include 1, 2, 3, 4, 5, 6, 7, 8, 9, 10, 11, 12 or more Cas5, Cas6, and/or Cas 7 proteins. In some embodiments, the Cas6 protein is an RNAse, which can be responsible for pre-crRNA processing. When present in a Class 1 CRISPR-Cas system, Cas6 can be optionally physically associated with the effector complex.

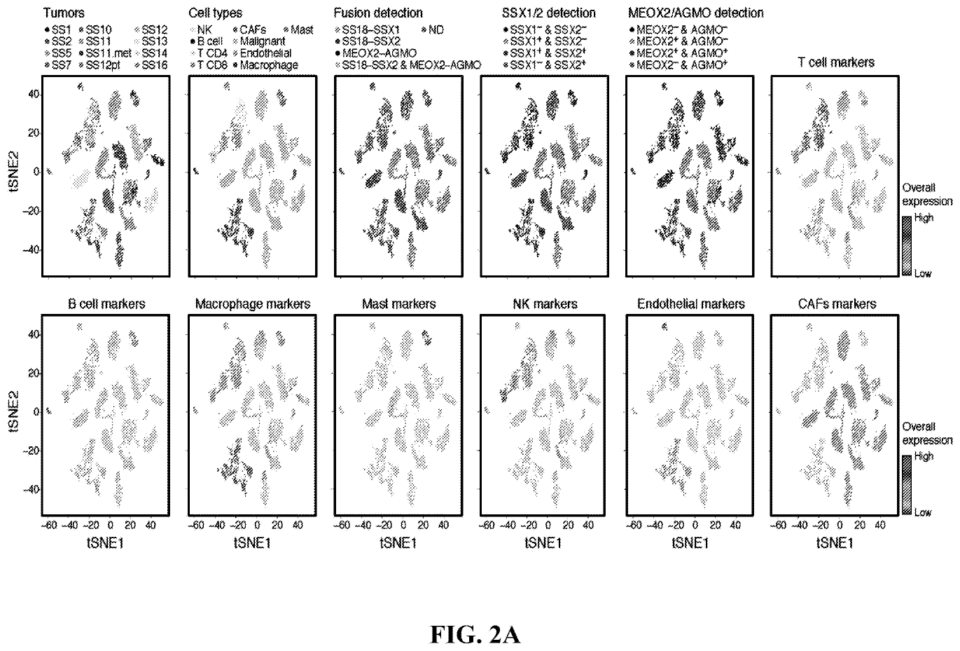

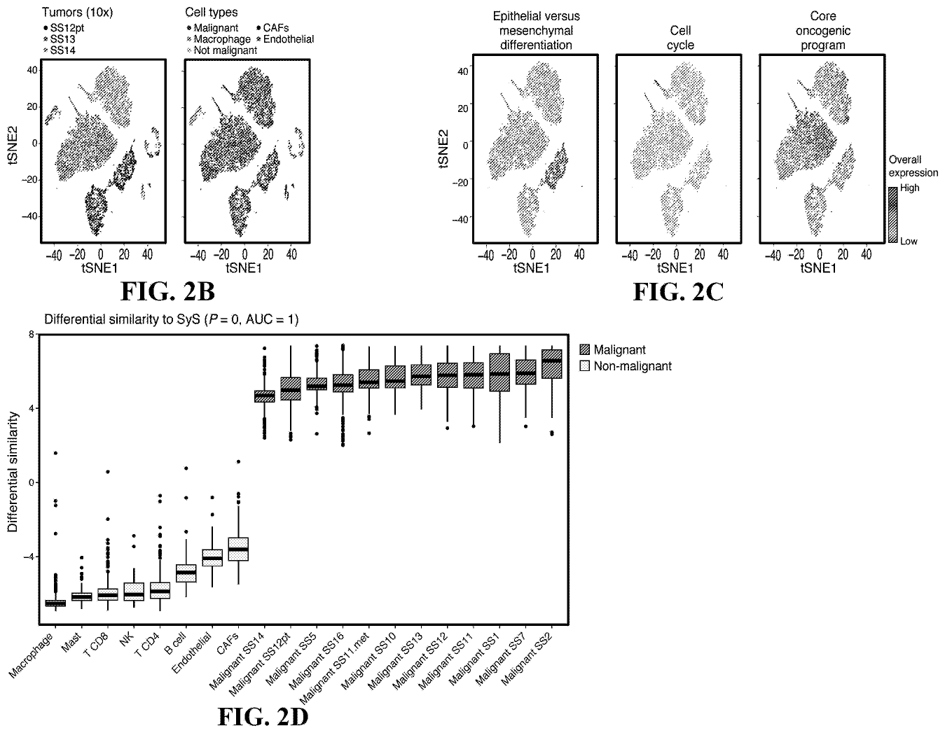

Class 1 CRISPR-Cas system effector complexes can, in some embodiments, also include a large subunit. The large subunit can be composed of or include a Cas8 and/or Cas10 protein. See, e.g., FIGS. 1 and 2. Koonin E V, Makarova K S. 2019. Phil. Trans. R. Soc. B 374: 20180087, DOI: 10.1098/rstb.2018.0087 and Makarova et al. 2020.

Class 1 CRISPR-Cas system effector complexes can, in some embodiments, include a small subunit (for example, Cas11). See, e.g., FIGS. 1 and 2. Koonin E V, Makarova K S. 2019 Origins and Evolution of CRISPR-Cas systems. Phil. Trans. R. Soc. B 374: 20180087, DOI: 10.1098/rstb.2018.0087.

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas5, a Cash, a Cas7, a Cas8, a Cas10, a Cas11, or a combination thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), CasX, and/or Cas14.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SETT/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (WO 2019/005884, WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other Suitable Functional Domains can be Found, for Example, in International Application Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C⋅G base pair into a T⋅A base pair (Komor et al. 2016. Nature. 533:420-424; Nishida et al. 2016. Science. 353; and Li et al. Nat. Biotech. 36:324-327) and ABEs convert an A⋅T base pair to a G⋅C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471.

Other Example Type V base editing systems are described in WO 2018/213708, WO 2018/213726, PCT/US2018/067207, PCT/US2018/067225, and PCT/US2018/067307 which are incorporated by referenced herein.

In certain example embodiments, the base editing system may be a RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA based editor may be used to delete or introduce a post-translation modification site in the expressed mRNA. In contrast to DNA base editors, whose edits are permanent in the modified cell, RNA base editors can provide edits where finer temporal control may be needed, for example in modulating a particular immune response. Example Type VI RNA-base editing systems are described in Cox et al. 2017. Science 358: 1019-1027, WO 2019/005884, WO 2019/005886, WO 2019/071048, PCT/US20018/05179, PCT/US2018/067207, which are incorporated herein by reference. An example FnCas9 system that may be adapted for RNA base editing purposes is described in WO 2016/106236, which is incorporated herein by reference.

An example method for delivery of base-editing systems, including use of a split-intein approach to divide CBE and ABE into reconstituble halves, is described in Levy et al. Nature Biomedical Engineering doi.org/10.1038/s41441-019-0505-5 (2019), which is incorporated herein by reference.

### Prime Editors

In some embodiments, a polynucleotide of the present invention described elsewhere herein can be modified using a prime editing system See e.g. Anzalone et al. 2019. Nature. 576: 149-157. Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion, and combinations thereof. Generally, a prime editing system, as exemplified by PE1, PE2, and PE3 (Id.), can include a reverse transcriptase fused or otherwise coupled or associated with an RNA-programmable nickase, and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g. sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g. a prime editing guide molecule or peg guide molecule) directly into the target site in the target polynucleotide. See e.g. Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g. is a Cas9 nickase). In some embodiments, the Cas polypeptide is fused to the reverse transcriptase. In some embodiments, the Cas polypeptide is linked to the reverse transcriptase.

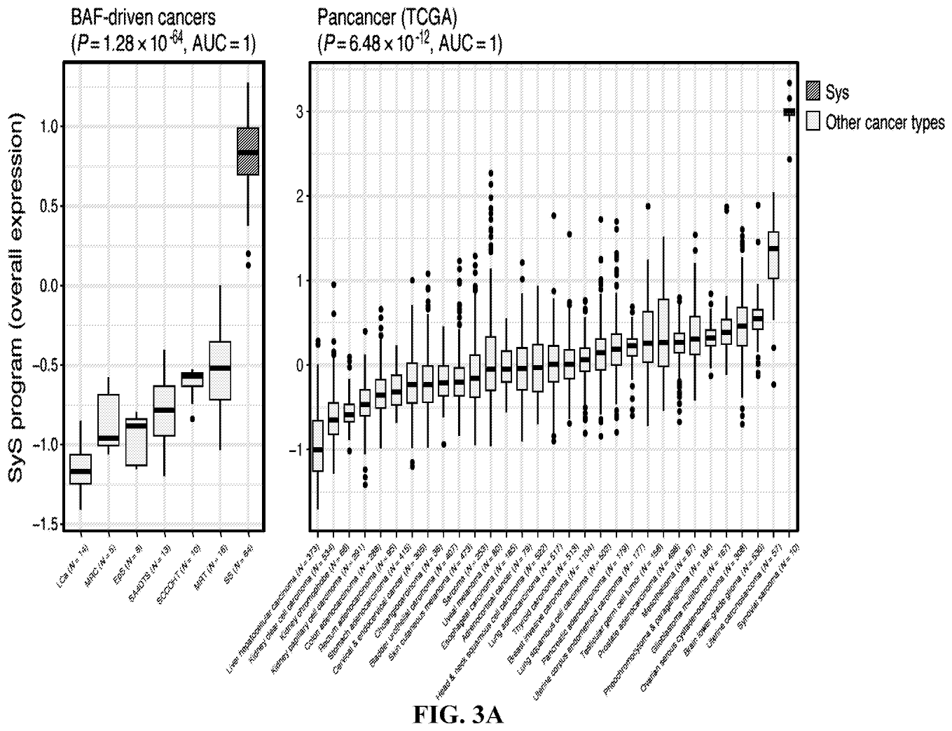

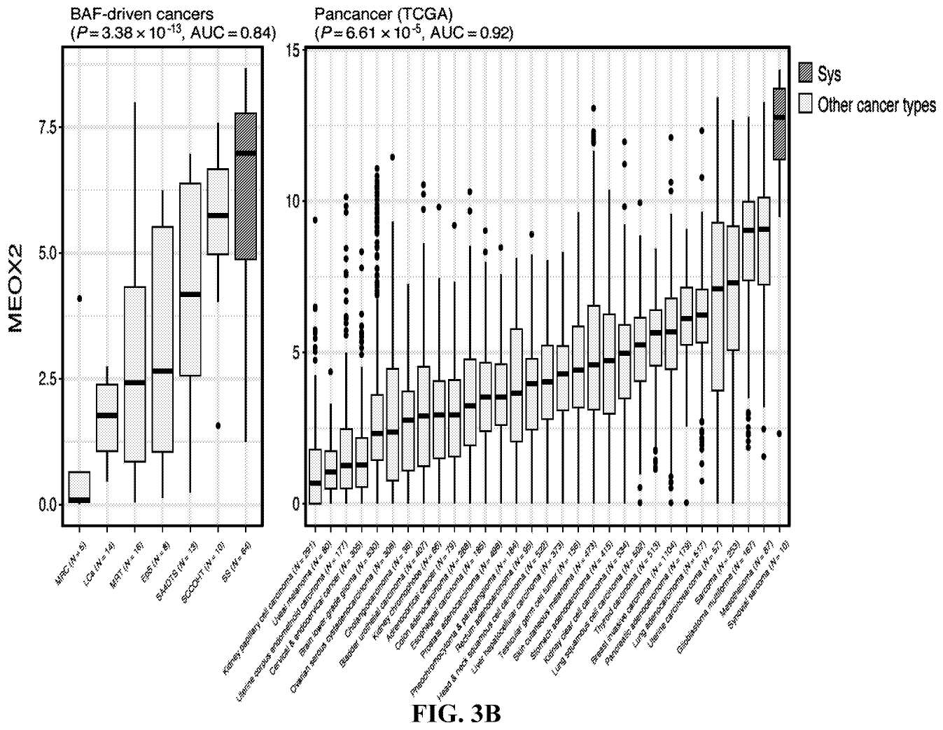

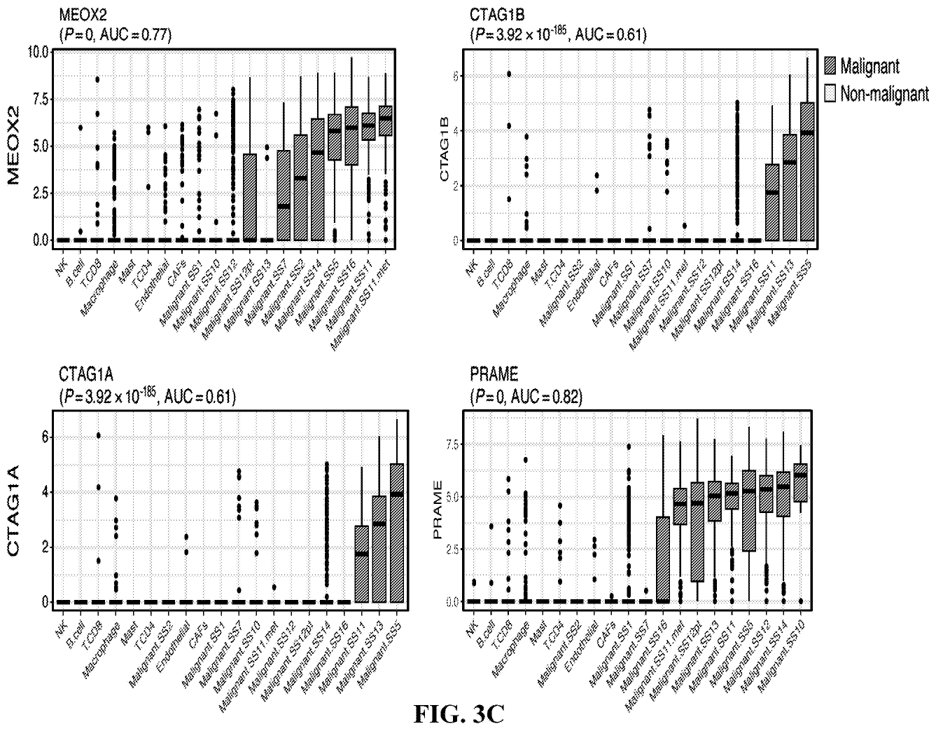

In some embodiments, the prime editing system can be a PE1 system or variant thereof, a PE2 system or variant thereof, or a PE3 (e.g. PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### Guide Molecules

The CRISPR-Cas or Cas-Based system described herein can, in some embodiments, include one or more guide molecules. The terms guide molecule, guide sequence and guide polynucleotide, refer to polynucleotides capable of guiding Cas to a target genomic locus and are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net).

A guide sequence, and hence a nucleic acid-targeting guide may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm, and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many Modifications to Guide Sequences are Known in the Art and are Further Contemplated within the Context of this Invention. Various Modifications May be Used to Increase the Specificity of Binding to the Target Sequence and/or Increase the Activity of the Cas Protein and/or Reduce Off-Target Effects. Example Guide Sequence Modifications are Described in PCT US2019/045582, Specifically Paragraphs [0178]-[0333]. Which is Incorporated Herein by Reference.

### Target Sequences, PAMs, and PFSs

### Target Sequences

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table 3 below shows several Cas polypeptides and the PAM sequence they recognize.

TABLE 3
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including LbCpf1 and | TTTV |
| AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a preferred embodiment, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′ end of the target RNA. The presence of a C at the corresponding crRNA repeat site can indicate that nucleotide pairing at this position is rejected. However, some Cas13 proteins (e.g., LwaCas13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Zinc Finger Nucleases

In some embodiments, the MARC polynucleotide is modified using a Zinc Finger nuclease or system thereof. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Sequences Related to Nucleus Targeting and Transportation

In some embodiments, one or more components (e.g., the Cas protein and/or deaminase) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID No. 7) or PKKKRKVEAS (SEQ ID No. 8); the NLS from nucleoplasmin (e.g., the nucleoplasmin bipartite NLS with the sequence KRPAATKKAGQAKKKK (SEQ ID No. 9)); the c-myc NLS having the amino acid sequence PAAKRVKLD (SEQ ID No. 10) or RQRRNELKRSP (SEQ ID No. 11); the hRNPA1 M9 NLS having the sequence NQSSNFGPMKGGNFGGRSSGPYGGGGQYFAKPRNQGGY (SEQ ID No. 12); the sequence RMRIZFKNKGKDTAELRRRRVEVSVELRKAKKDEQILKRRNV (SEQ ID No. 13) of the IBB domain from importin-alpha; the sequences VSRKRPRP (SEQ ID No. 14) and PPKKARED (SEQ ID No. 15) of the myoma T protein; the sequence PQPKKKPL (SEQ ID No. 16) of human p53; the sequence SALIKKKKKMAP (SEQ ID No. 17) of mouse c-abl IV; the sequences DRLRR (SEQ ID No. 18) and PKQKKRK (SEQ ID No. 19) of the influenza virus NS1; the sequence RKLKKKIKKL (SEQ ID No. 20) of the Hepatitis virus delta antigen; the sequence REKKKFLKRR (SEQ ID No. 21) of the mouse Mx1 protein; the sequence KRKGDEVDGVDEVAKKKSKK (SEQ ID No. 22) of the human poly(ADP-ribose) polymerase; and the sequence RKCLQAGMNLEARKTKK (SEQ ID No. 23) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein, or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g. aptamers) for adapter proteins, which may be linked to or fused to an nucleotide deaminase or catalytic domain thereof. When such a guide forms a CRISPR complex (e.g., CRISPR-Cas protein binding to guide and target) the adapter proteins bind and, the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g. due to steric hindrance within the three dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### Templates

In some embodiments, the composition for engineering cells comprise a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include sequence that corresponds to both, a first site on the target sequence that is cleaved in a first Cas protein mediated event, and a second site on the target sequence that is cleaved in a second Cas protein mediated event.

In certain embodiments, the template nucleic acid can include sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include sequence which, when integrated, results in: decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include sequence which results in: a change in sequence of 1, 2, 3, 4, 5, 6, 7, 8, 9, 10, 1 1, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 1 10+/−10, 120+/−10, 130+/−10, 140+/−10, 150+/−10, 160+/−10, 170+/−10, 1 80+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 1 10+/−20, 120+/−20, 130+/−20, 140+/−20, I 50+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149).

### TALE Nucleases

In some embodiments, a TALE nuclease or TALE nuclease system can be used to modify a MARC polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 3)
M D P I R S R T P S P A R E L L S G P Q P D G V Q
P T A D R G V S P P A G G P L D G L P A R R T M S
R T R L P S P P A P S P A F S A D S F S D L L R Q
F D P S L F N T S L F D S L P P F G A H H T E A A
T G E W D E V Q S G L R A A D A P P P T M R V A V
T A A R P P R A K P A P R R R A A Q P S D A S P A
A Q V D L R T L G Y S Q Q Q Q E K I K P K V R S T
V A Q H H E A L V G H G F T H A H I V A L S Q H P
A A L G T V A V K Y Q D M I A A L P E A T H E A I
V G V G K Q W S G A R A L E A L L T V A G E L R G
P P L Q L D T G Q L L K I A K R G G V T A V E A V
H A W R N A L T G A P L N

An exemplary amino acid sequence of a C-terminal capping region is:

(SEQ ID NO: 4)
R P A L E S I V A Q L S R P D P A L A A L T N D H
L V A L A C L G G R P A L D A V K K G L P H A P A
L I K R T N R R I P E R T S H R V A D H A Q V V R
V L G F F Q C H S H P A Q A F D D A M T Q F G M S
R H G L L Q L F R R V G V T E L E A R S G T L P P
A S Q R W D R I L Q A S G M K R A K P S P T S T Q
T P D Q A S L H A F A D S L E R D L D A P S P M H
E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

### Meganucleases

In some embodiments, a meganuclease or system thereof can be used to modify a MARC polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated by reference.

### Guide Molecules

The methods described herein may be used to screen inhibition of CRISPR systems employing different types of guide molecules. As used herein, the term “guide sequence” and “guide molecule” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. In some embodiments, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example embodiments, the guide molecule comprises a guide sequence that may be designed to have at least one mismatch with the target sequence, such that a RNA duplex formed between the guide sequence and the target sequence. Accordingly, the degree of complementarity is preferably less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer. In certain example embodiment, the guide sequence is 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the guide sequence is an RNA sequence of between 10 to 50 nt in length, but more particularly of about 20-30 nt advantageously about 20 nt, 23 to 25 nt or 24 nt. The guide sequence is selected so as to ensure that it hybridizes to the target sequence. This is described in greater detail below. Selection can encompass further steps which increase efficacy and specificity.

In some embodiments, the guide sequence has a canonical length (e.g., about 15-30 nt) is used to hybridize with the target RNA or DNA. In some embodiments, a guide molecule is longer than the canonical length (e.g., >30 nt) is used to hybridize with the target RNA or DNA, such that a region of the guide sequence hybridizes with a region of the RNA or DNA strand outside of the Cas-guide target complex. This can be of interest where additional modifications, such deamination of nucleotides is of interest. In alternative embodiments, it is of interest to maintain the limitation of the canonical guide sequence length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In some embodiments, it is of interest to reduce the susceptibility of the guide molecule to RNA cleavage, such as to cleavage by Cas13. Accordingly, in particular embodiments, the guide molecule is adjusted to avoid cleavage by Cas13 or other RNA-cleaving enzymes.

In certain embodiments, the guide molecule comprises non-naturally occurring nucleic acids and/or non-naturally occurring nucleotides and/or nucleotide analogs, and/or chemical modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′ phosphorothioate (MS), S-constrained ethyl(cEt), or 2′-O-methyl 3′ thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target RNA and one or more deoxyribonucletides and/or nucleotide analogs in a region that binds to Cas13. In an embodiment of the invention, deoxyribonucleotides and/or nucleotide analogs are incorporated in engineered guide structures, such as, without limitation, stem-loop regions, and the seed region. For Cas13 guide, in certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemically modified with 2′-O-methyl (M), 2′-O-methyl 3′ phosphorothioate (MS), S-constrained ethyl(cEt), or 2′-O-methyl 3′ thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemically modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6:e25312, DOI:10.7554).

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N1-methylpseudouridine (me1Ψ), 5-methoxyuridine(5moU), inosine, 7-methylguanosine, 2′-O-methyl 3′phosphorothioate (MS), S-constrained ethyl(cEt), phosphorothioate (PS), or 2′-O-methyl 3′thioPACE (MSP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 to 10 nucleotides in the 3′-terminus are chemically modified. Such chemical modifications at the 3′-terminus of the Cas13 CrRNA may improve Cas13 activity. In a specific embodiment, 1, 2, 3, 4, 5, 6, 7, 8, 9 or 10 nucleotides in the 3′-terminus are replaced with 2′-fluoro analogues. In a specific embodiment, 1, 2, 3, 4, 5, 6, 7, 8, 9 or 10 nucleotides in the 3′-terminus are replaced with 2′-O-methyl (M) analogs.

In some embodiments, the loop of the 5′-handle of the guide is modified. In some embodiments, the loop of the 5′-handle of the guide is modified to have a deletion, an insertion, a split, or chemical modifications. In certain embodiments, the modified loop comprises 3, 4, or 5 nucleotides. In certain embodiments, the loop comprises the sequence of UCUU, UUUU, UAUU, or UGUU.

In some embodiments, the guide molecule forms a stemloop with a separate non-covalently linked sequence, which can be DNA or RNA. In particular embodiments, the sequences forming the guide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once this sequence is functionalized, a covalent chemical bond or linkage can be formed between this sequence and the direct repeat sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, these stem-loop forming sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In certain embodiments, the guide molecule comprises (1) a guide sequence capable of hybridizing to a target locus and (2) a tracr mate or direct repeat sequence whereby the direct repeat sequence is located upstream (i.e., 5′) from the guide sequence. In a particular embodiment the seed sequence (i.e. the sequence essential critical for recognition and/or hybridization to the sequence at the target locus) of th guide sequence is approximately within the first 10 nucleotides of the guide sequence.

In a particular embodiment the guide molecule comprises a guide sequence linked to a direct repeat sequence, wherein the direct repeat sequence comprises one or more stem loops or optimized secondary structures. In particular embodiments, the direct repeat has a minimum length of 16 nts and a single stem loop. In further embodiments the direct repeat has a length longer than 16 nts, preferably more than 17 nts, and has more than one stem loops or optimized secondary structures. In particular embodiments the guide molecule comprises or consists of the guide sequence linked to all or part of the natural direct repeat sequence. A typical Type V or Type VI CRISPR-cas guide molecule comprises (in 3′ to 5′ direction or in 5′ to 3′ direction): a guide sequence a first complimentary stretch (the “repeat”), a loop (which is typically 4 or 5 nucleotides long), a second complimentary stretch (the “anti-repeat” being complimentary to the repeat), and a poly A (often poly U in RNA) tail (terminator). In certain embodiments, the direct repeat sequence retains its natural architecture and forms a single stem loop. In particular embodiments, certain aspects of the guide architecture can be modified, for example by addition, subtraction, or substitution of features, whereas certain other aspects of guide architecture are maintained. Preferred locations for engineered guide molecule modifications, including but not limited to insertions, deletions, and substitutions include guide termini and regions of the guide molecule that are exposed when complexed with the CRISPR-Cas protein and/or target, for example the stemloop of the direct repeat sequence.

In particular embodiments, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-10 and Y2-10 (wherein X and Y represent any complementary set of nucleotides) may be contemplated. In one aspect, the stem made of the X and Y nucleotides, together with the loop will form a complete hairpin in the overall secondary structure; and, this may be advantageous and the amount of base pairs can be any amount that forms a complete hairpin. In one aspect, any complementary X:Y basepairing sequence (e.g., as to length) is tolerated, so long as the secondary structure of the entire guide molecule is preserved. In one aspect, the loop that connects the stem made of X:Y basepairs can be any sequence of the same length (e.g., 4 or 5 nucleotides) or longer that does not interrupt the overall secondary structure of the guide molecule. In one aspect, the stemloop can further comprise, e.g. an MS2 aptamer. In one aspect, the stem comprises about 5-7 bp comprising complementary X and Y sequences, although stems of more or fewer basepairs are also contemplated. In one aspect, non-Watson Crick basepairing is contemplated, where such pairing otherwise generally preserves the architecture of the stemloop at that position.

In particular embodiments the natural hairpin or stemloop structure of the guide molecule is extended or replaced by an extended stemloop. It has been demonstrated that extension of the stem can enhance the assembly of the guide molecule with the CRISPR-Cas protein (Chen et al. Cell. (2013); 155(7): 1479-1491). In particular embodiments the stem of the stemloop is extended by at least 1, 2, 3, 4, 5 or more complementary basepairs (i.e. corresponding to the addition of 2, 4, 6, 8, 10 or more nucleotides in the guide molecule). In particular embodiments these are located at the end of the stem, adjacent to the loop of the stemloop.

In particular embodiments, the susceptibility of the guide molecule to RNAses or to decreased expression can be reduced by slight modifications of the sequence of the guide molecule which do not affect its function. For instance, in particular embodiments, premature termination of transcription, such as premature transcription of U6 Pol-III, can be removed by modifying a putative Pol-III terminator (4 consecutive U's) in the guide molecules sequence. Where such sequence modification is required in the stemloop of the guide molecule, it is preferably ensured by a basepair flip.

In a particular embodiment, the direct repeat may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In some embodiments, the guide molecule forms a duplex with a target RNA comprising at least one target cytosine residue to be edited. Upon hybridization of the guide RNA molecule to the target RNA, the cytidine deaminase binds to the single strand RNA in the duplex made accessible by the mismatch in the guide sequence and catalyzes deamination of one or more target cytosine residues comprised within the stretch of mismatching nucleotides.

A guide sequence, and hence a nucleic acid-targeting guide RNA may be selected to target any target nucleic acid sequence. The target sequence may be mRNA.

In certain embodiments, the target sequence should be associated with a PAM (protospacer adjacent motif) or PFS (protospacer flanking sequence or site); that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM. In the embodiments of the present invention where the CRISPR-Cas protein is a Cas13 protein, the complementary sequence of the target sequence is downstream or 3′ of the PAM or upstream or 5′ of the PAM. The precise sequence and length requirements for the PAM differ depending on the Cas13 protein used, but PAMs are typically 2-5 base pair sequences adjacent the protospacer (that is, the target sequence). Examples of the natural PAM sequences for different Cas13 orthologues are provided herein below and the skilled person will be able to identify further PAM sequences for use with a given Cas13 protein.

Further, engineering of the PAM Interacting (PI) domain may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously.

In particular embodiment, the guide is an escorted guide. By “escorted” is meant that the CRISPR-Cas system or complex or guide is delivered to a selected time or place within a cell, so that activity of the CRISPR-Cas system or complex or guide is spatially or temporally controlled. For example, the activity and destination of the 3 CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted CRISPR-Cas systems or complexes have a guide molecule with a functional structure designed to improve guide molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green fluorescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, in particular embodiments, the guide molecule is modified, e.g., by one or more aptamer(s) designed to improve guide molecule delivery, including delivery across the cellular membrane, to intracellular compartments, or into the nucleus. Such a structure can include, either in addition to the one or more aptamer(s) or without such one or more aptamer(s), moiety(ies) so as to render the guide molecule deliverable, inducible or responsive to a selected effector. The invention accordingly comprehends an guide molecule that responds to normal or pathological physiological conditions, including without limitation pH, hypoxia, O₂ concentration, temperature, protein concentration, enzymatic concentration, lipid structure, light exposure, mechanical disruption (e.g. ultrasound waves), magnetic fields, electric fields, or electromagnetic radiation. Inducible systems and energy application can be as described for example, in International Patent Publication WO2019232542 at [0275]-[0302], incorporated herein by reference.

In particular embodiments, the guide molecule is modified by a secondary structure to increase the specificity of the CRISPR-Cas system and the secondary structure can protect against exonuclease activity and allow for 5′ additions to the guide sequence also referred to herein as a protected guide molecule.

In one aspect, the invention provides for hybridizing a “protector RNA” to a sequence of the guide molecule, wherein the “protector RNA” is an RNA strand complementary to the 3′ end of the guide molecule to thereby generate a partially double-stranded guide RNA. In an embodiment of the invention, protecting mismatched bases (i.e. the bases of the guide molecule which do not form part of the guide sequence) with a perfectly complementary protector sequence decreases the likelihood of target RNA binding to the mismatched basepairs at the 3′ end. In particular embodiments of the invention, additional sequences comprising an extended length may also be present within the guide molecule such that the guide comprises a protector sequence within the guide molecule. This “protector sequence” ensures that the guide molecule comprises a “protected sequence” in addition to an “exposed sequence” (comprising the part of the guide sequence hybridizing to the target sequence). In particular embodiments, the guide molecule is modified by the presence of the protector guide to comprise a secondary structure such as a hairpin. Advantageously there are three or four to thirty or more, e.g., about 10 or more, contiguous base pairs having complementarity to the protected sequence, the guide sequence or both. It is advantageous that the protected portion does not impede thermodynamics of the CRISPR-Cas system interacting with its target. By providing such an extension including a partially double stranded guide molecule, the guide molecule is considered protected and results in improved specific binding of the CRISPR-Cas complex, while maintaining specific activity.

In particular embodiments, use is made of a truncated guide (tru-guide), i.e., a guide molecule which comprises a guide sequence which is truncated in length with respect to the canonical guide sequence length. As described by Nowak et al. (Nucleic Acids Res (2016) 44 (20): 9555-9564), such guides may allow catalytically active CRISPR-Cas enzyme to bind its target without cleaving the target RNA. In particular embodiments, a truncated guide is used which allows the binding of the target but retains only nickase activity of the CRISPR-Cas enzyme.

In addition to the above CRISPR-Cas systems, the CRISPR-Cas may be a base editor version, thereof i.e. a catalytically dead Cas linked or fused to a nucleotide deaminase domain. The Cas may be a RNA-binding (e.g. Type VI) on DNA-binding Cas (Type II or V). In certain embodiments, the compositions, systems, and methods may be designed for use with Class 2 systems. In certain example embodiments, the Class 2 systems may be Type II, Type V, and Type VI systems as described in Makarova et al. “Evolutionary classification of CRISPR-Cas systems: a burst of class 2 and derived variants” Nature Reviews Microbiology, 18:67-81 (February 2020), incorporated herein by reference. The distinguishing feature of these types is that their effector complexes consist of a single, large, multi-domain protein. Type V systems differ from Type II effectors (e.g. Cas9) contain two nuclear domains that are each responsible for the cleavage of one strand of the target DNA, with the HNH nuclease inserted inside the Ruv-C like nuclease domain sequence. The Type V systems (e.g. Cas12) only contain a RuvC-like nuclease domain that cleaves both strands. Type VI (Cas13) are unrelated to the effectors of type II and V systems, contain two HEPN domains and target RNA. Cas13 proteins also display collateral activity that is triggered by target recognition. Some Type V systems have also been found to possess this collateral activity two single-stranded DNA in in vitro contexts.

certain example embodiments, the Type V CRISPR-Cas is Cas12a, Cas12b, or Cas12c.

The present invention also contemplates use of the CRISPR-Cas system and the base editor described herein, for treatment in a variety of diseases and disorders. In some embodiments, the invention described herein relates to a method for therapy in which cells are edited ex vivo by CRISPR or the base editor to modulate at least one gene, with subsequent administration of the edited cells to a patient in need thereof. In some embodiments, the editing involves knocking in, knocking out or knocking down expression of at least one target gene in a cell. In particular embodiments, the editing inserts an exogenous, gene, minigene or sequence, which may comprise one or more exons and introns or natural or synthetic introns into the locus of a target gene, a hot-spot locus, a safe harbor locus of the gene genomic locations where new genes or genetic elements can be introduced without disrupting the expression or regulation of adjacent genes, or correction by insertions or deletions one or more mutations in DNA sequences that encode regulatory elements of a target gene. In some embodiment, the editing comprise introducing one or more point mutations in a nucleic acid (e.g., a genomic DNA) in a target cell.

The present disclosure also provides for a base editing system. In general, such a system may comprise a deaminase (e.g., an adenosine deaminase or cytidine deaminase) fused with a Cas protein. The Cas protein may be a dead Cas protein or a Cas nickase protein. In certain examples, the system comprises a mutated form of an adenosine deaminase fused with a dead CRISPR-Cas or CRISPR-Cas nickase. The mutated form of the adenosine deaminase may have both adenosine deaminase and cytidine deaminase activities.

In one aspect, the present disclosure provides an engineered adenosine deaminase. The engineered adenosine deaminase may comprise one or more mutations herein. In some embodiments, the engineered adenosine deaminase has cytidine deaminase activity. In certain examples, the engineered adenosine deaminase has both cytidine deaminase activity and adenosine deaminase. In some cases, the modifications by base editors herein may be used for targeting post-translational signaling or catalysis.

In one aspect, the invention provides a method of modifying or editing a target transcript in a eukaryotic cell. In some embodiments, the method comprises allowing a CRISPR-Cas effector module complex to bind to the target polynucleotide to effect RNA base editing, wherein the CRISPR-Cas effector module complex comprises a Cas effector module complexed with a guide sequence hybridized to a target sequence within said target polynucleotide, wherein said guide sequence is linked to a direct repeat sequence. In some embodiments, the Cas effector module comprises a catalytically inactive CRISPR-Cas protein. In some embodiments, the guide sequence is designed to introduce one or more mismatches to the RNA/RNA duplex formed between the target sequence and the guide sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytindine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof is capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof. See, e.g. Levy et al., doi:10.1038/s41551-019-0501-5, Rees et al, doi: 10.1038/s41467-019-09983-4; Komor et al, Nature 533(7603), 420-424, Gaudellim et al, Nature 551 (7681), 464-471, Lee, et al., Nature Commun. 9:4804 1-5(2018), Song et al., Biomed End. 36, 536-539 (2018), Lee et al., Sci. Rep. 9, 1662 (2019), Thuronyi, et al., Nat. Biotechnol. 37, 1070-1079 (2019), Anzalone, et al., nature 576 149-157 (2019), and Richter et al., Nat Biotechnol in press (2020), all incorporated herein by reference. Reference is also made to International Patent Publication Nos. WO 2019/005884, WO 2019/005886, WO 2020/028555, WO 2019/060746, WO 2019/071048, WO 2019/084063, and Abudayyeh et al., Science 365:6451, 382-386, doi: 10.1126/science.aax7063, incorporated herein by reference.

### RNAi

In certain embodiments, the genetic modifying agent is RNAi (e.g., shRNA). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

It will be understood by the skilled person that treating as referred to herein encompasses enhancing treatment, or improving treatment efficacy. Treatment may include inhibition of an inflammatory response, enhancing an immune response, tumor regression as well as inhibition of tumor growth, metastasis or tumor cell proliferation, or inhibition or reduction of otherwise deleterious effects associated with the tumor.

Efficaciousness of treatment is determined in association with any known method for diagnosing or treating the particular disease. The invention comprehends a treatment method comprising any one of the methods or uses herein discussed.

The phrase “therapeutically effective amount” as used herein refers to a sufficient amount of a drug, agent, or compound to provide a desired therapeutic effect.

As used herein “patient” refers to any human being receiving or who may receive medical treatment and is used interchangeably herein with the term “subject”.

Therapy or treatment according to the invention may be performed alone or in conjunction with another therapy, and may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, the stage of the cancer, and how the patient responds to the treatment. Additionally, a person having a greater risk of developing an inflammatory response (e.g., a person who is genetically predisposed or predisposed to allergies or a person having a disease characterized by episodes of inflammation) may receive prophylactic treatment to inhibit or delay symptoms of the disease.

### Administration

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous ab sorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

The medicaments of the invention are prepared in a manner known to those skilled in the art, for example, by means of conventional dissolving, lyophilizing, mixing, granulating or confectioning processes. Methods well known in the art for making formulations are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York.

Administration of medicaments of the invention may be by any suitable means that results in a compound concentration that is effective for treating or inhibiting (e.g., by delaying) the development of a disease. The compound is admixed with a suitable carrier substance, e.g., a pharmaceutically acceptable excipient that preserves the therapeutic properties of the compound with which it is administered. One exemplary pharmaceutically acceptable excipient is physiological saline. The suitable carrier substance is generally present in an amount of 1-95% by weight of the total weight of the medicament. The medicament may be provided in a dosage form that is suitable for administration. Thus, the medicament may be in form of, e.g., tablets, capsules, pills, powders, granulates, suspensions, emulsions, solutions, gels including hydrogels, pastes, ointments, creams, plasters, drenches, delivery devices, injectables, implants, sprays, or aerosols.

The agents disclosed herein may be used in a pharmaceutical composition when combined with a pharmaceutically acceptable carrier. Such compositions comprise a therapeutically-effective amount of the agent and a pharmaceutically acceptable carrier. Such a composition may also further comprise (in addition to an agent and a carrier) diluents, fillers, salts, buffers, stabilizers, solubilizers, and other materials well known in the art. Compositions comprising the agent can be administered in the form of salts provided the salts are pharmaceutically acceptable. Salts may be prepared using standard procedures known to those skilled in the art of synthetic organic chemistry.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. It will be understood that, as used herein, references to specific agents (e.g., neuromedin U receptor agonists or antagonists), also include the pharmaceutically acceptable salts thereof.

Methods of administrating the pharmacological compositions, including agonists, antagonists, antibodies or fragments thereof, to an individual include, but are not limited to, intradermal, intrathecal, intramuscular, intraperitoneal, intravenous, subcutaneous, intranasal, epidural, by inhalation, and oral routes. The compositions can be administered by any convenient route, for example by infusion or bolus injection, by absorption through epithelial or mucocutaneous linings (for example, oral mucosa, rectal and intestinal mucosa, and the like), ocular, and the like and can be administered together with other biologically-active agents. Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment; this may be achieved by, for example, and not by way of limitation, local infusion during surgery, topical application, by injection, by means of a catheter, by means of a suppository, or by means of an implant.

Various delivery systems are known and can be used to administer the pharmacological compositions including, but not limited to, encapsulation in liposomes, microparticles, microcapsules; minicells; polymers; capsules; tablets; and the like. In one embodiment, the agent may be delivered in a vesicle, in particular a liposome. In a liposome, the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028 and 4,737,323. In yet another embodiment, the pharmacological compositions can be delivered in a controlled release system including, but not limited to: a delivery pump (See, for example, Saudek, et al., New Engl. J. Med. 321: 574 (1989) and a semi-permeable polymeric material (See, for example, Howard, et al., J. Neurosurg. 71: 105 (1989)). Additionally, the controlled release system can be placed in proximity of the therapeutic target (e.g., a tumor), thus requiring only a fraction of the systemic dose. See, for example, Goodson, In: Medical Applications of Controlled Release, 1984. (CRC Press, Boca Raton, Fla.).

The amount of the agents which will be effective in the treatment of a particular disorder or condition will depend on the nature of the disorder or condition, and may be determined by standard clinical techniques by those of skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of the agent with which to treat each individual patient. In certain embodiments, the attending physician will administer low doses of the agent and observe the patient's response. Larger doses of the agent may be administered until the optimal therapeutic effect is obtained for the patient, and at that point the dosage is not increased further. In general, the daily dose range lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases. In certain embodiments, suitable dosage ranges for intravenous administration of the agent are generally about 5-500 micrograms (μg) of active compound per kilogram (Kg) body weight. Suitable dosage ranges for intranasal administration are generally about 0.01 pg/kg body weight to 1 mg/kg body weight. In certain embodiments, a composition containing an agent of the present invention is subcutaneously injected in adult patients with dose ranges of approximately 5 to 5000 μg/human and preferably approximately 5 to 500 μg/human as a single dose. It is desirable to administer this dosage 1 to 3 times daily. Effective doses may be extrapolated from dose-response curves derived from in vitro or animal model test systems. Suppositories generally contain active ingredient in the range of 0.5% to 10% by weight; oral formulations preferably contain 10% to 95% active ingredient. Ultimately the attending physician will decide on the appropriate duration of therapy using compositions of the present invention. Dosage will also vary according to the age, weight and response of the individual patient.

Methods for administering antibodies for therapeutic use is well known to one skilled in the art. In certain embodiments, small particle aerosols of antibodies or fragments thereof may be administered (see e.g., Piazza et al., J. Infect. Dis., Vol. 166, pp. 1422-1424, 1992; and Brown, Aerosol Science and Technology, Vol. 24, pp. 45-56, 1996). In certain embodiments, antibodies are administered in metered-dose propellant driven aerosols. In preferred embodiments, antibodies are used as agonists to depress inflammatory diseases or allergen-induced asthmatic responses. In certain embodiments, antibodies may be administered in liposomes, i.e., immunoliposomes (see, e.g., Maruyama et al., Biochim. Biophys. Acta, Vol. 1234, pp. 74-80, 1995). In certain embodiments, immunoconjugates, immunoliposomes or immunomicrospheres containing an agent of the present invention is administered by inhalation.

In certain embodiments, antibodies may be topically administered to mucosa, such as the oropharynx, nasal cavity, respiratory tract, gastrointestinal tract, eye such as the conjunctival mucosa, vagina, urogenital mucosa, or for dermal application. In certain embodiments, antibodies are administered to the nasal, bronchial or pulmonary mucosa. In order to obtain optimal delivery of the antibodies to the pulmonary cavity in particular, it may be advantageous to add a surfactant such as a phosphoglyceride, e.g. phosphatidylcholine, and/or a hydrophilic or hydrophobic complex of a positively or negatively charged excipient and a charged antibody of the opposite charge.

Other excipients suitable for pharmaceutical compositions intended for delivery of antibodies to the respiratory tract mucosa may be a) carbohydrates, e.g., monosaccharides such as fructose, galactose, glucose. D-mannose, sorbiose, and the like; disaccharides, such as lactose, trehalose, cellobiose, and the like; cyclodextrins, such as 2-hydroxypropyl-β-cyclodextrin; and polysaccharides, such as raffinose, maltodextrins, dextrans, and the like; b) amino acids, such as glycine, arginine, aspartic acid, glutamic acid, cysteine, lysine and the like; c) organic salts prepared from organic acids and bases, such as sodium citrate, sodium ascorbate, magnesium gluconate, sodium gluconate, tromethamine hydrochloride, and the like: d) peptides and proteins, such as aspartame, human serum albumin, gelatin, and the like; e) alditols, such mannitol, xylitol, and the like, and f) polycationic polymers, such as chitosan or a chitosan salt or derivative.

For dermal application, the antibodies of the present invention may suitably be formulated with one or more of the following excipients: solvents, buffering agents, preservatives, humectants, chelating agents, antioxidants, stabilizers, emulsifying agents, suspending agents, gel-forming agents, ointment bases, penetration enhancers, and skin protective agents.

Examples of solvents are e.g. water, alcohols, vegetable or marine oils (e.g. edible oils like almond oil, castor oil, cacao butter, coconut oil, corn oil, cottonseed oil, linseed oil, olive oil, palm oil, peanut oil, poppy seed oil, rapeseed oil, sesame oil, soybean oil, sunflower oil, and tea seed oil), mineral oils, fatty oils, liquid paraffin, polyethylene glycols, propylene glycols, glycerol, liquid polyalkylsiloxanes, and mixtures thereof.

Examples of buffering agents are e.g. citric acid, acetic acid, tartaric acid, lactic acid, hydrogenphosphoric acid, diethyl amine etc. Suitable examples of preservatives for use in compositions are parabenes, such as methyl, ethyl, propyl p-hydroxybenzoate, butylparaben, isobutylparaben, isopropylparaben, potassium sorbate, sorbic acid, benzoic acid, methyl benzoate, phenoxyethanol, bronopol, bronidox, MDM hydantoin, iodopropynyl butylcarbamate, EDTA, benzalconium chloride, and benzylalcohol, or mixtures of preservatives.

Examples of humectants are glycerin, propylene glycol, sorbitol, lactic acid, urea, and mixtures thereof.

Examples of antioxidants are butylated hydroxy anisole (BHA), ascorbic acid and derivatives thereof, tocopherol and derivatives thereof, cysteine, and mixtures thereof.

Examples of emulsifying agents are naturally occurring gums, e.g. gum acacia or gum tragacanth; naturally occurring phosphatides, e.g. soybean lecithin, sorbitan monooleate derivatives: wool fats; wool alcohols; sorbitan esters; monoglycerides; fatty alcohols; fatty acid esters (e.g. triglycerides of fatty acids); and mixtures thereof.

Examples of suspending agents are e.g. celluloses and cellulose derivatives such as, e.g., carboxymethyl cellulose, hydroxyethylcellulose, hydroxypropylcellulose, hydroxypropylmethylcellulose, carraghenan, acacia gum, arabic gum, tragacanth, and mixtures thereof.

Examples of gel bases, viscosity-increasing agents or components which are able to take up exudate from a wound are: liquid paraffin, polyethylene, fatty oils, colloidal silica or aluminum, zinc soaps, glycerol, propylene glycol, tragacanth, carboxyvinyl polymers, magnesium-aluminum silicates, Carbopol®, hydrophilic polymers such as, e.g. starch or cellulose derivatives such as, e.g., carboxymethylcellulose, hydroxyethylcellulose and other cellulose derivatives, water-swellable hydrocolloids, carragenans, hyaluronates (e.g. hyaluronate gel optionally containing sodium chloride), and alginates including propylene glycol alginate.

Examples of ointment bases are e.g. beeswax, paraffin, cetanol, cetyl palmitate, vegetable oils, sorbitan esters of fatty acids (Span), polyethylene glycols, and condensation products between sorbitan esters of fatty acids and ethylene oxide, e.g. polyoxyethylene sorbitan monooleate (Tween).

Examples of hydrophobic or water-emulsifying ointment bases are paraffins, vegetable oils, animal fats, synthetic glycerides, waxes, lanolin, and liquid polyalkylsiloxanes. Examples of hydrophilic ointment bases are solid macrogols (polyethylene glycols). Other examples of ointment bases are triethanolamine soaps, sulphated fatty alcohol and polysorbates.

Examples of other excipients are polymers such as carmelose, sodium carmelose, hydroxypropylmethylcellulose, hydroxyethylcellulose, hydroxypropylcellulose, pectin, xanthan gum, locust bean gum, acacia gum, gelatin, carbomer, emulsifiers like vitamin E, glyceryl stearates, cetanyl glucoside, collagen, carrageenan, hyaluronates and alginates and chitosans.

The dose of antibody required in humans to be effective in the treatment or prevention of allergic inflammation differs with the type and severity of the allergic condition to be treated, the type of allergen, the age and condition of the patient, etc. Typical doses of antibody to be administered are in the range of 1 μg to 1 g, preferably 1-1000 more preferably 2-500, even more preferably 5-50, most preferably 10-20 μg per unit dosage form. In certain embodiments, infusion of antibodies of the present invention may range from 10-500 mg/m².

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection.

In another aspect, provided is a pharmaceutical pack or kit, comprising one or more containers filled with one or more of the ingredients of the pharmaceutical compositions and HDAC and/or CDK4/6 inhibitors.

### Diagnostic and Screening Methods

The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The signatures of the present invention may be discovered by analysis of expression profiles of single-cells within a population of cells from isolated samples (e.g. Sys tumor samples), thus allowing the discovery of novel cell subtypes or cell states that were previously invisible or unrecognized. The presence of subtypes or cell states may be determined by subtype specific or cell state specific signatures. The presence of these specific cell (sub)types or cell states may be determined by applying the signature genes to bulk sequencing data in a sample. In certain embodiments, the signatures of the present invention may be microenvironment specific, such as their expression in a particular spatio-temporal context. In certain embodiments, signatures as discussed herein are specific to a particular pathological context. In certain embodiments, a combination of cell subtypes having a particular signature may indicate an outcome. In certain embodiments, the signatures can be used to deconvolute the network of cells present in a particular pathological condition. In certain embodiments, the presence of specific cells and cell subtypes are indicative of a particular response to treatment, such as including increased or decreased susceptibility to treatment. The signature may indicate the presence of one particular cell type. In one embodiment, the novel signatures are used to detect multiple cell states or hierarchies that occur in subpopulations of cells that are linked to particular pathological condition (e.g. inflammation), or linked to a particular outcome or progression of the disease, or linked to a particular response to treatment of the disease.

The invention provides biomarkers (e.g., phenotype specific or cell type) for the identification, diagnosis, prognosis and manipulation of cell properties, for use in a variety of diagnostic and/or therapeutic indications. Biomarkers in the context of the present invention encompasses, without limitation nucleic acids, proteins, reaction products, and metabolites, together with their polymorphisms, mutations, variants, modifications, subunits, fragments, and other analytes or sample-derived measures. In certain embodiments, biomarkers include the signature genes or signature gene products, and/or cells as described herein.

Biomarkers are useful in methods of diagnosing, prognosing and/or staging an immune response in a subject by detecting a first level of expression, activity and/or function of one or more biomarker and comparing the detected level to a control of level wherein a difference in the detected level and the control level indicates that the presence of an immune response in the subject.

The terms “diagnosis” and “monitoring” are commonplace and well-understood in medical practice. By means of further explanation and without limitation the term “diagnosis” generally refers to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The biomarkers of the present invention are useful in methods of identifying patient populations at risk or suffering from an immune response based on a detected level of expression, activity and/or function of one or more biomarkers. These biomarkers are also useful in monitoring subjects undergoing treatments and therapies for suitable or aberrant response(s) to determine efficaciousness of the treatment or therapy and for selecting or modifying therapies and treatments that would be efficacious in treating, delaying the progression of or otherwise ameliorating a symptom. The biomarkers provided herein are useful for selecting a group of patients at a specific state of a disease with accuracy that facilitates selection of treatments.

The term “monitoring” generally refers to the follow-up of a disease or a condition in a subject for any changes which may occur over time.

The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

Suitably, an altered quantity or phenotype of the immune cells in the subject compared to a control subject having normal immune status or not having a disease comprising an immune component indicates that the subject has an impaired immune status or has a disease comprising an immune component or would benefit from an immune therapy.

Hence, the methods may rely on comparing the quantity of immune cell populations, biomarkers, or gene or gene product signatures measured in samples from patients with reference values, wherein said reference values represent known predictions, diagnoses and/or prognoses of diseases or conditions as taught herein.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values can represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying severity.

In yet another example, distinct reference values may represent a good prognosis for a given disease or condition as taught herein vs. a poor prognosis for said disease or condition. In a further example, distinct reference values may represent varyingly favourable or unfavourable prognoses for such disease or condition.

Such comparison may generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other cell populations, biomarkers and gene or gene product signatures. For example, a reference value may be established in an individual or a population of individuals characterised by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value >second value; or decrease: first value <second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

In one embodiment, the signature genes, biomarkers, and/or cells may be detected or isolated by immunofluorescence, immunohistochemistry (IHC), fluorescence activated cell sorting (FACS), mass spectrometry (MS), mass cytometry (CyTOF), RNA-seq, single cell RNA-seq (described further herein), quantitative RT-PCR, single cell qPCR, FISH, RNA-FISH, MERFISH (multiplex (in situ) RNA FISH) and/or by in situ hybridization. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25).

In certain embodiments, diseases related to Sys as described further herein are diagnosed, prognosed, or monitored. For example, a tissue sample may be obtained and analyzed for specific cell markers (IHC) or specific transcripts (e.g., RNA-FISH). Tissue samples for diagnosis, prognosis or detecting may be obtained by endoscopy. In one embodiment, a sample may be obtained by endoscopy and analyzed by FACS. As used herein, “endoscopy” refers to a procedure that uses an endoscope to examine the interior of a hollow organ or cavity of the body. The endoscope may include a camera and a light source. The endoscope may include tools for dissection or for obtaining a biological sample. A cutting tool can be attached to the end of the endoscope, and the apparatus can then be used to perform surgery. Applications of endoscopy that can be used with the present invention include, but are not limited to examination of the oesophagus, stomach and duodenum (esophagogastroduodenoscopy); small intestine (enteroscopy); large intestine/colon (colonoscopy, sigmoidoscopy); bile duct; rectum (rectoscopy) and anus (anoscopy), both also referred to as (proctoscopy); respiratory tract; nose (rhinoscopy); lower respiratory tract (bronchoscopy); ear (otoscope); urinary tract (cystoscopy); female reproductive system (gynoscopy); cervix (colposcopy); uterus (hysteroscopy); fallopian tubes (falloposcopy); normally closed body cavities (through a small incision); abdominal or pelvic cavity (laparoscopy); interior of a joint (arthroscopy); or organs of the chest (thoracoscopy and mediastinoscopy).

In certain embodiments, the method provides for treating a patient with an HDAC inhibitor and CDK4/6 inhibitor or a combination thereof, or via ACT, wherein the patient is suffering from Sys. the method comprising the steps of: determining whether the patient expresses a gene signature, biological program or marker gene as described herein: obtaining or having obtained a biological sample from the patient; and performing or having performed an assay as described herein on the biological sample to determine if the patient expresses the gene signature, biological program or marker gene; and if the patient has a malignant gene signature, biological program or marker gene, then administering HDAC inhibitor and CDK4/6 inhibitor or a combination thereof to the patient, or treating the patient with ACT in an amount sufficient to selectively target synovial sarcoma cells, and if the patient does not have a malignant gene signature, biological program or marker gene, then not administering treatments to the patient, wherein a risk of having synovial sarcoma, and in some, instances, risk of metastatic disease, is increased if the patient has a malignant gene signature, biological program or marker gene. In an aspect, the administration of an effective amount of modulating agent reduces the malignant gene signature, treats the Synovial Sarcoma and/or tumor burden, and/or decreases the risk of malignancy.

In embodiments, methods of treatment may comprise administration of two or more agents, In particular embodiments, the administration of two or more modulating agents may provide a synergistic effect. A synergistic effect is defined herein as more than additive results of agents independently administered. In particular embodiments, the additive results may be measured by duration of repression/activation of one or more target genes, or by amount of repression/activation of one or more target genes, or, for example of tumor burden, immune resistance, or other indicator of treatment.

The present invention also may comprise a kit with a detection reagent that binds to one or more biomarkers or can be used to detect one or more biomarkers.

### MS Methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)2 fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854, 5,288,644, 5,324,633, 5,432,049, 5,470,710, 5,492,806, 5,503,980, 5,510,270, 5,525,464, 5,547,839, 5,580,732, 5,661,028, 5,800,992, the disclosures of which are incorporated herein by reference, as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65 C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif. (1992).

### Amplifying Target Molecules

Methods of screening can include amplification of target molecules of interest. The step of amplifying one or more target molecules can comprise amplification systems known in the art. In some embodiments, amplification is isothermal. In certain example embodiments, target RNAs and/or DNAs may be amplified prior to activating a CRISPR effector protein for detection, diagnosis or other uses as described herein. Any suitable RNA or DNA amplification technique may be used. In certain example embodiments, the RNA or DNA amplification is an isothermal amplification. In certain example embodiments, the isothermal amplification may be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In certain example embodiments, non-isothermal amplification methods may be used which include, but are not limited to, PCR, multiple displacement amplification (MDA), rolling circle amplification (RCA), ligase chain reaction (LCR), or ramification amplification method (RAM).

In certain example embodiments, the RNA or DNA amplification is NASBA, which is initiated with reverse transcription of target RNA by a sequence-specific reverse primer to create a RNA/DNA duplex. RNase H is then used to degrade the RNA template, allowing a forward primer containing a promoter, such as the T7 promoter, to bind and initiate elongation of the complementary strand, generating a double-stranded DNA product. The RNA polymerase promoter-mediated transcription of the DNA template then creates copies of the target RNA sequence. Importantly, each of the new target RNAs can be detected by the guide RNAs thus further enhancing the sensitivity of the assay. Binding of the target RNAs by the guide RNAs then leads to activation of the CRISPR effector protein and the methods proceed as outlined above. The NASBA reaction has the additional advantage of being able to proceed under moderate isothermal conditions, for example at approximately 41° C., making it suitable for systems and devices deployed for early and direct detection in the field and far from clinical laboratories.

In certain other example embodiments, a recombinase polymerase amplification (RPA) reaction may be used to amplify the target nucleic acids. RPA reactions employ recombinases which are capable of pairing sequence-specific primers with homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. The sequence specific primers are designed to amplify a sequence comprising the target nucleic acid sequence to be detected. In certain example embodiments, a RNA polymerase promoter, such as a T7 promoter, is added to one of the primers. This results in an amplified double-stranded DNA product comprising the target sequence and a RNA polymerase promoter. After, or during, the RPA reaction, a RNA polymerase is added that will produce RNA from the double-stranded DNA templates. The amplified target RNA can then in turn be detected by the CRISPR effector system. In this way target DNA can be detected using the embodiments disclosed herein. RPA reactions can also be used to amplify target RNA. The target RNA is first converted to cDNA using a reverse transcriptase, followed by second strand DNA synthesis, at which point the RPA reaction proceeds as outlined above.

In an embodiment of the invention may comprise nickase-based amplification. The nicking enzyme may be a CRISPR protein. Accordingly, the introduction of nicks into dsDNA can be programmable and sequence-specific. FIG. 115 depicts an embodiment of the invention, which starts with two guides designed to target opposite strands of a dsDNA target. According to the invention, the nickase can be Cpf1, C2c1, Cas9 or any ortholog or CRISPR protein that cleaves or is engineered to cleave a single strand of a DNA duplex. The nicked strands may then be extended by a polymerase. In an embodiment, the locations of the nicks are selected such that extension of the strands by a polymerase is towards the central portion of the target duplex DNA between the nick sites. In certain embodiments, primers are included in the reaction capable of hybridizing to the extended strands followed by further polymerase extension of the primers to regenerate two dsDNA pieces: a first dsDNA that includes the first strand Cpf1 guide site or both the first and second strand Cpf1 guide sites, and a second dsDNA that includes the second strand Cpf1 guide site or both the first and second strand Cprf guide sites. These pieces continue to be nicked and extended in a cyclic reaction that exponentially amplifies the region of the target between nicking sites.

The amplification can be isothermal and selected for temperature. In one embodiment, the amplification proceeds rapidly at 37 degrees. In other embodiments, the temperature of the isothermal amplification may be chosen by selecting a polymerase (e.g. Bsu, Bst, Phi29, klenow fragment etc.). operable at a different temperature.

Thus, whereas nicking isothermal amplification techniques use nicking enzymes with fixed sequence preference (e.g. in nicking enzyme amplification reaction or NEAR), which requires denaturing of the original dsDNA target to allow annealing and extension of primers that add the nicking substrate to the ends of the target, use of a CRISPR nickase wherein the nicking sites can be programed via guide RNAs means that no denaturing step is necessary, enabling the entire reaction to be truly isothermal. This also simplifies the reaction because these primers that add the nicking substrate are different than the primers that are used later in the reaction, meaning that NEAR requires two primer sets (i.e. 4 primers) while Cpf1 nicking amplification only requires one primer set (i.e. two primers). This makes nicking Cpf1 amplification much simpler and easier to operate without complicated instrumentation to perform the denaturation and then cooling to the isothermal temperature.

Accordingly, in certain example embodiments the systems disclosed herein may include amplification reagents. Different components or reagents useful for amplification of nucleic acids are described herein. For example, an amplification reagent as described herein may include a buffer, such as a Tris buffer. A Tris buffer may be used at any concentration appropriate for the desired application or use, for example including, but not limited to, a concentration of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 25 mM, 50 mM, 75 mM, 1 M, or the like. One of skill in the art will be able to determine an appropriate concentration of a buffer such as Tris for use with the present invention.

A salt, such as magnesium chloride (MgCl2), potassium chloride (KCl), or sodium chloride (NaCl), may be included in an amplification reaction, such as PCR, in order to improve the amplification of nucleic acid fragments. Although the salt concentration will depend on the particular reaction and application, in some embodiments, nucleic acid fragments of a particular size may produce optimum results at particular salt concentrations. Larger products may require altered salt concentrations, typically lower salt, in order to produce desired results, while amplification of smaller products may produce better results at higher salt concentrations. One of skill in the art will understand that the presence and/or concentration of a salt, along with alteration of salt concentrations, may alter the stringency of a biological or chemical reaction, and therefore any salt may be used that provides the appropriate conditions for a reaction of the present invention and as described herein.

Other components of a biological or chemical reaction may include a cell lysis component in order to break open or lyse a cell for analysis of the materials therein. A cell lysis component may include, but is not limited to, a detergent, a salt as described above, such as NaCl, KCl, ammonium sulfate [(NH4)2SO4], or others. Detergents that may be appropriate for the invention may include Triton X-100, sodium dodecyl sulfate (SDS), CHAPS (3-[(3-cholamidopropyl)dimethylammonio]-1-propanesulfonate), ethyl trimethyl ammonium bromide, nonyl phenoxypolyethoxylethanol (NP-40). Concentrations of detergents may depend on the particular application, and may be specific to the reaction in some cases. Amplification reactions may include dNTPs and nucleic acid primers used at any concentration appropriate for the invention, such as including, but not limited to, a concentration of 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like. Likewise, a polymerase useful in accordance with the invention may be any specific or general polymerase known in the art and useful or the invention, including Taq polymerase, Q5 polymerase, or the like.

In some embodiments, amplification reagents as described herein may be appropriate for use in hot-start amplification. Hot start amplification may be beneficial in some embodiments to reduce or eliminate dimerization of adaptor molecules or oligos, or to otherwise prevent unwanted amplification products or artifacts and obtain optimum amplification of the desired product. Many components described herein for use in amplification may also be used in hot-start amplification. In some embodiments, reagents or components appropriate for use with hot-start amplification may be used in place of one or more of the composition components as appropriate. For example, a polymerase or other reagent may be used that exhibits a desired activity at a particular temperature or other reaction condition. In some embodiments, reagents may be used that are designed or optimized for use in hot-start amplification, for example, a polymerase may be activated after transposition or after reaching a particular temperature. Such polymerases may be antibody-based or aptamer-based. Polymerases as described herein are known in the art. Examples of such reagents may include, but are not limited to, hot-start polymerases, hot-start dNTPs, and photo-caged dNTPs. Such reagents are known and available in the art. One of skill in the art will be able to determine the optimum temperatures as appropriate for individual reagents.

Amplification of nucleic acids may be performed using specific thermal cycle machinery or equipment, and may be performed in single reactions or in bulk, such that any desired number of reactions may be performed simultaneously. In some embodiments, amplification may be performed using microfluidic or robotic devices, or may be performed using manual alteration in temperatures to achieve the desired amplification. In some embodiments, optimization may be performed to obtain the optimum reactions conditions for the particular application or materials. One of skill in the art will understand and be able to optimize reaction conditions to obtain sufficient amplification.

In certain embodiments, detection of DNA with the methods or systems of the invention requires transcription of the (amplified) DNA into RNA prior to detection.

It will be evident that detection methods of the invention can involve nucleic acid amplification and detection procedures in various combinations. The nucleic acid to be detected can be any naturally occurring or synthetic nucleic acid, including but not limited to DNA and RNA, which may be amplified by any suitable method to provide an intermediate product that can be detected. Detection of the intermediate product can be by any suitable method including but not limited to binding and activation of a CRISPR protein which produces a detectable signal moiety by direct or collateral activity.

Helicase-Dependent Amplification

In helicase-dependent amplification, a helicase enzyme is used to unwind a double stranded nucleic acid to generate templates for primer hybridization and subsequent primer-extension. This process utilizes two oligonucleotide primers, each hybridizing to the 3′-end of either the sense strand containing the target sequence or the anti-sense strand containing the reverse-complementary target sequence. The HDA reaction is a general method for helicase-dependent nucleic acid amplification.

In combining this method with a CRISPR-SHERLOCK system, the target nucleic acid may be amplified by opening R-loops of the target nucleic acid using first and second CRISPR/Cas complexes. The first and second strand of the target nucleic acid may thus be unwound using a helicase, allowing primers and polymerase to bind and extend the DNA under isothermal conditions.

The term “helicase” refers here to any enzyme capable of unwinding a double stranded nucleic acid enzymatically. For example, helicases are enzymes that are found in all organisms and in all processes that involve nucleic acid such as replication, recombination, repair, transcription, translation and RNA splicing. (Kornberg and Baker, DNA Replication, W. H. Freeman and Company (2^{nd} ed. (1992)), especially chapter 11). Any helicase that translocates along DNA or RNA in a 5′ to 3′ direction or in the opposite 3′ to 5′ direction may be used in present embodiments of the invention. This includes helicases obtained from prokaryotes, viruses, archaea, and eukaryotes or recombinant forms of naturally occurring enzymes as well as analogues or derivatives having the specified activity. Examples of naturally occurring DNA helicases, described by Kornberg and Baker in chapter 11 of their book, DNA Replication, W. H. Freeman and Company (2^{nd} ed. (1992)), include E. coli helicase I, II, III, & IV, Rep, DnaB, PriA, PcrA, T4 Gp41helicase, T4 Dda helicase, T7 Gp4 helicases, SV40 Large T antigen, yeast RAD. Additional helicases that may be useful in HDA include RecQ helicase (Harmon and Kowalczykowski, J. Biol. Chem. 276:232-243 (2001)), thermostable UvrD helicases from T. tengcongensis (disclosed in this invention, Example XII) and T. thermophilus (Collins and McCarthy, Extremophiles. 7:35-41. (2003)), thermostable DnaB helicase from T. aquaticus (Kaplan and Steitz, J. Biol. Chem. 274:6889-6897 (1999)), and MCM helicase from archaeal and eukaryotic organisms ((Grainge et al., Nucleic Acids Res. 31:4888-4898 (2003)).

A traditional definition of a helicase is an enzyme that catalyzes the reaction of separating/unzipping/unwinding the helical structure of nucleic acid duplexes (DNA, RNA or hybrids) into single-stranded components, using nucleoside triphosphate (NTP) hydrolysis as the energy source (such as ATP). However, it should be noted that not all helicases fit this definition anymore. A more general definition is that they are motor proteins that move along the single-stranded or double stranded nucleic acids (usually in a certain direction, 3′ to 5′ or 5 to 3, or both), i.e. translocases, that can or cannot unwind the duplexed nucleic acid encountered. In addition, some helicases simply bind and “melt” the duplexed nucleic acid structure without an apparent translocase activity.

Helicases exist in all living organisms and function in all aspects of nucleic acid metabolism. Helicases are classified based on the amino acid sequences, directionality, oligomerization state and nucleic-acid type and structure preferences. The most common classification method was developed based on the presence of certain amino acid sequences, called motifs. According to this classification helicases are divided into 6 super families: SF1, SF2, SF3, SF4, SF5 and SF6. SF1 and SF2 helicases do not form a ring structure around the nucleic acid, whereas SF3 to SF6 do. Superfamily classification is not dependent on the classical taxonomy.

DNA helicases are responsible for catalyzing the unwinding of double-stranded DNA (dsDNA) molecules to their respective single-stranded nucleic acid (ssDNA) forms. Although structural and biochemical studies have shown how various helicases can translocate on ssDNA directionally, consuming one ATP per nucleotide, the mechanism of nucleic acid unwinding and how the unwinding activity is regulated remains unclear and controversial (T. M. Lohman, E. J. Tomko, C. G. Wu, “Non-hexameric DNA helicases and translocases: mechanisms and regulation,” Nat Rev Mol Cell Biol 9:391-401 (2008)). Since helicases can potentially unwind all nucleic acids encountered, understanding how their unwinding activities are regulated can lead to harnessing helicase functions for biotechnology applications.

The term “HDA” refers to Helicase Dependent Amplification, which is an in vitro method for amplifying nucleic acids by using a helicase preparation for unwinding a double stranded nucleic acid to generate templates for primer hybridization and subsequent primer-extension. This process utilizes two oligonucleotide primers, each hybridizing to the 3′-end of either the sense strand containing the target sequence or the anti-sense strand containing the reverse-complementary target sequence. The HDA reaction is a general method for helicase-dependent nucleic acid amplification.

The invention comprises use of any suitable helicase known in the art. These include, but are not necessarily limited to, UvrD helicase, CRISPR-Cas3 helicase, E. coli helicase I, E. coli helicase II, E. coli helicase III, E. coli helicase IV, Rep helicase, DnaB helicase, PriA helicase, PcrA helicase, T4 Gp41 helicase, T4 Dda helicase, SV40 Large T antigen, yeast RAD helicase, RecD helicase, RecQ helicase, thermostable T. tengcongensis UvrD helicase, thermostable T. thermophilus UvrD helicase, thermostable T. aquaticus DnaB helicase, Dda helicase, papilloma virus E1 helicase, archaeal MCM helicase, eukaryotic MCM helicase, and T7 Gp4 helicase.

An “individual discrete volume” is a discrete volume or discrete space, such as a container, receptacle, or other defined volume or space that can be defined by properties that prevent and/or inhibit migration of nucleic acids and reagents necessary to carry out the methods disclosed herein, for example a volume or space defined by physical properties such as walls, for example the walls of a well, tube, or a surface of a droplet, which may be impermeable or semipermeable, or as defined by other means such as chemical, diffusion rate limited, electro-magnetic, or light illumination, or any combination thereof. By “diffusion rate limited” (for example diffusion defined volumes) is meant spaces that are only accessible to certain molecules or reactions because diffusion constraints effectively defining a space or volume as would be the case for two parallel laminar streams where diffusion will limit the migration of a target molecule from one stream to the other. By “chemical” defined volume or space is meant spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. By “electro-magnetically” defined volume or space is meant spaces where the electro-magnetic properties of the target molecules or their supports such as charge or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the used of non-walled, or semipermeable is that some reagents, such as buffers, chemical activators, or other agents maybe passed in Applicants' through the discrete volume, while other material, such as target molecules, maybe maintained in the discrete volume or space. Typically, a discrete volume will include a fluid medium, (for example, an aqueous solution, an oil, a buffer, and/or a media capable of supporting cell growth) suitable for labeling of the target molecule with the indexable nucleic acid identifier under conditions that permit labeling. Exemplary discrete volumes or spaces useful in the disclosed methods include droplets (for example, microfluidic droplets and/or emulsion droplets), hydrogel beads or other polymer structures (for example poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In certain example embodiments, the individual discrete volumes are the wells of a microplate. In certain example embodiments, the microplate is a 96 well, a 384 well, or a 1536 well microplate.

### Single Cell Sequencing

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p666-6′73, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

In certain embodiments, the invention involves the Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1).

### Screening for Modulating Agents

A further aspect of the invention relates to a method for identifying an agent capable of modulating one or more phenotypic aspects of a cell or cell population as disclosed herein, comprising: a) applying a candidate agent to the cell or cell population; b) detecting modulation of one or more phenotypic aspects of the cell or cell population by the candidate agent, thereby identifying the agent. The phenotypic aspects of the cell or cell population that is modulated may be a gene signature or biological program specific to a cell type or cell phenotype or phenotype specific to a population of cells (e.g., an inflammatory phenotype or suppressive immune phenotype). In certain embodiments, steps can include administering candidate modulating agents to cells, detecting identified cell (sub)populations for changes in signatures, or identifying relative changes in cell (sub) populations which may comprise detecting relative abundance of particular gene signatures.

The term “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of an immune cell or immune cell population may be modulated without substantially altering other (unintended, undesired) phenotypic aspect(s).

The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place.

Agents may include any potential class of biologically active conditions, substances or agents, such as for instance antibodies, proteins, peptides, nucleic acids, oligonucleotides, small molecules, or combinations thereof, as described herein.

The methods of phenotypic analysis can be utilized for evaluating environmental stress and/or state, for screening of chemical libraries, and to screen or identify structural, syntenic, genomic, and/or organism and species variations. For example, a culture of cells, can be exposed to an environmental stress, such as but not limited to heat shock, osmolarity, hypoxia, cold, oxidative stress, radiation, starvation, a chemical (for example a therapeutic agent or potential therapeutic agent) and the like. After the stress is applied, a representative sample can be subjected to analysis, for example at various time points, and compared to a control, such as a sample from an organism or cell, for example a cell from an organism, or a standard value. By exposing cells, or fractions thereof, tissues, or even whole animals, to different members of the chemical libraries, and performing the methods described herein, different members of a chemical library can be screened for their effect on immune phenotypes thereof simultaneously in a relatively short amount of time, for example using a high throughput method.

Aspects of the present disclosure relate to the correlation of an agent with the spatial proximity and/or epigenetic profile of the nucleic acids in a sample of cells. In some embodiments, the disclosed methods can be used to screen chemical libraries for agents that modulate chromatin architecture epigenetic profiles, and/or relationships thereof.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

In certain embodiments, the present invention provides for gene signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The signatures or biological programs of the present invention may be used to screen for drugs that reduce the signature or biological program in cells as described herein. The signature or biological program may be used for GE-HTS. In certain embodiments, pharmacological screens may be used to identify drugs that are selectively toxic to cells having a signature.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). In certain embodiments, Cmap can be used to screen for small molecules capable of modulating a signature or biological program of the present invention in silico.

### MS Methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)2 fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65 C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif. (1992).

In certain embodiments, the gene signature includes surface expressed proteins. In certain embodiments, surface proteins may be targeted for detection and isolation of cell types, or may be targeted therapeutically to modulate an immune response.

In one embodiment, the signature genes and/or cells may be detected or isolated by immunofluorescence, immunohistochemistry, fluorescence activated cell sorting (FACS), mass cytometry (CyTOF), RNA-seq, scRNA-seq (e.g., Drop-seq, InDrop, 10× Genomics), single cell qPCR, MERFISH (multiplex (in situ) RNA FISH) and/or by in situ hybridization. Other methods including absorbance assays and colorimetric assays are known in the art and may be used herein.

### Sequencing and Nucleic Acid Analysis

In certain embodiments, the invention involves targeted nucleic acid profiling (e.g., sequencing, quantitative reverse transcription polymerase chain reaction, and the like) (see e.g., Geiss G K, et al., Direct multiplexed measurement of gene expression with color-coded probe pairs. Nat Biotechnol. 2008 March; 26(3):317-25). In certain embodiments, a target nucleic acid molecule (e.g., RNA molecule), may be sequenced by any method known in the art, for example, methods of high-throughput sequencing, also known as next generation sequencing or deep sequencing. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others.

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p666-6′73, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

In certain embodiments, the invention involves the Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1).

The invention is further described in the following examples, which do not limit the scope of the invention described in the claims.

### EXAMPLES

### Example 1—Single-Cell RNA-Seq Atlas of Synovial Sarcoma (SyS): Cell Type Inference from Expression and Genetic Features

Despite the relatively low number of secondary mutations, SyS tumors display different degrees of cellular differentiation and plasticity, and are classified accordingly as monophasic (mesenchymal cells), biphasic (mesenchymal and epithelial cells), or poorly differentiated (undifferentiated cells). The co-existence of distinct cellular phenotypes and morphologies in a single SyS tumor provides a unique opportunity to explore intratumor heterogeneity and cell state transitions. However, since human SyS has been studied primarily in established cellular models (Kadoch et al. Cell 153:71-85 (2013); McBride et al. Cancer Cell (2018) doi:10.1016/j.ccell.2018.05.002; Banito et al. Cancer Cell 33:527-541.e8 (2018)) and through bulk profiling of tumor tissues (Nakayama et al. Am J Surg Pathol 34:1599-1607 (2010); Lagarde et al. J Clin Oncol Of Am Soc Clin Oncol 31:608-615 (2013)), the molecular features of the different SyS subpopulations have so far remained elusive. In particular, it remains unclear how this malignant cellular diversity comes about, which malignant cell states drive tumor progression, and how to selectively target aggressive synovial sarcoma cells to blunt tumor growth and dissemination.

To address these questions, Applicants leveraged single-cell RNA-Seq (scRNA-Seq; FIG. 1A) and profiled 16,872 cells from 12 human SyS tumors. The data reveal a spectrum of cell states and a clear developmental hierarchy, where poorly differentiated cells cycle and replenish the tumor. Within each tumor they found a distinct subpopulation of malignant cells that express a core oncogenic program with features of immune evasion, resistance to apoptosis, oxidative metabolism, and poor differentiation. Applicants demonstrated that the program is associated with poor clinical outcomes and is controlled in part by the SS18-SSX oncoprotein and in part by the tumor microenvironment. Lastly, Applicants computationally modeled the program transcriptional regulation, highlighting HDAC3 and CDK4/6 as its key regulator and targets, respectively. In accordance with this, combining HDAC and CDK4/6 inhibitors Applicants were able to block the program and selectively target synovial sarcoma cells, while sparing non-malignant ones.

The workflow was as follows: (1) Mapped the transcriptional landscape of synovial sarcoma cells: characterized differentiation trajectories, revealed that stem-like cells are those that cycle, and discovered this new core oncogenic program. (2) These aggressive features (poor differentiation, cell cycle, and the different core oncogenic features) are tightly co-regulated and predictive of clinical outcomes. (3) The fusion and (TNF/IFN-secreting) immune cells promote/repress the aggressive features, respectively. (4) Lastly, Applicants selectively targeted the different aggressive cells by combining HDAC (core oncogenic) and CDK4/6 (cell cycle) inhibitors.

Using full-length (Picelli et al. Nat Protoc 9:171-181 (2014)) and droplet-based (Zheng et al. Nat Commun 8:14049 (2017)) scRNA-Seq, Applicants profiled 16,872 high quality malignant, immune, and stromal cells from 12 human SyS tumors (FIGS. 1A, 1B, 2A, Tables 1-3). Cells were assigned to different cell types according to both genetic and transcriptional features (FIG. 1B, 2A-2D, Methods): (1) Detection of the SS18-SSX fusion transcripts (Haas eta 1. bioRxiv (2017) doi:10.1101/120295); (2) inference of copy number alterations (CNAs) from scRNA-Seq profiles (Patel et al. Science 344:1396-1401 (2014)), which was validated in four tumors by bulk whole-exome sequencing (WES) (FIG. 1C); (3) expression-based clustering and post hoc annotation of non-malignant clusters based on canonical cell type markers (FIG. 2A, Tables 4 and 5); and (4) similarity of cells to bulk expression profiles of Sys tumors (Abeshouse et al. Cell 171:950-965.e28 (2017)). The four approaches were highly congruent (FIG. 2A). For example, the fusion was detected in 58.6% of cells defined as malignant by other analyses, but only in 0.89% of non-malignant cells. Notably, SSX1/2 expression was also very specific to malignant cells (detection rate of 66.64% and 1.49% in the malignant and non-malignant cells, respectively; FIG. 2A “SSX1/2 detection”), and in one of the tumors Applicants identified another malignant-specific fusion (MEOX2-AGMO) (FIGS. 3B, 3C). Similarly, CNAs were detected only in cells that were assigned as malignant by the other analyses (FIG. 1B, 1C), and the Sys similarity scores distinguished between malignant and non-malignant cells (as defined by the other methods) with 100% accuracy (FIGS. 1B, 2B). Cells discordant across these criteria (<0.05%) were excluded from all downstream analyses.

TABLE 2A
Clinical characteristics of the patients and samples in the scRNA-seq cohort.

| | | | | | | | | Metastatic/ |
|---|---|---|---|---|---|---|---|---|
| | | | | | | | Neoadjuvant | primary |
| Patient | Tumor | Mutation | Diagnostic | Sex | Age | Localization | Treatment | lesion |
| P1 | S1 | SS18-SSX1 | Biphasic | M | 23 | Knee | Chemotherapy | Primary |
| | | | | | | | (limp perfusion | |
| | | | | | | | with Melphalan | |
| | | | | | | | and TNFalpha) | |
| P2 | S2 | SS18-SSX1 | Monophasic | M | 52 | Thigh | Chemotherapy | Primary |
| | | | | | | | (AIM) + | |
| | | | | | | | Radiotherapy | |
| P5 | S5 | SS18-SSX2 | Monophasic | F | 62 | Lung | Chemotherapy | Metastatic |
| | | | | | | | (Ifosfamide) + | |
| | | | | | | | Radiotherapy | |
| P7 | S7 | SS18-SSX2 | Poorly | M | 45 | Para-aortic | Radiotherapy | Primary |
| | | | differentiated | | | | | |
| P10 | S10 | SS18-SSX1 | Monophasic | M | 22 | Lung | Chemotherapy | Metastatic |
| | | | | | | | (AIM) + | |
| | | | | | | | Radiotherapy | |
| P11 | S11 | SS18-SSX1 | Monophasic | F | 34 | Lung (L | None | Primary |
| | primary | | | | | inf lobe) | | |
| P11 | S11 | SS18-SSX1 | Monophasic | F | 34 | Lung (L | None | Metastatic |
| | Metastatic | | | | | sup lobe) | | |
| P12 | S12 | SS18-SSX1 | Biphasic | M | 24 | Chest | None | Primary |
| | | | | | | wall | | |
| P12 | S12 post | SS18-SSX1 | Biphasic | M | 24 | Chest | Chemotherapy | Primary |
| | treatment | | | | | wall | (AIM) + | |
| | | | | | | | Radiotherapy | |
| P13 | S13 | SS18-SSX2 | Monophasic | F | 29 | Thyroid | Chemotherapy | Metastatic |
| | | | | | | | (AIM) + | |
| | | | | | | | Radiotherapy | |
| P14 | S14 | SS18-SSX1 | Monophasic | F | 57 | Knee | None | Primary |
| P16 | S16 | SS18-SSX2 | Biphasic | M | | | | |

TABLE 2B
Quality measures of the scRNA-seq cohorts.
Synovial sarcoma human tumors

| | No. of | Median no. of | Median no. of | |
|---|---|---|---|---|
| Cell type | cells | detected genes | aligned reads | Sequencing |
| B.cell | 90 | 2558.5 | 241971.5 | Smart-Seq2 |
| CAFs | 81 | 3046 | 517671 | Smart-Seq2 |
| Endothelial | 80 | 3356.5 | 497703.5 | Smart-Seq2 |
| Macrophage | 943 | 3720 | 362523 | Smart-Seq2 |
| Malignant | 4371 | 4743 | 320982 | Smart-Seq2 |
| Mast | 185 | 3222 | 350962 | Smart-Seq2 |
| NK | 102 | 2601 | 177431.5 | Smart-Seq2 |
| CD4 T cell | 235 | 2618 | 177160 | Smart-Seq2 |
| CD8 T cell | 659 | 2527 | 153707 | Smart-Seq2 |
| T cell | 206 | 2582.5 | 182173.5 | Smart-Seq2 |
| Inconsistent | 157 | 5753 | 357370 | Smart-Seq2 |
| assignments | | | | |
| CAFs | 158 | 2665.5 | 9263 | 10x |
| Endothelial | 418 | 3650 | 14031.5 | 10x |
| Macrophage | 275 | 2089 | 7855 | 10x |
| Malignant | 8323 | 2850 | 9609 | 10x |
| Inconsistent | 589 | 2792 | 10110 | 10x |
| assignments | | | | |

TABLE 3
Quality measures of the scRNA-seq cohorts.
Synovial sarcoma cell lines/cultures

| | | No. of | Median no. of | Median no. of | |
|---|---|---|---|---|---|
| Experiment | Cell type | cells | detected genes | aligned reads | Sequencing |
| TNF/IFN | SS11metaexp | 185 | 7215 | 200412 | Smart-Seq2 |
| | SSexp1 | 263 | 6925 | 485893 | Smart-Seq2 |
| | SSexp2 | 256 | 6445 | 283369 | Smart-Seq2 |
| | SSexp4 | 391 | 7468 | 244698 | Smart-Seq2 |
| SS18-SXKD | ASKA, shCt | 1477 | 4743 | 27148 | 10x |
| | (day 3) | | | | |
| | ASKA, shCt | 1301 | 5346 | 30837 | 10x |
| | (day 7) | | | | |
| | ASKA, shSSX | 1503 | 4606 | 26029 | 10x |
| | (day 3) | | | | |
| | ASKA, shSSX | 1554 | 5172.5 | 32017 | 10x |
| | d(day 7) | | | | |
| | SYO1, shCt | 1284 | 3451.5 | 18852.5 | 10x |
| | (day 3) | | | | |
| | SYO1, shCt | 1742 | 3462.5 | 16348 | 10x |
| | (day 7) | | | | |
| | SYO1, shSSX | 2000 | 3295 | 17713 | 10x |
| | (day 3) | | | | |
| | SYO1, shSSX | 1402 | 2700 | 10737.5 | 10x |
| | (day 7) | | | | |

TABLE 4
Cell type signatures derived from the analysis of the synovial sarcoma scRNA-seq cohort.
Single-cell denovo signatures

| | | Endothelial | | |
|---|---|---|---|---|
| B cell | CAF | cell | Macrophage | Mastocyte |
| AFF3 | ACAN | ACVRL1 | ABCC3 | ABCA1 |
| BACH2 | ACTA2 | ADAM15 | ABHD12 | ABCC1 |
| BANK1 | ACTN1 | ADCY4 | ACSL1 | ABCC4 |
| BCL11A | ADAMTS12 | AFAP1L1 | ADAP2 | ACER3 |
| BLK | ADAMTS4 | AIF1L | ADIPOR1 | ACOT7 |
| C12orf42 | ADIRF | APLNR | ADORA3 | ACSL4 |
| CCR7 | AEBP1 | APOL3 | AIF1 | ADAM12 |
| CD19 | AGAP11 | AQP1 | AKR1B1 | ADCYAP1 |
| CD37 | ANGPT1 | ARAP3 | ALCAM | ADRB2 |
| CD55 | ANGPTL4 | ARHGAP29 | ALDH2 | AGTRAP |
| CD79A | ANO1 | ARHGAP31 | AMPD3 | AHR |
| CD79B | ARHGAP1 | ARHGEF15 | AP1B1 | ALDH1A1 |
| CDCA7L | ARHGAP42 | ARHGEF28 | APOC1 | ALOX5 |
| CLEC17A | ARHGEF17 | ARL15 | ATP13A3 | ALOX5AP |
| CXCR5 | ASPN | ARRDC3 | ATP6V0B | ALS2 |
| CYBASC3 | AVPR1A | BCAM | ATP6V1B2 | AMHR2 |
| DAPP1 | AXL | BCL6B | B3GNT5 | ANKRD27 |
| EEF1B2 | BGN | BDKRB2 | BCAT1 | AQP10 |
| EZR | C11orf96 | BMPR2 | BCL2A1 | ARHGAP18 |
| FAM129C | C1orf198 | BTNL9 | BEST1 | ARHGEF6 |
| FCER2 | C1QTNF1 | C8orf4 | BLVRB | ATP6V0A2 |
| FCRL1 | C1R | CA2 | C10orf54 | AURKA |
| FCRL2 | C1S | CALCRL | C15orf48 | B4GALT5 |
| FCRL5 | C21orf7 | CAPN11 | C1QA | BACE2 |
| FCRLA | C4A | CARD10 | C1QB | BATF |
| FGD2 | C4B | CASKIN2 | C1QC | BLVRA |
| GNG7 | C4B_2 | CCL14 | C2 | BMP2K |
| HLA-DOB | CALD1 | CD200 | C3 | BTK |
| HLA-DQA2 | CCDC102B | CD320 | C3AR1 | C1orf186 |
| HVCN1 | CCR10 | CD34 | C5AR1 | C20orf118 |
| ICOSLG | CD248 | CDH5 | C9orf72 | C6orf25 |
| IGJ | CDH6 | CFI | CARD9 | C8G |
| IGLL5 | CFH | CLDN5 | CASP1 | CACNA2D4 |
| IL16 | CHN1 | CLEC14A | CCL20 | CADPS |
| IRF8 | CLEC11A | CLEC1A | CCL3 | CALB2 |
| KDM4B | COL12A1 | CLEC3B | CCL3L1 | CAPG |
| KIAA0226 | COL14A1 | CLIC2 | CCL3L3 | CATSPER1 |
| KIAA0226L | COL16A1 | CLIC5 | CCL4L1 | CCDC115 |
| LILRA4 | COL18A1 | CNTNAP3B | CCL4L2 | CD274 |
| LOC283663 | COL1A1 | COL15A1 | CCR1 | CD33 |
| LY9 | COL1A2 | CRIM1 | CCRL2 | CD69 |
| LYN | COL3A1 | CSGALNACT1 | CD14 | CD82 |
| MS4A1 | COL5A2 | CTNND1 | CD163 | CDCP1 |
| MZB1 | COL5A3 | CTTNBP2NL | CD163L1 | CDH12 |
| NAPSB | COL6A1 | CX3CL1 | CD209 | CDK15 |
| NCF1 | COL6A3 | CXorf36 | CD300C | CKS2 |
| NCF1B | COX4I2 | CYYR1 | CD300E | CLCN3 |
| NCF1C | CPE | DLL4 | CD300LB | CLNK |
| NCOA3 | CRISPLD2 | DOCK6 | CD302 | CLU |
| PAX5 | CRYAB | DOCK9 | CD68 | CMA1 |
| RALGPS2 | CSPG4 | DYSF | CD80 | CPA3 |
| SELL | DAAM2 | ECSCR | CD86 | CPEB4 |
| SNX2 | DBNDD2 | EFNA1 | CEBPB | CPPED1 |
| SNX29P1 | DCN | EFNB2 | CECR1 | CRLF2 |
| SPIB | DKK3 | EGFL7 | CFD | CSF2RB |
| ST6GAL1 | DSTN | EHD4 | CLDN1 | CTNNBL1 |
| TCL1A | EBF1 | ELK3 | CLEC10A | CTSG |
| TLR9 | ECM2 | ELTD1 | CLEC4A | CTTNBP2 |
| WDFY4 | EDIL3 | EMCN | CLEC4E | DDX26B |
| | EDNRA | EMP1 | CLEC5A | DDX3Y |
| | EFEMP2 | ENG | CLEC7A | DENNDIB |
| | ENPEP | ENPP2 | CPVL | DIP2C |
| | EPS8 | EPB41L4A | CREG1 | DRD2 |
| | FHL5 | ESAM | CRYL1 | DUSP10 |
| | FILIP1L | ESM1 | CSF1R | DUSP14 |
| | FN1 | EXOC3L1 | CSF2RA | DUSP6 |
| | FOXS1 | EXOC3L2 | CSF3R | EDEM2 |
| | GALNT16 | FAM107A | CSTA | EFHC2 |
| | GEM | FAM167B | CTSB | ELL2 |
| | GJC1 | FAM198B | CTSL1 | ELOVL7 |
| | GPRC5C | FAM65A | CTSS | EMR2 |
| | GPX3 | FCN3 | CXCL16 | ENPP3 |
| | GUCY1A2 | FGD5 | CXCL3 | EPB41L1 |
| | GUCY1A3 | FGF12 | CYBB | ESYT1 |
| | GUCY1B3 | FKBP1A | CYP2S1 | EVPL |
| | HEPH | FLI1 | DAPK1 | EXOSC8 |
| | HEYL | FLJ41200 | DMXL2 | FAIM2 |
| | HIGD1B | FLT1 | DRAM2 | FAM157B |
| | HSPB2 | FLT4 | DSC2 | FCER1A |
| | ITGA7 | GABRD | DSE | FER |
| | KANK2 | GALNT15 | EBI3 | FOSB |
| | KCNE4 | GALNT18 | EPB41L3 | GALC |
| | KCNJ8 | GIMAP6 | EREG | GALNT6 |
| | KIRREL | GIMAP8 | F13A1 | GATA1 |
| | LDB3 | GIPC2 | FAM105A | GATA2 |
| | LGALS3BP | GJA1 | FAM26F | GCSAML |
| | LGI4 | GNG11 | FAM49A | GGT1 |
| | LHFP | GPRC5B | FCAR | GM2A |
| | LMOD1 | GRB10 | FCGBP | GMPR |
| | LPL | HECW2 | FCGR1A | GRAP2 |
| | LPP | HEG1 | FCGR1B | HDC |
| | LPPR4 | HID1 | FCGR1C | HPGD |
| | LTBP1 | HLX | FCGR2A | HPGDS |
| | LUM | HPCAL1 | FCGR2B | HS3ST1 |
| | LURAP1L | HSPA12B | FCGR2C | HS6ST1 |
| | LZTS1 | HSPG2 | FCGRT | IL18R1 |
| | MAP2 | HYAL2 | FCN1 | IL1RL1 |
| | MARK1 | ICAM2 | FGD4 | IL5RA |
| | MFGE8 | IFI27 | FGL2 | JPH4 |
| | MGP | IGFBP3 | FOLR2 | KCNMA1 |
| | MIR143HG | IL3RA | FPR1 | KCNQ1 |
| | MMP11 | INPP1 | FPR3 | KIAA1522 |
| | MRGPRF | INSR | FUCA1 | KIAA1549 |
| | MRVI1 | IPO11-LRRC70 | G0S2 | KIT |
| | MSRB3 | IQCK | GAA | KREMEN1 |
| | MT1A | ITGA2 | GAB2 | KRT1 |
| | MT2A | ITGA5 | GATM | LAT |
| | MYH11 | ITGA6 | GCA | LAT2 |
| | MYL9 | ITGB4 | GGTA1P | LAX1 |
| | MYLK | JAG2 | GK | LEO1 |
| | MYO1B | JAM2 | GPR137B | LIF |
| | NNMT | KANK3 | GPR34 | LOC100130264 |
| | NOTCH3 | KCNJ2 | GPR84 | LOC284454 |
| | NRIP2 | KCNK5 | GRINA | LOC339524 |
| | NTRK2 | KCNN3 | GRN | LPCAT2 |
| | NUPR1 | KDR | HCAR2 | LTC4S |
| | OLFML2B | KIAA0355 | HCAR3 | MAOB |
| | PALLD | KIAA1462 | HCK | MAPK6 |
| | PARM1 | KIAA1671 | HEIH | MAPRE1 |
| | PCDH18 | KL | HEXA | MBOAT7 |
| | PCOLCE | LDB2 | HEXB | MEIS2 |
| | PDE5A | LIMS2 | HK3 | MITF |
| | PDGFA | LOC100505495 | HLA-DMB | MLPH |
| | PDGFRB | LUZP1 | HLA-DPB2 | MRGPRX2 |
| | PHLDA1 | MALL | HMOX1 | MS4A2 |
| | PLAC9 | MANSC1 | HPSE | MSRA |
| | PLEKHH2 | MECOM | HSD17B11 | NDEL1 |
| | PLN | MGST2 | HSPA6 | NDST2 |
| | PODN | MKL2 | HSPA7 | NEK6 |
| | PPP1RUA | MMRN2 | IER3 | NFKBIZ |
| | PRELP | MPZL2 | IFI30 | NMT2 |
| | PRKG1 | MTMR9LP | IGF1 | NRCAM |
| | PTEN | MYCT1 | IGSF21 | NTM |
| | PTGIR | NDST1 | IGSF6 | NTRK1 |
| | RASD1 | NEDD9 | IL10 | OSBPL8 |
| | RASL12 | NOS3 | IL1A | P2RX1 |
| | RCN3 | NOSTRIN | IL1B | PADI2 |
| | REM1 | NOTCH4 | IL1R2 | PAK1 |
| | RERG | NOX4 | ILIRAP | PAQR5 |
| | RGS16 | NPDC1 | IL1RN | PEPD |
| | RGS5 | NPR1 | IL6R | PIGA |
| | S1PR3 | NRN1 | IL8 | PIK3R6 |
| | SELM | PALMD | INSIG1 | PLAT |
| | SEMA5A | PCDH12 | IRAK2 | PLGRKT |
| | 4-Sep | PCDH17 | IRAK3 | PLIN2 |
| | SERPINF1 | PDE10A | KCTD12 | PLXNA4 |
| | SERPING1 | PDE2A | KLF4 | PPM1H |
| | SGCA | PECAM1 | KYNU | PPP1R15B |
| | SLC2A4 | PIK3R3 | LGMN | PRDX1 |
| | SLC7A2 | PKN3 | LILRA1 | PRDX6 |
| | SMOC2 | PLCB1 | LILRA2 | PRELID2 |
| | SOD3 | PLVAP | LILRA3 | PRG2 |
| | SORBS3 | PLXNA2 | LILRA6 | PRKAB1 |
| | SSTR2 | PLXND1 | LILRB2 | PRKCA |
| | STEAP4 | PODXL | LILRB3 | PRR26 |
| | SUSD2 | PPM1F | LILRB4 | PTGS1 |
| | SYNPO2 | PREX2 | LILRB5 | RAB27B |
| | TAGLN | PRSS23 | LOC100505702 | RAB37 |
| | TFPI | PTPRB | LOC338758 | RAB38 |
| | TGFB1I1 | PTPRM | LOC731424 | RAB44 |
| | TGFB3 | PVR | LRRC25 | RASGRP4 |
| | THBS2 | RAMP2 | LST1 | RD3 |
| | THY1 | RAMP3 | LY86 | RGS13 |
| | TINAGL1 | RAPGEF3 | LYZ | RHBDD2 |
| | TMEM119 | RAPGEF4 | MAFB | RPS6KA5 |
| | TNFAIP6 | RAPGEF5 | MAN2B1 | SDPR |
| | TPM1 | RASA4 | MANBA | SERPINB1 |
| | TPM2 | RASGRF2 | MB21D2 | SIGLEC17P |
| | TPPP3 | RASGRP3 | MCOLN1 | SIGLEC6 |
| | TRPC6 | RASIP1 | ME1 | SIGLEC8 |
| | VCL | RBP7 | MERTK | SLC11A2 |
| | ZAK | RGS3 | MFSD1 | SLC18A2 |
| | | RND1 | MGAT1 | SLC1A5 |
| | | ROBO4 | MKNK1 | SLC24A3 |
| | | RPS6KA2 | MNDA | SLC26A2 |
| | | S100A16 | MPEG1 | SLC2A6 |
| | | S1PR1 | MRC1 | SLC39A11 |
| | | SCARB1 | MRO | SLC44A1 |
| | | SCHIP1 | MS4A14 | SLC45A3 |
| | | SEC14L1 | MS4A4A | SLC4A8 |
| | | SEMA3F | MS4A6A | SLC8A3 |
| | | SEMA6B | MS4A7 | SMIM3 |
| | | SH3BGRL2 | MSR1 | SMYD3 |
| | | SHANK3 | MXD1 | SSR4 |
| | | SHE | MYO7A | ST3GAL4 |
| | | SHROOM4 | NAAA | STMN1 |
| | | SLC29A1 | NAGA | STX3 |
| | | SLC9A3R2 | NAIP | STXBP2 |
| | | SLCO2A1 | NAMPT | STXBP5 |
| | | SMAGP | NCF2 | STXBP6 |
| | | SOCS2 | NFAM1 | SVOPL |
| | | SOX7 | NINJ1 | TBC1D14 |
| | | SPRY1 | NLRP3 | TDRD3 |
| | | SPTBN1 | NPL | TEC |
| | | STC1 | OGFRL1 | TESPA1 |
| | | SYNPO | OLR1 | TMEM154 |
| | | TEK | OR2B11 | TMOD1 |
| | | TGFBR2 | OSCAR | TPSAB1 |
| | | TGM2 | OSM | TPSB2 |
| | | THSD1 | P2RX7 | TPSD1 |
| | | THSD7A | P2RY13 | TPSG1 |
| | | TIE1 | PILRA | TPST2 |
| | | TJP1 | PLA2G7 | TRPV2 |
| | | TM4SF1 | PLB1 | TSC22D2 |
| | | TM4SF18 | PLBD1 | TSNAX |
| | | TMEM204 | PLEK | TSTD1 |
| | | TNFAIP1 | PLSCR1 | TTI1 |
| | | TNFAIP8L1 | PLXDC2 | UNC13D |
| | | TNFRSF10C | PPIF | VAT1 |
| | | TNXB | PPT1 | VWA5A |
| | | TSPAN18 | PYGL | ZNF48 |
| | | TSPAN7 | RAB20 | |
| | | USHBP1 | RASSF4 | |
| | | VAMP5 | RBM47 | |
| | | VWF | RGS18 | |
| | | WWTR1 | RNASE6 | |
| | | ZNF366 | S100A9 | |
| | | | SCIMP | |
| | | | SDS | |
| | | | SERPINA1 | |
| | | | SERPINB8 | |
| | | | SHMT1 | |
| | | | SIGLEC1 | |
| | | | SIGLEC10 | |
| | | | SIGLEC9 | |
| | | | SIRPA | |
| | | | SIRPB1 | |
| | | | SIRPB2 | |
| | | | SLAMF8 | |
| | | | SLC11A1 | |
| | | | SLC15A3 | |
| | | | SLC16A10 | |
| | | | SLC1A3 | |
| | | | SLC31A2 | |
| | | | SLC37A2 | |
| | | | SLC40A1 | |
| | | | SLC43A2 | |
| | | | SLC7A7 | |
| | | | SLC8A1 | |
| | | | SLCO2B1 | |
| | | | SNX8 | |
| | | | SOD2 | |
| | | | SPI1 | |
| | | | SPP1 | |
| | | | STAB1 | |
| | | | TBXAS1 | |
| | | | TFEC | |
| | | | TFRC | |
| | | | TGFBI | |
| | | | THEMIS2 | |
| | | | TKT | |
| | | | TLR1 | |
| | | | TLR2 | |
| | | | TLR4 | |
| | | | TM6SF1 | |
| | | | TMEM106A | |
| | | | TMEM176A | |
| | | | TMEM176B | |
| | | | TMEM86A | |
| | | | TNF | |
| | | | TNFSF13 | |
| | | | TNFSF13B | |
| | | | TOM1 | |
| | | | TREM1 | |
| | | | TREM2 | |
| | | | TRPM2 | |
| | | | VMO1 | |
| | | | VSIG4 | |
| | | | WDR91 | |
| | | | ZNF267 | |
| | | | ZNF385A | |

| | CD4 | CD8 | | Malignant |
|---|---|---|---|---|
| NK cell | T cell | T cell | T cell | cell |
| AOAH | ADAM19 | CD8A | ADAM19 | ABTB2 |
| B3GNT7 | CCR4 | CD8B | BCL11B | AK4 |
| C1orf21 | CD28 | GZMH | CAMK4 | ALDH1A3 |
| CD247 | CD4 | LAG3 | CCR4 | ALDH7A1 |
| CD7 | CD40LG | | CCR5 | ALKBH2 |
| CMC1 | CD5 | | CD2 | ALX4 |
| DENND2D | CTLA4 | | CD27 | ANKRD20A12P |
| EFHD2 | CXCR6 | | CD28 | APLP1 |
| FGFBP2 | DPP4 | | CD3D | ARC |
| GK5 | FLT3LG | | CD3E | ARMCX2 |
| GNLY | GPRIN3 | | CD3G | ATN1 |
| GZMB | ICOS | | CD4 | ATXN7L3B |
| HIPK2 | IL7R | | CD40LG | BAI2 |
| IL2RB | MAF | | CD5 | BAMBI |
| KIR2DL1 | OXNAD1 | | CD6 | BARX2 |
| KIR2DL3 | PBX4 | | CD8A | BEX2 |
| KIR2DL4 | PBXIP1 | | CD8B | BMP4 |
| KIR2DS4 | POLR3E | | CDKN1B | BMP5 |
| KIR3DL1 | RCAN3 | | CLEC2D | BMP7 |
| KIR3DL2 | SPOCK2 | | CTLA4 | BMPER |
| KLRC1 | TNFRSF25 | | CXCR6 | BNC2 |
| KLRC2 | TRAT1 | | DPP4 | BRD8 |
| KLRC3 | | | DUSP4 | C14orf39 |
| KLRD1 | | | EMB | C19orf48 |
| KLRF1 | | | EMBP1 | C5orf4 |
| KRT86 | | | EML4 | CA10 |
| LGALS9C | | | FAM102A | CA11 |
| MCTP2 | | | FLT3LG | CACNA1G |
| MLC1 | | | FYB | CAD |
| NCR1 | | | GALM | CADM1 |
| PRF1 | | | GPR171 | CBX1 |
| S1PR5 | | | GPRIN3 | CCBE1 |
| SH2D1B | | | GZMH | CCDC144B |
| SLFN13 | | | GZMK | CCDC144C |
| SPON2 | | | ICOS | CCDC171 |
| TXK | | | IL32 | CCNB1IP1 |
| ZBTB16 | | | IL7R | CDH11 |
| | | | LAG3 | CDH3 |
| | | | LCK | CDON |
| | | | LEPROTL1 | CES4A |
| | | | LIME1 | CHST8 |
| | | | MAF | CILP2 |
| | | | MIAT | CKB |
| | | | NLRC5 | CKS1B |
| | | | OXNAD1 | CLUL1 |
| | | | PBX4 | COL11A2 |
| | | | PBXIP1 | COL2A1 |
| | | | PDCD1 | COL4A5 |
| | | | PIK3IP1 | COL9A2 |
| | | | POLR3E | COL9A3 |
| | | | RCAN3 | COLEC12 |
| | | | SIRPG | CPT1C |
| | | | SPOCK2 | CRABP1 |
| | | | TC2N | CRABP2 |
| | | | THEMIS | CRISPLD1 |
| | | | TNFRSF25 | CRLF1 |
| | | | TRAT1 | CRNDE |
| | | | UBASH3A | CRTAC1 |
| | | | WNK1 | CSAD |
| | | | ZFP36L2 | CTAG1A |
| | | | | CTAG1B |
| | | | | CUL7 |
| | | | | DHRS3 |
| | | | | DLK1 |
| | | | | DLX1 |
| | | | | DLX2 |
| | | | | DMKN |
| | | | | DNAH14 |
| | | | | DNM3OS |
| | | | | DNPH1 |
| | | | | DPEP1 |
| | | | | EDN3 |
| | | | | EFNA2 |
| | | | | EFNA5 |
| | | | | EGFR |
| | | | | EPCAM |
| | | | | EPS8L2 |
| | | | | ETV4 |
| | | | | FAHD2B |
| | | | | FAM115A |
| | | | | FBLN1 |
| | | | | FBN2 |
| | | | | FBXO2 |
| | | | | FGF11 |
| | | | | FGF19 |
| | | | | FGF9 |
| | | | | FGFR1 |
| | | | | FGFR2 |
| | | | | FGFRL1 |
| | | | | FIBCD1 |
| | | | | FKBP10 |
| | | | | FKTN |
| | | | | FLNC |
| | | | | FLRT1 |
| | | | | FLRT3 |
| | | | | FOXD2-AS1 |
| | | | | FOXF1 |
| | | | | FSTL4 |
| | | | | FUZ |
| | | | | FZD1 |
| | | | | GADD45G |
| | | | | GATA6 |
| | | | | GFRA1 |
| | | | | GLT8D2 |
| | | | | GPC4 |
| | | | | GPR125 |
| | | | | GRIK3 |
| | | | | GRM4 |
| | | | | GSTA4 |
| | | | | HHAT |
| | | | | HIST1H2BK |
| | | | | HRNR |
| | | | | HS6ST2 |
| | | | | IFT88 |
| | | | | IGF2 |
| | | | | IGF2BP2 |
| | | | | IQCA1 |
| | | | | KDM5B |
| | | | | KIF1A |
| | | | | KIF26B |
| | | | | KLK10 |
| | | | | LINC00516 |
| | | | | LINC00665 |
| | | | | LOC100128881 |
| | | | | LOC100506123 |
| | | | | LOC101101776 |
| | | | | LOC339166 |
| | | | | LOC349196 |
| | | | | LPHN1 |
| | | | | LRIG3 |
| | | | | LTBP4 |
| | | | | MAGED4 |
| | | | | MAGED4B |
| | | | | MDK |
| | | | | MEG3 |
| | | | | MEG8 |
| | | | | MEOX2 |
| | | | | MFAP2 |
| | | | | MIR100HG |
| | | | | MLLT11 |
| | | | | MMP2 |
| | | | | MRC2 |
| | | | | MSLN |
| | | | | MSX2 |
| | | | | MTMR11 |
| | | | | MUC1 |
| | | | | MUC6 |
| | | | | NEFH |
| | | | | NET1 |
| | | | | NGFRAP1 |
| | | | | NIPSNAP1 |
| | | | | NIT2 |
| | | | | NKD2 |
| | | | | NLGN3 |
| | | | | NOG |
| | | | | NPTX2 |
| | | | | NPW |
| | | | | NRP2 |
| | | | | NSG1 |
| | | | | NSMF |
| | | | | NTN1 |
| | | | | OCA2 |
| | | | | OLFM1 |
| | | | | OSR1 |
| | | | | PAFAH1B3 |
| | | | | PAGR1 |
| | | | | PAICS |
| | | | | PARP2 |
| | | | | PCDHA6 |
| | | | | PCDHB10 |
| | | | | PCDHB14 |
| | | | | PCDHB2 |
| | | | | PCDHGA3 |
| | | | | PCDHGC3 |
| | | | | PCSK1N |
| | | | | PDGFRA |
| | | | | PHC1 |
| | | | | PHGDH |
| | | | | PIGC |
| | | | | PIGP |
| | | | | PIP5K1A |
| | | | | PKD1 |
| | | | | PNMAL1 |
| | | | | PRAME |
| | | | | PSAT1 |
| | | | | PSD3 |
| | | | | PTCH1 |
| | | | | PTPRF |
| | | | | PTPRU |
| | | | | RASL11B |
| | | | | RBP1 |
| | | | | RIPK4 |
| | | | | RNF212 |
| | | | | ROBO2 |
| | | | | ROR1 |
| | | | | RTL1 |
| | | | | RTN1 |
| | | | | SCRN1 |
| | | | | SCUBE1 |
| | | | | SERPINE2 |
| | | | | SERTAD4 |
| | | | | SGCD |
| | | | | SHANK2 |
| | | | | SHISA2 |
| | | | | SIM2 |
| | | | | SIX1 |
| | | | | SIX4 |
| | | | | SIX5 |
| | | | | SLC16A4 |
| | | | | SOHLH1 |
| | | | | SOX15 |
| | | | | SOX8 |
| | | | | SOX9 |
| | | | | SPDYE8P |
| | | | | SPOCK1 |
| | | | | SSX1 |
| | | | | SSX2 |
| | | | | SSX2B |
| | | | | STEAP2 |
| | | | | STRA6 |
| | | | | SUCO |
| | | | | SV2A |
| | | | | TARBP1 |
| | | | | TBX18 |
| | | | | TBX3 |
| | | | | TBX5 |
| | | | | TCEAL7 |
| | | | | TENM3 |
| | | | | TET1 |
| | | | | TGFB2 |
| | | | | THBS3 |
| | | | | THSD4 |
| | | | | TIMM13 |
| | | | | TLE1 |
| | | | | TMED3 |
| | | | | TMEM106C |
| | | | | TMEM25 |
| | | | | TMEM254 |
| | | | | TMEM30B |
| | | | | TMEM59L |
| | | | | TMEM67 |
| | | | | TMTC2 |
| | | | | TNC |
| | | | | TNNI3 |
| | | | | TNNT1 |
| | | | | TNPO2 |
| | | | | TRO |
| | | | | TRPS1 |
| | | | | TSPYL4 |
| | | | | TUBB2B |
| | | | | TUSC3 |
| | | | | UCHL1 |
| | | | | USP46 |
| | | | | WIF1 |
| | | | | WNT5A |
| | | | | ZFHX4-AS1 |
| | | | | ZIC2 |
| | | | | ZNF512 |
| | | | | ZNF608 |
| | | | | ZNF692 |
| | | | | ZNF711 |

TABLE 5
Canonical markers used for the initial cell type assignments in Table 4.
Canonical markers

| T cell | B cell | Macrophage | Mastocyte | NKcell | Endothelial cell | CAF |
|---|---|---|---|---|---|---|
| CD2 | CD19 | CD163 | ENPP3 | KLRA1 | PECAM1 | FAP |
| CD3D | CD79A | CD14 | KIT | NKG2 | VWF | THY1 |
| CD3E | CD79B | CSF1R | | KLRB1 | CDH5 | DCN |
| CD3G | BLK | | | KLRD1 | | COL1A1 |
| | | | | | | COL1A2 |
| | | | | | | COL6A1 |
| | | | | | | COL6A2 |
| | | | | | | COL6A3 |

Applicants assigned the cells to nine subsets: malignant cells, epithelial cells, Cancer Associated Fibroblasts (CAFs), CD8 and CD4 T cells, B cells, Natural Killer (NK) cells, macrophages, and mastocytes, and generated signatures for each subset (Tables 4, 5, FIGS. 1B, 3A). Malignant cells primarily grouped by their tumor of origin, while their non-malignant counterparts (immune and stroma) grouped primarily by cell type (FIG. 1B), as was observed in other tumor types (Puram et al. Cell 171:1611-1624.e24 (2017; Tirosh et al. Science 352:189-196 (2016); Venteicher et al. Science 355 (2017) doi:10.1126/science.aai8478). The malignant cells of each of the biphasic (BP) tumors (S1 and S12) formed two distinct subsets—epithelial and mesenchymal—which clustered together with malignant cells of the other biphasic tumors (FIG. 1B).

### Example 2—Developmental Hierarchies and a Repeating Pattern of Intratumor Variation

In the malignant cells, Applicants identified three major patterns of intratumor variation that were shared across multiple tumors (FIG. 4A): (de)differentiations, cell cycle, and a new cellular modality that were termed the core oncogenic program. First, Applicants charted the developmental hierarchy of synovial sarcoma cells, revealing a spectrum of cell states along two differentiation trajectories. To uncover this pattern, they identified mesenchymal and epithelial lineage programs based on intratumor variation within biphasic tumors (FIG. 4A, 3A, Tables 1, 6, and 7). The programs overlapped previous signatures of epithelial to mesenchymal transition (Taube et al. PNAS 107:15449-15454 (2010); Gröger et al. PLOS ONE 7:e51136 (2012)) (P<1.55*10⁻¹⁰, hypergeometric test), and included canonical markers of mesenchymal (ZEB1, ZEB2, PDGFRA and SNAI2) and epithelial (MUC1 and EPCAM) cells (FIG. 5A). Next, Applicants scored each cell for the mesenchymal and epithelial programs, and computed differentiation scores based on the overall expression of both programs (FIG. 4C, Methods). This analysis suggests that the cells gradually acquire (or lose) mesenchymal or epithelial features, stemming from a subpopulation of poorly differentiated cells, which underexpressed both programs (FIG. 4C).

### Cellular Plasticity and a Core Oncogenic Program Characterize Synovial Sarcoma Cells

To identify malignant cell functions that may impact immune cell infiltration, Applicants characterized the cellular programs in SyS malignant cells. Applicants identified three co-regulated gene modules, which repeatedly appeared across multiple tumors in Applicants' cohort (FIG. 4A, Table 6, METHODS). The first two modules reflected mesenchymal and epithelial cell states (FIG. 4A, 20A). These differentiation programs included canonical mesenchymal (ZEB1, ZEB2, PDGFRA and SNAI2) or epithelial (MUC1 and EPCAM) markers (36, 37) (P<1.55*10⁻¹⁰, hypergeometric test), and demonstrated that epithelial cells had a marked increase in antigen presentation and interferon (IFN) γ responses (P<8.49*10⁻⁶, hypergeometric test).

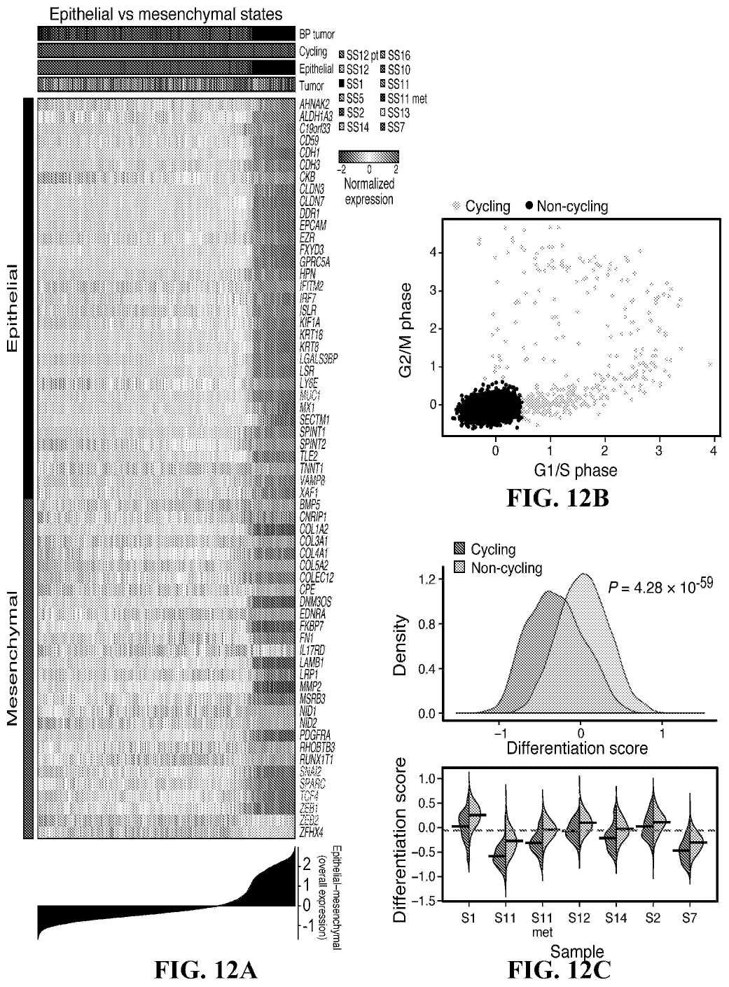

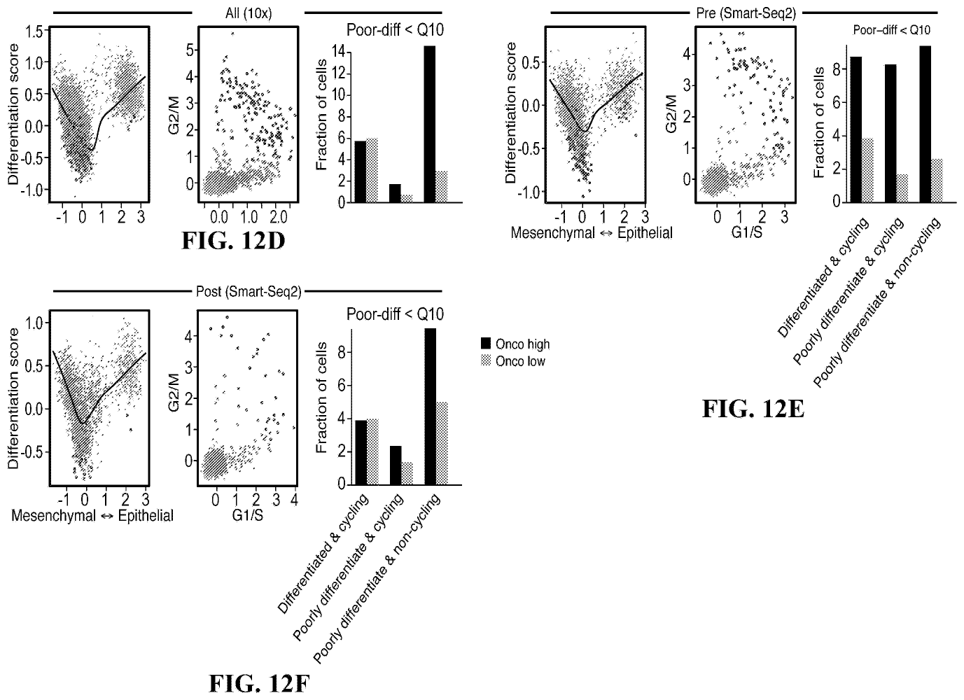

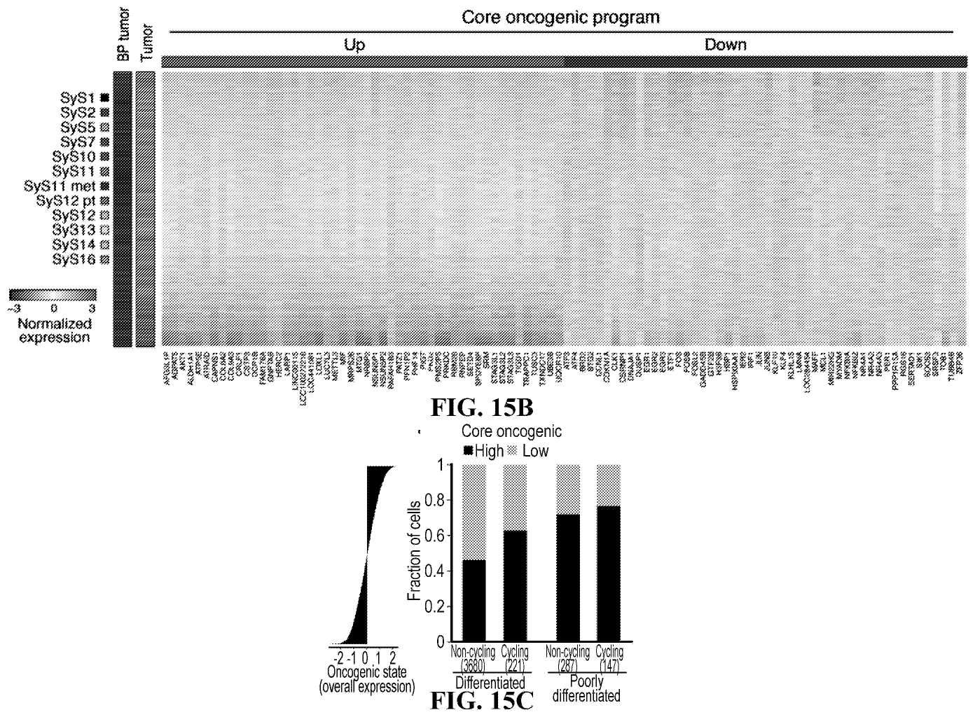

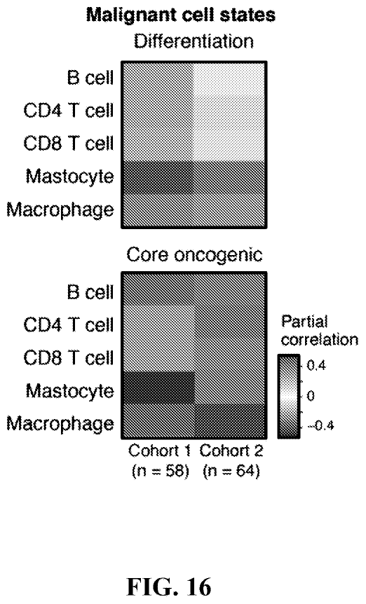

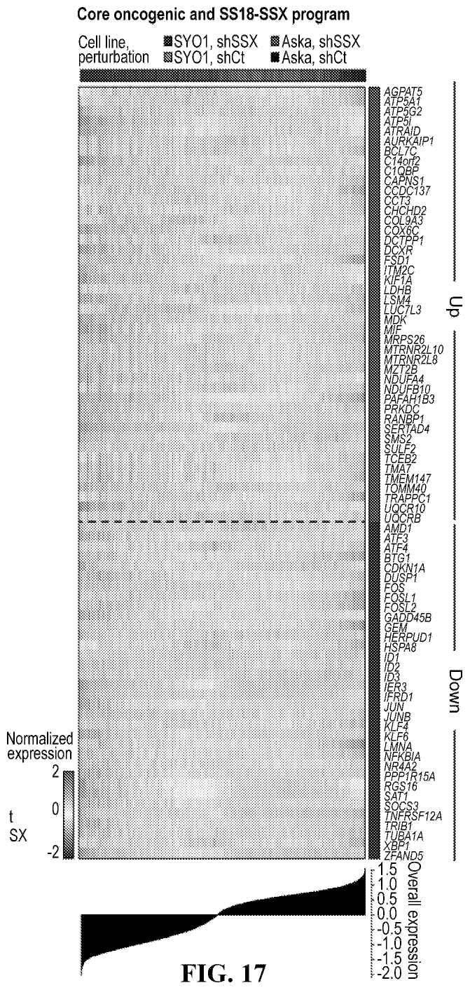

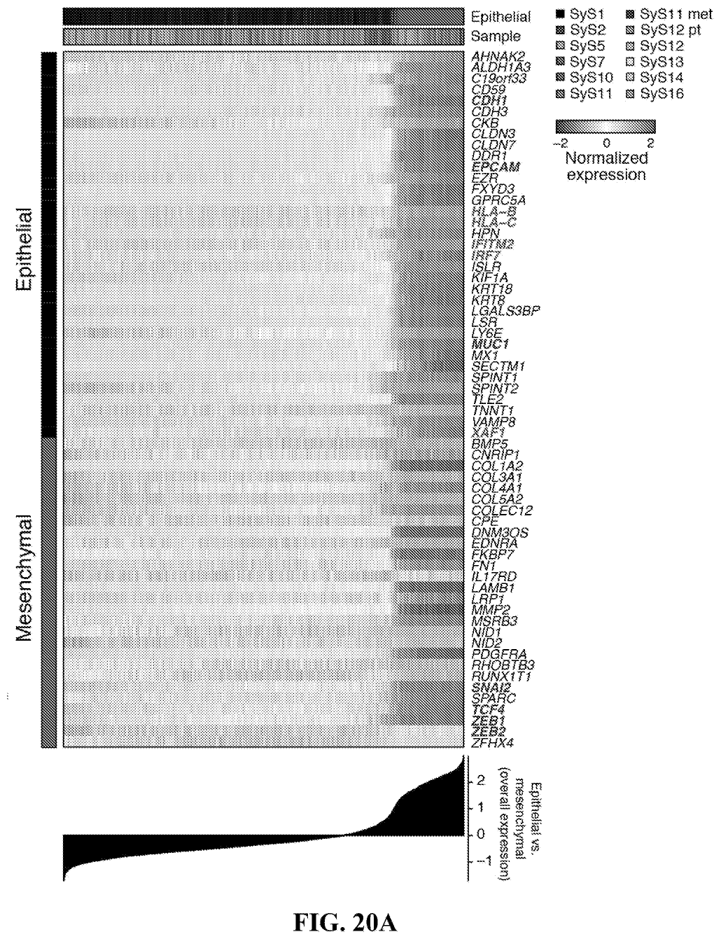

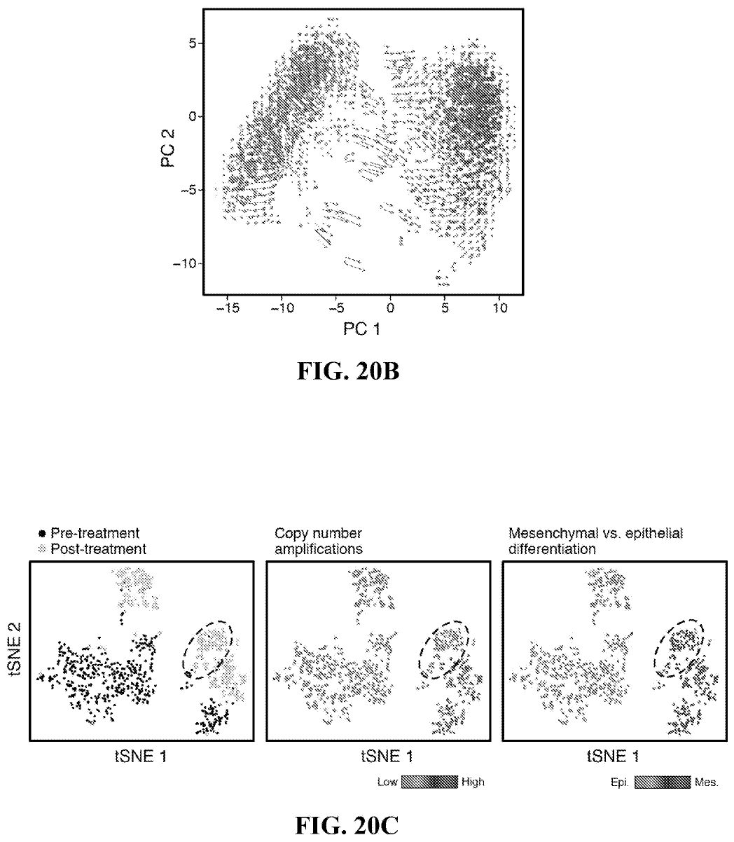

Among mesenchymal cells with a relatively low Overall Expression (METHODS) of the mesenchymal program, one subset also expressed epithelial markers, reminiscent of transitioning to/from an epithelial state, while another underexpressed both programs, reminiscent of a poorly differentiated state. These poorly differentiated cells were highly enriched with cycling cells (P=2.44*10⁻⁶°, mixed effects), indicating that they might function as the tumor progenitors, fueling tumor growth (FIG. 4C, FIG. 12B, 12C). Diffusion map analysis of the cells based on these two programs highlighted putative differentiation trajectories, and found structured differentiation patterns only in the biphasic tumors (FIG. 15A, METHODS). RNA velocity (38) demonstrated that epithelial to mesenchymal transitions may also take place (FIG. 20B), suggestive of cellular plasticity. Further supporting this hypothesis, the post-treatment sample of patient SyS12 includes a new subpopulation of mesenchymal cells, which was absent from the pre-treatment sample, and resembles the epithelial cells in terms of its CNAs (FIG. 20C).

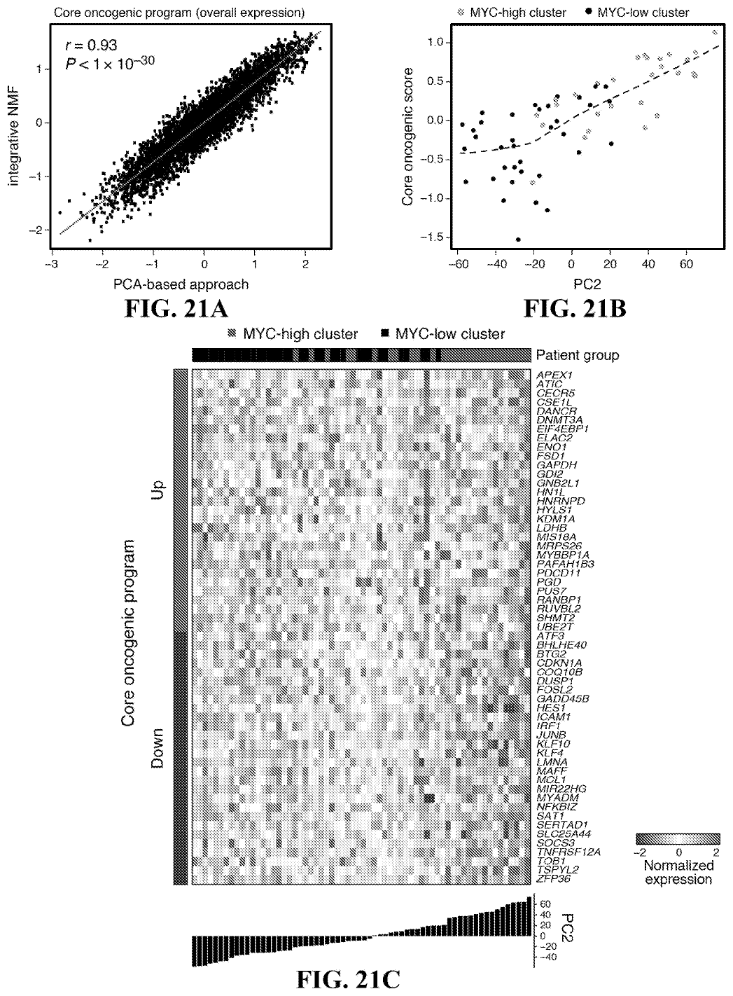

The third module highlighted a new program present in a subset of cells in each tumor (25.2-84.7% per tumor, FIG. 4A, 15B, FIG. 21A-21C), which Applicants named the core oncogenic program. The program is characterized by expression of genes from respiratory carbon metabolism (oxidative phosphorylation, citric acid cycle, and carbohydrate/protein metabolism, P<1*10⁻⁸, hypergeometric test, Table 6), and repression of genes involved in TNF signaling, apoptosis, p53 signaling, and hypoxia processes (P<1*10⁻¹⁰, hypergeometric test, Table 6), including known tumor suppressors, such as p21 (CDKN1A) and KLF4. The program was expressed in a higher proportion of cycling and poorly differentiated cells (P<2.94*10⁻⁴, mixed-effects, FIG. 15C).

To test the clinical value of these transcriptional programs, Applicants reanalyzed two independent bulk gene expression cohorts (21, 22). Both dedifferentiation (METHODS) and the core oncogenic program were substantially more pronounced in the more aggressive poorly differentiated SyS tumors (P<2.76*10⁻⁴, one-sided t-test, FIG. 5A, METHODS), and were associated with increased risk of metastatic disease (P<1.36*10⁻³, Cox regression, FIG. 5B).

TABLE 6
Malignant programs identified in the clinical scRNA-Seq cohort.

| | | | Cell | | | Core oncogenic |
|---|---|---|---|---|---|---|
| Epithelial | | Mesenchymal | cycle | Core oncogenic up | | down |
| ABCG1 | LBH | AASS | ANLN | AFG3L1P | MRPL28 | AKIRIN1 |
| ABHD11 | LECT1 | ADAM33 | ARHGAP11A | AGPAT2 | MRPL35 | AMD1 |
| ABRACL | LGALS3BP | AKAP13 | ATAD5 | AGPAT5 | MRPL4 | ARC |
| ACOT7 | LIME1 | ANKRD44 | BIRC5 | AHCY | MRPL52 | ATF3 |
| ACP5 | LLGL2 | ARMCX3 | BRCA2 | AKR1B1 | MRPS17 | ATF4 |
| ADAMTSL2 | LOC100505761 | ATP1B2 | BUB1B | AKR1C3 | MRPS21 | BHLHE40 |
| AES | LOC541471 | BMP5 | C21orf58 | AKT1 | MRPS26 | BRD2 |
| AGPAT2 | LOC646329 | C14orf37 | CASC5 | ALDH1A1 | MRPS34 | BTG1 |
| AGRN | LPAR2 | C14orf39 | CCNA2 | ALG3 | MTG1 | BTG2 |
| AGTRAP | LPIN3 | C16orf45 | CCNB2 | ALX4 | MTRNR2L1 | C12orf44 |
| AHNAK2 | LRRC16A | Clorf151-NBL1 | CCNE2 | ANAPC7 | MTRNR2L10 | C6orf62 |
| AIG1 | LSR | CACNB2 | CDC6 | ANKRD26P1 | MTRNR2L2 | CCNL1 |
| AKR1C3 | LY6E | CADM1 | CDKN3 | APEH | MTRNR2L6 | CDKN1A |
| ALDH1A3 | LYPD6B | CALD1 | CENPE | APEX1 | MTRNR2L8 | CKS2 |
| ALDH3A2 | MAG11 | CCBE1 | CENPF | APP | MYBBP1A | CLK1 |
| ALDH4A1 | MAL2 | CCDC88A | CENPH | APRT | MZT2B | COQ10B |
| ALOX15 | MAP7 | CD302 | CENPK | ARF5 | NACA | CSRNP1 |
| ANK3 | MBOAT1 | CLIP3 | CENPW | ARL6IP4 | NAT14 | CYCS |
| ANO9 | MCAM | CNRIP1 | CHAF1B | ARL6IP5 | NDUFA1 | DDIT3 |
| ANXA11 | MDK | CNTLN | CLSPN | ASB13 | NDUFA11 | DDX3X |
| ANXA3 | MFSD3 | COL1A2 | DHFR | ATF7IP | NDUFA13 | DDX3Y |
| AP1M2 | MGAT4B | COL21A1 | DNA2 | ATIC | NDUFA3 | DDX5 |
| APOE | MIF4GD | COL4A1 | DTL | ATP5A1 | NDUFA4 | DLX2 |
| APP | MLXIPL | COL4A2 | EZH2 | ATP5C1 | NDUFA7 | DNAJA1 |
| ARHGAP8 | MPZL2 | COL5A1 | FANCA | ATP5E | NDUFA8 | DNAJA4 |
| ARID5A | MSLN | COL5A2 | FANCD2 | ATP5G2 | NDUFAB1 | DNAJB1 |
| ARRDC1 | MSMO1 | COL6A3 | FANCI | ATP5I | NDUFB10 | DNAJB9 |
| ASS1 | MSX2 | COL8A1 | FOXM1 | ATP5J | NDUFB11 | DUSP1 |
| ATHL1 | MUC1 | CPXM1 | GINS2 | ATP5J2 | NDUFB2 | DUSP2 |
| ATP6V0E2 | MX1 | CRTAP | HELLS | ATP5O | NDUFB3 | EGR1 |
| BAIAP2L1 | MYH9 | CXCL12 | KIAA0101 | ATR | NDUFB4 | EGR2 |
| BARX2 | MYO6 | CYGB | KIF11 | ATRAID | NDUFB7 | EGR3 |
| BCAM | NCOA7 | DAB2 | KIF14 | AUP1 | NDUFB9 | EIF1 |
| BSCL2 | NDUFA4L2 | DCN | KIF18A | AURKAIP1 | NDUFS6 | EIF4A3 |
| C14orf1 | NDUFS8 | DEGS1 | KIF20B | BCAP31 | NDUFS8 | EIF5 |
| C19orf21 | NET1 | DNAJA4 | KIF2C | BCL7C | NEDD8 | ERF |
| C19orf33 | NPNT | DNAJC12 | KNSTRN | BMP1 | NEFL | ETF1 |
| C1GALT1C1 | NSMF | DNM3OS | KNTC1 | BOP1 | NHP2 | FAM53C |
| C1orf210 | NT5DC1 | DZIP1 | MAD2L1 | BRK1 | NIPSNAP3A | FOS |
| CAP2 | NT5E | EDNRA | MCM2 | BSG | NKAIN4 | FOSB |
| CAPN6 | NUDT14 | EGFR | MCM3 | BTF3 | NME1 | FOSL1 |
| CARD16 | OAS1 | EMP1 | MCM4 | C11orf48 | NME2 | FOSL2 |
| CARNS1 | OCIAD2 | F2R | MCM5 | C14orf2 | NNT | GADD45B |
| CBLC | OCLN | FBXO32 | MKI67 | C16orf88 | NOMO1 | GEM |
| CCDC153 | ORMDL2 | FERMT2 | MLF1IP | C17orf76-AS1 | NOMO2 | GTF2B |
| CCDC24 | P4HTM | FGF1O | NCAPD2 | C1QBP | NPEPL1 | H3F3B |
| CCND1 | PARD6B | FHL1 | NCAPG2 | C2orf68 | NRBP2 | HBP1 |
| CD151 | PARP8 | FKBP7 | NUSAP1 | C4orf48 | NREP | HERPUD1 |
| CD55 | PARP9 | FLJ42709 | OAS3 | C7orf73 | NSMF | HES1 |
| CD59 | PARVG | FLNB | OIP5 | C9orfl6 | NSUN5 | HSP90AA1 |
| CD7 | PCBD1 | FN1 | ORC6 | CAD | NSUN5P1 | HSP90AB1 |
| CD74 | PDGFB | FOSL2 | PRC1 | CALML3 | NSUN5P2 | HSPA1A |
| CD9 | PDHX | FRZB | PSMC3IP | CAPNS1 | NT5DC2 | HSPA1B |
| CDCP1 | PDLIM1 | FSTL1 | PTTG1 | CBX6 | NUBP2 | HSPA8 |
| CDH1 | PDLIM2 | GALNT18 | RACGAP1 | CCDC137 | NUDT5 | HSPH1 |
| CDH3 | PERP | GEM | RFC4 | CCDC140 | NUTF2 | ICAM1 |
| CDH4 | PHYHD1 | GFPT2 | RNASEH2A | CCT3 | OBSL1 | ID1 |
| CDK2AP2 | PIGV | GFRA1 | RRM2 | CD320 | OGG1 | ID2 |
| CHST9 | PIM1 | GPM6B | SGOL2 | CD63 | OST4 | ID3 |
| CKB | PKP3 | GPX7 | SMC4 | CD7 | OXLD1 | IER2 |
| CLDN3 | PKP4 | GSTA4 | SPAG5 | CDK2AP1 | PAFAH1B3 | IER3 |
| CLDN4 | PLEKHB1 | GSTM5 | SPDL1 | CECR5 | PARK7 | IFRD1 |
| CLDN7 | PLEKHG1 | GYPC | STIL | CHCHD1 | PATZ1 | IRF1 |
| CLIC3 | PLEKHN1 | HAAO | TCF19 | CHCHD2 | PAX3 | JUN |
| CLU | PLLP | HCG11 | TIMELESS | CIAPIN1 | PAX9 | JUNB |
| COL12A1 | PLXDC2 | HENMT1 | TK1 | CKAP5 | PCDHA3 | JUND |
| CRB3 | PLXNA2 | HMGCLL1 | TOP2A | CLDN4 | PDCD11 | KLF10 |
| CRIP1 | PLXNB1 | HOXC10 | TPX2 | CLNS1A | PDCD5 | KLF4 |
| CRIP2 | PNOC | HOXC9 | TYMS | CNPY2 | PDIA4 | KLF6 |
| CXADR | PNP | HSD17B11 | UBE2C | COA5 | PEBP1 | KLHL15 |
| CXCL1 | PPL | IFFO1 | UBE2T | COL18A1 | PET100 | LMNA |
| CYB561 | PPP1CA | IL17RD | UHRF1 | COL5A1 | PFKL | LOC284454 |
| CYBA | PPP1R16A | IL1R1 | WDHD1 | COL6A2 | PFKP | MAFF |
| CYFIP2 | PPP1R1B | INHBA | ZWINT | COL9A3 | PFN1 | MCL1 |
| CYHR1 | PPP1R9A | INPP4B | | COX4I1 | PFN1P2 | MIR22HG |
| CYP39A1 | PRKCG | ITPRIPL2 | | COX5A | PGD | MLF1 |
| CYP4X1 | PRPH | KIF26B | | COX5B | PGLS | MXD1 |
| CYSTM1 | PRR15 | LAMA2 | | COX6A1 | PHF14 | MYADM |
| DBNDD2 | PRR15L | LAMB1 | | COX6B1 | PIGM | NFATC1 |
| DCXR | PRSS8 | LEF1 | | COX6C | PIGQ | NFATC2 |
| DDR1 | PSME1 | LEPRE1 | | COX7C | PIGT | NFKBIA |
| DDX58 | PSME2 | LOXL2 | | CRIP1 | PKD2 | NFKBIZ |
| DHCR7 | PTGER4 | LRP1 | | CRLF1 | PLP2 | NR4A1 |
| DMKN | PTGES | LUM | | CRMP1 | PMS2P5 | NR4A2 |
| DRD1 | PTN | MEF2A | | CSAG3 | POLD2 | NR4A3 |
| DSP | PTPRF | MEOX2 | | CSE1L | POLR1B | PAFAH1B2 |
| EFCAB4A | PTRH1 | MFAP4 | | CSRP2BP | POLR2F | PER1 |
| EFNA5 | RAB3IP | MLF1 | | CST3 | PPIA | PER2 |
| ELOVL1 | RALGPS1 | MMP2 | | CSTB | PPIB | PPP1R15A |
| ELOVL7 | RASSF7 | MSN | | CSTF3 | PPIP5K2 | RGS16 |
| EMB | RBM47 | MSRB3 | | CTAG1A | PPP1R16A | RHOB |
| ENO2 | REC8 | MXRA5 | | CTAG1B | PRDX2 | RIPK4 |
| ENPP5 | REEP2 | MYL9 | | CYC1 | PRDX4 | RRP12 |
| ENTPD3 | RGL3 | NCAM1 | | CYHR1 | PRELID1 | SAT1 |
| EPB41L5 | RHBDF2 | NDNF | | DAD1 | PRKDC | SELK |
| EPCAM | RHBDL1 | NDOR1 | | DANCR | PSMA5 | SERTAD1 |
| EPHA2 | RIPK4 | NEDD4 | | DBNDD1 | PSMA7 | SF1 |
| EPS8L2 | ROBO3 | NEFH | | DCHS1 | PSMB7 | SIK1 |
| ERBB2 | RTN3 | NID1 | | DCP1B | PSMD4 | SLC25A25 |
| ERBB3 | S100A16 | NID2 | | DCTPP1 | PSMG3 | SLC25A44 |
| ESRP1 | S100A4 | NR4A2 | | DCXR | PTPRF | SOCS3 |
| ESRP2 | S100A6 | NUDT11 | | DGCR6L | PTPRS | SRSF3 |
| EZR | SAMD12 | OXER1 | | DHFR | PUS7 | TNFAIP3 |
| F11R | SCG5 | PALLD | | DNMT3A | PXDN | TNFRSF12A |
| F2RL1 | SCNN1A | PDGFRA | | DPEP3 | PYCR1 | TOB1 |
| FAAH | SCRN2 | PDIA5 | | DPYSL2 | RABAC1 | TRIB1 |
| FAAH2 | SEC11C | PDLIM4 | | DYNLRB1 | RABL6 | TSPYL1 |
| FAM111A | SECTM1 | PDZRN3 | | DYNLT1 | RANBP1 | TSPYL2 |
| FAM167A | SELENBP1 | PLIN2 | | EDF1 | RBM26 | TUBA1A |
| FAM213A | SEMA3B | PLK1S1 | | EEF1B2 | RBM6 | TUBA1B |
| FAM221A | SGPL1 | PLSCR4 | | EEF1D | RBX1 | TUBB2A |
| FAM65C | SH3YL1 | PMP22 | | EEF1G | REST | TUBB4B |
| FAM84B | SHANK2 | PPP1R15B | | EIF2AK1 | RGMA | UBB |
| FBXO2 | SHANK2-AS3 | PROS1 | | EIF3C | RGS10 | UBC |
| FBXO44 | SIM2 | QKI | | EIF3H | RHOBTB3 | XBP1 |
| FGF19 | SLC11A2 | QPRT | | EIF3K | RNASEK | YWHAG |
| FGFRL1 | SLC12A2 | RAB31 | | EIF4EBP1 | RNPC3 | ZBTB21 |
| FMO2 | SLC16A5 | RAI14 | | ELAC2 | RNPEP | ZFAND5 |
| FXYD3 | SLC25A25 | RASL11B | | ELOVL1 | ROMO1 | ZFP36 |
| FXYD5 | SLC25A29 | RBMS3 | | EML3 | RUVBL1 | |
| FZD6 | SLC29A1 | RCBTB2 | | ENO1 | RUVBL2 | |
| GALNT3 | SLC35F2 | RCN3 | | EPRS | SARS2 | |
| GAS6 | SLC50A1 | RGL1 | | ERGIC3 | SELENBP1 | |
| GCHFR | SLC6A9 | RGS3 | | ETAA1 | SEMA3A | |
| GPR56 | SLC7A5 | RHOJ | | EXOSC4 | SERF2 | |
| GPRC5A | SLC7A8 | RUNX1T1 | | EXOSC7 | SERTAD4 | |
| GPRC5C | SLFN5 | SEMA6A | | FADD | SETD4 | |
| GRB7 | SLPI | SERTAD1 | | FADS2 | SFN | |
| GSDMD | SMAD1 | SESN1 | | FAM178A | SGK196 | |
| HERC6 | SMPDL3B | SH3PXD2A | | FAM19A5 | SH2D4A | |
| HIGD2A | S0RT1 | SIX1 | | FAM213B | SH3PXD2B | |
| HLA-B | SOX14 | SLC2A10 | | FAM50B | SHMT2 | |
| HMGA1 | SPINT1 | SNAI2 | | FARSA | SIGIRR | |
| HOOK2 | SPINT2 | SPARC | | FARSB | SIM2 | |
| HPN | ST14 | ST3GAL3 | | FBN3 | SIX1 | |
| HSPB2 | ST3GAL5 | STARD13 | | FGF19 | SLC25A23 | |
| IFITM1 | STAP2 | TCF12 | | FGF9 | SLC25A6 | |
| IFITM2 | STRA13 | TCF4 | | FLAD1 | SLC35B4 | |
| IFITM5 | STRA6 | TGFB1I1 | | FMO1 | SLC6A15 | |
| IGFBP6 | STXBP2 | TMEM30B | | FRG1B | SMARCA4 | |
| IGSF9 | SULF1 | TMEM45A | | FSD1 | SMC2 | |
| INADL | SULF2 | TNFRSF19 | | G6PC3 | SMC3 | |
| INF2 | SUMF1 | TSC22D3 | | GABPB1-AS1 | SNHG6 | |
| IQGAP1 | SVIP | UBE2E2 | | GADD45GIP1 | SNRPD2 | |
| IRF6 | SYNGR2 | UBL3 | | GAPDH | SNRPD3 | |
| IRF7 | SYTL1 | UNC5B | | GCN1L1 | SNRPF | |
| ISLR | TACSTD2 | WIF1 | | GDI2 | SOX11 | |
| ITGA3 | TAPBPL | WNT16 | | GEMIN7 | SPCS1 | |
| ITGB4 | TCF7L2 | ZEB1 | | GGH | SPDYE8P | |
| ITGB8 | TENM1 | ZEB2 | | GLB1L | SRI | |
| ITPR2 | TFAP2B | ZFHX4 | | GLB1L2 | SRM | |
| ITPR3 | TFAP2C | ZNF3O2 | | GLI1 | SRSF9 | |
| JUP | TLE2 | | | GNAS | SSNA1 | |
| KIAA1522 | TLE6 | | | GNB2L1 | SSR4 | |
| KIAA1598 | TM4SF1 | | | GNPTAB | SSX2 | |
| KIF1A | TM7SF2 | | | GOLM1 | SSX2B | |
| KLF5 | TMC4 | | | GPR124 | STAG3L1 | |
| KLK1 | TMCC3 | | | GPR126 | STAG3L2 | |
| KLK10 | TMEM125 | | | GPRC5B | STAG3L3 | |
| KLK11 | TMEM176B | | | GSTO2 | STAG3L4 | |
| KLK7 | TNFAIP2 | | | GUSB | STARD4-AS1 | |
| KLK8 | TNFRSF12A | | | H19 | SULF2 | |
| KRT18 | TNFRSF14 | | | HERC2 | SULT1A1 | |
| KRT19 | TNFRSF21 | | | HERC2P7 | SUMF2 | |
| KRT7 | TNFSF13 | | | HIGD2A | SYNPR | |
| KRT8 | TNKS1BP1 | | | HINT1 | TBCD | |
| KRTCAP3 | TNNI3 | | | HMG20B | TCEB2 | |
| | TNNT1 | | | HN1L | TELO2 | |
| | TOM1L1 | | | HNRNPD | TFAP2A | |
| | TPD52 | | | HOXD11 | THY1 | |
| | TSPO | | | HOXD9 | TIGD1 | |
| | TUBB2B | | | HSD17B10 | TIMM13 | |
| | TUBB3 | | | HYAL2 | TIMM8B | |
| | UCP2 | | | HYLS1 | TKT | |
| | VAMP8 | | | ICT1 | TMA7 | |
| | WDR34 | | | IFT81 | TMC6 | |
| | WDR54 | | | IMP3 | TMEM101 | |
| | WFDC2 | | | ING4 | TMEM147 | |
| | XAF1 | | | IRS4 | TMEM177 | |
| | ZDHHC12 | | | ITM2C | TMSB10 | |
| | ZMAT1 | | | ITPA | TMTC2 | |
| | ZNF165 | | | JMJD8 | TOMM40 | |
| | ZNF423 | | | KDM1A | TOMM6 | |
| | ZNF664 | | | KIAA0020 | TOMM7 | |
| | | | | KIF1A | TRAPPC1 | |
| | | | | KRT14 | TSPAN3 | |
| | | | | KRT15 | TSR3 | |
| | | | | KRT8 | TSTA3 | |
| | | | | KRTCAP2 | TTYH3 | |
| | | | | LAMA2 | TUBG1 | |
| | | | | LARP1 | TUFM | |
| | | | | LDHB | TUSC3 | |
| | | | | LECT1 | TWIST2 | |
| | | | | LGALS1 | TXN | |
| | | | | LINC00115 | TXNDC17 | |
| | | | | LINC00116 | TXNDC5 | |
| | | | | LINC00516 | TXNDC9 | |
| | | | | LINC00665 | UBA52 | |
| | | | | LOC100131234 | UBE2T | |
| | | | | LOC100272216 | UBE3B | |
| | | | | LOC101101776 | UCK2 | |
| | | | | LOC202781 | UCP2 | |
| | | | | LOC375295 | UPK3B | |
| | | | | LOC441081 | UQCR10 | |
| | | | | LOC654433 | UQCR11 | |
| | | | | LOXL1 | UQCRB | |
| | | | | LSM4 | UQCRC1 | |
| | | | | LSM7 | UQCRQ | |
| | | | | LUC7L3 | USMG5 | |
| | | | | LY6E | USP5 | |
| | | | | MAB21L1 | VARS | |
| | | | | MAGEA4 | VCAN | |
| | | | | MAGEA9 | VKORC1 | |
| | | | | MAGEC2 | VPS28 | |
| | | | | MAP1B | VPS72 | |
| | | | | MATN3 | VSNL1 | |
| | | | | MBD6 | WDR12 | |
| | | | | MDH2 | YWHAB | |
| | | | | MDK | ZNF212 | |
| | | | | METTL3 | ZNF605 | |
| | | | | MFSD3 | | |
| | | | | MGC21881 | | |
| | | | | MGST1 | | |
| | | | | MGST3 | | |
| | | | | MIF | | |
| | | | | MIS18A | | |
| | | | | MKKS | | |
| | | | | MMP14 | | |
| | | | | MRPL12 | | |
| | | | | MRPL15 | | |
| | | | | MRPL17 | | |

TABLE 7
Malignant programs enrichment with pre-defined gene
sets (hypergeometric p-values: −log10 transformed).

| | Hypergeometric p-values (−log10 transformed) | | | | |
|---|---|---|---|---|---|
| | | | | Core | Core |
| | | | Cell | oncogenic | oncogenic |
| Gene set | Epithelial | Mesenchymal | cycle | up | down |
| HALLMARK TNFA SIGNALING | 0.46 | 1.24 | 0.00 | 0.00 | 17.00 |
| VIA NFKB | | | | | |
| HALLMARK APOPTOSIS | 1.99 | 2.61 | 0.27 | 0.01 | 12.10 |
| HALLMARK HYPOXIA | 0.59 | 1.61 | 0.00 | 0.31 | 9.74 |
| HALLMARK P53 PATHWAY | 2.50 | 0.16 | 0.00 | 0.15 | 9.41 |
| GO CELL CYCLE PROCESS | 0.00 | 0.01 | 17.00 | 0.05 | 2.86 |
| GO NUCLEOSIDE | 0.08 | 0.00 | 0.19 | 17.00 | 1.36 |
| TRIPHOSPHATE METABOLIC | | | | | |
| PROCESS | | | | | |
| GO GLYCOSYL COMPOUND | 0.21 | 0.00 | 0.34 | 17.00 | 1.17 |
| METABOLIC PROCESS | | | | | |
| EMT Up Groger et al. 2012) | 0.00 | 10.84 | 0.00 | 0.38 | 0.33 |
| EMT Up (Taube et al. 2010) | 0.00 | 9.81 | 0.00 | 0.10 | 0.27 |
| GO OXIDATIVE | 0.04 | 0.00 | 0.00 | 17.00 | 0.25 |
| PHOSPHORYLATION | | | | | |
| HALLMARK E2F TARGETS | 0.00 | 0.00 | 17.00 | 1.13 | 0.23 |
| HALLMARK OXIDATIVE | 0.01 | 0.00 | 0.00 | 17.00 | 0.05 |
| PHOSPHORYLATION | | | | | |
| EMT Down (Groger et al. 2012) | 17.00 | 0.32 | 0.00 | 0.06 | 0.00 |
| EMT Down (Taube et al. 2010) | 17.00 | 0.18 | 0.00 | 0.21 | 0.00 |
| GO OXIDOREDUCTASE | 0.34 | 0.00 | 0.43 | 11.46 | 0.00 |
| COMPLEX | | | | | |
| GO POSITIVE REGULATION OF | 0.73 | 2.58 | 0.05 | 0.13 | 17.00 |
| GENE EXPRESSION | | | | | |
| GO POSITIVE REGULATION OF | 0.29 | 2.65 | 0.15 | 0.40 | 17.00 |
| TRANSCRIPTION FROM RNA | | | | | |
| POLYMERASE II PROMOTER | | | | | |
| GO REGULATION OF | 0.05 | 1.21 | 0.18 | 0.14 | 17.00 |
| TRANSCRIPTION FROM RNA | | | | | |
| POLYMERASE II PROMOTER | | | | | |
| GO RNA POLYMERASE II | 0.25 | 2.03 | 0.06 | 0.04 | 17.00 |
| TRANSCRIPTION FACTOR | | | | | |
| ACTIVITY SEQUENCE SPECIFIC | | | | | |
| DNA BINDING | | | | | |
| GO NEGATIVE REGULATION OF | 0.14 | 0.29 | 0.66 | 0.30 | 15.65 |
| NITROGEN COMPOUND | | | | | |
| METABOLIC PROCESS | | | | | |
| GO REGULATION OF CELL | 0.58 | 1.68 | 0.03 | 1.13 | 15.35 |
| DEATH | | | | | |
| GO TRANSCRIPTION FACTOR | 0.43 | 2.98 | 0.00 | 0.06 | 15.35 |
| ACTIVITY RNA POLYMERASE II | | | | | |
| CORE PROMOTER PROXIMAL | | | | | |
| REGION SEQUENCE SPECIFIC | | | | | |
| BINDING | | | | | |
| GO NEGATIVE REGULATION OF | 0.11 | 0.30 | 0.45 | 0.52 | 14.81 |
| GENE EXPRESSION | | | | | |
| GO TRANSCRIPTIONAL | 0.32 | 2.55 | 0.00 | 0.24 | 14.72 |
| ACTIVATOR ACTIVITY RNA | | | | | |
| POLYMERASE II | | | | | |
| TRANSCRIPTION REGULATORY | | | | | |
| REGION SEQUENCE SPECIFIC | | | | | |
| BINDING | | | | | |
| GO POSITIVE REGULATION OF | 0.81 | 1.70 | 0.10 | 0.15 | 14.31 |
| BIOSYNTHETIC PROCESS | | | | | |
| GO NEGATIVE REGULATION OF | 0.10 | 0.63 | 0.50 | 0.20 | 13.59 |
| TRANSCRIPTION FROM RNA | | | | | |
| POLYMERASE II PROMOTER | | | | | |
| GO RESPONSE TO ABIOTIC | 1.40 | 2.28 | 0.53 | 0.83 | 13.31 |
| STIMULUS | | | | | |
| GO SEQUENCE SPECIFIC DNA | 0.07 | 0.93 | 0.89 | 0.15 | 13.07 |
| BINDING | | | | | |
| GO RESPONSE TO | 2.37 | 4.09 | 0.66 | 0.85 | 12.58 |
| ENDOGENOUS STIMULUS | | | | | |
| GO REGULATORY REGION | 0.09 | 0.60 | 0.10 | 0.04 | 12.49 |
| NUCLEIC ACID BINDING | | | | | |
| GO NUCLEIC ACID BINDING | 0.01 | 0.92 | 0.22 | 0.00 | 12.36 |
| TRANSCRIPTION FACTOR | | | | | |
| ACTIVITY | | | | | |
| GO RESPONSE TO NITROGEN | 1.80 | 1.88 | 0.86 | 1.13 | 12.26 |
| COMPOUND | | | | | |
| GO DOUBLE STRANDED DNA | 0.31 | 0.63 | 0.50 | 0.10 | 11.87 |
| BINDING | | | | | |
| GO TRANSCRIPTION FACTOR | 0.01 | 0.57 | 0.05 | 0.42 | 11.80 |
| BINDING | | | | | |
| GO CELLULAR RESPONSE TO | 4.08 | 4.86 | 0.05 | 0.18 | 11.78 |
| ORGANIC SUBSTANCE | | | | | |
| GO TRANSCRIPTIONAL | 0.27 | 2.87 | 0.00 | 0.14 | 11.62 |
| ACTIVATOR ACTIVITY RNA | | | | | |
| POLYMERASE II CORE | | | | | |
| PROMOTER PROXIMAL REGION | | | | | |
| SEQUENCE SPECIFIC BINDING | | | | | |
| HALLMARK UV RESPONSE UP | 0.04 | 0.00 | 0.29 | 0.40 | 11.40 |
| GO REGULATION OF SEQUENCE | 0.71 | 0.02 | 0.13 | 0.04 | 11.38 |
| SPECIFIC DNA BINDING | | | | | |
| TRANSCRIPTION FACTOR | | | | | |
| ACTIVITY | | | | | |
| GO NEGATIVE REGULATION OF | 0.40 | 2.56 | 0.07 | 0.94 | 10.88 |
| CELL DEATH | | | | | |
| GO RESPONSE TO | 0.08 | 0.07 | 0.00 | 0.00 | 10.71 |
| TOPOLOGICALLY INCORRECT | | | | | |
| PROTEIN | | | | | |
| GO RESPONSE TO ORGANIC | 1.88 | 2.32 | 1.54 | 0.35 | 10.58 |
| CYCLIC COMPOUND | | | | | |
| GO TRANSCRIPTION FROM | 0.01 | 0.66 | 0.11 | 0.05 | 10.45 |
| RNA POLYMERASE II | | | | | |
| PROMOTER | | | | | |
| GO REGULATION OF CELL | 0.01 | 0.19 | 14.45 | 0.13 | 10.33 |
| CYCLE | | | | | |
| GO RESPONSE TO EXTERNAL | 7.02 | 1.11 | 0.10 | 0.21 | 10.21 |
| STIMULUS | | | | | |
| GO NEGATIVE REGULATION OF | 1.61 | 2.13 | 0.02 | 0.47 | 10.20 |
| RESPONSE TO STIMULUS | | | | | |
| GO POSITIVE REGULATION OF | 0.47 | 0.17 | 0.04 | 1.28 | 10.18 |
| CELL DEATH | | | | | |
| GO RESPONSE TO OXYGEN | 2.62 | 4.80 | 0.81 | 1.50 | 10.14 |
| CONTAINING COMPOUND | | | | | |
| GO NEGATIVE REGULATION OF | 0.72 | 2.36 | 0.03 | 0.56 | 10.13 |
| CELL COMMUNICATION | | | | | |
| GO CORE PROMOTER | 0.27 | 1.52 | 0.14 | 0.02 | 9.85 |
| PROXIMAL REGION DNA | | | | | |
| BINDING | | | | | |
| GO NEGATIVE REGULATION OF | 0.67 | 0.39 | 1.25 | 0.57 | 9.42 |
| PROTEIN METABOLIC PROCESS | | | | | |
| GO TISSUE DEVELOPMENT | 5.77 | 9.95 | 0.12 | 0.80 | 9.34 |
| GO RESPONSE TO PEPTIDE | 0.29 | 0.37 | 0.38 | 0.71 | 9.07 |
| GO RHYTHMIC PROCESS | 0.28 | 0.14 | 1.67 | 0.33 | 9.02 |
| GO RESPONSE TO OXIDATIVE | 0.54 | 2.58 | 0.36 | 2.79 | 8.80 |
| STRESS | | | | | |
| GO CIRCADIAN RHYTHM | 0.11 | 0.00 | 1.03 | 0.54 | 8.64 |
| GO RESPONSE TO INORGANIC | 4.05 | 2.06 | 0.60 | 1.93 | 8.50 |
| SUBSTANCE | | | | | |
| GO REGULATION OF CELL | 3.76 | 5.75 | 0.02 | 0.27 | 8.26 |
| DIFFERENTIATION | | | | | |
| GO REGULATION OF DNA | 0.08 | 0.27 | 0.00 | 0.72 | 8.17 |
| TEMPLATED TRANSCRIPTION | | | | | |
| IN RESPONSE TO STRESS | | | | | |
| GO REGULATION OF CELL | 4.40 | 3.02 | 1.53 | 0.18 | 8.03 |
| PROLIFERATION | | | | | |
| GO RESPONSE TO | 1.53 | 0.17 | 0.37 | 0.35 | 7.98 |
| EXTRACELLULAR STIMULUS | | | | | |
| GO NEGATIVE REGULATION OF | 0.17 | 0.13 | 0.93 | 0.33 | 7.96 |
| PROTEIN MODIFICATION | | | | | |
| PROCESS | | | | | |
| GO CELLULAR RESPONSE TO | 0.65 | 0.08 | 0.00 | 0.11 | 7.91 |
| EXTRACELLULAR STIMULUS | | | | | |
| GO POSITIVE REGULATION OF | 1.69 | 0.35 | 0.00 | 0.16 | 7.80 |
| IMMUNE SYSTEM PROCESS | | | | | |
| GO PROTEIN REFOLDING | 0.00 | 0.67 | 0.00 | 0.00 | 7.78 |
| GO REGULATION OF PROTEIN | 0.57 | 1.41 | 1.71 | 0.05 | 7.73 |
| MODIFICATION PROCESS | | | | | |
| GO CELLULAR RESPONSE TO | 2.02 | 4.61 | 0.06 | 0.39 | 7.71 |
| ENDOGENOUS STIMULUS | | | | | |
| GO REGULATION OF | 0.07 | 1.12 | 0.00 | 0.70 | 7.61 |
| APOPTOTIC SIGNALING | | | | | |
| PATHWAY | | | | | |
| GO RESPONSE TO CAMP | 1.84 | 0.33 | 0.63 | 0.94 | 7.61 |
| GO ENZYME BINDING | 0.13 | 0.30 | 2.50 | 0.59 | 7.61 |
| GO NEGATIVE REGULATION OF | 2.69 | 0.09 | 1.06 | 0.28 | 7.57 |
| MOLECULAR FUNCTION | | | | | |
| GO CELLULAR RESPONSE TO | 0.01 | 0.03 | 4.16 | 0.51 | 7.50 |
| STRESS | | | | | |
| GO NEGATIVE REGULATION OF | 0.83 | 0.00 | 0.40 | 0.06 | 7.49 |
| SEQUENCE SPECIFIC DNA | | | | | |
| BINDING TRANSCRIPTION | | | | | |
| FACTOR ACTIVITY | | | | | |
| GO RESPONSE TO RADIATION | 0.80 | 0.76 | 0.33 | 0.65 | 7.48 |
| GO NEGATIVE REGULATION OF | 0.18 | 0.20 | 0.08 | 0.08 | 7.46 |
| INTRACELLULAR SIGNAL | | | | | |
| TRANSDUCTION | | | | | |
| GO CELLULAR RESPONSE TO | 0.69 | 0.15 | 0.00 | 0.13 | 7.27 |
| EXTERNAL STIMULUS | | | | | |
| GO RESPONSE TO HORMONE | 0.72 | 1.79 | 1.04 | 0.55 | 7.27 |
| GO RESPONSE TO PURINE | 1.13 | 0.20 | 0.47 | 0.79 | 7.22 |
| CONTAINING COMPOUND | | | | | |
| GO RESPONSE TO LIPID | 1.14 | 3.32 | 0.49 | 0.25 | 7.18 |
| GO NEGATIVE REGULATION OF | 0.24 | 0.25 | 0.30 | 0.02 | 7.11 |
| PHOSPHORYLATION | | | | | |
| GO REGULATION OF CELLULAR | 0.06 | 0.72 | 0.09 | 0.49 | 6.75 |
| RESPONSE TO STRESS | | | | | |
| GO RESPONSE TO | 1.42 | 0.25 | 1.34 | 0.66 | 6.71 |
| ORGANOPHOSPHORUS | | | | | |
| GO RESPONSE TO STARVATION | 0.17 | 0.11 | 0.00 | 0.19 | 6.66 |
| GO UNFOLDED PROTEIN | 0.03 | 0.17 | 0.42 | 0.61 | 6.66 |
| BINDING | | | | | |
| GO RESPONSE TO | 0.43 | 0.00 | 0.00 | 0.35 | 6.58 |
| CORTICOSTERONE | | | | | |
| GO REGULATION OF | 3.14 | 6.59 | 0.03 | 0.80 | 6.54 |
| MULTICELLULAR ORGANISMAL | | | | | |
| DEVELOPMENT | | | | | |
| GO RESPONSE TO REACTIVE | 0.54 | 1.35 | 0.71 | 1.53 | 6.50 |
| OXYGEN SPECIES | | | | | |
| GO RESPONSE TO | 0.00 | 1.51 | 0.40 | 0.01 | 6.48 |
| TEMPERATURE STIMULUS | | | | | |
| GO RESPONSE TO HYDROGEN | 0.03 | 0.15 | 0.40 | 0.15 | 6.39 |
| PEROXIDE | | | | | |
| GO REGULATION OF RESPONSE | 0.90 | 1.05 | 0.04 | 0.14 | 6.35 |
| TO STRESS | | | | | |
| GO INTRINSIC APOPTOTIC | 0.51 | 0.00 | 0.00 | 0.47 | 6.33 |
| SIGNALING PATHWAY | | | | | |
| GO TRANSCRIPTION FACTOR | 0.17 | 0.29 | 0.04 | 0.36 | 6.33 |
| ACTIVITY PROTEIN BINDING | | | | | |
| GO NEGATIVE REGULATION OF | 0.16 | 1.80 | 0.00 | 0.41 | 6.31 |
| APOPTOTIC SIGNALING | | | | | |
| PATHWAY | | | | | |
| GO REGULATION OF | 0.18 | 1.24 | 0.28 | 0.02 | 6.23 |
| INTRACELLULAR SIGNAL | | | | | |
| TRANSDUCTION | | | | | |
| GO REGULATION OF DNA | 0.05 | 0.21 | 0.00 | 1.69 | 6.22 |
| BINDING | | | | | |
| GO RESPONSE TO | 0.47 | 0.64 | 0.00 | 0.26 | 6.19 |
| MECHANICAL STIMULUS | | | | | |
| GO REGULATION OF IMMUNE | 2.43 | 0.67 | 0.01 | 0.02 | 6.08 |
| SYSTEM PROCESS | | | | | |
| GO SKELETAL MUSCLE CELL | 0.20 | 0.00 | 0.00 | 0.43 | 6.06 |
| DIFFERENTIATION | | | | | |
| GO CELL DEATH | 1.16 | 1.30 | 0.25 | 0.52 | 6.06 |
| GO NEGATIVE REGULATION OF | 0.37 | 0.28 | 0.49 | 0.04 | 6.05 |
| PHOSPHORUS METABOLIC | | | | | |
| PROCESS | | | | | |
| GO MUSCLE STRUCTURE | 1.37 | 4.79 | 0.10 | 0.71 | 6.05 |
| DEVELOPMENT | | | | | |
| GO VASCULATURE | 1.99 | 8.85 | 0.00 | 0.09 | 5.95 |
| DEVELOPMENT | | | | | |
| GO CIRCULATORY SYSTEM | 2.69 | 8.13 | 0.00 | 0.44 | 5.63 |
| DEVELOPMENT | | | | | |
| GO LOCOMOTION | 7.80 | 8.09 | 0.01 | 0.26 | 5.30 |
| GO NEGATIVE REGULATION OF | 0.09 | 0.34 | 7.54 | 0.12 | 5.06 |
| CELL CYCLE | | | | | |
| HALLMARK ESTROGEN | 11.52 | 0.86 | 2.02 | 0.27 | 3.80 |
| RESPONSE LATE | | | | | |
| GO BLOOD VESSEL | 1.43 | 8.47 | 0.00 | 0.07 | 3.66 |
| MORPHOGENESIS | | | | | |
| GO CELL MOTILITY | 6.24 | 7.45 | 0.03 | 0.14 | 3.55 |
| HALLMARK EPITHELIAL | 0.57 | 17.00 | 0.00 | 1.44 | 3.41 |
| MESENCHYMAL TRANSITION | | | | | |
| GO MOVEMENT OF CELL OR | 6.60 | 6.93 | 0.63 | 0.16 | 3.20 |
| SUBCELLULAR COMPONENT | | | | | |
| GO ANGIOGENESIS | 0.43 | 6.61 | 0.00 | 0.06 | 3.06 |
| GO CELL CYCLE | 0.01 | 0.00 | 17.00 | 0.02 | 3.04 |
| HALLMARK INTERFERON | 6.14 | 0.00 | 0.24 | 0.03 | 3.02 |
| GAMMA RESPONSE | | | | | |
| GO CELL CYCLE PHASE | 0.00 | 0.02 | 15.65 | 0.18 | 2.63 |
| TRANSITION | | | | | |
| GO EPITHELIUM | 4.11 | 6.29 | 0.20 | 0.59 | 2.60 |
| DEVELOPMENT | | | | | |
| GO ANATOMICAL STRUCTURE | 1.07 | 6.38 | 0.06 | 0.02 | 2.37 |
| FORMATION INVOLVED IN | | | | | |
| MORPHOGENESIS | | | | | |
| GO DNA CONFORMATION | 0.01 | 0.00 | 17.00 | 0.10 | 2.34 |
| CHANGE | | | | | |
| GO PROTEIN DNA COMPLEX | 0.02 | 0.05 | 7.62 | 0.19 | 2.12 |
| SUBUNIT ORGANIZATION | | | | | |
| GO ENERGY DERIVATION BY | 0.04 | 0.04 | 0.00 | 15.65 | 1.96 |
| OXIDATION OF ORGANIC | | | | | |
| COMPOUNDS | | | | | |
| GO REGULATION OF CELL | 7.12 | 2.03 | 0.00 | 0.04 | 1.87 |
| ADHESION | | | | | |
| GO RESPONSE TO WOUNDING | 6.31 | 2.97 | 0.27 | 0.43 | 1.75 |
| GO REGULATION OF MITOTIC | 0.07 | 0.12 | 11.69 | 0.57 | 1.70 |
| CELL CYCLE | | | | | |
| GO REGULATION OF CELL | 0.15 | 0.32 | 6.66 | 0.77 | 1.67 |
| CYCLE PHASE TRANSITION | | | | | |
| GO MITOTIC CELL CYCLE | 0.16 | 0.00 | 6.67 | 0.08 | 1.50 |
| CHECKPOINT | | | | | |
| GO GENERATION OF | 0.08 | 0.09 | 0.00 | 15.65 | 1.49 |
| PRECURSOR METABOLITES | | | | | |
| AND ENERGY | | | | | |
| GO CHROMATIN ASSEMBLY OR | 0.05 | 0.00 | 7.70 | 0.07 | 1.43 |
| DISASSEMBLY | | | | | |
| GO DNA PACKAGING | 0.04 | 0.00 | 14.22 | 0.05 | 1.36 |
| GO NUCLEOSIDE | 0.08 | 0.00 | 0.55 | 17.00 | 1.34 |
| MONOPHOSPHATE METABOLIC | | | | | |
| PROCESS | | | | | |
| GO ORGAN MORPHOGENESIS | 1.71 | 6.40 | 0.02 | 0.48 | 1.33 |
| GO NUCLEAR CHROMOSOME | 0.00 | 0.12 | 7.64 | 0.07 | 1.30 |
| GO ADENYL NUCLEOTIDE | 0.00 | 0.00 | 6.63 | 0.18 | 1.29 |
| BINDING | | | | | |
| GO PURINE CONTAINING | 0.34 | 0.00 | 0.00 | 17.00 | 1.19 |
| COMPOUND METABOLIC | | | | | |
| PROCESS | | | | | |
| GO MICROTUBULE | 0.08 | 0.02 | 10.01 | 0.42 | 1.19 |
| CYTOSKELETON | | | | | |
| GO MITOTIC CELL CYCLE | 0.00 | 0.00 | 17.00 | 0.12 | 1.10 |
| GO CELL CYCLE CHECKPOINT | 0.06 | 0.00 | 11.53 | 0.12 | 1.08 |
| GO NEGATIVE REGULATION OF | 0.13 | 0.05 | 7.73 | 0.21 | 1.06 |
| MITOTIC CELL CYCLE | | | | | |
| GO CELLULAR RESPONSE TO | 0.01 | 0.00 | 6.20 | 0.14 | 1.05 |
| DNA DAMAGE STIMULUS | | | | | |
| GO CYTOSKELETAL PART | 0.94 | 0.46 | 7.88 | 0.25 | 1.00 |
| GO CELL MORPHOGENESIS | 4.13 | 7.91 | 0.00 | 0.02 | 0.89 |
| INVOLVED IN | | | | | |
| DIFFERENTIATION | | | | | |
| GO MITOCHONDRIAL | 0.00 | 0.00 | 0.00 | 7.71 | 0.85 |
| ELECTRON TRANSPORT | | | | | |
| CYTOCHROME C TO OXYGEN | | | | | |
| GO CYTOSKELETON | 1.24 | 0.66 | 7.67 | 0.09 | 0.83 |
| GO NEGATIVE REGULATION OF | 6.41 | 0.61 | 0.00 | 0.05 | 0.82 |
| CELL ADHESION | | | | | |
| GO CELLULAR RESPIRATION | 0.04 | 0.00 | 0.00 | 17.00 | 0.80 |
| GO MICROTUBULE | 0.46 | 0.01 | 6.49 | 0.39 | 0.79 |
| GO REGULATION OF CELL | 0.16 | 0.16 | 11.87 | 0.70 | 0.77 |
| CYCLE PROCESS | | | | | |
| HALLMARK MYC TARGETS V1 | 0.00 | 0.00 | 3.82 | 6.78 | 0.77 |
| GO CHROMOSOME | 0.00 | 0.00 | 17.00 | 0.02 | 0.74 |
| ORGANIZATION | | | | | |
| GO SPINDLE MIDZONE | 0.91 | 0.63 | 7.19 | 0.26 | 0.72 |
| GO BIOLOGICAL ADHESION | 10.74 | 5.62 | 0.00 | 0.19 | 0.70 |
| GO NUCLEOBASE CONTAINING | 0.29 | 0.02 | 0.78 | 17.00 | 0.68 |
| SMALL MOLECULE METABOLIC | | | | | |
| PROCESS | | | | | |
| GO REGULATION OF CELLULAR | 6.19 | 7.06 | 0.03 | 0.14 | 0.63 |
| COMPONENT MOVEMENT | | | | | |
| GO MEMBRANE REGION | 10.84 | 1.70 | 0.02 | 0.00 | 0.61 |
| GO BASOLATERAL PLASMA | 8.42 | 0.87 | 0.00 | 0.05 | 0.58 |
| MEMBRANE | | | | | |
| GO CELL CYCLE G1 S PHASE | 0.02 | 0.14 | 11.42 | 0.14 | 0.58 |
| TRANSITION | | | | | |
| HALLMARK G2M CHECKPOINT | 0.18 | 0.04 | 17.00 | 0.01 | 0.54 |
| GO ENVELOPE | 0.30 | 0.00 | 0.14 | 14.40 | 0.54 |
| GO CELL SURFACE | 7.57 | 1.03 | 0.06 | 0.14 | 0.53 |
| GO RECEPTOR ACTIVITY | 7.72 | 4.16 | 0.00 | 0.02 | 0.52 |
| GO AMIDE BIOSYNTHETIC | 0.01 | 0.01 | 0.04 | 6.93 | 0.50 |
| PROCESS | | | | | |
| GO ORGANONITROGEN | 0.55 | 0.14 | 0.03 | 17.00 | 0.46 |
| COMPOUND METABOLIC | | | | | |
| PROCESS | | | | | |
| GO DNA REPLICATION | 0.14 | 0.00 | 6.98 | 0.30 | 0.45 |
| INDEPENDENT NUCLEOSOME | | | | | |
| ORGANIZATION | | | | | |
| GO MITOCHONDRIAL | 0.03 | 0.00 | 0.02 | 17.00 | 0.42 |
| ENVELOPE | | | | | |
| GO MITOCHONDRION | 0.00 | 0.00 | 0.03 | 9.53 | 0.41 |
| ORGANIZATION | | | | | |
| GO PEPTIDE METABOLIC | 0.01 | 0.05 | 0.00 | 6.25 | 0.40 |
| PROCESS | | | | | |
| GO CHROMOSOME | 0.00 | 0.02 | 17.00 | 0.01 | 0.40 |
| SEGREGATION | | | | | |
| GO MULTICELLULAR | 0.35 | 7.87 | 0.00 | 1.36 | 0.39 |
| ORGANISMAL | | | | | |
| MACROMOLECULE METABOLIC | | | | | |
| PROCESS | | | | | |
| GO PHOSPHATE CONTAINING | 0.19 | 0.16 | 0.20 | 9.55 | 0.39 |
| COMPOUND METABOLIC | | | | | |
| PROCESS | | | | | |
| GO CHROMOSOME | 0.00 | 0.05 | 17.00 | 0.04 | 0.38 |
| GO APICAL PLASMA | 9.04 | 0.57 | 0.00 | 0.04 | 0.38 |
| MEMBRANE | | | | | |
| GO MULTICELLULAR | 0.30 | 7.46 | 0.00 | 1.20 | 0.36 |
| ORGANISM METABOLIC | | | | | |
| PROCESS | | | | | |
| GO ORGANONITROGEN | 0.10 | 0.13 | 0.08 | 7.92 | 0.35 |
| COMPOUND BIOSYNTHETIC | | | | | |
| PROCESS | | | | | |
| GO REGULATION OF SISTER | 0.00 | 0.00 | 6.08 | 0.04 | 0.34 |
| CHROMATID SEGREGATION | | | | | |
| GO CELL JUNCTION | 8.40 | 0.62 | 0.00 | 0.03 | 0.33 |
| GO PLASMA MEMBRANE | 12.18 | 0.64 | 0.04 | 0.00 | 0.32 |
| REGION | | | | | |
| GO MACROMOLECULAR | 0.02 | 0.00 | 6.21 | 1.91 | 0.27 |
| COMPLEX ASSEMBLY | | | | | |
| GO REGULATION OF | 0.00 | 0.00 | 9.59 | 0.02 | 0.27 |
| CHROMOSOME SEGREGATION | | | | | |
| GO RESPIRATORY CHAIN | 0.17 | 0.00 | 0.00 | 17.00 | 0.27 |
| GO SMALL MOLECULE | 1.56 | 0.24 | 0.05 | 12.25 | 0.26 |
| METABOLIC PROCESS | | | | | |
| GO REGULATION OF | 0.03 | 0.44 | 6.15 | 0.05 | 0.26 |
| ORGANELLE ORGANIZATION | | | | | |
| GO CELL DIVISION | 0.08 | 0.00 | 17.00 | 0.01 | 0.25 |
| GO APICAL PART OF CELL | 9.64 | 0.37 | 0.00 | 0.24 | 0.25 |
| GO EXTRACELLULAR SPACE | 7.39 | 5.48 | 0.01 | 1.55 | 0.23 |
| GO MITOCHONDRION | 0.00 | 0.02 | 0.00 | 12.65 | 0.23 |
| GO EXTRACELLULAR | 4.58 | 12.06 | 0.00 | 3.21 | 0.22 |
| STRUCTURE ORGANIZATION | | | | | |
| GO ELECTRON TRANSPORT | 0.12 | 0.00 | 0.00 | 17.00 | 0.22 |
| CHAIN | | | | | |
| GO OXIDATION REDUCTION | 0.77 | 0.97 | 0.07 | 14.18 | 0.22 |
| PROCESS | | | | | |
| GO CELL CELL ADHESION | 6.70 | 1.30 | 0.00 | 0.12 | 0.21 |
| GO PHOSPHORYLATION | 0.04 | 0.19 | 0.07 | 6.13 | 0.21 |
| GO MEIOTIC CELL CYCLE | 0.27 | 0.00 | 7.40 | 0.30 | 0.21 |
| PROCESS | | | | | |
| GO TRANSLATIONAL | 0.00 | 0.00 | 0.00 | 6.01 | 0.20 |
| TERMINATION | | | | | |
| GO ORGANELLE INNER | 0.06 | 0.00 | 0.04 | 17.00 | 0.17 |
| MEMBRANE | | | | | |
| GO MEIOTIC CELL CYCLE | 0.17 | 0.00 | 7.98 | 0.19 | 0.16 |
| GO SKIN DEVELOPMENT | 5.05 | 7.51 | 0.00 | 0.54 | 0.16 |
| GO MITOCHONDRIAL PART | 0.00 | 0.00 | 0.00 | 17.00 | 0.14 |
| GO REGULATION OF NUCLEAR | 0.13 | 0.00 | 11.48 | 0.14 | 0.14 |
| DIVISION | | | | | |
| GO MESENCHYME | 0.72 | 6.07 | 0.00 | 0.91 | 0.13 |
| DEVELOPMENT | | | | | |
| GO ENDOPLASMIC RETICULUM | 0.23 | 8.64 | 0.00 | 1.09 | 0.12 |
| LUMEN | | | | | |
| GO PROTEIN COMPLEX | 0.11 | 0.01 | 7.23 | 1.32 | 0.12 |
| BIOGENESIS | | | | | |
| GO CELL JUNCTION | 7.55 | 0.97 | 0.00 | 0.04 | 0.12 |
| ORGANIZATION | | | | | |
| GO CARBOHYDRATE | 0.68 | 0.02 | 0.24 | 17.00 | 0.12 |
| DERIVATIVE METABOLIC | | | | | |
| PROCESS | | | | | |
| GO DNA METABOLIC PROCESS | 0.00 | 0.00 | 17.00 | 0.35 | 0.12 |
| GO ORGANOPHOSPHATE | 0.58 | 0.24 | 0.34 | 17.00 | 0.11 |
| METABOLIC PROCESS | | | | | |
| GO PROTEIN COMPLEX | 0.12 | 0.08 | 8.57 | 3.24 | 0.08 |
| SUBUNIT ORGANIZATION | | | | | |
| GO NUCLEAR CHROMOSOME | 0.01 | 0.04 | 17.00 | 0.01 | 0.07 |
| SEGREGATION | | | | | |
| GO REGULATION OF CELL | 0.28 | 0.00 | 10.53 | 0.32 | 0.06 |
| DIVISION | | | | | |
| GO SINGLE ORGANISM | 0.99 | 0.38 | 0.76 | 6.33 | 0.05 |
| BIOSYNTHETIC PROCESS | | | | | |
| GO CELL CELL JUNCTION | 11.12 | 0.10 | 0.00 | 0.18 | 0.04 |
| GO ORGANELLE FISSION | 0.00 | 0.00 | 17.00 | 0.02 | 0.04 |
| GO SPINDLE | 0.06 | 0.02 | 10.38 | 0.32 | 0.04 |
| GO EXTRACELLULAR MATRIX | 1.08 | 15.65 | 0.00 | 1.70 | 0.03 |
| GO CHROMOSOMAL REGION | 0.02 | 0.00 | 17.00 | 0.21 | 0.03 |
| GO MITOTIC NUCLEAR | 0.00 | 0.01 | 17.00 | 0.05 | 0.02 |
| DIVISION | | | | | |
| GO MEMBRANE PROTEIN | 0.94 | 0.02 | 0.00 | 6.76 | 0.00 |
| COMPLEX | | | | | |
| GO INTRINSIC COMPONENT OF | 15.65 | 1.45 | 0.00 | 0.07 | 0.00 |
| PLASMA MEMBRANE | | | | | |
| GO APICAL JUNCTION | 8.93 | 0.19 | 0.00 | 0.09 | 0.00 |
| COMPLEX | | | | | |
| GO APICOLATERAL PLASMA | 7.63 | 0.81 | 0.00 | 0.40 | 0.00 |
| MEMBRANE | | | | | |
| GO ATP DEPENDENT | 0.08 | 0.00 | 6.08 | 0.70 | 0.00 |
| CHROMATIN REMODELING | | | | | |
| GO BASEMENT MEMBRANE | 0.88 | 9.21 | 0.00 | 1.40 | 0.00 |
| GO CELL CELL JUNCTION | 6.40 | 0.00 | 0.00 | 0.06 | 0.00 |
| ASSEMBLY | | | | | |
| GO CENTROMERE COMPLEX | 0.17 | 0.00 | 10.84 | 0.38 | 0.00 |
| ASSEMBLY | | | | | |
| GO CHROMOSOME | 0.03 | 0.00 | 17.00 | 0.03 | 0.00 |
| CENTROMERIC REGION | | | | | |
| GO CHROMOSOME | 0.00 | 0.00 | 6.95 | 0.23 | 0.00 |
| CONDENSATION | | | | | |
| GO COLLAGEN BINDING | 1.20 | 6.63 | 0.00 | 0.23 | 0.00 |
| GO COLLAGEN TRIMER | 0.13 | 11.23 | 0.00 | 1.06 | 0.00 |
| GO COMPLEX OF COLLAGEN | 0.00 | 12.16 | 0.00 | 0.27 | 0.00 |
| TRIMERS | | | | | |
| GO CONDENSED | 0.02 | 0.00 | 17.00 | 0.03 | 0.00 |
| CHROMOSOME | | | | | |
| GO CONDENSED | 0.14 | 0.00 | 17.00 | 0.00 | 0.00 |
| CHROMOSOME CENTROMERIC | | | | | |
| REGION | | | | | |
| GO CYTOCHROME COMPLEX | 0.00 | 0.00 | 0.00 | 6.02 | 0.00 |
| GO DNA DEPENDENT DNA | 0.14 | 0.00 | 10.67 | 0.08 | 0.00 |
| REPLICATION | | | | | |
| GO DNA REPLICATION | 0.11 | 0.04 | 15.05 | 0.04 | 0.00 |
| GO DNA REPLICATION | 0.00 | 0.00 | 10.56 | 0.00 | 0.00 |
| INITIATION | | | | | |
| GO ENDODERMAL CELL | 0.28 | 6.26 | 0.00 | 0.21 | 0.00 |
| DIFFERENTIATION | | | | | |
| GO ENERGY COUPLED PROTON | 0.00 | 0.00 | 0.00 | 6.39 | 0.00 |
| TRANSPORT DOWN | | | | | |
| ELECTROCHEMICAL GRADIENT | | | | | |
| GO EXTRACELLULAR MATRIX | 0.83 | 14.61 | 0.00 | 0.82 | 0.00 |
| COMPONENT | | | | | |
| GO HISTONE EXCHANGE | 0.15 | 0.00 | 7.19 | 0.71 | 0.00 |
| GO HYDROGEN ION | 0.19 | 0.00 | 0.00 | 13.81 | 0.00 |
| TRANSMEMBRANE | | | | | |
| TRANSPORT | | | | | |
| GO HYDROGEN TRANSPORT | 0.54 | 0.16 | 0.00 | 13.65 | 0.00 |
| GO INNER MITOCHONDRIAL | 0.02 | 0.00 | 0.00 | 17.00 | 0.00 |
| MEMBRANE PROTEIN | | | | | |
| COMPLEX | | | | | |
| GO INORGANIC CATION | 0.73 | 0.12 | 0.00 | 6.26 | 0.00 |
| TRANSMEMBRANE | | | | | |
| TRANSPORTER ACTIVITY | | | | | |
| GO KINETOCHORE | 0.09 | 0.00 | 17.00 | 0.00 | 0.00 |
| GO KINETOCHORE | 0.55 | 0.00 | 6.66 | 0.46 | 0.00 |
| ORGANIZATION | | | | | |
| GO LATERAL PLASMA | 7.75 | 0.00 | 0.00 | 0.45 | 0.00 |
| MEMBRANE | | | | | |
| GO MCM COMPLEX | 0.00 | 0.00 | 6.88 | 0.00 | 0.00 |
| GO MITOCHONDRIAL ATP | 0.00 | 0.00 | 0.00 | 6.85 | 0.00 |
| SYNTHESIS COUPLED PROTON | | | | | |
| TRANSPORT | | | | | |
| GO MITOCHONDRIAL | 0.00 | 0.06 | 0.00 | 17.00 | 0.00 |
| MEMBRANE PART | | | | | |
| GO MITOCHONDRIAL PROTEIN | 0.01 | 0.00 | 0.00 | 17.00 | 0.00 |
| COMPLEX | | | | | |
| GO MITOCHONDRIAL | 0.06 | 0.00 | 0.00 | 10.25 | 0.00 |
| RESPIRATORY CHAIN COMPLEX | | | | | |
| ASSEMBLY | | | | | |
| GO MITOCHONDRIAL | 0.09 | 0.00 | 0.00 | 9.97 | 0.00 |
| RESPIRATORY CHAIN COMPLEX | | | | | |
| I BIOGENESIS | | | | | |
| GO MITOTIC SISTER | 0.00 | 0.00 | 10.78 | 0.08 | 0.00 |
| CHROMATID SEGREGATION | | | | | |
| GO MONOVALENT INORGANIC | 0.63 | 0.08 | 0.00 | 9.34 | 0.00 |
| CATION TRANSMEMBRANE | | | | | |
| TRANSPORTER ACTIVITY | | | | | |
| GO MONOVALENT INORGANIC | 0.71 | 0.36 | 0.00 | 9.33 | 0.00 |
| CATION TRANSPORT | | | | | |
| GO NADH DEHYDROGENASE | 0.13 | 0.00 | 0.00 | 14.12 | 0.00 |
| COMPLEX | | | | | |
| GO NUCLEOSIDE | 0.00 | 0.00 | 0.62 | 6.63 | 0.00 |
| TRIPHOSPHATE BIOSYNTHETIC | | | | | |
| PROCESS | | | | | |
| GO OXIDOREDUCTASE | 1.07 | 1.32 | 0.13 | 13.28 | 0.00 |
| ACTIVITY | | | | | |
| GO OXIDOREDUCTASE | 0.00 | 0.00 | 0.00 | 6.18 | 0.00 |
| ACTIVITY ACTING ON A HEME | | | | | |
| GROUP OF DONORS | | | | | |
| GO OXIDOREDUCTASE | 0.73 | 0.20 | 0.00 | 10.49 | 0.00 |
| ACTIVITY ACTING ON NAD P H | | | | | |
| GO OXIDOREDUCTASE | 0.72 | 0.00 | 0.00 | 11.71 | 0.00 |
| ACTIVITY ACTING ON NAD P H | | | | | |
| QUINONE OR SIMILAR | | | | | |
| COMPOUND AS ACCEPTOR | | | | | |
| GO PROTEINACEOUS | 0.51 | 15.65 | 0.00 | 1.76 | 0.00 |
| EXTRACELLULAR MATRIX | | | | | |
| GO PROTON TRANSPORTING | 0.00 | 0.00 | 0.00 | 8.77 | 0.00 |
| ATP SYNTHASE COMPLEX | | | | | |
| GO REGULATION OF EXIT | 0.00 | 0.00 | 6.46 | 0.00 | 0.00 |
| FROM MITOSIS | | | | | |
| GO RENAL SYSTEM PROCESS | 6.80 | 0.87 | 0.00 | 0.92 | 0.00 |
| GO RIBONUCLEOSIDE | 0.00 | 0.00 | 0.00 | 7.80 | 0.00 |
| TRIPHOSPHATE BIOSYNTHETIC | | | | | |
| PROCESS | | | | | |
| GO SISTER CHROMATID | 0.02 | 0.00 | 14.18 | 0.04 | 0.00 |
| COHESION | | | | | |
| GO SISTER CHROMATID | 0.00 | 0.00 | 17.00 | 0.02 | 0.00 |
| SEGREGATION | | | | | |
| GO SPINDLE CHECKPOINT | 0.00 | 0.00 | 6.84 | 0.00 | 0.00 |
| GO SPINDLE MICROTUBULE | 0.09 | 0.00 | 7.79 | 0.05 | 0.00 |
| GO SPINDLE POLE | 0.02 | 0.00 | 8.32 | 0.23 | 0.00 |
| GO TRANSLATIONAL | 0.00 | 0.00 | 0.00 | 7.31 | 0.00 |
| ELONGATION | | | | | |
| HALLMARK MITOTIC SPINDLE | 0.04 | 0.68 | 12.22 | 0.04 | 0.00 |

Second, transcriptional module analysis across all three tumor subtypes (using weighted-PCA via PAGODA (Fan et al. Nat Methods 13:241-244 (2016)) and clustering of gene-gene co-expression networks), also identified a cell cycle program that distinguished cycling from non-cycling cells (P<1*10−30, mixed-effects test, Tables 6, 7, FIG. 4C). Overall, 8.6% of malignant cells were cycling (1.1-23.6% per tumor), such that cycling cells were more frequent in treatment-naïve vs. post-treatment tumors (P=5.21*10−11, hypergeometric test; P=1.33*10−6 logistic mixed-effects test). Interestingly, the cycling cells where substantially less differentiated (P=2.44*10−60, mixed effects), revealing a tumor structure where the poorly differentiated (or stem-like) cells substantially more prone to cycle and replenish the tumor (FIGS. 4B-4F). These findings support a model of malignant cell differentiation, as opposed to dedifferentiation in SyS.

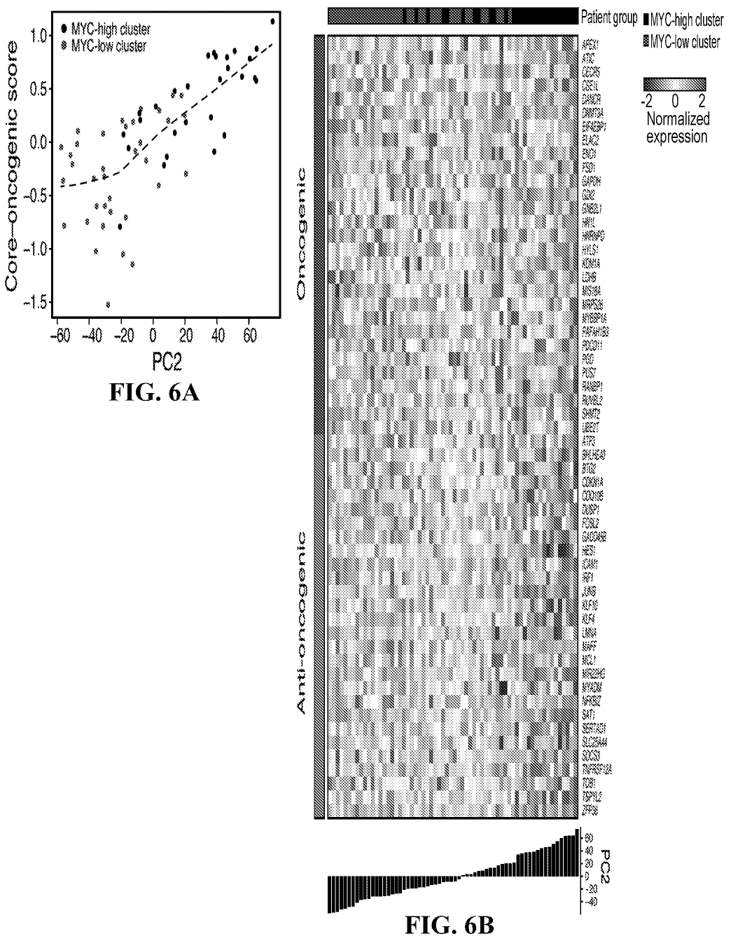

The third module identified a new core oncogenic program present in a subset of cells in each tumor, and characterized by the modulation of several cancer-promoting pathways (FIGS. 4A, 4B). The program induced genes from respiratory carbon metabolism (oxidative phosphorylation, citric acid cycle, and carbohydrate/protein metabolism, P<1*10−8, hypergeometric test), and repressed genes involved in TNF signaling, apoptosis, p53 signaling, and hypoxia processes (P<1*10−10, hypergeometric test, Tables 6 and 7), including known tumor suppressors (e.g., CDKN1A and KLF6). The program was enhanced in cycling and poorly differentiated cells (P<2.94*10−4, mixed-effects, FIG. 4D), and significantly overlapped an immunotherapy resistance program that Applicants recently found in melanoma (Jerby-Arnon et al. Cell 175:984-997.e24 (2018)) (P<7.16*10−10, hypergeometric test). Applicants confirmed the presence of the program in situ at the protein level by immunohistochemistry (IHC) and multiplexed immunofluorescence (t-CyCIF) (Lin et al. eLife 7:e31657 (2018)) (FIGS. 4E-4F); and detected its expression and variation across bulk RNA-Seq data of 64 primary SyS tumors (McBride et al. Cancer Cell (2018) doi:10.1016/j.ccell.2018.05.002) (FIG. 6). Taken together, the core oncogenic program captures intra- and inter-tumor variation, manifests multiple cancer hallmarks, and highlights a yet unappreciated subpopulation of cells in SyS.

### Example 3—the Core Oncogenic Program is Associated with Poor Clinical Outcomes

To test the generalizability and clinical relevance of the above findings, Applicants analyzed two independent bulk RNA-Seq cohorts (Nakayama et al. Am J Surg Pathol 34:1599-1607 (2010); Lagarde et al. J Clin Oncol Off J Am Soc Clin Oncol 31:608-615 (2013)). The first cohort included 34 SyS tumors (Nakayama et al. Am J Surg Pathol 34:1599-1607 (2010)), spanning monophasic, biphasic and poorly differentiated morphologies (FIG. 5A). Whereas the epithelial program was significantly higher in biphasic compared to monophasic tumors (P=4.14*10−6, one-sided t-test, FIG. 5A), poorly differentiated tumors had lower differentiation scores and higher proliferation and core oncogenic scores (P<2.76*10−4, one-sided t-test, FIG. 5A), consistent with their poor clinical prognosis. Next, Applicants examined the prognostic value of the programs in another independent cohort of 58 primary SyS tumors from treatment naïve patients with metastasis-free survival information (Lagarde et al. J Clin Oncol Off J Am Soc Clin Oncol 31:608-615 (2013)). The differentiation scores were associated with higher metastasis-free survival rates (P=1.49*10−4, Cox regression, FIG. 5B), while cell cycle and the core oncogenic programs were associated with the risk of metastatic disease (P=5.89*10−6 and 1.36*10−3, respectively, Cox regression, FIG. 5B). These findings support the notion that poor differentiation features and the core oncogenic program mark the aggressive subpopulation of malignant cells, which are more prone to metastasize.

### Example 4—SS18-SSX Sustains the Core Oncogenic Program and Blocks Differentiation

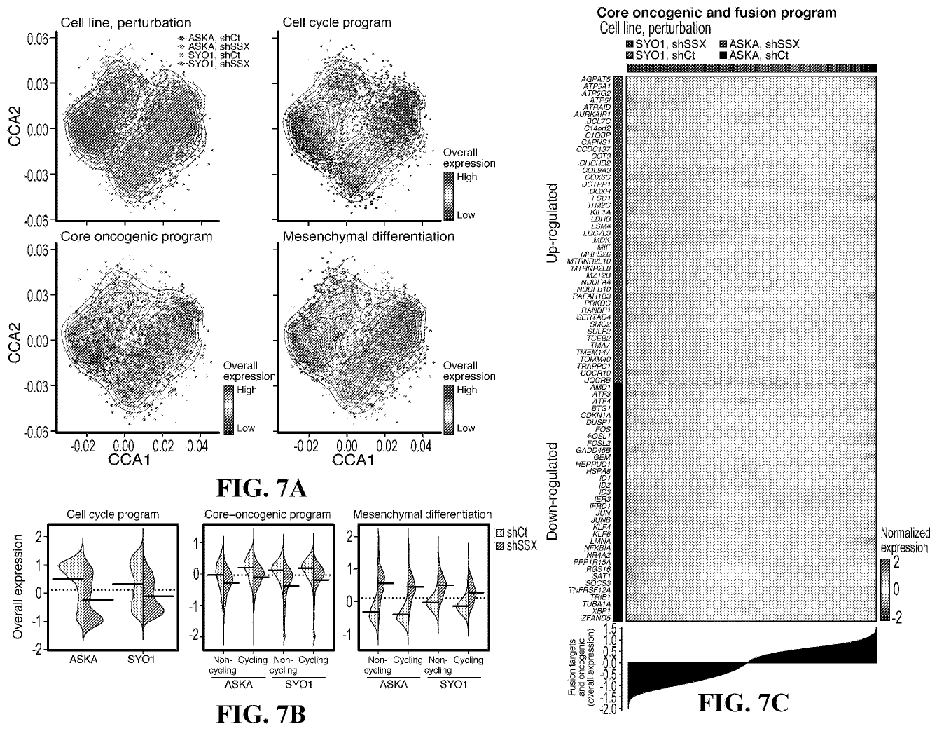

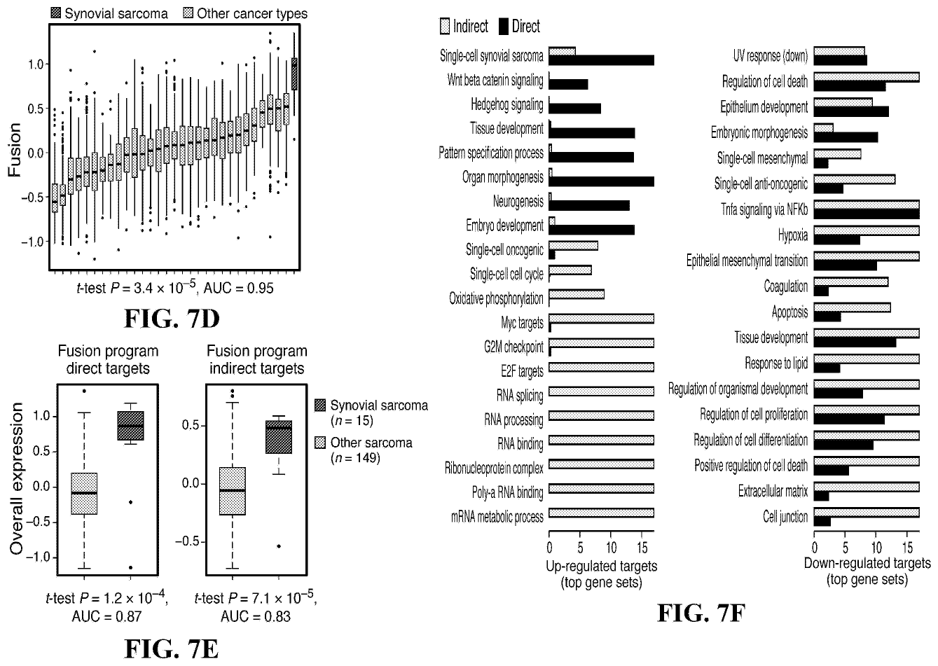

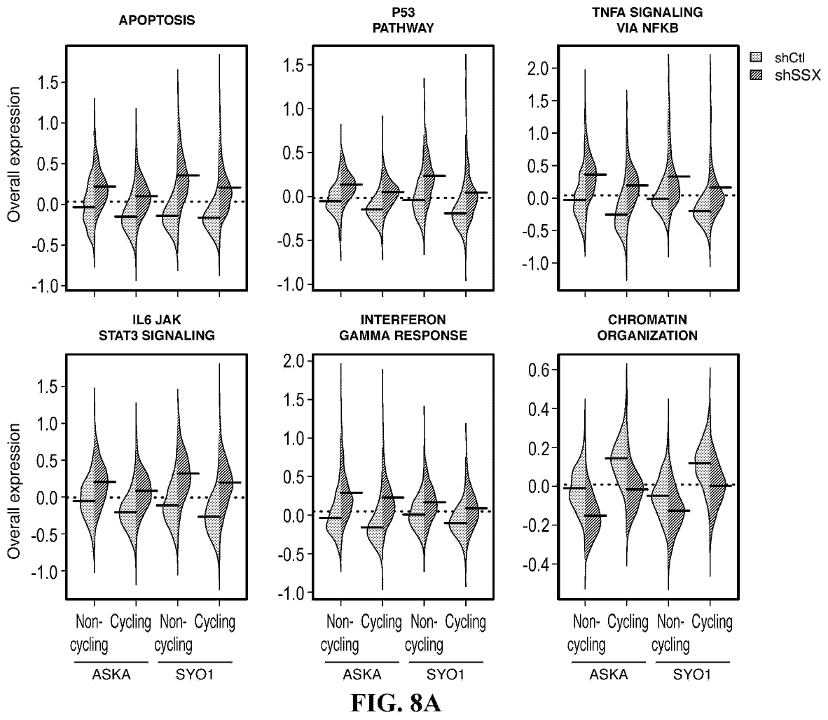

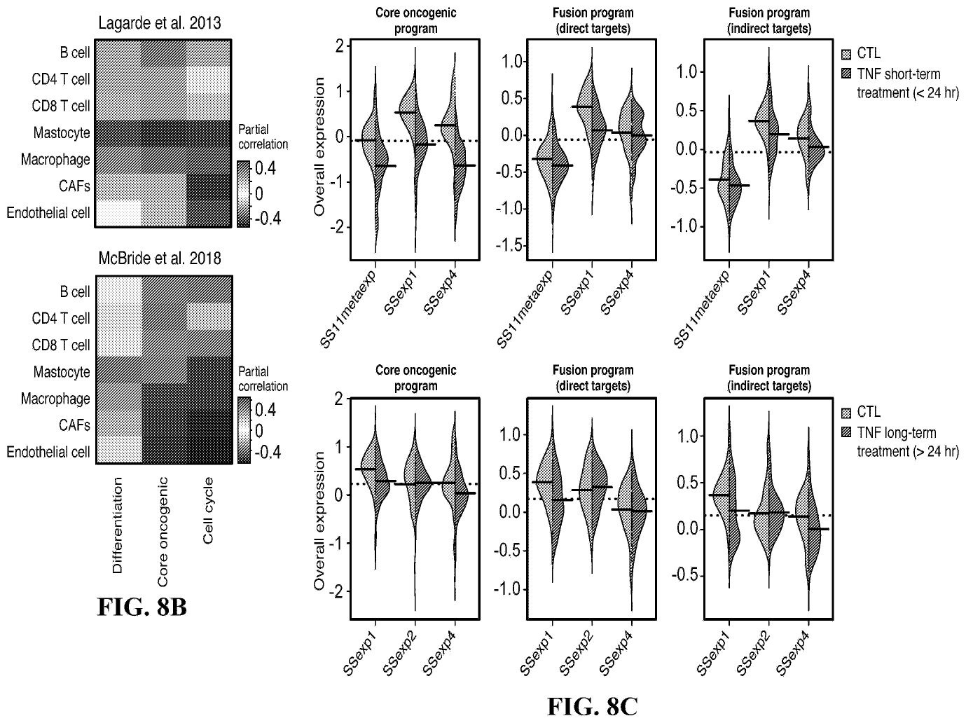

To decouple the intrinsic and extrinsic factor determining the malignant cell states in SyS Applicants first tested whether the core oncogenic and other programs were co-regulated by the genetic fusion driving SyS. Applicants turned to explore the potential regulators of these cellular programs, starting from the genetic driver. To this end, they depleted SS18-SSX in two SyS cell lines (SYO1 and Aska) using shRNA and profiled 12,263 cells with scRNA-Seq. The fusion KD led to massive and highly consistent transcriptional alternation in both cell lines (FIG. 7A, Tables 8, 9). It substantially repressed the core oncogenic program and cellular proliferation (P<8.05*10-¹⁰⁷, t-test, FIG. 7A-7C), while inducing mesenchymal differentiation programs and markers, including ZEB1 and VIM (P<1*10⁻⁵⁰, t-test and likelihood-ratio test FIGS. 7A-7B, 8A). Leveraging the single-cell readout Applicants confirmed that the KD impact on the core oncogenic and differentiation programs was decoupled from, and not secondary to, the repression of cellular proliferation (FIG. 7B), such that the impact on the core oncogenic and differentiation programs was observed even when controlling for the cycling status of the cells, and when considering only cycling or non-cycling cells (P<1.54*10⁻¹³, t-test, FIG. 5B, METHODS). Thus, the fusion's impact on cell cycle may be secondary or downstream to its impact on the core oncogenic program. In addition, the fusion KD led to an induction of antigen presentation and cell autonomous immune responses, such as TNF and IFN signaling (P<1*10⁻³⁰, mixed-effects, FIG. 8A).

TABLE 8
The SS18-SSX fusion program.

| Fusion UP | | | Fusion DOWN | | |
|---|---|---|---|---|---|
| Direct targets | Indirect targets | | Direct targets | Indirect targets | |
| ABLIM1 | ACADVL | H1F0 | ADM | AAED1 | LRRC59 |
| ADD3 | ADAM9 | H1FX | ANXA2 | ABL2 | LRRFIP2 |
| ALDH1A3 | ADIPOR1 | H2AFJ | ATF3 | ABRACL | MAFB |
| APBB2 | AGPAT5 | H2AFV | ATOH8 | AC093673 | MAGED1 |
| ARC | AIMP1 | H2AFZ | BAMBI | ACAN | MAGED2 |
| ARID5B | AKAP9 | H3F3A | BCL2L11 | ACTA1 | MALL |
| AUTS2 | AKR7A2 | HCFC1R1 | CCND1 | ACTA2 | MALT1 |
| BMP7 | AKTIP | HDDC2 | CDH2 | ACTB | MAP1A |
| CADM1 | ANAPC16 | HDGF | CHST2 | ACTG2 | MAP1B |
| CASC10 | ANP32A | HELLS | CRIP2 | ACTN1 | MAP1LC3B |
| CCDC140 | ANP32B | HIST1H4C | CRLF1 | ADAM19 | MARCKS |
| CCND2 | ANP32E | HMGB1 | CTSB | ADRM1 | MDFIC |
| CDK6 | ARL6IP1 | HMGB2 | CTSD | AK5 | MED19 |
| CDX2 | ARPC1A | HMGN1 | CXCR4 | AKAP12 | MEST |
| CELF2 | ART5 | HMGN2 | DKK2 | AKR1B1 | METTL9 |
| CKB | ASF1B | HMGN3 | DUSP4 | AMD1 | MGLL |
| CLMN | ATAD2 | HNRNPA2B1 | DUSP5 | ANGPT2 | MGP |
| COL8A1 | ATP5A1 | HNRNPC | EGFR | ANKH | MICAL2 |
| COL9A3 | ATP5G1 | HNRNPH1 | ETV4 | ANKRD11 | MIR4435-1HG |
| COLEC12 | ATP5G2 | HNRNPU | FLNA | ANXA1 | MMP1 |
| COPS8 | ATP5I | HNRNPUL1 | FSCN1 | ANXA5 | MMP10 |
| CRABP2 | ATP5L | HOXA3 | GADD45B | AP2S1 | MMP24-AS1 |
| CRNDE | ATP6V0B | HOXC10 | GAS1 | APCDD1 | MMP3 |
| CRTAC1 | ATRAID | HOXC6 | HTRA1 | ARF4 | MORF4L2 |
| CTNNB1 | AURKAIP1 | HPS4 | IGFBP4 | ARF6 | MPC2 |
| DUT | AURKB | HSP90AB1 | INSIG1 | ARG2 | MRPL13 |
| FGFR1 | BCL7C | IFIH1 | KLF4 | ARHGAP22 | MRPL36 |
| FJX1 | BIRC5 | IGFBP5 | KLF6 | ARHGDIA | MRPS6 |
| FLRT2 | BLOC1S1 | IMPDH2 | LGALS3 | ARL2BP | MSN |
| FOXC1 | BMP4 | INSIG2 | LMO4 | ARL4C | MSRA |
| GAMT | BOLA3 | IPO7 | LMO7 | ARPC1B | MT1E |
| GAS6 | BRD2 | ISG20L2 | LPAR1 | ARPC2 | MT1F |
| GPM6B | BRD7 | JPH4 | MAP2K3 | ARPC5L | MT1X |
| HAND2 | BTBD1 | KCNQ1OT1 | MSX1 | ARRDC3 | MT2A |
| HES1 | C11orf31 | KIAA0101 | MYC | ASAP1 | MYL12A |
| HES4 | C14orf2 | KIF1A | NR3C1 | ASPH | MYL12B |
| HEY2 | C19orf53 | KIF23 | NR4A2 | ATF4 | MYL6 |
| HHIP | C1QBP | KPNA2 | NSG1 | ATOX1 | MYL9 |
| HMCN1 | C20orf24 | KPNB1 | PHLDA1 | ATP1B1 | MYLIP |
| HOTAIRM1 | C6orf48 | LAGE3 | PHLDA2 | ATP1B3 | MYLK |
| HOXA10 | C7orf50 | LAMTOR2 | PLOD2 | ATP6V0E1 | MYO10 |
| HOXC8 | CA11 | LAPTM4B | PMP22 | ATP6V1G1 | MYO1B |
| HOXD1 | CAPNS1 | LDHB | PTEN | AXL | MYOF |
| IGF2 | CBFB | LINC01116 | RND3 | B2M | NAA10 |
| IRX3 | CBX1 | LIX1 | SAMD11 | BACE2 | NABP1 |
| ITM2C | CBX3 | LNPEP | SIRPA | BCAR1 | NANS |
| JAG1 | CBX5 | LSM3 | SLC35D3 | BID | NBL1 |
| LIMCH1 | CCDC137 | LSM4 | SOCS3 | BNIP3L | NDRG1 |
| LSAMP | CCDC85B | LSMD1 | SOX4 | BPGM | NEAT1 |
| LTBP4 | CCL2 | LUC7L3 | SPHK1 | BRI3 | NETO2 |
| LYPLA1 | CCNB1 | LYAR | TBX3 | BTG1 | NF2 |
| MEOX1 | CCNE1 | MATR3 | TCF4 | C11orf96 | NFKBIA |
| MEOX2 | CCT3 | MCTP2 | TNMD | C12orf75 | NHP2L1 |
| METRN | CCT6A | MDK | VGF | C16orf45 | NNMT |
| MRGBP | CDC20 | MET | WLS | C19orf24 | NOP10 |
| NFIA | CDCA5 | MIF | XYLT1 | C1orf198 | NPC2 |
| NFIB | CENPA | MKI67 | ZFP36L1 | CALD1 | NPTX2 |
| NR2F2 | CENPF | MLF2 | ZNF704 | CALM1 | NQO1 |
| NRP2 | CEP112 | MMADHC | | CALU | NR1H2 |
| OSR2 | CEP78 | MOB3B | | CAP1 | NREP |
| PAX3 | CHCHD2 | MRPS26 | | CAPN2 | NT5E |
| PCDH9 | CHD1 | MT-CO1 | | CAV1 | NTMT1 |
| PDZRN3 | CHD9 | MT-CO2 | | CAV2 | NUPR1 |
| PEX2 | CIRBP | MT-CO3 | | CBLN1 | OAF |
| PIGC | CKLF | MT-ND1 | | CCDC71L | OASL |
| PIM3 | CKS1B | MT-ND2 | | CCDC80 | OLFM2 |
| PRRT2 | CKS2 | MT-ND3 | | CCDC92 | PAWR |
| PTCH1 | CLPTM1L | MT-ND4 | | CCL5 | PCOLCE |
| PTHLH | CLSPN | MT-ND5 | | CCL7 | PDCD6 |
| RBM20 | CMTM6 | MTERF | | CD44 | PDLIM2 |
| RBMS3 | CNOT7 | MTF2 | | CD59 | PDLIM4 |
| REEP3 | COX6C | MTRNR2L10 | | CD63 | PDLIM7 |
| ROBO1 | COX8A | MTRNR2L8 | | CD9 | PEA15 |
| SERTAD4 | CPSF6 | MZT2A | | CD99 | PELO |
| SERTAD4-AS1 | CRELD2 | MZT2B | | CDC25B | PERP |
| SHISA2 | CSDE1 | NAV2 | | CDC37 | PGF |
| SMARCD3 | CTDSPL | NBEAL1 | | CDC42EP3 | PHC2 |
| SULF2 | CXCL14 | NCAM1 | | CDC42EP5 | PHLDA3 |
| TENM3 | CYCS | NCL | | CDKN1A | PIM1 |
| TLE1 | CYP24A1 | NDUFA4 | | CDV3 | PKIG |
| TLE3 | DAXX | NDUFB1 | | CEBPB | PKM |
| TLE4 | DBF4 | NDUFB10 | | CEBPD | PLAU |
| TMEM47 | DBP | NDUFC1 | | CFL1 | PLAUR |
| TRPS1 | DCBLD2 | NME3 | | CHCHD10 | PLIN2 |
| WNT16 | DCTPP1 | NME4 | | CHMP1B | PLOD1 |
| ZFHX3 | DCUN1D4 | NOLC1 | | CITED2 | PMAIP1 |
| ZFHX4 | DCXR | NOP56 | | CITED4 | PNP |
| ZFHX4-AS1 | DDX18 | NRAS | | CKAP4 | POMP |
| ZIC1 | DDX60L | NT5C3B | | CLIC1 | PPFIBP1 |
| ZNF385D | DEK | NTM | | CLIC4 | PPME1 |
| | DENR | NUCKS1 | | CLMP | PPP1R14B |
| | DFFA | NUDT1 | | CLTA | PPP1R15A |
| | DHRS3 | NUDT3 | | CMTM3 | PRR13 |
| | DKC1 | ODC1 | | CNN3 | PRRX1 |
| | DMKN | ORC6 | | COL12A1 | PRRX2 |
| | DNAJC2 | PA2G4 | | COL1A1 | PRSS23 |
| | DNMT1 | PABPC1 | | COL3A1 | PSMA7 |
| | DNPH1 | PAFAH1B3 | | COL5A1 | PSME1 |
| | DUSP9 | PAMR1 | | COL5A2 | PSME2 |
| | EBF1 | PARP1 | | COL5A3 | PTGDS |
| | EDNRA | PBK | | COL6A1 | PTN |
| | EEF2 | PCBP2 | | COL6A2 | PTRF |
| | EFHD2 | PCSK1N | | COPRS | PTTG1IP |
| | EI24 | PFN2 | | CORO1C | PXDC1 |
| | EIF3L | PHF19 | | COTL1 | RAB32 |
| | EIF4B | PHGDH | | CPE | RAB3B |
| | EIF4G2 | PIGP | | CREB3L1 | RAB7A |
| | ERH | PIN1 | | CREM | RABAC1 |
| | ERVMER34-1 | PLK1 | | CSNK1A1 | RAC1 |
| | ESF1 | PMVK | | CSRP1 | RAI14 |
| | ETF1 | PNISR | | CSRP2 | RALA |
| | ETFB | PNN | | CST3 | RAP1B |
| | F12 | POLR2I | | CTA-29F11 | RASD1 |
| | FAM3C | POLR2J3 | | CTGF | RBM8A |
| | FAM49B | POLR2L | | CTHRC1 | RBMS1 |
| | FAM64A | PON2 | | CTSA | RCN3 |
| | FBN1 | POP7 | | CTSC | RECK |
| | FDPS | PPDPF | | CTSL | REXO2 |
| | FHL1 | PPIC | | CXCL3 | RGCC |
| | FSD1 | PPP2CB | | CXXC5 | RGMB |
| | FUS | PPP2R5C | | CYB5R3 | RGS10 |
| | FZD10 | PRDX3 | | CYBA | RGS16 |
| | GCSH | PRKDC | | CYR61 | RGS2 |
| | GGCT | PRPF4 | | CYSTM1 | RHOBTB3 |
| | GLO1 | PTK2 | | CYTL1 | RHOC |
| | GLTSCR2 | PTMS | | DAB2 | RIPK2 |
| | GMFB | PTRHD1 | | DAP | RNF114 |
| | GNL1 | RAB34 | | DBI | RNF115 |
| | GPR125 | RAC3 | | DCUN1D5 | RNF149 |
| | GTF2I | RAD21 | | DDIT4 | RP11-395G23 |
| | | RANBP1 | | DDR2 | RPL10 |
| | | RBM39 | | DIRAS3 | RPL10A |
| | | RBMX | | DKK3 | RPL11 |
| | | RBP1 | | DNAJB6 | RPL13A |
| | | RHNO1 | | DOK1 | RPL15 |
| | | RNF187 | | DSTN | RPL27 |
| | | RNPS1 | | DSTYK | RPL28 |
| | | RP11-357H14 | | DUS1L | RPL6 |
| | | RP11-410K21 | | DUSP1 | RPL7 |
| | | RPL12 | | DUSP14 | RPL7A |
| | | RPL17 | | DYNLRB1 | RPS13 |
| | | RPL21 | | EBNA1BP2 | RPS15A |
| | | RPL31 | | ECM1 | RPS18 |
| | | RPL36A | | EEF1A1 | RPS27A |
| | | RPL37A | | EEF1B2 | RPS3 |
| | | RPL39L | | EFEMP2 | RPS4X |
| | | RPS16 | | EHD2 | RPS5 |
| | | RPS19BP1 | | EID1 | RPS7 |
| | | RPS2 | | EIF2AK4 | RRBP1 |
| | | RPS26 | | EIF4EBP1 | RSU1 |
| | | RPS27 | | EMP2 | RTN4 |
| | | RPS27L | | EMP3 | S100A10 |
| | | RPS28 | | ENO1 | S100A11 |
| | | RPS29 | | EPN1 | S100A13 |
| | | RPSA | | ERCC1 | S100A2 |
| | | RPSAP58 | | ERRFI1 | S100A4 |
| | | RRAS | | EVA1A | SAA1 |
| | | RRM2 | | F3 | SAT1 |
| | | RRP1B | | FABP4 | SDC2 |
| | | RUSC1 | | FABP5 | 11-Sep |
| | | SCD | | FAM107B | SERINC2 |
| | | SCT | | FAM114A1 | SERPINE1 |
| | | SELT | | FAM127A | SERPINE2 |
| | | SEPW1 | | FAM171B | SERPINH1 |
| | | SF3B14 | | FAM43A | SESTD1 |
| | | SFPQ | | FAM46A | SFRP1 |
| | | SGCB | | FAM89A | SFRP4 |
| | | SKA2 | | FBXO32 | SGK1 |
| | | SLBP | | FGF1 | SGK223 |
| | | SLC25A13 | | FHL2 | SH3BGRL3 |
| | | SLC25A39 | | FN1 | SH3GL1 |
| | | SLC39A6 | | FOS | SH3GLB1 |
| | | SLC50A1 | | FOSL1 | SLC16A3 |
| | | SLIT3 | | FOSL2 | SLC16A6 |
| | | SMC2 | | FST | SLC17A5 |
| | | SMC4 | | FSTL1 | SLC18A2 |
| | | SMIM19 | | FTH1 | SLC20A2 |
| | | SNHG7 | | FTL | SLC3A2 |
| | | SNRNP70 | | FUCA2 | SLC4A7 |
| | | SNRPB | | FXYD5 | SLC52A2 |
| | | SNRPD1 | | G0S2 | SLN |
| | | SOCS1 | | GADD45A | SMAGP |
| | | SOX8 | | GDF15 | SMAP2 |
| | | SQLE | | GEM | SMYD3 |
| | | SRGAP3 | | GIPC1 | SNAI2 |
| | | SRRM1 | | GLIPR1 | SNHG15 |
| | | SRRM2 | | GLIPR2 | SNX3 |
| | | SRSF1 | | GLIS3 | SNX9 |
| | | SRSF11 | | GLRX | SOCS2 |
| | | SRSF2 | | GNG11 | SPARC |
| | | SRSF3 | | GNG12 | SPATS2L |
| | | SRSF6 | | GNG5 | SPOCD1 |
| | | SRSF7 | | GPR56 | SPOCK1 |
| | | SSRP1 | | GSN | SPRY2 |
| | | STARD3NL | | GSTO1 | SRGAP1 |
| | | STARD7 | | HBEGF | SRM |
| | | STMN1 | | HERPUD1 | SSR2 |
| | | STOML2 | | HEXIM1 | SSR3 |
| | | STRA13 | | HEY1 | STAT1 |
| | | SUCO | | HIC1 | STC1 |
| | | SUMO2 | | HIST1H2AC | STC2 |
| | | SUPT16H | | HLA-A | STK17A |
| | | SURF2 | | HLA-C | STK38L |
| | | SUZ12 | | HM13 | STRAP |
| | | TAF9 | | HMOX1 | SUGCT |
| | | TCEA1 | | HOMER3 | TAF7 |
| | | TCEB2 | | HSF1 | TAGLN |
| | | TEX30 | | HSPA8 | TAGLN2 |
| | | TGFBR1 | | HSPB1 | TAX1BP1 |
| | | THAP5 | | ID1 | TBCC |
| | | TMA7 | | ID2 | TCEB1 |
| | | TMEM100 | | ID3 | TGFB1 |
| | | TMEM106C | | ID4 | TGFB1I1 |
| | | TMEM134 | | IER3 | TGFBI |
| | | TMEM147 | | IFI44 | TGIF1 |
| | | TMEM14A | | IFI6 | TGM2 |
| | | TMEM14B | | IFIT1 | THBS1 |
| | | TMEM160 | | IFIT2 | THY1 |
| | | TMEM184C | | IFIT3 | TIMP2 |
| | | TMEM256 | | IFITM3 | TIMP3 |
| | | TMEM30B | | IFRD1 | TIPARP |
| | | TNFAIP2 | | IGFBP3 | TMEM123 |
| | | TOMM22 | | IGFBP7 | TMEM173 |
| | | TOMM40 | | IGFL2 | TMEM45A |
| | | TOP2A | | IL11 | TMEM70 |
| | | TPD52L1 | | IL8 | TMSB10 |
| | | TPM1 | | IMP4 | TMSB4X |
| | | TPX2 | | INA | TNC |
| | | TRAPPC1 | | INHBA | TNFRSF12A |
| | | TRMT112 | | IQCD | TNFRSF1A |
| | | TSEN54 | | ISG15 | TNFRSF21 |
| | | TSHZ2 | | ITGA10 | TP53I11 |
| | | TYMS | | ITGA5 | TPBG |
| | | U2SURP | | ITGAV | TPM2 |
| | | UBE2C | | JUN | TPM3 |
| | | UBE2Q1 | | JUNB | TPM4 |
| | | UBE2S | | KCNG1 | TPT1 |
| | | UCHL1 | | KCNMA1 | TRAM1 |
| | | UHMK1 | | KDELR2 | TRIB1 |
| | | UNKL | | KDELR3 | TSC22D1 |
| | | UQCR10 | | KIAA0040 | TSPAN5 |
| | | UQCRB | | KIFC3 | TSPO |
| | | USP1 | | KISS1 | TUBA1A |
| | | USP46 | | KLHDC3 | TUBA1C |
| | | VMA21 | | KLHL42 | TWSG1 |
| | | WDR34 | | KRT10 | TXNDC17 |
| | | WDR45B | | KRT19 | TXNRD1 |
| | | WIF1 | | KRT81 | UACA |
| | | WRB | | KRTAP2-3 | UAP1 |
| | | WWC3 | | KXD1 | UBL3 |
| | | XRCC6 | | LAMA4 | UBTD1 |
| | | YBX1 | | LAMTOR1 | UXT |
| | | YRDC | | LAPTM4A | VASN |
| | | YWHAH | | LARP6 | VAT1 |
| | | ZDHHC12 | | LBH | VIM |
| | | ZFP36 | | LDHA | VMP1 |
| | | ZNF22 | | LGALS1 | VOPP1 |
| | | ZWINT | | LIMA1 | WBP5 |
| | | | | LINC00152 | WDR1 |
| | | | | LINC00704 | WIPI1 |
| | | | | LMNA | WISP1 |
| | | | | LOX | WWTR1 |
| | | | | LOXL1 | XBP1 |
| | | | | LOXL2 | YES1 |
| | | | | | YIF1A |
| | | | | | YIPF3 |
| | | | | | YPEL5 |
| | | | | | YWHAB |
| | | | | | ZC3HAV1 |
| | | | | | ZEB1 |
| | | | | | ZFAND5 |
| | | | | | ZFP36L2 |
| | | | | | ZNF259 |
| | | | | | ZNF503 |
| | | | | | ZNF706 |
| | | | | | ZYX |

TABLE 9
The SS18-SSX fusion program enrichment with pre-defined gene sets
(hypergeometric p-values: −log10 transformed, capped at 17)

| | Fusion UP | | Fusion DOWN | |
|---|---|---|---|---|
| | Direct | Indirect | Direct | Indirect |
| Gene set | targets | targets | targets | targets |
| GO_TISSUE_DEVELOPMENT | 13.87 | 0.02 | 13.22 | 17.00 |
| HALLMARK_TNFA_SIGNALING_VIA_NFKB | 1.39 | 0.62 | 17.00 | 17.00 |
| EMT_Up (Groger et al. 2012) | 0.65 | 0.51 | 1.90 | 17.00 |
| HALLMARK_HYPOXIA | 0.00 | 0.07 | 7.40 | 17.00 |
| EMT_Up (Taube et al. 2010) | 0.00 | 0.39 | 5.63 | 17.00 |
| HALLMARK_APOPTOSIS | 0.90 | 1.27 | 4.26 | 12.36 |
| GO_ORGAN_MORPHOGENESIS | 17.00 | 0.48 | 6.85 | 9.38 |
| GO_NEUROGENESIS | 12.98 | 0.32 | 5.10 | 5.47 |
| GO_EMBRYO_DEVELOPMENT | 13.77 | 0.95 | 8.38 | 4.89 |
| GO_SKELETAL_SYSTEM_DEVELOPMENT | 10.86 | 0.64 | 2.99 | 3.36 |
| GO_STEM_CELL_DIFFERENTIATION | 11.13 | 1.00 | 1.07 | 1.87 |
| HALLMARK_MYC_TARGETS_V1 | 0.27 | 17.00 | 0.40 | 1.02 |
| HALLMARK_G2M_CHECKPOINT | 0.27 | 17.00 | 1.04 | 0.50 |
| GO_PATTERN_SPECIFICATION_PROCESS | 13.71 | 0.40 | 1.05 | 0.20 |
| HALLMARK_E2F_TARGETS | 0.27 | 17.00 | 0.40 | 0.02 |
| GO_REGULATION_OF_CELL_DIFFERENTIATION | 8.35 | 0.22 | 9.55 | 17.00 |
| HALLMARK_INTERFERON_GAMMA_RESPONSE | 0.76 | 0.37 | 0.40 | 8.77 |
| GO_REGULATION_OF_MULTICELLULAR_ORGANISMAL_DEVELOPMENT | 9.57 | 0.03 | 7.86 | 17.00 |
| GO_REGULATION_OF_CELL_PROLIFERATION | 8.32 | 0.91 | 11.38 | 17.00 |
| GO_REGULATION_OF_ANATOMICAL_STRUCTURE_MORPHOGENESIS | 5.22 | 0.27 | 5.10 | 17.00 |
| GO_EXTRACELLULAR_STRUCTURE_ORGANIZATION | 4.69 | 0.24 | 0.74 | 17.00 |
| GO_EXTRACELLULAR_MATRIX | 3.67 | 0.03 | 2.35 | 17.00 |
| GO_REGULATION_OF_CELL_DEATH | 2.92 | 2.30 | 11.52 | 17.00 |
| GO_NEGATIVE_REGULATION_OF_CELL_COMMUNICATION | 2.76 | 0.11 | 8.45 | 17.00 |
| GO_NEGATIVE_REGULATION_OF_RESPONSE_TO_STIMULUS | 2.76 | 0.23 | 8.45 | 17.00 |
| GO_POSITIVE_REGULATION_OF_RESPONSE_TO_STIMULUS | 2.66 | 0.33 | 9.24 | 17.00 |
| GO_CELL_JUNCTION | 2.39 | 0.14 | 2.63 | 17.00 |
| GO_REGULATION_OF_CELLULAR_COMPONENT_MOVEMENT | 2.05 | 0.83 | 5.49 | 17.00 |
| GO_EXTRACELLULAR_SPACE | 1.84 | 0.36 | 3.22 | 17.00 |
| GO_POSITIVE_REGULATION_OF_CELL_DEATH | 1.56 | 1.32 | 5.58 | 17.00 |
| GO_CELLULAR_RESPONSE_TO_ORGANIC_SUBSTANCE | 1.05 | 1.12 | 6.42 | 17.00 |
| GO_RESPONSE_TO_ENDOGENOUS_STIMULUS | 1.01 | 1.55 | 7.19 | 17.00 |
| GO_ANCHORING_JUNCTION | 0.94 | 1.45 | 2.10 | 17.00 |
| GO_RESPONSE_TO_ORGANIC_CYCLIC_COMPOUND | 0.87 | 2.49 | 5.58 | 17.00 |
| GO_RESPONSE_TO_OXYGEN_CONTAINING_COMPOUND | 0.82 | 1.82 | 4.48 | 17.00 |
| HALLMARK_EPITHELIAL_MESENCHYMAL_TRANSITION | 0.76 | 0.93 | 10.12 | 17.00 |
| GO_RESPONSE_TO_LIPID | 0.61 | 2.39 | 4.15 | 17.00 |
| GO_CELL_SUBSTRATE_JUNCTION | 0.35 | 1.78 | 2.47 | 17.00 |
| GO_ACTIN_CYTOSKELETON | 0.30 | 0.21 | 0.99 | 17.00 |
| GO_POSITIVE_REGULATION_OF_DEVELOPMENTAL_PROCESS | 8.16 | 0.27 | 5.36 | 15.95 |
| GO_REGULATION_OF_CELL_ADHESION | 2.01 | 0.08 | 1.67 | 15.95 |
| GO_RESPONSE_TO_EXTERNAL_STIMULUS | 1.72 | 1.05 | 3.85 | 15.95 |
| GO_RESPONSE_TO_ABIOTIC_STIMULUS | 0.26 | 0.27 | 5.09 | 15.95 |
| GO_POSITIVE_REGULATION_OF_CELL_COMMUNICATION | 2.75 | 0.18 | 9.35 | 15.65 |
| GO_RESPONSE_TO_NITROGEN_COMPOUND | 0.65 | 0.72 | 5.05 | 15.26 |
| GO_CELLULAR_RESPONSE_TO_ENDOGENOUS_STIMULUS | 0.74 | 1.97 | 3.69 | 15.18 |
| GO_RESPONSE_TO_HORMONE | 0.60 | 2.36 | 5.70 | 15.05 |
| GO_NEGATIVE_REGULATION_OF_MOLECULAR_FUNCTION | 1.68 | 0.36 | 2.83 | 14.75 |
| GO_CELLULAR_RESPONSE_TO_OXYGEN_CONTAINING_COMPOUND | 0.73 | 1.81 | 2.40 | 14.42 |
| GO_STRUCTURAL_MOLECULE_ACTIVITY | 0.28 | 3.21 | 0.00 | 14.35 |
| GO_BIOLOGICAL_ADHESION | 3.88 | 0.02 | 2.36 | 14.15 |
| GO_NEGATIVE_REGULATION_OF_MULTICELLULAR_ORGANISMAL_{—} | 6.87 | 0.54 | 3.11 | 14.12 |
| PROCESS | | | | |
| GO_PROTEIN_LOCALIZATION | 0.61 | 1.94 | 1.43 | 14.03 |
| GO_MOVEMENT_OF_CELL_OR_SUBCELLULAR_COMPONENT | 4.09 | 0.19 | 4.15 | 14.00 |
| GO_POSITIVE_REGULATION_OF_MULTICELLULAR_{—} | 7.43 | 0.42 | 2.61 | 13.80 |
| ORGANISMAL_PROCESS | | | | |
| GO_CELLULAR_MACROMOLECULE_LOCALIZATION | 1.02 | 2.54 | 1.44 | 13.77 |
| GO_PROTEINACEOUS_EXTRACELLULAR_MATRIX | 3.39 | 0.07 | 0.22 | 13.58 |
| GO_RESPONSE_TO_STEROID_HORMONE | 1.39 | 2.36 | 4.46 | 13.53 |
| GO_RESPONSE_TO_WOUNDING | 0.44 | 0.83 | 1.26 | 13.47 |
| GO_RESPONSE_TO_INORGANIC_SUBSTANCE | 0.26 | 0.55 | 2.14 | 13.44 |
| GO_ESTABLISHMENT_OF_PROTEIN_LOCALIZATION | 0.53 | 1.46 | 1.57 | 13.33 |
| GO_CELL_DEATH | 1.45 | 3.13 | 3.72 | 13.23 |
| GO_INTERSPECIES_INTERACTION_BETWEEN_ORGANISMS | 0.14 | 7.08 | 0.63 | 13.21 |
| GO_POSITIVE_REGULATION_OF_PROTEIN_METABOLIC_{—} | 1.99 | 3.48 | 7.83 | 13.12 |
| PROCESS | | | | |
| GO_NEGATIVE_REGULATION_OF_CELL_PROLIFERATION | 5.27 | 0.20 | 5.35 | 13.11 |
| GO_VASCULATURE_DEVELOPMENT | 7.63 | 1.02 | 5.58 | 13.10 |
| GO_PROTEIN_LOCALIZATION_TO_ENDOPLASMIC_RETICULUM | 0.00 | 6.83 | 0.00 | 13.04 |
| GO_CIRCULATORY_SYSTEM_DEVELOPMENT | 10.04 | 0.77 | 5.40 | 13.01 |
| GO_CYTOSKELETON | 0.20 | 1.09 | 0.13 | 12.99 |
| GO_ENZYME_BINDING | 0.68 | 6.19 | 1.94 | 12.88 |
| GO_REGULATION_OF_PROTEIN_MODIFICATION_PROCESS | 1.92 | 2.56 | 7.70 | 12.85 |
| GO_POSITIVE_REGULATION_OF_CELL_DIFFERENTIATION | 6.37 | 0.09 | 5.22 | 12.77 |
| GO_POSITIVE_REGULATION_OF_MOLECULAR_FUNCTION | 1.12 | 2.65 | 5.20 | 12.54 |
| GO_REGULATION_OF_INTRACELLULAR_SIGNAL_TRANSDUCTION | 1.32 | 0.92 | 8.76 | 12.37 |
| GO_CYTOSKELETAL_PROTEIN_BINDING | 0.42 | 0.41 | 1.76 | 12.32 |
| GO_REGULATION_OF_HYDROLASE_ACTIVITY | 0.05 | 1.08 | 0.61 | 12.17 |
| HALLMARK_COAGULATION | 0.00 | 0.39 | 2.28 | 11.98 |
| GO_RESPONSE_TO_BIOTIC_STIMULUS | 0.07 | 0.64 | 1.13 | 11.96 |
| GO_CYTOSOLIC_RIBOSOME | 0.00 | 9.18 | 0.00 | 11.82 |
| GO_RESPONSE_TO_OXYGEN_LEVELS | 0.16 | 0.23 | 4.87 | 11.69 |
| GO_NEGATIVE_REGULATION_OF_DEVELOPMENTAL_PROCESS | 8.16 | 1.10 | 5.34 | 11.65 |
| GO_NEGATIVE_REGULATION_OF_PROTEIN_METABOLIC_PROCESS | 0.64 | 2.58 | 5.60 | 11.61 |
| GO_ACTIN_BINDING | 1.21 | 0.48 | 1.75 | 11.58 |
| GO_ESTABLISHMENT_OF_PROTEIN_LOCALIZATION_TO_{—} | 0.00 | 7.71 | 0.00 | 11.52 |
| ENDOPLASMIC_RETICULUM | | | | |
| GO_REGULATION_OF_CELL_DEVELOPMENT | 6.29 | 0.08 | 4.37 | 11.44 |
| GO_ENZYME_LINKED_RECEPTOR_PROTEIN_SIGNALING_PATHWAY | 3.56 | 0.75 | 1.52 | 11.43 |
| GO_POSITIVE_REGULATION_OF_CATALYTIC_ACTIVITY | 0.45 | 3.32 | 4.06 | 11.42 |
| GO_REGULATION_OF_RESPONSE_TO_STRESS | 0.31 | 1.14 | 2.99 | 11.31 |
| GO_NEGATIVE_REGULATION_OF_CELL_DEATH | 2.78 | 2.20 | 7.58 | 11.23 |
| GO_RECEPTOR_BINDING | 2.02 | 1.64 | 1.94 | 11.19 |
| GO_POSITIVE_REGULATION_OF_CELLULAR_COMPONENT_{—} | 2.39 | 2.21 | 3.23 | 10.97 |
| ORGANIZATION | | | | |
| GO_CELL_MOTILITY | 3.51 | 0.19 | 5.16 | 10.95 |
| GO_RESPONSE_TO_METAL_ION | 0.14 | 0.32 | 1.28 | 10.90 |
| GO_REGULATION_OF_PHOSPHORUS_METABOLIC_PROCESS | 1.73 | 1.69 | 8.93 | 10.84 |
| GO_PROTEIN_LOCALIZATION_TO_MEMBRANE | 0.38 | 3.67 | 2.58 | 10.81 |
| GO_RESPONSE_TO_EXTRACELLULAR_STIMULUS | 0.63 | 0.50 | 4.83 | 10.76 |
| GO_NEGATIVE_REGULATION_OF_LOCOMOTION | 0.58 | 1.13 | 0.84 | 10.66 |
| GO_RESPONSE_TO_ALCOHOL | 1.91 | 2.92 | 4.45 | 10.61 |
| GO_INTRACELLULAR_VESICLE | 0.47 | 0.02 | 2.94 | 10.60 |
| GO_NEGATIVE_REGULATION_OF_CATALYTIC_ACTIVITY | 0.41 | 0.65 | 2.31 | 10.57 |
| GO_WOUND_HEALING | 0.27 | 0.58 | 0.47 | 10.56 |
| GO_BLOOD_VESSEL_MORPHOGENESIS | 7.90 | 1.07 | 3.50 | 10.54 |
| GO_PROTEIN_TARGETING | 2.35 | 5.28 | 0.19 | 10.53 |
| GO_PROTEIN_COMPLEX_BINDING | 0.55 | 1.38 | 3.96 | 10.53 |
| GO_NEGATIVE_REGULATION_OF_CELL_DIFFERENTIATION | 7.18 | 0.85 | 3.84 | 10.52 |
| GO_INTRACELLULAR_PROTEIN_TRANSPORT | 1.12 | 2.50 | 0.23 | 10.51 |
| GO_NUCLEAR_TRANSCRIBED_MRNA_CATABOLIC_PROCESS_NONSENSE_{—} | 0.00 | 10.90 | 0.00 | 10.51 |
| MEDIATED_DECAY | | | | |
| GO_RESPONSE_TO_CYTOKINE | 0.29 | 0.28 | 0.97 | 10.35 |
| HALLMARK_MYOGENESIS | 0.76 | 0.93 | 1.04 | 10.30 |
| GO_POSITIVE_REGULATION_OF_CELL_ADHESION | 1.85 | 0.22 | 0.21 | 10.17 |
| GO_IMMUNE_SYSTEM_PROCESS | 0.47 | 0.69 | 4.03 | 10.07 |
| GO_CELLULAR_RESPONSE_TO_EXTRACELLULAR_STIMULUS | 0.29 | 0.22 | 1.91 | 10.07 |
| GO_EXTRACELLULAR_MATRIX_COMPONENT | 1.09 | 0.06 | 0.56 | 10.06 |
| GO_PROTEIN_TARGETING_TO_MEMBRANE | 0.92 | 6.39 | 0.48 | 9.98 |
| GO_ACTIN_FILAMENT_BASED_PROCESS | 0.09 | 0.06 | 0.97 | 9.90 |
| GO_CELLULAR_RESPONSE_TO_EXTERNAL_STIMULUS | 1.10 | 0.35 | 2.34 | 9.90 |
| GO_ESTABLISHMENT_OF_PROTEIN_LOCALIZATION_TO_MEMBRANE | 0.58 | 5.62 | 2.34 | 9.90 |
| GO_REGULATION_OF_PROTEOLYSIS | 0.29 | 2.69 | 1.47 | 9.90 |
| GO_RESPONSE_TO_CORTICOSTEROID | 0.00 | 3.11 | 2.98 | 9.84 |
| GO_MACROMOLECULAR_COMPLEX_BINDING | 2.20 | 5.42 | 2.60 | 9.81 |
| GO_POSITIVE_REGULATION_OF_INTRACELLULAR_SIGNAL_{—} | 1.75 | 0.53 | 9.48 | 9.70 |
| TRANSDUCTION | | | | |
| GO_REGULATION_OF_PEPTIDASE_ACTIVITY | 0.36 | 0.48 | 1.11 | 9.69 |
| GO_APOPTOTIC_SIGNALING_PATHWAY | 0.17 | 0.45 | 0.28 | 9.69 |
| GO_OSSIFICATION | 2.57 | 1.59 | 0.32 | 9.67 |
| GO_RIBOSOMAL_SUBUNIT | 0.00 | 7.81 | 0.00 | 9.67 |
| GO_VIRAL_LIFE_CYCLE | 0.52 | 7.68 | 0.77 | 9.66 |
| GO_ENZYME_REGULATOR_ACTIVITY | 0.15 | 0.91 | 1.02 | 9.65 |
| GO_CYTOPLASMIC_VESICLE_PART | 0.71 | 0.03 | 0.34 | 9.62 |
| GO_ANGIOGENESIS | 4.81 | 1.59 | 0.77 | 9.55 |
| GO_CELLULAR_RESPONSE_TO_ORGANIC_CYCLIC_COMPOUND | 1.50 | 1.60 | 2.19 | 9.51 |
| GO_EPITHELIUM_DEVELOPMENT | 10.45 | 0.06 | 12.07 | 9.43 |
| GO_LOCOMOTION | 4.77 | 0.23 | 5.48 | 9.37 |
| GO_NEGATIVE_REGULATION_OF_GENE_EXPRESSION | 6.26 | 4.69 | 2.39 | 9.35 |
| GO_ESTABLISHMENT_OF_LOCALIZATION_IN_CELL | 0.34 | 7.06 | 0.09 | 9.33 |
| GO_MULTI_ORGANISM_METABOLIC_PROCESS | 0.00 | 7.96 | 0.53 | 9.30 |
| GO_RESPONSE_TO_GROWTH_FACTOR | 3.36 | 1.25 | 4.60 | 9.26 |
| GO_ANATOMICAL_STRUCTURE_FORMATION_INVOLVED_IN_{—} | 12.18 | 0.14 | 8.93 | 9.23 |
| MORPHOGENESIS | | | | |
| GO_RIBOSOME | 0.00 | 6.56 | 0.00 | 9.20 |
| GO_CELLULAR_RESPONSE_TO_STRESS | 0.07 | 5.14 | 4.57 | 9.19 |
| GO_POSITIVE_REGULATION_OF_HYDROLASE_ACTIVITY | 0.06 | 1.09 | 0.70 | 9.19 |
| GO_CELLULAR_RESPONSE_TO_LIPID | 1.02 | 1.66 | 2.22 | 9.14 |
| GO_CELLULAR_RESPONSE_TO_NITROGEN_COMPOUND | 0.52 | 0.85 | 1.41 | 9.09 |
| GO_REGULATION_OF_CELLULAR_RESPONSE_TO_GROWTH_FACTOR_{—} | 3.63 | 0.27 | 1.69 | 9.08 |
| STIMULUS | | | | |
| GO_POSITIVE_REGULATION_OF_PROTEIN_COMPLEX_ASSEMBLY | 0.28 | 0.19 | 1.05 | 8.89 |
| GO_TRANSLATIONAL_INITIATION | 0.00 | 11.31 | 0.00 | 8.88 |
| GO_MEMBRANE_ORGANIZATION | 0.18 | 1.79 | 2.13 | 8.84 |
| GO_REGULATION_OF_MULTI_ORGANISM_PROCESS | 0.00 | 1.02 | 0.47 | 8.81 |
| HALLMARK_P53_PATHWAY | 0.27 | 1.29 | 3.81 | 8.77 |
| GO_NEGATIVE_REGULATION_OF_CELL_DEVELOPMENT | 3.81 | 0.13 | 1.38 | 8.57 |
| GO_CYTOSKELETAL_PART | 0.10 | 2.01 | 0.30 | 8.54 |
| GO_PROTEIN_LOCALIZATION_TO_ORGANELLE | 1.22 | 6.09 | 0.38 | 8.48 |
| GO_POSITIVE_REGULATION_OF_CELL_PROLIFERATION | 4.26 | 1.73 | 7.96 | 8.46 |
| GO_REGULATION_OF_CELL_SUBSTRATE_ADHESION | 1.55 | 0.26 | 1.14 | 8.40 |
| GO_REGULATION_OF_RESPONSE_TO_EXTERNAL_STIMULUS | 0.16 | 0.42 | 2.06 | 8.40 |
| GO_CELLULAR_RESPONSE_TO_INORGANIC_SUBSTANCE | 0.35 | 0.32 | 0.00 | 8.39 |
| GO_HEPARIN_BINDING | 2.52 | 1.32 | 0.00 | 8.35 |
| GO_REGULATION_OF_VASCULATURE_DEVELOPMENT | 1.92 | 0.71 | 2.54 | 8.22 |
| HALLMARK_UV_RESPONSE_DN | 2.66 | 0.36 | 8.52 | 8.16 |
| GO_TRANSMEMBRANE_RECEPTOR_PROTEIN_TYROSINE_KINASE_{—} | 2.55 | 0.35 | 0.44 | 8.16 |
| SIGNALING_PATHWAY | | | | |
| GO_ESTABLISHMENT_OF_PROTEIN_LOCALIZATION_TO_{—} | 1.32 | 5.50 | 0.22 | 8.13 |
| ORGANELLE | | | | |
| GO_POSITIVE_REGULATION_OF_CELLULAR_COMPONENT_{—} | 0.70 | 0.09 | 1.71 | 8.12 |
| BIOGENESIS | | | | |
| GO_REGULATION_OF_TRANSFERASE_ACTIVITY | 2.02 | 3.89 | 5.44 | 8.09 |
| GO_REGULATION_OF_APOPTOTIC_SIGNALING_PATHWAY | 0.80 | 0.40 | 3.51 | 8.07 |
| GO_CELL_SURFACE | 0.26 | 0.70 | 1.37 | 8.06 |
| GO_REGULATION_OF_CATABOLIC_PROCESS | 0.00 | 2.87 | 0.94 | 8.05 |
| GO_REGULATION_OF_CELL_MORPHOGENESIS | 1.23 | 0.15 | 1.89 | 8.05 |
| HALLMARK_KRAS_SIGNALING_UP | 1.39 | 0.37 | 1.84 | 8.04 |
| HALLMARK_IL2_STAT5_SIGNALING | 0.76 | 0.37 | 4.94 | 8.04 |
| GO_RESPONSE_TO_MOLECULE_OF_BACTERIAL_ORIGIN | 0.00 | 0.77 | 0.71 | 8.03 |
| GO_RESPONSE_TO_KETONE | 0.30 | 2.45 | 4.00 | 8.02 |
| GO_IDENTICAL_PROTEIN_BINDING | 1.80 | 0.80 | 1.07 | 8.02 |
| GO_VESICLE_MEDIATED_TRANSPORT | 0.29 | 0.01 | 0.21 | 8.01 |
| GO_SINGLE_ORGANISM_CELLULAR_LOCALIZATION | 0.90 | 3.25 | 1.11 | 7.97 |
| GO_CELLULAR_RESPONSE_TO_CYTOKINE_STIMULUS | 0.17 | 0.10 | 0.34 | 7.97 |
| GO_COLLAGEN_FIBRIL_ORGANIZATION | 0.87 | 0.33 | 1.04 | 7.97 |
| GO_POSITIVE_REGULATION_OF_GENE_EXPRESSION | 11.55 | 5.54 | 7.61 | 7.96 |
| GO_NEGATIVE_REGULATION_OF_CELLULAR_COMPONENT_{—} | 0.93 | 3.27 | 3.50 | 7.95 |
| ORGANIZATION | | | | |
| GO_NEGATIVE_REGULATION_OF_NITROGEN_COMPOUND_{—} | 6.15 | 4.84 | 2.35 | 7.93 |
| METABOLIC_PROCESS | | | | |
| GO_REGULATION_OF_CELLULAR_COMPONENT_BIOGENESIS | 0.78 | 0.13 | 2.50 | 7.89 |
| GO_CYTOSOLIC_PART | 0.00 | 9.73 | 0.00 | 7.89 |
| GO_CATABOLIC_PROCESS | 0.09 | 6.96 | 0.53 | 7.87 |
| GO_CELL_ADHESION_MOLECULE_BINDING | 2.26 | 0.09 | 0.00 | 7.86 |
| GO_GLYCOSAMINOGLYCAN_BINDING | 2.96 | 1.25 | 0.00 | 7.84 |
| GO_REGULATION_OF_IMMUNE_SYSTEM_PROCESS | 1.08 | 0.19 | 0.84 | 7.84 |
| GO_NEGATIVE_REGULATION_OF_PROTEIN_MODIFICATION_{—} | 0.69 | 1.52 | 4.63 | 7.78 |
| PROCESS | | | | |
| GO_REGULATION_OF_TRANSMEMBRANE_RECEPTOR_PROTEIN_{—} | 1.36 | 0.58 | 1.80 | 7.77 |
| SERINE_THREONINE_KINASE_SIGNALING_PATHWAY | | | | |
| GO_RNA_CATABOLIC_PROCESS | 0.00 | 11.26 | 0.36 | 7.75 |
| GO_RESPONSE_TO_VIRUS | 0.21 | 0.64 | 0.89 | 7.74 |
| GO_REGULATION_OF_KINASE_ACTIVITY | 2.04 | 2.23 | 6.34 | 7.74 |
| GO_NEGATIVE_REGULATION_OF_PHOSPHORYLATION | 0.67 | 1.59 | 4.97 | 7.73 |
| GO_REGULATION_OF_NERVOUS_SYSTEM_DEVELOPMENT | 6.09 | 0.03 | 3.23 | 7.72 |
| GO_POSITIVE_REGULATION_OF_PEPTIDASE_ACTIVITY | 0.35 | 0.59 | 1.23 | 7.70 |
| GO_INTEGRIN_BINDING | 0.48 | 0.28 | 0.00 | 7.70 |
| GO_ENZYME_ACTIVATOR_ACTIVITY | 0.27 | 0.28 | 0.00 | 7.70 |
| GO_RESPONSE_TO_NUTRIENT | 0.79 | 1.00 | 1.07 | 7.66 |
| GO_CYTOSKELETON_ORGANIZATION | 0.41 | 0.78 | 0.45 | 7.62 |
| GO_RUFFLE | 0.35 | 0.13 | 1.22 | 7.61 |
| GO_ACTIN_FILAMENT_ORGANIZATION | 0.00 | 0.02 | 1.14 | 7.61 |
| GO_STRUCTURAL_CONSTITUENT_OF_RIBOSOME | 0.00 | 6.96 | 0.00 | 7.59 |
| GO_COLLAGEN_BINDING | 0.00 | 0.18 | 0.82 | 7.55 |
| GO_CELLULAR_RESPONSE_TO_HORMONE_STIMULUS | 0.80 | 1.35 | 1.89 | 7.54 |
| GO_CELLULAR_RESPONSE_TO_AMINO_ACID_STIMULUS | 0.00 | 1.23 | 0.90 | 7.53 |
| GO_MULTICELLULAR_ORGANISM_METABOLIC_PROCESS | 0.53 | 0.00 | 1.63 | 7.52 |
| GO_MULTICELLULAR_ORGANISMAL_MACROMOLECULE_{—} | 0.59 | 0.00 | 1.76 | 7.50 |
| METABOLIC_PROCESS | | | | |
| GO_POSITIVE_REGULATION_OF_PROTEOLYSIS | 0.12 | 2.48 | 1.19 | 7.49 |
| GO_NEGATIVE_REGULATION_OF_TRANSPORT | 1.52 | 0.01 | 2.22 | 7.45 |
| GO_POSITIVE_REGULATION_OF_PROTEIN_MODIFICATION_{—} | 1.98 | 2.57 | 7.01 | 7.45 |
| PROCESS | | | | |
| GO_REGULATION_OF_MAPK_CASCADE | 1.41 | 1.06 | 8.09 | 7.44 |
| GO_CELLULAR_RESPONSE_TO_OXYGEN_LEVELS | 0.38 | 0.37 | 1.29 | 7.42 |
| GO_VESICLE_MEMBRANE | 0.24 | 0.03 | 0.43 | 7.34 |
| GO_INTRACELLULAR_SIGNAL_TRANSDUCTION | 0.26 | 0.61 | 3.90 | 7.32 |
| GO_RESPONSE_TO_AMINO_ACID | 0.00 | 1.95 | 0.60 | 7.31 |
| GO_PERINUCLEAR_REGION_OF_CYTOPLASM | 0.65 | 0.56 | 1.10 | 7.30 |
| GO_CELL_SUBSTRATE_ADHESION | 0.89 | 0.03 | 0.47 | 7.27 |
| GO_CELL_LEADING_EDGE | 1.36 | 0.16 | 3.59 | 7.24 |
| GO_RESPONSE_TO_VITAMIN | 1.27 | 2.22 | 0.66 | 7.23 |
| GO_REGULATION_OF_TRANSPORT | 1.41 | 0.07 | 3.89 | 7.22 |
| GO_ACTIN_FILAMENT_BUNDLE | 0.71 | 0.61 | 0.87 | 7.18 |
| GO_REGULATION_OF_SYMBIOSIS_ENCOMPASSING_{—} | 0.00 | 0.59 | 0.39 | 7.15 |
| MUTUALISM_THROUGH_PARASITISM | | | | |
| GO_MACROMOLECULE_CATABOLIC_PROCESS | 0.06 | 9.35 | 0.38 | 7.14 |
| HALLMARK_ANDROGEN_RESPONSE | 2.17 | 1.05 | 1.56 | 7.06 |
| GO_REGULATION_OF_CELL_MORPHOGENESIS_INVOLVED_IN_{—} | 1.41 | 0.31 | 1.98 | 7.00 |
| DIFFERENTIATION | | | | |
| GO_RESPONSE_TO_DRUG | 1.62 | 0.73 | 1.62 | 6.99 |
| GO_RESPONSE_TO_MECHANICAL_STIMULUS | 1.34 | 0.33 | 0.38 | 6.97 |
| GO_EXTRINSIC_COMPONENT_OF_MEMBRANE | 0.21 | 0.10 | 1.58 | 6.93 |
| GO_TISSUE_MORPHOGENESIS | 7.90 | 0.10 | 8.12 | 6.93 |
| GO_SINGLE_ORGANISM_CELL_ADHESION | 1.52 | 0.02 | 2.21 | 6.91 |
| GO_RESPONSE_TO_ACID_CHEMICAL | 0.15 | 1.37 | 0.71 | 6.88 |
| GO_NEGATIVE_REGULATION_OF_INTRACELLULAR_SIGNAL_{—} | 0.64 | 0.71 | 3.94 | 6.85 |
| TRANSDUCTION | | | | |
| GO_SULFUR_COMPOUND_BINDING | 1.92 | 1.01 | 0.00 | 6.85 |
| GO_CONNECTIVE_TISSUE_DEVELOPMENT | 5.04 | 0.39 | 1.06 | 6.85 |
| GO_RRNA_METABOLIC_PROCESS | 0.00 | 8.66 | 0.00 | 6.84 |
| GO_POSITIVE_REGULATION_OF_CYTOSKELETON_ORGANIZATION | 0.00 | 0.77 | 0.44 | 6.84 |
| GO_ACTOMYOSIN | 0.68 | 0.56 | 0.84 | 6.79 |
| GO_PROTEIN_COMPLEX_SUBUNIT_ORGANIZATION | 0.66 | 6.51 | 0.46 | 6.79 |
| GO_NEGATIVE_REGULATION_OF_PHOSPHORUS_METABOLIC_{—} | 0.47 | 1.41 | 5.08 | 6.77 |
| PROCESS | | | | |
| GO_GROWTH_FACTOR_BINDING | 1.94 | 0.47 | 1.41 | 6.76 |
| GO_POSITIVE_REGULATION_OF_IMMUNE_SYSTEM_PROCESS | 1.34 | 0.23 | 0.42 | 6.76 |
| GO_REGULATION_OF_OSSIFICATION | 7.60 | 0.46 | 0.00 | 6.73 |
| GO_RESPONSE_TO_ESTROGEN | 2.84 | 0.80 | 2.64 | 6.71 |
| GO_MEMBRANE_REGION | 0.86 | 0.15 | 2.12 | 6.70 |
| GO_POSITIVE_REGULATION_OF_LOCOMOTION | 0.67 | 0.78 | 2.37 | 6.70 |
| GO_CELLULAR_RESPONSE_TO_NUTRIENT | 0.86 | 0.86 | 0.00 | 6.69 |
| GO_MUSCLE_SYSTEM_PROCESS | 1.04 | 0.16 | 0.79 | 6.69 |
| GO_NEGATIVE_REGULATION_OF_TRANSFERASE_ACTIVITY | 0.83 | 1.46 | 2.71 | 6.65 |
| GO_NEGATIVE_REGULATION_OF_NERVOUS_SYSTEM_{—} | 3.31 | 0.19 | 1.54 | 6.64 |
| DEVELOPMENT | | | | |
| GO_BASEMENT_MEMBRANE | 1.31 | 0.10 | 0.68 | 6.64 |
| GO_MOLECULAR_FUNCTION_REGULATOR | 0.12 | 0.72 | 0.59 | 6.64 |
| GO_REGULATION_OF_WOUND_HEALING | 0.00 | 0.45 | 0.56 | 6.62 |
| GO_REGULATION_OF_EPITHELIAL_CELL_PROLIFERATION | 7.97 | 0.98 | 6.23 | 6.60 |
| GO_REGULATION_OF_OSTEOBLAST_DIFFERENTIATION | 6.64 | 0.54 | 0.00 | 6.49 |
| GO_POSITIVE_REGULATION_OF_EXTRINSIC_APOPTOTIC_{—} | 0.74 | 0.00 | 2.09 | 6.49 |
| SIGNALING_PATHWAY | | | | |
| GO_HOMEOSTATIC_PROCESS | 0.24 | 0.07 | 1.72 | 6.47 |
| GO_REGULATION_OF_EPITHELIAL_CELL_MIGRATION | 1.60 | 1.22 | 1.18 | 6.47 |
| GO_POSITIVE_REGULATION_OF_PHOSPHORUS_METABOLIC_{—} | 2.24 | 1.18 | 8.43 | 6.44 |
| PROCESS | | | | |
| HALLMARK_TGF_BETA_SIGNALING | 0.73 | 0.22 | 0.00 | 6.41 |
| GO_GROWTH | 1.15 | 0.28 | 1.69 | 6.39 |
| GO_NEGATIVE_REGULATION_OF_KINASE_ACTIVITY | 1.16 | 1.23 | 3.37 | 6.37 |
| GO_REGULATION_OF_CELLULAR_RESPONSE_TO_TRANSFORMING_{—} | 0.50 | 0.64 | 1.58 | 6.32 |
| GROWTH_FACTOR_BETA_STIMULUS | | | | |
| GO_DEFENSE_RESPONSE | 0.00 | 0.17 | 1.92 | 6.28 |
| GO_POSITIVE_REGULATION_OF_BIOSYNTHETIC_PROCESS | 8.86 | 4.71 | 8.11 | 6.27 |
| GO_REGULATION_OF_EXTRINSIC_APOPTOTIC_SIGNALING_{—} | 0.94 | 0.14 | 2.16 | 6.24 |
| PATHWAY | | | | |
| GO_REGULATION_OF_METAL_ION_TRANSPORT | 0.15 | 0.52 | 0.70 | 6.16 |
| GO_CELL_PROJECTION | 2.16 | 0.29 | 1.09 | 6.14 |
| GO_CELLULAR_RESPONSE_TO_ACID_CHEMICAL | 0.00 | 1.13 | 1.14 | 6.14 |
| GO_LEUKOCYTE_MIGRATION | 0.20 | 0.58 | 1.55 | 6.12 |
| HALLMARK_IL6_JAK_STAT3_SIGNALING | 0.00 | 0.11 | 0.70 | 6.11 |
| GO_REGULATION_OF_PROTEIN_COMPLEX_ASSEMBLY | 0.38 | 0.36 | 1.82 | 6.10 |
| GO_CELL_CORTEX | 1.21 | 0.25 | 2.50 | 6.10 |
| GO_REGULATION_OF_NEURON_DIFFERENTIATION | 5.11 | 0.09 | 3.31 | 6.07 |
| GO_ENDOPLASMIC_RETICULUM | 0.79 | 0.12 | 0.40 | 6.06 |
| GO_RESPONSE_TO_BACTERIUM | 0.00 | 0.18 | 0.82 | 6.06 |
| GO_SIDE_OF_MEMBRANE | 0.00 | 0.03 | 0.53 | 6.02 |
| GO_RIBOSOME_BIOGENESIS | 0.00 | 8.61 | 0.00 | 6.01 |
| GO_REGULATION_OF_TRANSCRIPTION_FROM_RNA_POLYMERASE_{—} | 10.46 | 3.16 | 5.22 | 5.86 |
| II_PROMOTER | | | | |
| GO_PEPTIDE_METABOLIC_PROCESS | 0.05 | 9.79 | 0.00 | 5.34 |
| GO_POLY_A_RNA_BINDING | 0.08 | 17.00 | 0.76 | 5.25 |
| GO_CYTOSOLIC_SMALL_RIBOSOMAL_SUBUNIT | 0.00 | 6.24 | 0.00 | 5.17 |
| GO_RNA_BINDING | 0.14 | 17.00 | 0.42 | 5.17 |
| GO_RIBONUCLEOPROTEIN_COMPLEX | 0.00 | 17.00 | 0.07 | 4.82 |
| GO_EPITHELIAL_CELL_DIFFERENTIATION | 6.45 | 0.23 | 3.60 | 4.82 |
| GO_TUBE_DEVELOPMENT | 6.81 | 0.36 | 6.92 | 4.79 |
| GO_POSITIVE_REGULATION_OF_TRANSCRIPTION_FROM_RNA_{—} | 6.74 | 1.74 | 5.98 | 4.70 |
| POLYMERASE_II_PROMOTER | | | | |
| GO_ORGANIC_CYCLIC_COMPOUND_CATABOLIC_PROCESS | 0.09 | 12.54 | 0.18 | 4.61 |
| GO_NCRNA_PROCESSING | 0.00 | 8.70 | 0.00 | 4.41 |
| GO_HEAD_DEVELOPMENT | 8.95 | 0.86 | 6.76 | 4.24 |
| GO_AMIDE_BIOSYNTHETIC_PROCESS | 0.00 | 9.24 | 0.00 | 4.22 |
| GO_CENTRAL_NERVOUS_SYSTEM_DEVELOPMENT | 6.06 | 0.68 | 7.58 | 4.12 |
| GO_CELL_DEVELOPMENT | 7.98 | 0.37 | 3.69 | 4.10 |
| GO_TRANSCRIPTION_FACTOR_BINDING | 6.19 | 1.81 | 5.19 | 3.98 |
| GO_RIBONUCLEOPROTEIN_COMPLEX_BIOGENESIS | 0.00 | 14.41 | 0.00 | 3.97 |
| GO_NEGATIVE_REGULATION_OF_TRANSCRIPTION_FROM_RNA_{—} | 8.67 | 0.59 | 2.58 | 3.88 |
| POLYMERASE_II_PROMOTER | | | | |
| GO_CELLULAR_CATABOLIC_PROCESS | 0.05 | 9.01 | 0.18 | 3.85 |
| GO_MESENCHYME_DEVELOPMENT | 9.83 | 0.67 | 1.07 | 3.83 |
| GO_CELLULAR_AMIDE_METABOLIC_PROCESS | 0.03 | 7.66 | 0.00 | 3.69 |
| GO_ORGANONITROGEN_COMPOUND_BIOSYNTHETIC_PROCESS | 0.12 | 8.02 | 0.93 | 3.58 |
| GO_MUSCLE_STRUCTURE_DEVELOPMENT | 6.15 | 0.73 | 3.11 | 3.28 |
| GO_EMBRYONIC_MORPHOGENESIS | 8.79 | 0.10 | 10.27 | 3.06 |
| GO_GLAND_DEVELOPMENT | 5.61 | 1.17 | 6.20 | 3.00 |
| GO_ORGANONITROGEN_COMPOUND_METABOLIC_PROCESS | 0.28 | 7.88 | 0.31 | 2.98 |
| GO_MORPHOGENESIS_OF_AN_EPITHELIUM | 6.48 | 0.10 | 7.23 | 2.94 |
| GO_MESENCHYMAL_CELL_DIFFERENTIATION | 10.06 | 1.13 | 0.54 | 2.94 |
| GO_EMBRYONIC_ORGAN_DEVELOPMENT | 10.49 | 0.17 | 4.14 | 2.86 |
| GO_PLACENTA_DEVELOPMENT | 2.72 | 0.39 | 7.23 | 2.84 |
| GO_REPRODUCTIVE_SYSTEM_DEVELOPMENT | 4.62 | 1.09 | 9.42 | 2.84 |
| GO_NUCLEOLUS | 0.40 | 10.02 | 1.69 | 2.83 |
| GO_UROGENITAL_SYSTEM_DEVELOPMENT | 7.76 | 0.42 | 3.02 | 2.75 |
| GO_HEART_DEVELOPMENT | 7.67 | 0.80 | 3.76 | 2.51 |
| GO_NCRNA_METABOLIC_PROCESS | 0.00 | 8.13 | 0.00 | 2.46 |
| GO_MRNA_METABOLIC_PROCESS | 0.17 | 17.00 | 0.34 | 2.33 |
| GO_DEVELOPMENTAL_PROCESS_INVOLVED_IN_REPRODUCTION | 3.34 | 0.52 | 7.53 | 2.13 |
| GO_HEART_MORPHOGENESIS | 6.95 | 0.84 | 1.78 | 1.96 |
| GO_SENSORY_ORGAN_DEVELOPMENT | 9.32 | 0.36 | 2.08 | 1.94 |
| GO_TUBE_MORPHOGENESIS | 4.51 | 0.10 | 6.93 | 1.72 |
| GO_EMBRYO_DEVELOPMENT_ENDING_IN_BIRTH_OR_EGG_{—} | 9.60 | 0.86 | 5.00 | 1.71 |
| HATCHING | | | | |
| GO_RNA_PROCESSING | 0.08 | 17.00 | 0.05 | 1.69 |
| GO_TRANSCRIPTION_FACTOR_ACTIVITY_RNA_POLYMERASE_{—} | 4.46 | 0.10 | 8.04 | 1.67 |
| II_CORE_PROMOTER_PROXIMAL_REGION_SEQUENCE_SPECIFIC_{—} | | | | |
| BINDING | | | | |
| GO_MRNA_BINDING | 0.35 | 9.91 | 0.49 | 1.55 |
| GO_NEURON_DIFFERENTIATION | 8.43 | 0.84 | 2.81 | 1.46 |
| GO_REGULATION_OF_ORGAN_FORMATION | 6.89 | 0.38 | 0.00 | 1.43 |
| GO_CELL_CYCLE_PROCESS | 0.65 | 10.70 | 2.24 | 1.35 |
| HALLMARK_HEDGEHOG_SIGNALING | 8.36 | 0.00 | 0.00 | 1.30 |
| GO_SENSORY_ORGAN_MORPHOGENESIS | 7.62 | 0.25 | 0.91 | 1.28 |
| GO_MESONEPHROS_DEVELOPMENT | 7.30 | 0.11 | 1.66 | 1.24 |
| GO_CELL_CYCLE | 0.90 | 10.00 | 1.76 | 1.16 |
| GO_NUCLEAR_TRANSPORT | 0.82 | 6.20 | 0.00 | 1.15 |
| GO_MACROMOLECULAR_COMPLEX_ASSEMBLY | 0.56 | 11.35 | 0.55 | 1.14 |
| GO_FOREBRAIN_DEVELOPMENT | 6.00 | 0.07 | 6.57 | 1.13 |
| GO_EYE_DEVELOPMENT | 6.36 | 0.34 | 0.70 | 1.10 |
| GO_GLYCOSYL_COMPOUND_METABOLIC_PROCESS | 0.39 | 7.16 | 0.00 | 1.05 |
| GO_NUCLEOSIDE_MONOPHOSPHATE_METABOLIC_PROCESS | 0.22 | 6.92 | 0.00 | 0.98 |
| GO_NEPHRON_DEVELOPMENT | 6.56 | 0.24 | 0.59 | 0.89 |
| GO_NEGATIVE_REGULATION_OF_CELL_CYCLE | 3.62 | 6.49 | 2.32 | 0.87 |
| GO_NUCLEOSIDE_TRIPHOSPHATE_METABOLIC_PROCESS | 0.24 | 9.55 | 0.00 | 0.79 |
| GO_KIDNEY_EPITHELIUM_DEVELOPMENT | 7.60 | 0.21 | 1.39 | 0.78 |
| GO_MITOTIC_CELL_CYCLE | 0.10 | 10.28 | 1.35 | 0.75 |
| GO_CELLULAR_MACROMOLECULAR_COMPLEX_ASSEMBLY | 0.03 | 12.19 | 0.07 | 0.75 |
| GO_CANONICAL_WNT_SIGNALING_PATHWAY | 0.52 | 0.10 | 6.82 | 0.75 |
| GO_CELL_DIVISION | 1.01 | 7.15 | 0.16 | 0.74 |
| GO_NUCLEOSIDE_TRIPHOSPHATE_BIOSYNTHETIC_PROCESS | 0.00 | 7.00 | 0.00 | 0.73 |
| GO_REGULATION_OF_RNA_SPLICING | 0.51 | 7.11 | 0.00 | 0.73 |
| GO_ENVELOPE | 0.01 | 6.38 | 0.52 | 0.72 |
| GO_TRANSCRIPTIONAL_ACTIVATOR_ACTIVITY_RNA_{—} | 6.50 | 0.22 | 7.03 | 0.70 |
| POLYMERASE_II_TRANSCRIPTION_REGULATORY_REGION_{—} | | | | |
| SEQUENCE_SPECIFIC_BINDING | | | | |
| GO_EYE_MORPHOGENESIS | 6.07 | 0.40 | 0.00 | 0.68 |
| GO_RNA_POLYMERASE_II_TRANSCRIPTION_FACTOR_ACTIVITY_{—} | 9.75 | 0.04 | 5.43 | 0.67 |
| SEQUENCE_SPECIFIC_DNA_BINDING | | | | |
| GO_CHROMATIN | 2.18 | 9.40 | 0.51 | 0.65 |
| GO_REGULATORY_REGION_NUCLEIC_ACID_BINDING | 6.41 | 0.30 | 6.10 | 0.57 |
| GO_METANEPHROS_DEVELOPMENT | 6.19 | 0.82 | 0.00 | 0.54 |
| GO_REGULATION_OF_MRNA_METABOLIC_PROCESS | 0.00 | 7.96 | 0.00 | 0.53 |
| GO_RIBONUCLEOSIDE_TRIPHOSPHATE_BIOSYNTHETIC_PROCESS | 0.00 | 6.75 | 0.00 | 0.47 |
| GO_NUCLEOBASE_CONTAINING_SMALL_MOLECULE_{—} | 0.48 | 6.96 | 0.13 | 0.44 |
| METABOLIC_PROCESS | | | | |
| GO_DOUBLE_STRANDED_DNA_BINDING | 6.78 | 0.53 | 7.34 | 0.39 |
| GO_CAMERA_TYPE_EYE_MORPHOGENESIS | 6.95 | 0.30 | 0.00 | 0.38 |
| GO_REGULATION_OF_MRNA_SPLICING_VIA_SPLICEOSOME | 0.00 | 7.14 | 0.00 | 0.37 |
| GO_NEGATIVE_REGULATION_OF_RNA_SPLICING | 0.00 | 6.90 | 0.00 | 0.35 |
| GO_REGULATION_OF_CHROMOSOME_ORGANIZATION | 1.05 | 6.61 | 0.80 | 0.33 |
| GO_RIBONUCLEOPROTEIN_COMPLEX_SUBUNIT_ORGANIZATION | 0.00 | 8.15 | 0.00 | 0.31 |
| HALLMARK_OXIDATIVE_PHOSPHORYLATION | 0.00 | 8.92 | 0.00 | 0.31 |
| GO_NUCLEIC_ACID_BINDING_TRANSCRIPTION_FACTOR_{—} | 7.81 | 0.03 | 5.90 | 0.29 |
| ACTIVITY | | | | |
| GO_SEQUENCE_SPECIFIC_DNA_BINDING | 9.71 | 0.33 | 4.29 | 0.27 |
| HALLMARK_WNT_BETA_CATENIN_SIGNALING | 6.28 | 0.00 | 0.99 | 0.20 |
| GO_DNA_TEMPLATED_TRANSCRIPTION_TERMINATION | 0.00 | 6.92 | 0.00 | 0.17 |
| GO_HYDROGEN_ION_TRANSMEMBRANE_TRANSPORT | 0.00 | 6.64 | 0.00 | 0.15 |
| GO_APPENDAGE_DEVELOPMENT | 9.06 | 0.11 | 3.05 | 0.12 |
| GO_SPLICEOSOMAL_COMPLEX | 0.00 | 10.96 | 0.45 | 0.12 |
| GO_TERMINATION_OF_RNA_POLYMERASE_II_TRANSCRIPTION | 0.00 | 7.14 | 0.00 | 0.12 |
| GO_NUCLEAR_CHROMOSOME | 2.43 | 7.73 | 0.41 | 0.11 |
| GO_REGULATION_OF_GENE_EXPRESSION_EPIGENETIC | 0.67 | 6.48 | 0.00 | 0.11 |
| GO_MRNA_3_END_PROCESSING | 0.00 | 7.54 | 0.00 | 0.09 |
| GO_DNA_METABOLIC_PROCESS | 0.49 | 9.85 | 0.07 | 0.07 |
| GO_MITOTIC_NUCLEAR_DIVISION | 0.13 | 7.96 | 0.63 | 0.07 |
| GO_REGIONALIZATION | 12.14 | 0.58 | 1.35 | 0.07 |
| GO_RNA_SPLICING_VIA_TRANSESTERIFICATION_REACTIONS | 0.00 | 17.00 | 0.00 | 0.06 |
| GO_CHROMOSOME | 1.74 | 12.88 | 0.41 | 0.06 |
| GO_CATALYTIC_STEP_2_SPLICEOSOME | 0.00 | 9.61 | 0.00 | 0.06 |
| GO_DNA_CONFORMATION_CHANGE | 0.00 | 8.87 | 0.00 | 0.06 |
| GO_ORGANELLE_FISSION | 0.07 | 7.07 | 0.88 | 0.05 |
| GO_RNA_3_END_PROCESSING | 0.00 | 8.03 | 0.00 | 0.05 |
| GO_NUCLEAR_BODY | 0.42 | 6.32 | 0.65 | 0.04 |
| GO_MITOCHONDRIAL_MEMBRANE_PART | 0.00 | 6.65 | 0.00 | 0.04 |
| GO_CHROMOSOME_CENTROMERIC_REGION | 0.00 | 10.86 | 0.00 | 0.04 |
| GO_RNA_SPLICING | 0.12 | 15.18 | 0.21 | 0.03 |
| GO_RIBONUCLEOPROTEIN_COMPLEX_LOCALIZATION | 0.00 | 6.17 | 0.00 | 0.03 |
| GO_DNA_PACKAGING | 0.00 | 8.32 | 0.00 | 0.03 |
| GO_CONDENSED_CHROMOSOME | 0.77 | 8.28 | 0.00 | 0.03 |
| GO_CHROMOSOME_SEGREGATION | 0.00 | 6.09 | 0.00 | 0.02 |
| GO_NUCLEAR_CHROMOSOME_SEGREGATION | 0.00 | 7.23 | 0.00 | 0.01 |
| GO_MRNA_PROCESSING | 0.31 | 14.65 | 0.17 | 0.01 |
| GO_CHROMOSOMAL_REGION | 0.00 | 9.44 | 0.00 | 0.01 |
| GO_ORGANELLE_INNER_MEMBRANE | 0.00 | 7.13 | 0.13 | 0.01 |
| GO_SISTER_CHROMATID_SEGREGATION | 0.00 | 8.15 | 0.00 | 0.01 |
| GO_HISTONE_BINDING | 0.00 | 8.96 | 0.00 | 0.01 |
| GO_ANTERIOR_POSTERIOR_PATTERN_SPECIFICATION | 9.73 | 0.97 | 1.88 | 0.00 |
| GO_CHROMOSOME_ORGANIZATION | 0.27 | 12.92 | 0.13 | 0.00 |
| GO_CHROMATIN_ORGANIZATION | 0.34 | 8.12 | 0.30 | 0.00 |
| GO_CARDIAC_RIGHT_VENTRICLE_MORPHOGENESIS | 6.47 | 0.00 | 1.40 | 0.00 |
| GO_MITOCHONDRIAL_PROTEIN_COMPLEX | 0.00 | 8.05 | 0.00 | 0.00 |
| GO_INNER_MITOCHONDRIAL_MEMBRANE_PROTEIN_COMPLEX | 0.00 | 7.61 | 0.00 | 0.00 |
| GO_MITOCHONDRIAL_ATP_SYNTHESIS_COUPLED_PROTON_{—} | 0.00 | 6.66 | 0.00 | 0.00 |
| TRANSPORT | | | | |
| GO_NEGATIVE_REGULATION_OF_MRNA_SPLICING_VIA_{—} | 0.00 | 6.33 | 0.00 | 0.00 |
| SPLICEOSOME | | | | |
| GO_ELECTRON_TRANSPORT_CHAIN | 0.00 | 6.32 | 0.00 | 0.00 |
| GO_CELLULAR_COMPONENT_DISASSEMBLY_INVOLVED_IN_{—} | 0.00 | 6.32 | 0.00 | 0.00 |
| EXECUTION_PHASE_OF_APOPTOSIS | | | | |
| GO_RNA_STABILIZATION | 0.00 | 6.20 | 0.00 | 0.00 |
| GO_KINETOCHORE_ORGANIZATION | 0.00 | 6.05 | 0.00 | 0.00 |

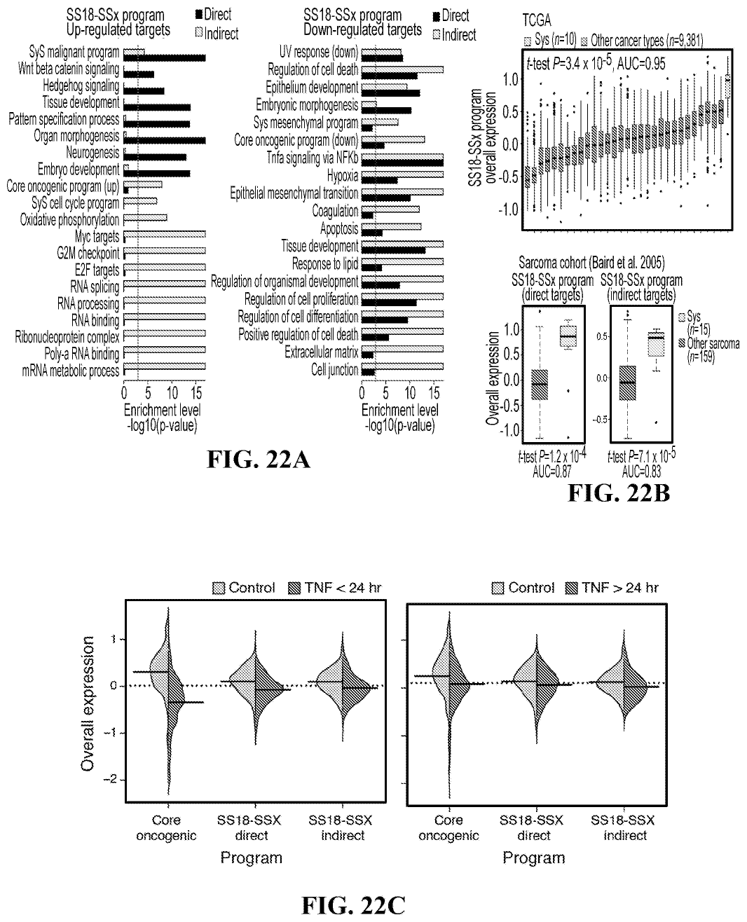

Using these SS18-SSX KD experiments Applicants defined the SS18-SSX program, which Applicants then stratified to direct and indirect fusion targets based on available SS18-SSX ChIP-Seq profiles (13, 28) (Methods; FIGS. 22A-22B, Table 8). Analyzing these functional single-cell data Applicants identified SS18-SSX transcriptional targets/program (FIG. 7A; Tables 8 and 9). Reassuringly, the SS18-SSX program captured bulk transcriptional alterations that followed SS18-SSX KD in another cell line (HS-SY-II, P<1*10−17, hypergeometric test) (Banito et al. Cancer Cell 33:524-541.e8 (2018)). It was enriched with SS18-SSX direct targets (McBride et al. Cancer Cell (2018) doi:10.1016/j.ccell.2018.05.002; Banito et al. Cancer Cell 33:524-541.e8 (2018)) (P=6.66*10−16, hypergeometric test), and repressed genes which are suppressed by the ATF2-SS18/SSX-TLE1 complex (P=2.94*10−8, hypergeometric test) (Su et al. Cancer Cell 21:333-347 (2012)). It was overexpressed in SyS malignant cells compared to non-malignant cells (P<1*10−30, t-test), and in SyS tumors compared to other cancer and sarcoma types (Baird et al. Cancer Res 65:9226-9235 (2005)) (FIGS. 7D-7E). It included IGF2, which is critical for SyS tumorigenesis (Sun et al. Oncogene 25:1042-1052 (2006)), and TLE1, a diagnostic marker of SyS (Banito et al. Cancer Cell 33:524-541.e8 (2018)).

Then, using available SS18-SSX ChIP-Seq profiles (McBride et al. Cancer Cell (2018) doi:10.1016/j.ccell.2018.05.002; Banito et al. Cancer Cell 33:524-541.e8 (2018)), Applicants stratified the SS18-SSX program to its direct and indirect targets and found that the fusion directly dysregulates developmental programs (P<5.28*10−7, hypergeometric test), while its impact on cell cycle is mostly indirect (P<1.2*10−9, hypergeometric test, Tables 8 and 9, FIG. 7E), and mediated by cyclin D2 (CCND2) and CDK6—the only cell cycle genes that are members of the direct SS18-SSX program. Taken together, Applicants' findings support a model in which SS18-SSX directly promotes the core oncogenic program, blocks differentiations, and drives cell cycle progression.

The oncoprotein also directly promotes the core oncogenic program, by directly dysregulating many of its genes (P=2.51*10−5, hypergeometric test) and gene modules, including TNF signaling, hypoxia, apoptosis, and p53 signaling (P<1.4*10−5, FIGS. 7E, 8A, Tables 8 and 9). Lastly, modeling the transcriptional regulation of the core oncogenic program (Methods), Applicants found that it includes a large number of transcription factors (TFs, 47 of 119 repressed genes, P=1.89*10−15, hypergeometric test), with 193 TF-target interactions between its genes. The fusion directly dysregulated the most dominant TFs in the resulting regulatory network, including JUN, ATF4, EGR1, and ATF3.

### Example 5—TNF and IFNγ Synergistically Repress the Core Oncogenic Program and SS18-SSX Program

The association between the core oncogenic program and the cold phenotype suggest that the program promotes T cell exclusion in SyS. Another (non-mutually exclusive) hypothesis is that, despite their low numbers, the immune cells in the tumor microenvironment may nonetheless impact the state of the malignant cells, for example, through the secretion of different molecules and cytokines. To test this, Applicants implemented a mixed-effects inference approach that uses scRNA-Seq data to find associations between the expression of secreted molecules and ligands in immune cells and the state of the malignant cells, as described below. First, Applicants used single-cell immune signatures to estimate the composition of bulk SyS tumors in two published cohorts (Banito et al. Cancer Cell 33:524-541.e8 (2018); Lagarde et al. J Clin Oncol Off J Am Soc Clin Oncol 31:608-615 (2013)) (Methods), and stratified them into “hot” or “cold”, based on their relative inferred proportions of immune cells. “Hot” tumors, with relatively high levels of immune cells, showed repression of the core oncogenic and proliferation programs and had significantly higher differentiation scores (P<5.34*10−3, r=−0.44, −0.36 and 0.48, respectively, partial Pearson correlation, conditioning on inferred tumor purity; FIG. 8B).

Supporting the generalizability of these findings, the core oncogenic program overlapped a transcriptional signature Applicants recently associated with T cell exclusion in melanoma (32) (P<7.16*10⁻¹⁰, hypergeometric test). Among the overlapping genes Applicants find the induction of the CTAMA GEA4, the BAF complex unit SMARCA4 and genes involved in oxidative phosphorylation, as well as the repression of apoptosis and p53 signaling (e.g., ATF3, JUN, KLF4, and SAT1). The melanoma T cell exclusion signature also overlapped the mesenchymal state defined here, inducing SNAI2 and repressing 23 epithelial genes, including CDH1 (P=6.33*10⁻⁸, hypergeometric test).

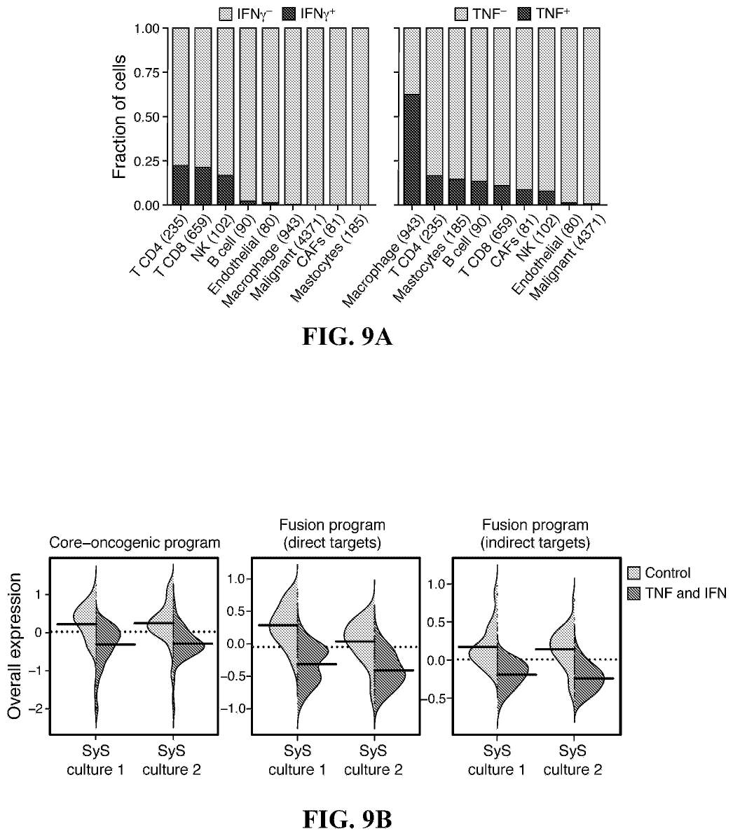

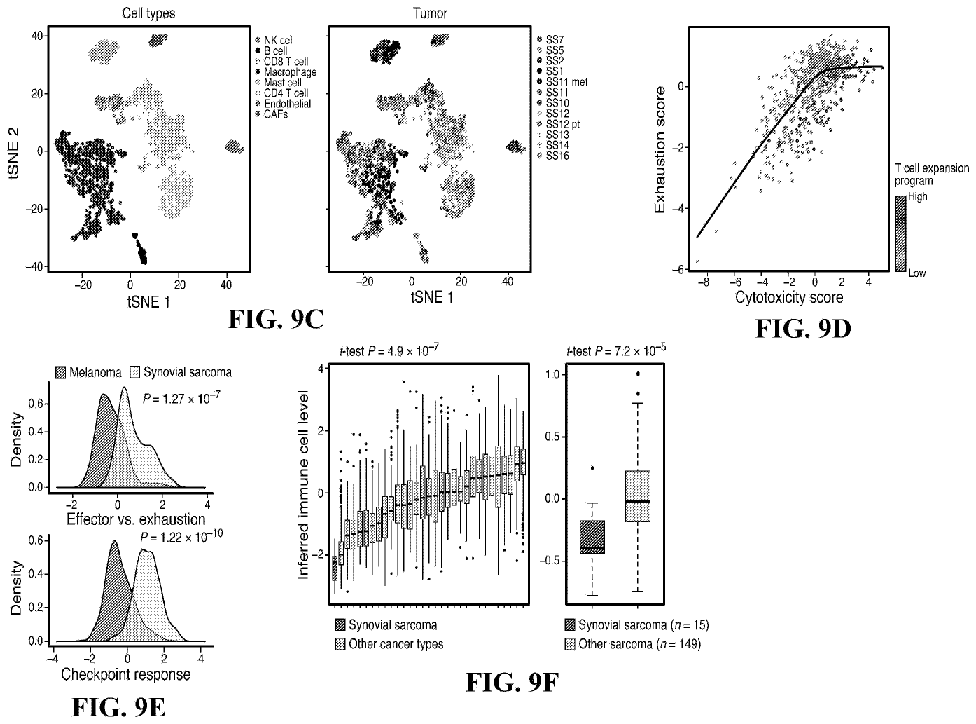

To examine whether immune cells impact SyS cells through physical interactions (ligand-receptor bindings) and the secretion of certain cytokines, Applicants developed a mixed-effects inference approach that uses scRNA-Seq data to find associations between the expression of ligands in immune cells and the state of the malignant cells (Methods). This analysis revealed that the expression of IFNγ and TNF in CD8 T cells and macrophages, respectively (FIG. 9A), was strongly associated with the repression of the core oncogenic program in the malignant cells (P<9.4*10⁻³⁹, mixed-effects). In accordance with these findings, TNF receptors and multiple genes involved in TNF and IFN responses were repressed in the core oncogenic program, and according to the connectivity map (CMap) (Subramanian et al. Cell 171:1437-1452.e17 (2017))—the overexpression of IFNs and TNF receptors repressed the program in various cancer cell lines. Applicants further stratified the core oncogenic program to its predicted TNF/IFNy-dependent and -independent components, by the association of each gene's expression in the malignant cells with the TNF and IFNy expression levels in the corresponding macrophages and CD8 T cells, respectively (Methods, Table 10A).

To examine these associations, Applicants treated primary SyS cell cultures with TNF and IFNγ, both separately and in combination, and profiled 1,050 cells by scRNA-Seq. As predicted, combined TNF and IFNγ treatment repressed the core oncogenic program (P=6.66*10⁻¹⁸, mixed-effects, FIG. 9B) in a synergistic manner (P=9.49*10⁻⁴, interaction term, mixed-effects). Moreover, the treatment repressed the SS18-SSX program (P<3.12*10⁻¹⁶, both direct and indirect components, including TLE1; FIG. 9B, Tables 10 and 11), while inducing multiple genes from the epithelial program (P=1.95*10−9, hypergeometric test, Tables 10 and 11). Short-term (4-6 hours) treatment with TNF alone substantially repressed homeobox genes (e.g., MEOX2, Tables 10 and 11), which are directly bound by SS18-SSX (McBride et al. Cancer Cell (2018) doi:10.1016/j.ccell.2018.05.002; Banito et al. Cancer Cell 33:524-541.e8 (2018)) (P<1*10⁻¹⁷, hypergeometric test). It also repressed the core oncogenic program, but only temporarily (P=8.73*10−18, mixed-effects; FIG. 8C), suggesting that IFNγ is require to sustain the effect. Interestingly, TNF also induced TNF expression in the Sys cells (P<5.57*¹⁰⁻8, mixed-effects, FIG. 22C), suggesting that autocrine signaling might induce the effect as well. Taken together these findings demonstrate that macrophages and T cells can suppress the SS18-SSX program by secreting TNFγ and IFNγ.

TABLE 10A
Predicted TNF/IFN-dependent and independent components of the core
oncogenic program according to the cell-cell interaction analyses.

| | | | | Core oncogenic | |
|---|---|---|---|---|---|
| Core oncogenic TNF/IFN-independent | | | | TNF/IFN-dependent | |
| UP | | | DOWN | UP | DOWN |
| AFG3L1P | HERC2 | PFN1 | AMD1 | AHCY | ATF3 |
| AGPAT2 | HIGD2A | PFN1P2 | ATF4 | BTF3 | BHLHE40 |
| AGPAT5 | HINT1 | PGD | BRD2 | DBNDD1 | CDKN1A |
| AKR1B1 | HMG20B | PGLS | BTG1 | EEF1G | CSRNP1 |
| AKR1C3 | HN1L | PHF14 | C12orf44 | FADS2 | DDX5 |
| AKT1 | HNRNPD | PIGQ | C6orf62 | FGF9 | DUSP1 |
| ALG3 | HOXD11 | PIGT | CCNL1 | GLB1L2 | DUSP2 |
| ALX4 | HOXD9 | PKD2 | CKS2 | GNB2L1 | FOSL1 |
| ANAPC7 | HSD17B10 | PLP2 | CLK1 | LDHB | JUND |
| ANKRD26P1 | HYAL2 | PMS2P5 | COQ10B | MDH2 | KLF4 |
| APEH | HYLS1 | POLD2 | CYCS | NACA | KLF6 |
| APRT | ICT1 | POLR1B | DDX3X | PPIA | LMNA |
| ARF5 | IFT81 | POLR2F | DDX3Y | PTPRS | MAFF |
| ARL6IP4 | IRS4 | PPIB | DLX2 | PXDN | MIR22HG |
| ARL6IP5 | ITM2C | PPIP5K2 | DNAJA1 | SEMA3A | NFKBIA |
| ATF7IP | ITPA | PPP1R16A | DNAJA4 | SLC25A6 | NFKBIZ |
| ATP5A1 | JMJD8 | PRDX4 | DNAJB1 | TKT | NR4A1 |
| ATP5E | KDM1A | PRELID1 | DNAJB9 | UBA52 | PER1 |
| ATP5J | KIAA0020 | PSMA5 | EGR1 | VCAN | SAT1 |
| ATP5J2 | KRT14 | PSMA7 | EGR2 | | SIK1 |
| ATR | KRT15 | PSMB7 | EGR3 | | TNFAIP3 |
| ATRAID | KRT8 | PSMD4 | EIF4A3 | | TNFRSF12A |
| AUP1 | KRTCAP2 | PSMG3 | EIF5 | | UBC |
| AURKAIP1 | LAMA2 | PTPRF | ERF | | |
| BCAP31 | LARP1 | PUS7 | FAM53C | | |
| BCL7C | LECT1 | RABAC1 | FOS | | |
| BMP1 | LGALS1 | RABL6 | FOSB | | |
| BOP1 | LINC00115 | RANBP1 | GADD45B | | |
| BRK1 | LINC00116 | RBM26 | GEM | | |
| BSG | LOC100272216 | RBM6 | GTF2B | | |
| C11orf48 | LOC202781 | RBX1 | H3F3B | | |
| C16orf88 | LOC375295 | REST | HBP1 | | |
| C2orf68 | LOC654433 | RGMA | HERPUD1 | | |
| C4orf48 | LOXL1 | RGS10 | HES1 | | |
| C7orf73 | LSM4 | RHOBTB3 | HSP90AA1 | | |
| C9orf16 | LSM7 | RNASEK | HSP90AB1 | | |
| CALML3 | LUC7L3 | RNPC3 | HSPA1A | | |
| CAPNS1 | LY6E | RNPEP | HSPA1B | | |
| CBX6 | MAB21L1 | ROMO1 | HSPA8 | | |
| CCDC137 | MAGEA4 | RUVBL1 | HSPH1 | | |
| CCDC140 | MAGEA9 | SARS2 | ICAM1 | | |
| CD63 | MAGEC2 | SELENBP1 | ID1 | | |
| CD7 | MAP1B | SERF2 | ID2 | | |
| CDK2AP1 | MATN3 | SERTAD4 | ID3 | | |
| CECR5 | MBD6 | SETD4 | IER2 | | |
| CHCHD1 | MDK | SH2D4A | IRF1 | | |
| CHCHD2 | METTL3 | SH3PXD2B | JUN | | |
| CIAPIN1 | MFSD3 | SIM2 | JUNB | | |
| CKAP5 | MGC21881 | SLC25A23 | KLHL15 | | |
| CLNS1A | MGST1 | SLC35B4 | LOC284454 | | |
| CNPY2 | MGST3 | SLC6A15 | MCL1 | | |
| COL18A1 | MIS18A | SMARCA4 | MLF1 | | |
| COL5A1 | MKKS | SMC2 | MXD1 | | |
| COL6A2 | MMP14 | SMC3 | NR4A2 | | |
| COL9A3 | MRPL12 | SNRPD3 | NR4A3 | | |
| COX4I1 | MRPL17 | SNRPF | PAFAH1B2 | | |
| COX5A | MRPL28 | SPCS1 | RGS16 | | |
| COX5B | MRPL35 | SRI | RIPK4 | | |
| COX6A1 | MRPL4 | SRM | RRP12 | | |
| COX6B1 | MRPL52 | SRSF9 | SERTAD1 | | |
| COX6C | MRPS17 | SSNA1 | SF1 | | |
| CRIP1 | MRPS21 | SSR4 | SLC25A25 | | |
| CRLF1 | MRPS34 | SSX2 | SLC25A44 | | |
| CRMP1 | MTG1 | SSX2B | SOCS3 | | |
| CSAG3 | MTRNR2L1 | STAG3L2 | SRSF3 | | |
| CSRP2BP | MTRNR2L10 | STAG3L3 | TOB1 | | |
| CST3 | MTRNR2L2 | STAG3L4 | TRIB1 | | |
| CSTB | MTRNR2L6 | STARD4- | TSPYL1 | | |
| | | AS1 | | | |
| CSTF3 | MTRNR2L8 | SULF2 | TUBA1A | | |
| CTAG1A | MYBBP1A | SULT1A1 | TUBA1B | | |
| CTAG1B | NAT14 | SUMF2 | TUBB2A | | |
| CYHR1 | NDUFA1 | SYNPR | TUBB4B | | |
| DAD1 | NDUFA13 | TBCD | UBB | | |
| DANCR | NDUFA4 | TCEB2 | YWHAG | | |
| DCP1B | NDUFA7 | TELO2 | | | |
| DCXR | NDUFA8 | TFAP2A | | | |
| DGCR6L | NDUFAB1 | THY1 | | | |
| DHFR | NDUFB10 | TIGD1 | | | |
| DNMT3A | NDUFB11 | TIMM8B | | | |
| DPEP3 | NDUFB2 | TMA7 | | | |
| DYNLRB1 | NDUFB3 | TMC6 | | | |
| DYNLT1 | NDUFB4 | TMEM101 | | | |
| EDF1 | NDUFB7 | TMEM147 | | | |
| EEF1D | NDUFB9 | TMEM177 | | | |
| EIF2AK1 | NDUFS6 | TOMM40 | | | |
| EIF3K | NDUFS8 | TOMM6 | | | |
| ELAC2 | NEDD8 | TOMM7 | | | |
| ELOVL1 | NEFL | TRAPPC1 | | | |
| EML3 | NIPSNAP3A | TSR3 | | | |
| EPRS | NKAIN4 | TSTA3 | | | |
| ERGIC3 | NME1 | TTYH3 | | | |
| ETAA1 | NNT | TUFM | | | |
| EXOSC4 | NOMO1 | TUSC3 | | | |
| EXOSC7 | NOMO2 | TWIST2 | | | |
| FADD | NPEPL1 | TXN | | | |
| FAM178A | NRBP2 | TXNDC17 | | | |
| FAM19A5 | NSMF | TXNDC5 | | | |
| FAM213B | NSUN5 | TXNDC9 | | | |
| FAM50B | NSUN5P1 | UBE2T | | | |
| FARSA | NSUN5P2 | UBE3B | | | |
| FARSB | NT5DC2 | UPK3B | | | |
| FBN3 | NUBP2 | UQCR10 | | | |
| FLAD1 | NUDT5 | UQCR11 | | | |
| FRG1B | NUTF2 | UQCRC1 | | | |
| G6PC3 | OBSL1 | UQCRQ | | | |
| GADD45GIP1 | OGG1 | USMG5 | | | |
| GCN1L1 | OST4 | USP5 | | | |
| GEMIN7 | OXLD1 | VARS | | | |
| GLB1L | PATZ1 | VKORC1 | | | |
| GLI1 | PAX3 | VPS28 | | | |
| GNAS | PCDHA3 | VPS72 | | | |
| GNPTAB | PDCD11 | VSNL1 | | | |
| GOLM1 | PDCD5 | WDR12 | | | |
| GPR124 | PDIA4 | YWHAB | | | |
| GPR126 | PEBP1 | ZNF212 | | | |
| GPRC5B | PET100 | ZNF605 | | | |
| GSTO2 | PFKL | | | | |
| GUSB | PFKP | | | | |
| H19 | | | | | |

TABLE 10B
Differentially expressed genes following TNF and IFN-gamma treatment.

| | | TNF | TNF & | IFN | TNF | TNF short | TNF & IFN |
|---|---|---|---|---|---|---|---|
| IFN up | TNF up | short up | IFN up | down | down | down | down |
| A2M | ABTB2 | ABCA5 | ABCG1 | ACTG1 | ACTG1 | AASDHPPT | ABI2 |
| ABHD16A | ACAT2 | ABCF1 | ABHD16A | AKAP9 | ADAMTS9 | ABHD10 | ACTB |
| ACSL1 | ACHE | ABR | ABTB1 | APCDD1L | ANO1 | ACN9 | ACTG1 |
| ACTA2 | ACOX1 | ALCAM | ACSL1 | BTF3 | APCDD1L | ADAMTS14 | ACTR2 |
| ACY3 | ADAR | ALOX15B | ACTA2 | CD248 | ARHGAP10 | ADH5 | ADH5 |
| ADAMTS3 | AIFM2 | ARID4B | ADAMTSL4 | CYGB | ARL5A | AGPAT1 | AGAP2 |
| ADAR | AKIP1 | ATF3 | ADRB2 | DAZ2 | BAIAP2 | AGPAT2 | ALDH18A1 |
| ADC | ALCAM | ATP13A3 | AFAP1L2 | DAZ4 | BCHE | AGPAT5 | ALDOA |
| ALPK1 | ANO9 | BAZ1A | AHRR | DCBLD2 | BOP1 | AHNAK2 | ANP32E |
| APOBEC3D | ANXA7 | BCL3 | AIFM2 | DHRS3 | BZW2 | AJUBA | ANXA6 |
| APOBEC3F | APBA3 | BHLHE40 | AIM2 | FSTL1 | C17orf76-AS1 | ALDH3A2 | AP2M1 |
| APOBEC3G | APLF | BID | AKAP2 | FUS | C6orf48 | ALDOA | AP3S1 |
| APOL1 | APOBEC3F | BIRC3 | ALOX5AP | GAL | CA10 | ALG1 | APEX1 |
| APOL2 | APOBEC3G | BTG2 | ALPK1 | GATA6 | CALCRL | ALKBH2 | ARCN1 |
| APOL3 | APOL2 | BTG3 | ANPEP | GSE1 | CCDC178 | AMZ2P1 | ARF1 |
| APOL4 | APOL6 | C10orf10 | AP1G2 | HOXA6 | CD248 | ANKZF1 | ARF4 |
| APOL6 | ATXN2L | C11orf96 | AP5Z1 | HOXB5 | CHODL | ANP32A | ARF5 |
| ARHGAP18 | B2M | C15orf48 | APOBEC3C | IPO5 | CHST8 | AP2M1 | ARPC1A |
| ATF3 | BARX2 | C20orf111 | APOBEC3D | JMJD1C | CIRBP | ARAP1 | ARPC2 |
| B2M | BCL6B | C2CD4B | APOBEC3F | KIAA1467 | COL25A1 | ARHGEF3 | ARPC3 |
| BATF2 | BHLHE41 | CAV1 | APOBEC3G | LAMA2 | CXCL12 | ARMCX6 | ARPC4 |
| BATF3 | BID | CBR3 | APOD | NID1 | DANCR | ARSK | ARPP19 |
| BST2 | BIRC2 | CCL1 | APOL1 | OSBPL8 | DAZ2 | ARV1 | ATG12 |
| BTN2A2 | BIRC3 | CCL2 | APOL2 | PCDH11X | DLX1 | ASB13 | ATP5A1 |
| BTN3A1 | BST2 | CCL5 | APOL3 | PKM | EBF1 | ATIC | ATP5B |
| BTN3A2 | BTG2 | CCNL1 | APOL6 | RBM3 | EBF3 | ATP5G2 | ATP5C1 |
| BTN3A3 | BTN2A2 | CCRN4L | AREG | RPL10 | EEF1B2 | ATPIF1 | ATP5F1 |
| C14orf159 | BTN3A1 | CD40 | ARHGEF2 | RPL6 | EGR1 | B3GALNT1 | ATP5G2 |
| C19orf12 | BTN3A2 | CD82 | ARIH2OS | SCN4B | EIF3L | B3GNT1 | ATP5G3 |
| C19orf66 | C10orf10 | CD83 | ARRDC2 | SHISA2 | EIF4EBP1 | BAG4 | ATP5J |
| C1R | C16orf46 | CDK6 | ATAD3C | SLC35F2 | EMP1 | BCOR | ATP5L |
| C1RL | C19orf66 | CEBPD | ATF3 | SMAD9 | ENAH | BIVM | ATP5O |
| C15 | C1R | CFLAR | ATF5 | SSBP2 | EPHA3 | BMP3 | ATP6V1D |
| C5orf56 | C1S | CHST15 | ATG2A | SVEP1 | EPHA7 | BMP4 | ATP6V1G1 |
| CALCOC02 | CASP4 | CNKSR3 | ATHL1 | TAGLN | ERCC1 | BNIP3L | ATXN10 |
| CARD16 | CAV1 | COTL1 | ATP6V0D2 | TMSB15A | ETV1 | BOP1 | AZIN1 |
| CASP1 | CCDC88C | CREB5 | ATXN2L | TNFRSF10D | F2R | BRAT1 | BAI3 |
| CASP4 | CCL2 | CSF2 | B2M | TOMM20 | FAM49A | BRK1 | BANF1 |
| CASP7 | CCL20 | CX3CL1 | BATF2 | TSPAN13 | FARP1 | C11orf48 | BGN |
| CCDC178 | CCL5 | CXCL1 | BATF3 | U2SURP | FHL2 | C12orf52 | BMP5 |
| CCL2 | CCND1 | CXCL2 | BCAR1 | UNC5C | FLI1 | C12orf73 | BNC2 |
| CD200 | CCR10 | CXCL3 | BCL3 | | FLJ41200 | C14orf1 | BNIP3L |
| CD274 | CD40 | CXCR7 | BDKRB2 | | FOS | C17orf58 | BRK1 |
| CD40 | CD44 | CYB5A | BIRC3 | | FZD1 | C18orf32 | BSG |
| CD44 | CD47 | CYLD | BST2 | | GAS2 | C19orf24 | BTF3 |
| CD47 | CD58 | DENND4A | BTG1 | | GAS5 | C20orf112 | BTF3L4 |
| CD74 | CD59 | DOT1L | BTN3A1 | | GNAI1 | C22orf29 | BZW1 |
| CDKN2A | CD70 | DSEL | BTN3A2 | | GNB2L1 | C6orf57 | BZW2 |
| CEACAM1 | CD82 | DUSP5 | BTN3A3 | | GRIK3 | CABIN1 | C11orf58 |
| CFH | CD83 | DYSF | C10orf10 | | H19 | CACYBP | C14orf166 |
| CIITA | CDC42EP4 | ECE1 | C14orf159 | | HOXA10 | CALHM2 | C17orf76-AS1 |
| CLDN1 | CDH13 | EFNA1 | C15orf48 | | HOXD-AS1 | CAPRIN1 | C19orf10 |
| CMPK2 | CFLAR | EGR3 | C19orf12 | | ID2 | CASP2 | C1orf43 |
| CNN3 | CHST15 | EIF5 | C19orf66 | | IMPDH2 | CASP6 | C1QBP |
| CPQ | CNTNAP1 | ELF3 | C1R | | ITGA4 | CBFB | C4orf3 |
| CTSO | COTL1 | ELL2 | C1RL | | ITM2C | CBR1 | C9orf16 |
| CTSS | CPXM2 | ELOVL7 | C1RL-AS1 | | KAL1 | CBX2 | CALM2 |
| CX3CL1 | CREB3 | EPC1 | C1S | | KAZALD1 | CBX3 | CALR |
| CXCL1 | CRIM1 | ETS1 | C2 | | KIAA1467 | CBX8 | CALU |
| CXCL10 | CRYAA | EVA1A | C3 | | KIF26B | CCDC8 | CASP2 |
| CXCL11 | CSF1 | EXT1 | C5orf56 | | KLF10 | CCT8 | CCND2 |
| CXCL16 | CSPG4 | F3 | C8orf4 | | KLHL14 | CECR5 | CCT2 |
| CXCL6 | CTSS | FAM107B | C9orf3 | | LDHB | CES2 | CCT3 |
| CXCL9 | CX3CL1 | FAM19A3 | CA4 | | LHX8 | CHD7 | CCT4 |
| DDX58 | CXCL1 | FAM65A | CALCOCO2 | | LINC00478 | CHD9 | CCT5 |
| DDX60 | CXCL2 | FJX1 | CARD16 | | LOC100506474 | CHST8 | CCT6A |
| DDX60L | CXCL3 | FMNL3 | CASP1 | | LOC644961 | CISD2 | CCT7 |
| DHX58 | CXCR4 | FNDC3B | CASP4 | | LPAR1 | CLASP1 | CCT8 |
| DNPEP | CXCR7 | FOSB | CAV1 | | LRIG3 | CLIP3 | CD248 |
| DPYD | CYB5A | FOSL1 | CAV2 | | LSP1 | CMBL | CD63 |
| DSC2 | CYFIP2 | FTH1 | CCDC130 | | LZTS1 | CNP | CD99 |
| DSG2 | DAG1 | GADD45A | CCDC88C | | LZTS1-AS1 | COMMD3 | CDC42 |
| DTX3L | DBI | GADD45B | CCL2 | | MEG3 | COX20 | CDK4 |
| EBI3 | DCLK1 | GFPT2 | CCL20 | | MITF | CRYZ | CELF2 |
| EDARADD | DDX58 | GPATCH2L | CCL5 | | MLLT11 | CSNK1G3 | CFDP1 |
| ENG | DDX60 | HAS2 | CCL8 | | MMP16 | CSTB | CFL1 |
| ENOX1 | DHCR7 | HIVEP1 | CCNL1 | | MYC | CXXC5 | CHCHD2 |
| EPSTI1 | DHRS3 | HIVEP2 | CCRL1 | | MYL9 | DAG1 | CHMP3 |
| ERAP1 | DHX58 | HLA-A | CD274 | | NDUFA4L2 | DBN1 | CIRBP |
| ERAP2 | DMD | HLA-B | CD38 | | NFIB | DCTD | CNBP |
| ETV7 | DPYSL3 | HLA-C | CD40 | | NR2F2 | DLAT | CNN3 |
| EYA4 | DTX3L | HLA-F | CD47 | | NR5A2 | DLL1 | COL5A2 |
| FAM111A | DTX4 | HLA-H | CD70 | | NRP1 | DLX1 | COPA |
| FAM129A | EBI3 | HMOX1 | CD74 | | OLFM3 | DLX2 | COPZ1 |
| FBXO6 | ECE1 | HSPG2 | CD82 | | PAFAH1B3 | DNAAF2 | CORO1A |
| FIBIN | EDARADD | ICAM1 | CDCP1 | | PAG1 | DNAJC4 | COX5A |
| FLT3LG | EFNA1 | ICOSLG | CDK11A | | PAR-SN | DOLK | COX6C |
| FTH1 | ELF3 | IER3 | CDK11B | | PCDH18 | DPYSL2 | COX7A2 |
| GBP1 | EMP3 | IER5 | CDKN2A | | PCDH7 | DTWD1 | COX7A2L |
| GBP1P1 | EPS8L2 | IFIH1 | CEACAM1 | | PCDH9 | EEF2K | CRABP2 |
| GBP2 | EPSTI1 | IL15 | CEBPB | | PCOLCE2 | EGFLAM | CRIP2 |
| GBP3 | ERAP1 | IL18R1 | CFB | | PCSK1 | EHMT2 | CRTAP |
| GBP4 | ETV7 | IL32 | CFD | | PDLIM7 | ENAH | CSNK1A1 |
| GBP5 | EVA1A | IL34 | CFLAR | | PPIC | ENDOD1 | CSNK2A1 |
| GIMAP2 | EXOC3L4 | IL6 | CH25H | | PRICKLE1 | ENO2 | CSNK2B |
| GPC3 | FADS2 | IL7 | CHKA | | RASL11B | EPHB3 | DAD1 |
| GRM4 | FAM129A | ING3 | CHRNA1 | | REEP2 | EPN2 | DANCR |
| GSDMD | FAM65A | INHBA | CIITA | | RGMB | EXOSC4 | DARS |
| GSTO1 | FMNL3 | IRAK2 | CLCN7 | | RND3 | FAM115A | DAZ2 |
| GTPBP1 | FNDC3B | IRF1 | CLDN1 | | RPL10 | FAM131A | DAZ4 |
| HAPLN3 | FRMD4A | ITGAV | CLIP1 | | RPL10A | FAM136A | DCAF13 |
| HCP5 | FSTL3 | JUN | CLSTN3 | | RPL11 | FAM156A | DCBLD2 |
| HEXDC | FTH1 | JUNB | CMPK2 | | RPL12 | FAM175A | DCTN3 |
| HEY2 | FXYD6 | KDM6B | COL15A1 | | RPL13 | FAM210A | DDB1 |
| HLA-A | GBP1 | KLF5 | CPT1B | | RPL14 | FAM217B | DDOST |
| HLA-B | GBP3 | KLF6 | CPZ | | RPL15 | FANCF | DDX39B |
| HLA-C | GBP4 | KLF7 | CSF1 | | RPL17 | FARSA | DPYSL2 |
| HLA-DMA | GFPT2 | KLF9 | CTSC | | RPL18 | FBXO17 | DSTN |
| HLA-DMB | GFRA1 | LACC1 | CTSD | | RPL18A | FDFT1 | EBF1 |
| HLA-DOA | GPR133 | LAMC2 | CTSO | | RPL19 | FOXK1 | EBF2 |
| HLA-DOB | GPX4 | LIF | CTSS | | RPL22 | FRMD8 | EBF3 |
| HLA-DPA1 | GRAMD1A | LOC100126784 | CX3CL1 | | RPL22L1 | FTSJ2 | EDNRA |
| HLA-DPB1 | GRINA | LOC100862671 | CXCL1 | | RPL23 | FZD1 | EEF1A1 |
| HLA-DQA1 | GSDMD | LOC387895 | CXCL10 | | RPL23A | FZD3 | EEF1B2 |
| HLA-DQB1 | HAPLN3 | LOC440896 | CXCL11 | | RPL24 | GALNT2 | EEF1G |
| HLA-DRA | HAS2 | MAFF | CXCL16 | | RPL26 | GAPDH | EEF2 |
| HLA-DRB1 | HERC6 | MAML2 | CXCL9 | | RPL27 | GIT2 | EFNA5 |
| HLA-DRB5 | HIP1 | MAP2K3 | CYBA | | RPL27A | GLG1 | EID1 |
| HLA-DRB6 | HIPK2 | MAP3K8 | DDIT4L | | RPL28 | GNPDA1 | EIF1 |
| HLA-E | HLA-A | MASTL | DDX58 | | RPL29 | GPI | EIF2S3 |
| HLA-F | HLA-B | MIR155HG | DDX60 | | RPL3 | GRSF1 | EIF3D |
| HLA-H | HLA-C | MIR22HG | DENND3 | | RPL30 | GTF3C2 | EIF3E |
| HRH1 | HLA-E | MTRNR2L1 | DHX58 | | RPL31 | GTF3C6 | EIF3F |
| HS3ST1 | HLA-F | MTRNR2L6 | DLGAP1 | | RPL32 | GTPBP3 | EIF3H |
| ICAM1 | HLA-H | MTRNR2L8 | DNPEP | | RPL34 | HLTF | EIF3I |
| IDO1 | HORMAD1 | NDRG4 | DOCK9 | | RPL35A | HMG20A | EIF3K |
| IFI27 | HSPG2 | NEDD4L | DPP7 | | RPL36A | HMOX2 | EIF3L |
| IFI30 | HYAL3 | NFATC2 | DRAM1 | | RPL37 | HOXA10 | EIF3M |
| IFI35 | ICAM1 | NFE2L2 | DTX2 | | RPL37A | HOXA6 | EIF4A1 |
| IFI44 | ICOSLG | NFKB1 | DTX2P1-UPK3BP1- | | RPL38 | HOXA9 | EIF4A2 |
| | | | PMS2P11 | | | | |
| IFI44L | ID4 | NFKB2 | DTX3L | | RPL39 | HOXB5 | EIF4B |
| IFI6 | IER3 | NFKBIA | DUSP5 | | RPL4 | HOXB6 | EIF4H |
| IFIH1 | IFI27 | NFKBIB | DYRK2 | | RPL41 | HOXB7 | EIF5A |
| IFIT2 | IFI27L2 | NFKBID | EBI3 | | RPL5 | HOXC10 | EMC4 |
| IFIT3 | IFI30 | NFKBIE | ECE1 | | RPL6 | HOXD-AS1 | ENAH |
| IFIT5 | IFI35 | NFKBIZ | EPSTI1 | | RPL7A | HPCAL1 | ENO1 |
| IFITM1 | IFI6 | NINJ1 | ERAP1 | | RPL8 | HSD11B1L | EPHA3 |
| IFITM3 | IFIH1 | NPAS2 | ERAP2 | | RPL9 | HSPA8 | EPHA4 |
| IL15 | IFIT1 | OPTN | ETV7 | | RPLP0 | HSPE1 | ERCC1 |
| IL17RC | IFIT3 | PIM1 | EZH1 | | RPLP1 | HTRA1 | ERGIC3 |
| IL18BP | IFITM1 | PIM3 | FADS3 | | RPS10 | ID1 | ESD |
| IL32 | IFITM3 | PLA2G4C | FAM111A | | RPS11 | ID3 | FAM162A |
| IL3RA | IGF2 | PLAU | FAM129A | | RPS12 | IDH1 | FBL |
| IL8 | IKBKE | PMAIP1 | FAM193A | | RPS13 | IFT88 | FIBP |
| IRF1 | IL18R1 | PPP1R15A | FAS | | RPS14 | IMP3 | FKBP1A |
| IRF2 | IL27RA | PPP4R4 | FBXL19-AS1 | | RPS15A | IMPDH1 | FLJ41200 |
| IRF3 | IL32 | PPRC1 | FBXO32 | | RPS16 | INSIG2 | FSTL1 |
| IRF7 | IL34 | PSMB8 | FBXO6 | | RPS17 | INTS3 | FUS |
| IRF8 | IL4I1 | PSMB9 | FENDRR | | RPS17L | IVD | FZD1 |
| IRF9 | IL8 | PSME2 | FLJ14186 | | RPS18 | KANK2 | G3BP2 |
| ISG15 | INHBA | RAPH1 | FLJ39739 | | RPS2 | KDM4B | GABARAP |
| ISG20 | IRF1 | RARB | FLJ45340 | | RPS20 | KIAA1430 | GANAB |
| JAK2 | IRF2 | REL | FLT3LG | | RPS23 | KIF26B | GAPDH |
| LAP3 | IRF7 | RELB | FNDC1 | | RPS24 | KLHDC3 | GAS2 |
| LGALS17A | IRF9 | RGS16 | FOXF1 | | RPS25 | L3HYPDH | GATA6 |
| LGALS3BP | ISG15 | RHOB | FRMD4A | | RPS27 | LAGE3 | GCSH |
| LGALS9 | ITGA5 | S1PR1 | FRMD6-AS1 | | RPS28 | LDHA | GDI2 |
| LITAF | ITGAV | SDC4 | FTH1 | | RPS3 | LINC00094 | GNAI1 |
| LOC100507463 | ITIH5 | SELE | FTL | | RPS3A | LINC00516 | GNAS |
| LRP10 | JAK2 | SEMA4C | GATM-AS1 | | RPS4X | LOC100294145 | GNB2L1 |
| LRRTM2 | JAM2 | SERPINA3 | GBP1 | | RPS5 | LOC101101776 | GNG10 |
| LY6E | KCNQ3 | SGPP2 | GBP1P1 | | RPS6 | LOC441081 | GNL3 |
| MAN2B2 | KIF25 | SLC12A7 | GBP2 | | RPS7 | LOXL4 | GPI |
| MARCKS | KLF7 | SLC41A2 | GBP3 | | RPS8 | LRRFIP1 | GPX7 |
| MDK | KLHL5 | SLC7A1 | GBP4 | | RPS9 | LSM2 | GSTA4 |
| MEST | LAD1 | SLC7A2 | GBP5 | | RRP15 | LYPLA1 | H19 |
| MFSD12 | LAMC1 | SNAP25 | GDF15 | | RUNX1T1 | LZTS2 | H2AFZ |
| MIA | LAMC2 | SOCS2 | GGT5 | | SCN4B | MALSU1 | H3F3AP4 |
| MICB | LAP3 | SOD2 | GIMAP2 | | SEMA3E | MAP4K5 | HADHA |
| MKX | LDLR | SOX9 | GIMAP5 | | 6-Sep | MAT2B | HADHB |
| MLKL | LGALS1 | SQSTM1 | GLI1 | | SETBP1 | MBLAC2 | HDAC2 |
| MOV10 | LGALS3 | SRCAP | GPR133 | | SIX1 | MEA1 | HDLBP |
| MR1 | LGALS3BP | ST5 | GPX4 | | SLC25A6 | MEAF6 | HMGCLL1 |
| MT2A | LITAF | STAT5A | GRIPAP1 | | SMAD9 | MEOX2 | HMGN1 |
| MVP | LOC100130093 | STK40 | GSDMD | | SNAI2 | METAP1 | HMGN2 |
| MX1 | LOC400043 | SUSD4 | GTPBP1 | | SNHG16 | METTL5 | HNRNPA1 |
| MYD88 | LOC440896 | TAP1 | GTPBP2 | | SNHG6 | MINA | HNRNPA1P10 |
| NINJ2 | LRRC32 | TAPBP | GYPC | | SNHG8 | MLH3 | HNRNPA2B1 |
| NLRC5 | LRRN3 | TBC1D10A | HAPLN3 | | SOX11 | MLLT11 | HNRNPA3 |
| NMI | LSR | TFPI2 | HAS2 | | SPOCK1 | MLLT3 | HNRNPC |
| NQO1 | MAN2B1 | TGIF1 | HCFC1 | | SPRED1 | MLX | HNRNPD |
| NRN1 | MAP4K2 | TIFA | HCP5 | | SPRY2 | MMACHC | HNRNPK |
| NUB1 | MATN2 | TNF | HDAC10 | | STEAP2 | MMP2 | HNRNPR |
| OAS1 | MIA | TNFAIP1 | HERC2P2 | | SYTL5 | MRP63 | HNRPDL |
| OAS2 | MIA-RAB4B | TNFAIP2 | HERC6 | | TAG LN | MRPL15 | HOXC10 |
| OAS3 | MIR155HG | TNFAIP3 | HIST1H2BJ | | TIMM13 | MRPL17 | HSP90AB1 |
| OASL | MMP10 | TNFAIP8 | HLA-A | | TMEFF2 | MRPL34 | HSP90B1 |
| OCA2 | MMP9 | TNFRSF10B | HLA-B | | TMEM100 | MRPL55 | HSPD1 |
| OGFR | MOV10 | TNFRSF18 | HLA-C | | TMEM130 | MRPS23 | ILF2 |
| OLFML2B | MSRB1 | TNFRSF6B | HLA-DMA | | TMPRSS15 | MRPS26 | IMPDH2 |
| OPTN | MT2A | TP53INP2 | HLA-DMB | | TPBG | MRPS27 | IP6K2 |
| PARP10 | MTMR4 | TRAF1 | HLA-DOA | | TRIB1 | MRPS34 | IPO5 |
| PARP12 | MTRNR2L1 | TRAF3 | HLA-DOB | | TRIL | MRPS6 | ITGB1 |
| PARP14 | MTRNR2L2 | TRIO | HLA-DPA1 | | TSPAN13 | MTA2 | ITM2B |
| PARP3 | MTRNR2L8 | TUBB2A | HLA-DPB1 | | UBE2E3 | MTCH2 | ITM2C |
| PARP9 | MVD | TUBB2B | HLA-DQA1 | | UNC5C | MTX2 | KDELR1 |
| PDCD1LG2 | MVP | TYK2 | HLA-DQB1 | | ZC3HAV1L | MUM1 | KDELR2 |
| PDE4B | MX1 | VCAM1 | HLA-DRA | | ZEB1 | NAE1 | KLHDC3 |
| PLSCR1 | NBEAL1 | ZBTB21 | HLA-DRB1 | | ZFHX4 | NARF | LAMA2 |
| PML | NCCRP1 | ZC3H12A | HLA-DRB5 | | | NCKIPSD | LAPTM4A |
| PPP2R2B | NCKAP5 | ZEB2 | HLA-DRB6 | | | NDFIP2 | LAPTM4B |
| PSMA3 | NFE2L3 | ZFP36 | HLA-E | | | NDRG3 | LDHA |
| PSMA4 | NFKB2 | ZFP36L1 | HLA-F | | | NDUFA8 | LDHB |
| PSMA5 | NFKBIA | ZNF217 | HLA-H | | | NDUFAF6 | LMAN1 |
| PSMB10 | NFKBIB | ZNF267 | HMGA1 | | | NDUFS6 | LSM7 |
| PSMB8 | NFKBIZ | ZNFX1 | HMHA1 | | | NELFCD | LTA4H |
| PSMB9 | NINJ1 | | HORMAD1 | | | NET1 | LZTS1 |
| PSME1 | NLRC5 | | HPS3 | | | NFATC3 | MAGED1 |
| PSME2 | NMI | | HTRA3 | | | NGLY1 | 6-Mar |
| PTHLH | NMNAT2 | | ICAM1 | | | NISCH | MCTP2 |
| PTN | NOD2 | | ICOSLG | | | NKX2-5 | MDH1 |
| PYCARD | NPAS2 | | IDO1 | | | NOL3 | MDH2 |
| RARRES3 | NPL | | IFI27 | | | NR2F2 | MEOX2 |
| RBCK1 | NR0B1 | | IFI27L2 | | | NR2F6 | METAP2 |
| RBMXL1 | NRP2 | | IFI30 | | | NR5A2 | MGST3 |
| RFX5 | NUAK2 | | IFI35 | | | NSD1 | MIF |
| RNF213 | OAS1 | | IFI6 | | | NSF | MINOS1 |
| RSAD2 | OAS2 | | IFIH1 | | | NUDT22 | MLF2 |
| RTP4 | OAS3 | | IFIT3 | | | NUMA1 | MMADHC |
| RUFY4 | OASL | | IFITM1 | | | NYNRIN | MORF4L1 |
| SAMD9 | OCA2 | | IFITM3 | | | OGFOD2 | MORF4L2 |
| SAMD9L | ODF3B | | IFNLR1 | | | PAFAH1B2 | MRFAP1 |
| SAMHD1 | OPTN | | IGF2 | | | PAFAH1B3 | MRPL15 |
| SDSL | PAPSS2 | | IGFBP4 | | | PAGR1 | MRPL21 |
| SECTM1 | PARP10 | | IKBKE | | | PAQR4 | MRPL51 |
| SEMA4D | PARP12 | | IKZF2 | | | PARP16 | MRPS21 |
| SERPING1 | PARP14 | | IL15 | | | PDCD2L | MTDH |
| SHISA5 | PARP4 | | IL15RA | | | PDCL3 | MYH10 |
| SLC15A3 | PARP9 | | IL18BP | | | PDS5B | MYL12A |
| SLC37A1 | PDE4B | | IL32 | | | PFKFB4 | MYL12B |
| SLC6A15 | PDPN | | IL3RA | | | PFN2 | MYL9 |
| SLFN5 | PFKL | | IL4I1 | | | PIGP | NACA |
| SMIM14 | PHF11 | | IL7 | | | PIP4K2C | NAP1L1 |
| SOAT1 | PHLDA3 | | IL8 | | | PKN1 | NCBP2 |
| SOCS1 | PIK3IP1 | | INGX | | | PMS1 | NCL |
| SP100 | PIM1 | | INHBA | | | PNMA2 | NDUFA12 |
| SP110 | PLA2G4C | | IRAK2 | | | PNMAL1 | NDUFA4 |
| SP140L | PLAT | | IRF1 | | | PODNL1 | NDUFA4L2 |
| SSPN | PLCB4 | | IRF2 | | | POLD4 | NDUFAB1 |
| SSTR2 | PLEKHA1 | | IRF3 | | | POLR1B | NDUFB11 |
| STAT1 | PLEKHG1 | | IRF7 | | | POLR2G | NDUFB6 |
| STAT2 | PLSCR1 | | IRF8 | | | POLR3H | NDUFB8 |
| TAP1 | PPP1R14C | | IRF9 | | | POLR3K | NDUFB9 |
| TAP2 | PRCP | | IRX6 | | | POLRMT | NDUFV2 |
| TAPBP | PRDM1 | | ISG15 | | | PPAT | NFIB |
| TAPBPL | PSMA3 | | ISG20 | | | PPCS | NGFRAP1 |
| TCIRG1 | PSMA5 | | ITGA1 | | | PPIP5K2 | NHP2 |
| TMEM140 | PSMB10 | | ITGA2 | | | PPP1CA | NHP2L1 |
| TMSB4X | PSMB8 | | ITGB2 | | | PPP2R4 | NIDI |
| TNFAIP2 | PSMB9 | | ITK | | | PRAME | NME1 |
| TNFRSF14 | PSME1 | | JAK2 | | | PRICKLE1 | NME2 |
| TNFRSF1B | PSME2 | | JUNB | | | PRMT6 | NOB1 |
| TPP1 | PTGER4 | | KAT2A | | | PRUNE | NONO |
| TRAFD1 | PTGES | | KIAA1217 | | | PTEN | NPM1 |
| TRIL | QPCT | | KIAA1462 | | | PTPLA | NR2F2 |
| TRIM21 | RAI14 | | KIAA1755 | | | PTPLAD1 | NR5A2 |
| TRIM22 | RANGAP1 | | KIF1A | | | PTPRG | NREP |
| TRIM25 | RARB | | KLHDC7B | | | PYCR2 | NRP1 |
| TRIM38 | RARRES3 | | KYNU | | | PYGB | NUCB2 |
| TRIM56 | RASSF4 | | LAMP3 | | | RAD50 | NUCKS1 |
| TYMP | REL | | LAP3 | | | RAI1 | NUTF2 |
| UBA7 | RELB | | LBX2-AS1 | | | RASSF7 | OCIAD1 |
| UBB | RGS16 | | LGALS17A | | | REEP2 | OST4 |
| UBD | RGS3 | | LGALS3 | | | RFT1 | OSTC |
| UBE2L6 | RIPK2 | | LGALS3BP | | | RGS19 | P4HB |
| UBR2 | RNF19A | | LGALS9 | | | RIN1 | PABPC1 |
| USF1 | RNF213 | | LOC100132247 | | | RMDN1 | PAFAH1B2 |
| USP18 | RQCD1 | | LOC100132891 | | | RNF135 | PAFAH1B3 |
| USP30-AS1 | RTP4 | | LOC100133331 | | | RNF168 | PAICS |
| VAMP5 | RUNX3 | | LOC100288069 | | | RNF216 | PAIP2 |
| VCAM1 | SALL4 | | LOC100289019 | | | RNH1 | PAPSS1 |
| WARS | SAMD9 | | LOC100507463 | | | RPP21 | PARK7 |
| XAF1 | SAMD9L | | LOC284260 | | | RPP25 | PCDH7 |
| XIRP1 | SCARF1 | | LOC399744 | | | RPS19BP1 | PCDH9 |
| XRN1 | SCD | | LOC731275 | | | RRP1B | PCMT1 |
| ZNF672 | SCN1B | | LOC732275 | | | RUNX1T1 | PCOLCE |
| ZNFX1 | SDC4 | | LTBP2 | | | SAMD11 | PCOLCE2 |
| | SEC23B | | LY6E | | | SCAMP1 | PDHB |
| | SELE | | LYZ | | | SCCPDH | PDIA3 |
| | SELM | | MALAT1 | | | SDHAF1 | PDIA4 |
| | SERPINA3 | | MAP3K8 | | | SERTM1 | PDIA6 |
| | SGPP2 | | MAST3 | | | SET | PDLIM7 |
| | SLC11A2 | | MBOAT1 | | | SFXN1 | PFN2 |
| | SLC12A7 | | MFSD12 | | | SGK196 | PGAM1 |
| | SLC2A6 | | MGLL | | | SHISA2 | PGK1 |
| | SLC37A1 | | MICAL1 | | | SIKE1 | PHB |
| | SLC43A2 | | MICB | | | SIX1 | PHB2 |
| | SLC7A2 | | MIR155HG | | | SLBP | PLOD1 |
| | SLFN5 | | MKNK2 | | | SLC16A3 | PLP2 |
| | SMG7 | | MLKL | | | SLC20A1 | PPA2 |
| | SOCS2 | | MLL2 | | | SLC29A2 | PPIA |
| | SORCS1 | | MMP14 | | | SLC2A10 | PPIB |
| | SOX9 | | MMP17 | | | SLC35B2 | PPME1 |
| | SP100 | | MOB3C | | | SLC35B4 | PPP1CA |
| | SPAG1 | | MOV10 | | | SMA4 | PPP1CB |
| | SPIB | | MSC | | | SMAP1 | PPP2R1A |
| | SPPL2A | | MT2A | | | SMIM15 | PPT1 |
| | SQSTM1 | | MTRNR2L1 | | | SNAP29 | PRDX2 |
| | SRR | | MTRNR2L10 | | | SNAPC2 | PRDX4 |
| | SSPN | | MTRNR2L2 | | | SNRNP25 | PRDX6 |
| | ST5 | | MTRNR2L6 | | | SNX2 | PRKAR1A |
| | ST6GAL2 | | MTRNR2L8 | | | SNX27 | PSMB1 |
| | STAP2 | | MVP | | | SPICE1 | PSMD8 |
| | STARD10 | | MX1 | | | SPRY2 | PTDSS1 |
| | STAT1 | | MYEOV | | | SSR1 | PTGES3 |
| | STAT5A | | MYO10 | | | ST13 | PTMA |
| | STRA6 | | MYO1B | | | STEAP2 | PTPLAD1 |
| | SYNGR2 | | MYO9B | | | SUOX | RAB1A |
| | SYNGR3 | | NAAA | | | SUPT20H | RAB2A |
| | TAGLN2 | | NDOR1 | | | TAPT1-AS1 | RAN |
| | TANK | | NEAT1 | | | TBC1D5 | RBBP4 |
| | TAP1 | | NETO1 | | | TBC1D9B | RBFOX2 |
| | TAP2 | | NEURL3 | | | TBX18 | RBM3 |
| | TAPBP | | NFE2L3 | | | TCEAL8 | RBM4 |
| | TAPBPL | | NFKB2 | | | TET1 | RBMX |
| | TBC1D17 | | NFKBIA | | | TFCP2 | RBPJ |
| | THY1 | | NFKBIZ | | | THNSL1 | RCBTB2 |
| | TIFA | | NLRC5 | | | THYN1 | RHOA |
| | TMEM123 | | NMI | | | TIA1 | RHOC |
| | TMEM173 | | NNMT | | | TIMM21 | RNF5P1 |
| | TMEM205 | | NOD2 | | | TIMM22 | RPL10 |
| | TMPRSS2 | | NPIPL3 | | | TIMM9 | RPL10A |
| | TNF | | NPTX2 | | | TMEM134 | RPL11 |
| | TNFAIP1 | | NRP2 | | | TMEM216 | RPL12 |
| | TNFAIP2 | | NT5E | | | TMEM223 | RPL14 |
| | TNFAIP3 | | NUAK2 | | | TNRC6B | RPL15 |
| | TNFAIP6 | | NUB1 | | | TPI1 | RPL17 |
| | TNFAIP8 | | OAS1 | | | TRAM2 | RPL18 |
| | TNFRSF14 | | OAS2 | | | TRAPPC2L | RPL22 |
| | TNFRSF18 | | OAS3 | | | TRERF1 | RPL22L1 |
| | TNFRSF4 | | OGFR | | | TRIL | RPL23 |
| | TNFRSF6B | | OPTN | | | TRPT1 | RPL24 |
| | TNIP1 | | OSGIN1 | | | TSEN54 | RPL26 |
| | TNKS1BP1 | | P2RX4 | | | TSPAN3 | RPL27 |
| | TNN | | PABPC1L | | | TTC3 | RPL29 |
| | TRADD | | PACS2 | | | TUBB | RPL3 |
| | TRAF1 | | PAPPA | | | TUBB3 | RPL32 |
| | TRIM21 | | PARP10 | | | TXNDC15 | RPL34 |
| | TRIM25 | | PARP12 | | | UBL4A | RPL35A |
| | TYK2 | | PARP14 | | | UBXN2B | RPL36A |
| | TYMP | | PARP3 | | | UCK2 | RPL39 |
| | UBA7 | | PARP8 | | | VAT1 | RPL4 |
| | UBD | | PARP9 | | | VDAC3 | RPL5 |
| | UBE2L6 | | PAWR | | | VPS35 | RPL6 |
| | USP18 | | PCGF5 | | | WDR12 | RPL7 |
| | VAMP5 | | PDCD1LG2 | | | WDR41 | RPL7A |
| | VCAM1 | | PDGFA | | | WNT5B | RPL7L1 |
| | WARS | | PERP | | | XYLB | RPL8 |
| | WWC3 | | PHLDA1 | | | YEATS2 | RPL9 |
| | XAF1 | | PHLDA2 | | | ZC3HAV1L | RPLP0 |
| | XRN1 | | PILRA | | | ZFHX4 | RPN2 |
| | ZBTB5 | | PIM1 | | | ZNF174 | RPS10 |
| | ZC3H7B | | PKD1 | | | ZNF232 | RPS13 |
| | ZEB2 | | PKD1P1 | | | ZNF280D | RPS14 |
| | ZFP36L1 | | PLA1A | | | ZNF32 | RPS15A |
| | ZNFX1 | | PLA2G16 | | | ZNF395 | RPS17 |
| | | | PLA2G4C | | | ZNF532 | RPS17L |
| | | | PLAUR | | | ZNF692 | RPS2 |
| | | | PLD1 | | | ZNF74 | RPS23 |
| | | | PLD2 | | | ZNF816 | RPS24 |
| | | | PLEC | | | ZSWIM7 | RPS27A |
| | | | PLSCR1 | | | | RPS3 |
| | | | PML | | | | RPS3A |
| | | | POM121L9P | | | | RPS4X |
| | | | POU5F1 | | | | RPS4Y1 |
| | | | PP7080 | | | | RPS6 |
| | | | PRDM1 | | | | RPS7 |
| | | | PRLR | | | | RPS8 |
| | | | PSMB10 | | | | RPSA |
| | | | PSMB8 | | | | RPSAP58 |
| | | | PSMB9 | | | | RSL1D1 |
| | | | PSME1 | | | | RSL24D1 |
| | | | PSME2 | | | | RSU1 |
| | | | PTHLH | | | | RTN3 |
| | | | PTPRJ | | | | RUNX1T1 |
| | | | PYCARD | | | | SAP18 |
| | | | RAB38 | | | | SARNP |
| | | | RAMP1 | | | | SDCBP |
| | | | RARRES1 | | | | SEC11A |
| | | | RARRES3 | | | | SEC13 |
| | | | RASD1 | | | | SEC22B |
| | | | RBCK1 | | | | SEC31A |
| | | | RELB | | | | SEC61B |
| | | | RGCC | | | | SEC61G |
| | | | RGS11 | | | | SEMA3E |
| | | | RGS16 | | | | SEMA6D |
| | | | RHBDF2 | | | | SEP15 |
| | | | RHEBL1 | | | | SEP2 |
| | | | RHOB | | | | SEP7 |
| | | | RIPK2 | | | | SEPW1 |

| RNF144A-AS1 | SERBP1 |
|---|---|
| RNF213 | SERINC1 |
| ROBO3 | SERP1 |
| RPLP0P2 | SERPINH1 |
| RSAD2 | SET |
| RTP4 | SF3B14 |
| RUFY4 | SHISA2 |
| RUNX3 | SIX1 |
| SAA1 | SKP1 |
| SAMD9L | SLC25A3 |
| SAT1 | SLC25A5 |
| SCARA5 | SLC25A6 |
| SCARF1 | SLIT2 |
| SCO2 | SMAD9 |
| SCRIB | SMARCA1 |
| SECTM1 | SND1 |
| SEMA4D | SNHG16 |
| SERPINA1 | SNRPE |
| SERPING1 | SNRPF |
| SIGIRR | SNRPN |
| SLC15A3 | SNURF |
| SLC25A28 | SOX11 |
| SLC37A1 | SPARC |
| SLC7A2 | SPCS1 |
| SLC9A3 | SPCS2 |
| SLFN5 | SPIN1 |
| SOCS1 | SPOCK1 |
| SOD2 | SRP14 |
| SOX8 | SRP72 |
| SP100 | SRP9 |
| SP110 | SRSF1 |
| SP140L | SRSF2 |
| SQRDL | SRSF3 |
| SQSTM1 | SRSF6 |
| SSH1 | SSB |
| SSTR2 | SSBP2 |
| SSUH2 | SSR1 |
| STAT1 | SSR2 |
| STAT2 | SSR3 |
| STAT5A | ST13 |
| SUSD2 | STMN1 |
| TAC3 | STRAP |
| TAP1 | STT3A |
| TAP2 | SUB1 |
| TAPBP | SUMO1 |
| TAPBPL | SUMO2 |
| TBX2 | SURF4 |
| TCIRG1 | SYNCRIP |
| TEP1 | TCEAL8 |
| TF | TCEB1 |
| TIMP3 | TCP1 |
| TLCD2 | TFPI |
| TMEM140 | TIMM13 |
| TMEM158 | TMA7 |
| TMEM194A | TMBIM6 |
| TMEM205 | TMED10 |
| TMEM8A | TMED2 |
| TMPRSS3 | TMED3 |
| TNFAIP2 | TMEM100 |
| TNFAIP3 | TMEM258 |
| TNFAIP8 | TMEM59 |
| TNFRSF14 | TMEM66 |
| TNFSF10 | TMEM70 |
| TNFSF9 | TMEM98 |
| TNS3 | TNFRSF10D |
| TRAF1 | TOMM20 |
| TRAFD1 | TOMM22 |
| TREX1 | TOMM6 |
| TRIM14 | TPI1 |
| TRIM21 | TPM1 |
| TRIM22 | TPM2 |
| TRIM25 | TPM4 |
| TRIM38 | TRAP1 |
| TRIM56 | TRPS1 |
| TRIM69 | TSPAN13 |
| TRPM4 | TUBA1A |
| TXNIP | TUBA1B |
| TYMP | TUBB |
| UACA | TWISTNB |
| UBA7 | TXN2 |
| UBD | TXNL1 |
| UBE2L6 | TXNL4A |
| UBR4 | U2AF1 |
| ULK1 | UBE2E3 |
| UNKL | UBE2V1 |
| UPP1 | UBE2V2 |
| USF1 | UFC1 |
| USP18 | UGDH |
| USP30-AS1 | UNC5C |
| UTRN | UQCRC1 |
| VAMP5 | UQCRH |
| VCAM1 | VAPA |
| VPS13C | VDAC1 |
| WARS | VDAC3 |
| WASH1 | VIM |
| WASH7P | VKORC1 |
| WDR25 | VPS28 |
| WDR81 | WASF1 |
| XAF1 | WDR61 |
| XIRP1 | WDR83OS |
| XRN1 | XRCC5 |
| ZBP1 | XRCC6 |
| ZBTB3 | YIPF3 |
| ZC3H3 | YWHAE |
| ZC3HAV1 | YWHAQ |
| ZFC3H1 | YWHAZ |
| ZFP36L1 | ZC3H15 |
| ZFYVE26 | ZFHX4 |
| ZNFX1 | ZNF652 |
| ZSWIM8 | ZNF706 |

TABLE 11
The TNF/IFN programs enrichment with pre-defined gene sets (hypergeometric p-values: -log10 transformed, capped at 17).

| | | | | TNF | | | | TNF |
|---|---|---|---|---|---|---|---|---|
| | | | TNF | & | | | TNF | & |
| | IFN | TNF | short | IFN | IFN | TNF | short | IFN |
| Gene Set | up | up | up | up | down | down | down | down |
| HALLMARK_TNFA_SIGNALING_VIA_NFKB | 5.54 | 17.00 | 17.00 | 17.00 | 0.00 | 0.97 | 0.00 | 0.00 |
| HALLMARK_INTERFERON_GAMMA_RESPONSE | 17.00 | 17.00 | 14.02 | 17.00 | 0.00 | 0.00 | 0.00 | 0.00 |
| HALLMARK_APOPTOSIS | 2.57 | 5.61 | 13.97 | 5.93 | 0.00 | 0.14 | 0.51 | 0.12 |
| HALLMARK_P53_PATHWAY | 0.86 | 2.03 | 6.18 | 5.59 | 0.00 | 1.46 | 0.00 | 0.10 |
| HALLMARK_HYPOXIA | 0.42 | 1.83 | 12.68 | 0.35 | 0.00 | 0.10 | 1.09 | 1.51 |
| Homeobox | 0.00 | 0.07 | 0.17 | 0.03 | 1.68 | 3.15 | 7.31 | 0.54 |
| HALLMARK_OXIDATIVE_PHOSPHORYLATION | 0.02 | 0.01 | 0.01 | 0.00 | 0.00 | 0.16 | 1.55 | 15.35 |
| HALLMARK_MYC_TARGETS_V1 | 0.00 | 0.00 | 0.00 | 0.00 | 0.28 | 5.56 | 0.20 | 17.00 |
| GO_CELLULAR_RESPONSE_TO_ORGANIC_SUBSTANCE | 17.00 | 17.00 | 11.94 | 17.00 | 0.62 | 0.93 | 0.00 | 2.75 |
| GO_POSITIVE_REGULATION_OF_RESPONSE_TO_STIMULUS | 17.00 | 17.00 | 17.00 | 17.00 | 0.09 | 0.12 | 0.10 | 1.70 |
| GO_ACTIVATION_OF_IMMUNE_RESPONSE | 15.95 | 9.14 | 3.80 | 17.00 | 0.23 | 0.20 | 0.02 | 1.61 |
| GO_POSITIVE_REGULATION_OF_IMMUNE_RESPONSE | 17.00 | 13.32 | 6.46 | 17.00 | 0.16 | 0.09 | 0.01 | 1.14 |
| GO_IMMUNE_EFFECTOR_PROCESS | 17.00 | 15.65 | 1.97 | 17.00 | 0.20 | 0.15 | 0.30 | 1.03 |
| GO_REGULATION_OF_IMMUNE_RESPONSE | 17.00 | 17.00 | 10.46 | 17.00 | 0.08 | 0.13 | 0.01 | 0.78 |
| GO_IMMUNE_SYSTEM_PROCESS | 17.00 | 17.00 | 17.00 | 17.00 | 0.11 | 0.39 | 0.19 | 0.77 |
| GO_REGULATION_OF_MULTI_ORGANISM_PROCESS | 17.00 | 17.00 | 3.83 | 17.00 | 0.00 | 0.04 | 0.12 | 0.71 |
| GO_REGULATION_OF_IMMUNE_SYSTEM_PROCESS | 17.00 | 17.00 | 17.00 | 17.00 | 0.09 | 0.44 | 0.01 | 0.68 |
| GO_RESPONSE_TO_EXTERNAL_STIMULUS | 17.00 | 17.00 | 17.00 | 17.00 | 0.30 | 1.55 | 0.10 | 0.62 |
| GO_REGULATION_OF_CELL_ADHESION | 12.20 | 10.72 | 10.21 | 17.00 | 0.39 | 0.86 | 0.16 | 0.60 |
| GO_ANTIGEN_BINDING | 17.00 | 4.66 | 5.53 | 17.00 | 0.00 | 0.23 | 0.09 | 0.42 |
| GO_POSITIVE_REGULATION_OF_IMMUNE_SYSTEM_PROCESS | 17.00 | 17.00 | 15.95 | 17.00 | 0.07 | 0.38 | 0.00 | 0.38 |
| GO_CELLULAR_RESPONSE_TO_CYTOKINE_STIMULUS | 17.00 | 17.00 | 17.00 | 17.00 | 0.12 | 0.09 | 0.00 | 0.37 |
| GO_RESPONSE_TO_VIRUS | 17.00 | 17.00 | 4.06 | 17.00 | 0.00 | 0.09 | 0.09 | 0.29 |
| GO_RESPONSE_TO_CYTOKINE | 17.00 | 17.00 | 17.00 | 17.00 | 0.09 | 0.09 | 0.00 | 0.26 |
| GO_REGULATION_OF_INNATE_IMMUNE_RESPONSE | 17.00 | 17.00 | 7.69 | 17.00 | 0.00 | 0.01 | 0.01 | 0.19 |
| GO_REGULATION_OF_DEFENSE_RESPONSE | 17.00 | 17.00 | 10.24 | 17.00 | 0.00 | 0.02 | 0.01 | 0.18 |
| GO_NEGATIVE_REGULATION_OF_MULTI_ORGANISM_PROCESS | 17.00 | 17.00 | 2.54 | 17.00 | 0.00 | 0.07 | 0.01 | 0.17 |
| GO_RESPONSE_TO_BACTERIUM | 17.00 | 15.05 | 17.00 | 17.00 | 0.21 | 0.33 | 0.00 | 0.15 |
| GO_RESPONSE_TO_BIOTIC_STIMULUS | 17.00 | 17.00 | 15.65 | 17.00 | 0.07 | 0.29 | 0.00 | 0.15 |
| GO_REGULATION_OF_LEUKOCYTE_PROLIFERATION | 13.06 | 7.55 | 4.99 | 17.00 | 0.45 | 0.06 | 0.05 | 0.11 |
| GO_POSITIVE_REGULATION_OF_CYTOKINE_PRODUCTION | 15.65 | 9.63 | 6.73 | 17.00 | 0.00 | 0.01 | 0.19 | 0.09 |
| GO_ADAPTIVE_IMMUNE_RESPONSE | 15.05 | 9.09 | 4.60 | 17.00 | 0.00 | 0.25 | 0.01 | 0.07 |
| GO_CYTOKINE_MEDIATED_SIGNALING_PATHWAY | 17.00 | 17.00 | 17.00 | 17.00 | 0.20 | 0.02 | 0.00 | 0.06 |
| GO_REGULATION_OF_CYTOKINE_PRODUCTION | 17.00 | 17.00 | 10.94 | 17.00 | 0.15 | 0.03 | 0.20 | 0.06 |
| GO_CELLULAR_RESPONSE_TO_INTERFERON_GAMMA | 17.00 | 17.00 | 7.48 | 17.00 | 0.00 | 0.00 | 0.03 | 0.05 |
| GO_INNATE_IMMUNE_RESPONSE | 17.00 | 17.00 | 7.15 | 17.00 | 0.00 | 0.04 | 0.05 | 0.05 |
| GO_RESPONSE_TO_TYPE_I_INTERFERON | 17.00 | 17.00 | 5.33 | 17.00 | 0.00 | 0.21 | 0.00 | 0.04 |
| GO_IMMUNE_RESPONSE | 17.00 | 17.00 | 17.00 | 17.00 | 0.07 | 0.15 | 0.01 | 0.03 |
| GO_DEFENSE_RESPONSE | 17.00 | 17.00 | 17.00 | 17.00 | 0.17 | 0.03 | 0.01 | 0.03 |
| GO_RESPONSE_TO_INTERFERON_GAMMA | 17.00 | 17.00 | 6.75 | 17.00 | 0.00 | 0.00 | 0.02 | 0.03 |
| GO_INFLAMMATORY_RESPONSE | 13.60 | 17.00 | 17.00 | 17.00 | 0.71 | 0.12 | 0.00 | 0.03 |
| GO_POSITIVE_REGULATION_OF_CELL_CELL_ADHESION | 12.96 | 9.85 | 8.66 | 17.00 | 0.00 | 0.17 | 0.01 | 0.03 |
| GO_REGULATION_OF_HOMOTYPIC_CELL_CELL_ADHESION | 15.00 | 7.69 | 7.05 | 17.00 | 0.00 | 0.09 | 0.00 | 0.02 |
| GO_REGULATION_OF_CELL_CELL_ADHESION | 14.27 | 8.56 | 7.98 | 17.00 | 0.00 | 1.09 | 0.01 | 0.02 |
| GO_REGULATION_OF_CELL_ACTIVATION | 12.52 | 9.03 | 9.36 | 17.00 | 0.61 | 0.16 | 0.00 | 0.01 |
| GO_DEFENSE_RESPONSE_TO_OTHER_ORGANISM | 17.00 | 17.00 | 2.76 | 17.00 | 0.00 | 0.03 | 0.10 | 0.01 |
| GO_DEFENSE_RESPONSE_TO_VIRUS | 17.00 | 17.00 | 1.76 | 17.00 | 0.00 | 0.00 | 0.31 | 0.00 |
| GO_INTERFERON_GAMMA_MEDIATED_SIGNALING_PATHWAY | 17.00 | 17.00 | 5.08 | 17.00 | 0.00 | 0.00 | 0.00 | 0.00 |
| GO_MHC_PROTEIN_COMPLEX | 17.00 | 5.58 | 4.47 | 17.00 | 0.00 | 0.00 | 0.00 | 0.00 |
| GO_MHC_CLASS_II_PROTEIN_COMPLEX | 17.00 | 0.00 | 0.00 | 17.00 | 0.00 | 0.00 | 0.00 | 0.00 |
| HALLMARK_INTERFERON_ALPHA_RESPONSE | 17.00 | 17.00 | 4.32 | 17.00 | 0.00 | 0.00 | 0.02 | 0.00 |
| HALLMARK_INFLAMMATORY_RESPONSE | 14.18 | 17.00 | 17.00 | 17.00 | 0.42 | 1.10 | 0.00 | 0.01 |
| HALLMARK_COMPLEMENT | 9.99 | 9.65 | 4.43 | 17.00 | 0.00 | 0.04 | 0.00 | 0.00 |
| HALLMARK_ALLOGRAFT_REJECTION | 17.00 | 14.88 | 9.58 | 17.00 | 0.00 | 1.28 | 0.00 | 1.16 |
| GO_EXTRACELLULAR_SPACE | 11.88 | 10.41 | 6.41 | 15.95 | 0.39 | 0.50 | 0.06 | 2.53 |
| GO_NEGATIVE_REGULATION_OF_VIRAL_PROCESS | 17.00 | 15.65 | 1.38 | 15.95 | 0.00 | 0.00 | 0.00 | 0.08 |
| GO_REGULATION_OF_T_CELL_PROLIFERATION | 13.44 | 5.97 | 3.22 | 15.65 | 0.00 | 0.00 | 0.03 | 0.06 |
| GO_POSITIVE_REGULATION_OF_CELL_ACTIVATION | 11.99 | 7.48 | 5.85 | 15.48 | 0.00 | 0.00 | 0.00 | 0.03 |
| GO_PEPTIDE_ANTIGEN_BINDING | 17.00 | 6.64 | 7.02 | 15.18 | 0.00 | 0.00 | 0.00 | 0.00 |
| GO_REGULATION_OF_SYMBIOSIS_ENCOMPASSING_MUTUALISM_THROUGH_PARASITISM | 17.00 | 14.81 | 2.06 | 14.81 | 0.00 | 0.03 | 0.05 | 0.77 |
| GO_ANTIGEN_PROCESSING_AND_PRESENTATION | 17.00 | 8.50 | 4.88 | 14.75 | 0.00 | 0.00 | 0.00 | 0.47 |
| GO_POSITIVE_REGULATION_OF_CELL_ADHESION | 9.81 | 11.70 | 9.27 | 14.57 | 0.26 | 0.12 | 0.08 | 0.41 |
| GO_ANTIGEN_PROCESSING_AND_PRESENTATION_OF_PEPTIDE_ANTIGEN | 17.00 | 8.32 | 4.17 | 14.48 | 0.00 | 0.00 | 0.00 | 0.71 |
| GO_NEGATIVE_REGULATION_OF_VIRAL_GENOME_REPLICATION | 17.00 | 15.65 | 1.37 | 14.48 | 0.00 | 0.00 | 0.00 | 0.07 |
| GO_REGULATION_OF_CELL_PROLIFERATION | 7.07 | 9.67 | 14.19 | 14.03 | 0.36 | 1.02 | 0.03 | 1.80 |
| GO_CELL_SURFACE | 9.46 | 13.95 | 8.60 | 13.95 | 0.13 | 0.71 | 0.00 | 2.20 |
| GO_LUMENAL_SIDE_OF_MEMBRANE | 17.00 | 6.15 | 5.29 | 13.93 | 0.00 | 0.00 | 0.25 | 0.16 |
| GO_CYTOKINE_ACTIVITY | 8.54 | 7.93 | 11.45 | 13.51 | 0.00 | 0.10 | 0.27 | 0.12 |
| GO_ANTIGEN_PROCESSING_AND_PRESENTATION_OF_ENDOGENOUS_PEPTIDE_ANTIGEN | 17.00 | 12.49 | 6.30 | 13.49 | 0.00 | 0.00 | 0.00 | 0.00 |
| GO_HUMORAL_IMMUNE_RESPONSE | 11.01 | 5.64 | 4.91 | 13.40 | 0.00 | 0.18 | 0.07 | 0.56 |
| GO_POSITIVE_REGULATION_OF_DEFENSE_RESPONSE | 14.22 | 13.52 | 8.21 | 13.31 | 0.00 | 0.04 | 0.00 | 0.16 |
| GO_NEGATIVE_REGULATION_OF_IMMUNE_SYSTEM_PROCESS | 14.06 | 9.72 | 6.64 | 13.11 | 0.24 | 0.65 | 0.06 | 0.15 |
| GO_POSITIVE_REGULATION_OF_LEUKOCYTE_PROLIFERATION | 9.41 | 5.37 | 5.01 | 13.02 | 0.00 | 0.00 | 0.00 | 0.07 |
| GO_ADAPTIVE_IMMUNE_RESPONSE_BASED_ON_SOMATIC_RECOMBINATION_OF_IMMUNE_RECEPTORS_BUILT_FROM_{—} | 10.05 | 3.51 | 3.59 | 13.02 | 0.00 | 0.15 | 0.05 | 0.22 |
| IMMUNOGLOBULIN_SUPERFAMILY_DOMAINS | | | | | | | | |
| GO_ANTIGEN_PROCESSING_AND_PRESENTATION_OF_ENDOGENOUS_ANTIGEN | 15.65 | 10.67 | 5.61 | 12.98 | 0.00 | 0.00 | 0.00 | 0.00 |
| GO_POSITIVE_REGULATION_OF_MULTICELLULAR_ORGANISMAL_PROCESS | 8.12 | 10.57 | 9.25 | 12.57 | 0.10 | 1.01 | 0.11 | 1.23 |
| GO_REGULATION_OF_VIRAL_GENOME_REPLICATION | 17.00 | 13.06 | 0.92 | 12.44 | 0.00 | 0.16 | 0.20 | 1.57 |
| GO_SIDE_OF_MEMBRANE | 11.05 | 10.76 | 9.66 | 12.43 | 0.00 | 0.14 | 0.01 | 0.23 |
| GO_REGULATION_OF_RESPONSE_TO_EXTERNAL_STIMULUS | 8.01 | 6.44 | 6.48 | 12.33 | 0.00 | 0.40 | 0.10 | 0.29 |
| GO_NEGATIVE_REGULATION_OF_CYTOKINE_PRODUCTION | 13.58 | 9.57 | 8.14 | 12.23 | 0.44 | 0.00 | 0.25 | 0.09 |
| GO_REGULATION_OF_TYPE_I_INTERFERON_PRODUCTION | 11.10 | 11.17 | 4.32 | 11.96 | 0.00 | 0.00 | 0.75 | 0.23 |
| GO_CELL_ACTIVATION | 5.92 | 6.61 | 6.92 | 11.85 | 0.18 | 1.41 | 0.13 | 0.94 |
| GO_REGULATION_OF_IMMUNE_EFFECTOR_PROCESS | 12.53 | 9.75 | 5.60 | 11.58 | 0.00 | 0.03 | 0.17 | 0.11 |
| GO_RECEPTOR_BINDING | 8.21 | 9.18 | 6.87 | 11.50 | 0.78 | 0.17 | 0.09 | 0.72 |
| GO_REGULATION_OF_INTERFERON_GAMMA_PRODUCTION | 11.28 | 5.52 | 4.12 | 11.46 | 0.00 | 0.00 | 0.00 | 0.27 |
| GO_RESPONSE_TO_MOLECULE_OF_BACTERIAL_ORIGIN | 11.81 | 11.59 | 17.00 | 11.34 | 0.29 | 0.17 | 0.01 | 0.12 |
| GO_LEUKOCYTE_ACTIVATION | 7.08 | 7.06 | 7.81 | 11.29 | 0.00 | 1.05 | 0.16 | 0.30 |
| GO_POSITIVE_REGULATION_OF_CELL_COMMUNICATION | 9.48 | 7.75 | 11.53 | 11.07 | 0.34 | 0.04 | 0.09 | 1.23 |
| GO_REGULATION_OF_RESPONSE_TO_STRESS | 12.49 | 9.26 | 8.62 | 11.02 | 0.00 | 0.04 | 0.04 | 0.54 |
| GO_POSITIVE_REGULATION_OF_T_CELL_PROLIFERATION | 8.82 | 4.73 | 3.61 | 10.92 | 0.00 | 0.00 | 0.00 | 0.05 |
| GO_POSITIVE_REGULATION_OF_RESPONSE_TO_EXTERNAL_STIMULUS | 8.16 | 7.01 | 5.53 | 10.81 | 0.00 | 0.23 | 0.04 | 0.46 |
| GO_ER_TO_GOLGI_TRANSPORT_VESICLE | 11.37 | 4.18 | 2.42 | 10.70 | 0.00 | 0.00 | 0.00 | 1.40 |
| GO_NEGATIVE_REGULATION_OF_TYPE_I_INTERFERON_PRODUCTION | 11.16 | 11.19 | 4.63 | 10.56 | 0.00 | 0.00 | 0.55 | 0.11 |
| GO_ER_TO_GOLGI_TRANSPORT_VESICLE_MEMBRANE | 13.10 | 5.06 | 2.89 | 10.51 | 0.00 | 0.00 | 0.00 | 1.87 |
| GO_RESPONSE_TO_TUMOR_NECROSIS_FACTOR | 12.57 | 15.95 | 12.57 | 10.45 | 0.34 | 0.00 | 0.00 | 0.03 |
| GO_LYMPHOCYTE_MEDIATED_IMMUNITY | 8.59 | 1.34 | 0.98 | 10.01 | 0.00 | 0.17 | 0.22 | 0.85 |
| HALLMARK_IL6_JAK_STAT3_SIGNALING | 10.67 | 6.28 | 10.08 | 9.87 | 0.00 | 0.17 | 0.00 | 0.00 |
| GO_LEUKOCYTE_CELL_CELL_ADHESION | 5.27 | 8.92 | 7.67 | 9.80 | 0.00 | 0.67 | 0.01 | 0.26 |
| GO_PLASMA_MEMBRANE_PROTEIN_COMPLEX | 10.95 | 6.04 | 3.86 | 9.64 | 0.75 | 0.29 | 0.04 | 0.33 |
| GO_EXTERNAL_SIDE_OF_PLASMA_MEMBRANE | 6.53 | 9.47 | 7.07 | 9.55 | 0.00 | 0.10 | 0.00 | 0.24 |
| GO_NEGATIVE_REGULATION_OF_INNATE_IMMUNE_RESPONSE | 12.47 | 8.45 | 3.99 | 9.46 | 0.00 | 0.00 | 0.00 | 0.00 |
| GO_NEGATIVE_REGULATION_OF_CELL_ACTIVATION | 5.59 | 1.57 | 3.54 | 9.36 | 1.35 | 0.65 | 0.10 | 0.04 |
| GO_CYTOKINE_RECEPTOR_BINDING | 7.24 | 11.81 | 13.07 | 9.36 | 0.00 | 0.34 | 0.18 | 0.36 |
| GO_POSITIVE_REGULATION_OF_LEUKOCYTE_MIGRATION | 7.44 | 8.84 | 5.93 | 9.26 | 0.00 | 0.41 | 0.00 | 0.18 |
| GO_RESPONSE_TO_LIPID | 7.71 | 6.74 | 15.65 | 9.21 | 0.53 | 0.49 | 0.15 | 0.76 |
| GO_CELL_DEATH | 1.74 | 8.74 | 10.95 | 9.19 | 0.13 | 0.21 | 0.54 | 0.35 |
| GO_ENDOCYTIC_VESICLE_MEMBRANE | 9.94 | 3.10 | 2.11 | 9.17 | 0.00 | 0.00 | 0.44 | 0.21 |
| GO_HUMORAL_IMMUNE_RESPONSE_MEDIATED_BY_CIRCULATING_IMMUNOGLOBULIN | 6.84 | 0.95 | 1.36 | 8.99 | 0.00 | 0.00 | 0.00 | 0.25 |
| GO_ANTIGEN_PROCESSING_AND_PRESENTATION_OF_PEPTIDE_ANTIGEN_VIA_MHC_CLASS_I | 17.00 | 12.52 | 6.73 | 8.95 | 0.00 | 0.00 | 0.00 | 0.85 |
| GO_VACUOLE | 6.15 | 2.91 | 1.99 | 8.87 | 0.00 | 0.01 | 0.01 | 0.21 |
| GO_REGULATION_OF_RESPONSE_TO_WOUNDING | 4.10 | 4.82 | 8.66 | 8.85 | 0.00 | 0.05 | 0.00 | 0.13 |
| GO_RESPONSE_TO_INTERLEUKIN_1 | 3.42 | 6.46 | 10.44 | 8.81 | 0.00 | 0.14 | 0.00 | 0.02 |
| GO_REGULATION_OF_INFLAMMATORY_RESPONSE | 5.44 | 5.36 | 7.92 | 8.80 | 0.00 | 0.03 | 0.00 | 0.19 |
| GO_B_CELL_MEDIATED_IMMUNITY | 6.87 | 0.99 | 0.86 | 8.78 | 0.00 | 0.33 | 0.16 | 0.68 |
| GO_REGULATION_OF_LEUKOCYTE_MIGRATION | 6.47 | 7.63 | 7.01 | 8.63 | 0.00 | 0.32 | 0.00 | 0.41 |
| GO_REGULATION_OF_ANTIGEN_PROCESSING_AND_PRESENTATION | 8.76 | 3.54 | 0.73 | 8.62 | 0.00 | 0.00 | 0.00 | 0.00 |
| GO_MHC_CLASS_II_RECEPTOR_ACTIVITY | 10.35 | 0.00 | 0.00 | 8.55 | 0.00 | 0.00 | 0.00 | 0.00 |
| GO_POSITIVE_REGULATION_OF_NF_KAPPAB_TRANSCRIPTION_FACTOR_ACTIVITY | 3.32 | 4.44 | 5.99 | 8.46 | 0.00 | 0.09 | 0.00 | 0.39 |
| GO_NEGATIVE_REGULATION_OF_MULTICELLULAR_ORGANISMAL_PROCESS | 8.67 | 6.14 | 7.13 | 8.36 | 0.49 | 2.05 | 0.77 | 2.10 |
| GO_AMIDE_BINDING | 9.13 | 4.12 | 3.01 | 8.34 | 0.91 | 0.47 | 0.10 | 2.13 |
| GO_POSITIVE_REGULATION_OF_INTRACELLULAR_SIGNAL_TRANSDUCTION | 6.86 | 8.68 | 12.31 | 8.25 | 0.06 | 0.13 | 0.03 | 0.65 |
| GO_ENDOSOME | 6.17 | 2.59 | 2.81 | 8.24 | 0.00 | 0.04 | 0.04 | 0.19 |
| HALLMARK_IL2_STAT5_SIGNALING | 3.55 | 8.16 | 9.74 | 8.19 | 0.38 | 0.31 | 0.15 | 0.00 |
| GO_REGULATION_OF_RESPONSE_TO_CYTOKINE_STIMULUS | 6.48 | 8.25 | 3.84 | 8.15 | 0.00 | 0.07 | 0.35 | 0.29 |
| GO_POSITIVE_REGULATION_OF_CELL_PROLIFERATION | 3.99 | 5.50 | 9.32 | 8.15 | 0.26 | 0.98 | 0.19 | 2.06 |
| GO_LYMPHOCYTE_COSTIMULATION | 7.56 | 1.45 | 1.45 | 8.11 | 0.00 | 0.00 | 0.00 | 0.08 |
| GO_POSITIVE_REGULATION_OF_INNATE_IMMUNE_RESPONSE | 9.73 | 10.64 | 4.83 | 8.11 | 0.00 | 0.02 | 0.01 | 0.19 |
| GO_CHEMOKINE_ACTIVITY | 7.97 | 5.86 | 6.34 | 8.01 | 0.00 | 0.41 | 0.00 | 0.00 |
| GO_NEGATIVE_REGULATION_OF_LEUKOCYTE_PROLIFERATION | 6.33 | 1.41 | 0.28 | 7.96 | 0.86 | 0.29 | 0.42 | 0.26 |
| GO_NEGATIVE_REGULATION_OF_DEFENSE_RESPONSE | 8.32 | 5.31 | 4.40 | 7.92 | 0.00 | 0.10 | 0.02 | 0.04 |
| GO_MHC_CLASS_I_PROTEIN_COMPLEX | 11.91 | 8.94 | 6.60 | 7.91 | 0.00 | 0.00 | 0.00 | 0.00 |
| GO_LYMPHOCYTE_CHEMOTAXIS | 4.34 | 2.74 | 3.64 | 7.89 | 0.00 | 0.00 | 0.00 | 0.00 |
| GO_REGULATION_OF_LEUKOCYTE_MEDIATED_CYTOTOXICITY | 8.90 | 6.64 | 4.36 | 7.89 | 0.00 | 0.00 | 0.00 | 0.00 |
| GO_REGULATION_OF_I_KAPPAB_KINASE_NF_KAPPAB_SIGNALING | 7.24 | 8.20 | 7.70 | 7.88 | 0.00 | 0.09 | 0.31 | 0.29 |
| GO_POSITIVE_REGULATION_OF_LOCOMOTION | 4.32 | 7.36 | 8.24 | 7.79 | 0.00 | 0.94 | 0.01 | 0.35 |
| GO_VACUOLAR_PART | 7.75 | 1.82 | 0.70 | 7.78 | 0.00 | 0.00 | 0.09 | 0.10 |
| GO_LEUKOCYTE_MEDIATED_IMMUNITY | 7.88 | 0.91 | 1.18 | 7.74 | 0.00 | 0.11 | 0.12 | 0.51 |
| GO_ENDOSOMAL_PART | 8.14 | 2.43 | 1.27 | 7.66 | 0.00 | 0.00 | 0.19 | 0.03 |
| GO_TRANSPORT_VESICLE_MEMBRANE | 8.76 | 4.18 | 2.35 | 7.65 | 0.00 | 0.00 | 0.03 | 0.76 |
| GO_LYMPHOCYTE_MIGRATION | 4.21 | 2.65 | 4.82 | 7.61 | 0.00 | 0.00 | 0.00 | 0.00 |
| GO_RESPONSE_TO_INTERFERON_BETA | 6.84 | 8.90 | 0.54 | 7.61 | 0.00 | 0.00 | 0.00 | 0.00 |
| GO_NEGATIVE_REGULATION_OF_IMMUNE_RESPONSE | 9.18 | 8.73 | 4.54 | 7.60 | 0.00 | 0.00 | 0.00 | 0.00 |
| GO_CELL_CHEMOTAXIS | 7.67 | 4.79 | 5.43 | 7.48 | 0.00 | 0.71 | 0.00 | 0.04 |
| GO_CYTOPLASMIC_VESICLE_PART | 4.73 | 3.11 | 0.71 | 7.43 | 0.00 | 0.02 | 0.06 | 2.41 |
| GO_POSITIVE_REGULATION_OF_SEQUENCE_SPECIFIC_DNA_BINDING_TRANSCRIPTION_FACTOR_ACTIVITY | 4.10 | 5.32 | 5.46 | 7.41 | 0.00 | 0.13 | 0.02 | 0.46 |
| GO_NEGATIVE_REGULATION_OF_CELL_KILLING | 7.35 | 5.13 | 3.16 | 7.40 | 0.00 | 0.00 | 0.00 | 0.00 |
| GO_ENDOCYTIC_VESICLE | 7.06 | 2.28 | 2.54 | 7.36 | 0.00 | 0.02 | 0.12 | 0.23 |
| GO_NEGATIVE_REGULATION_OF_HOMOTYPIC_CELL_CELL_ADHESION | 7.64 | 0.90 | 1.00 | 7.35 | 0.00 | 0.18 | 0.06 | 0.03 |
| GO_REGULATION_OF_CYTOKINE_SECRETION | 9.96 | 3.46 | 2.04 | 7.30 | 0.00 | 0.15 | 0.00 | 0.08 |
| GO_INTRINSIC_COMPONENT_OF_PLASMA_MEMBRANE | 5.49 | 9.36 | 5.47 | 7.29 | 1.72 | 2.07 | 0.01 | 0.04 |
| GO_CHEMOKINE_MEDIATED_SIGNALING_PATHWAY | 6.17 | 8.81 | 7.16 | 7.27 | 0.00 | 0.37 | 0.00 | 0.00 |
| GO_RESPONSE_TO_OXYGEN_CONTAINING_COMPOUND | 4.54 | 4.93 | 13.64 | 7.26 | 0.48 | 0.94 | 0.07 | 1.71 |
| GO_REGULATION_OF_ANTIGEN_PROCESSING_AND_PRESENTATION_OF_PEPTIDE_ANTIGEN | 8.80 | 2.98 | 0.91 | 7.25 | 0.00 | 0.00 | 0.00 | 0.00 |
| GO_REGULATION_OF_CELL_KILLING | 8.31 | 6.15 | 4.08 | 7.24 | 0.00 | 0.00 | 0.00 | 0.00 |
| GO_LYMPHOCYTE_ACTIVATION | 4.54 | 4.94 | 5.38 | 7.22 | 0.00 | 1.43 | 0.30 | 0.59 |
| GO_PEPTIDASE_ACTIVITY | 4.91 | 4.13 | 0.64 | 7.13 | 0.00 | 0.09 | 0.05 | 0.12 |
| GO_INTERSPECIES_INTERACTION_BETWEEN_ORGANISMS | 8.67 | 5.32 | 2.93 | 7.12 | 0.49 | 17.00 | 0.00 | 17.00 |
| GO_CELLULAR_RESPONSE_TO_INTERLEUKIN_1 | 2.66 | 6.04 | 9.66 | 7.12 | 0.00 | 0.20 | 0.00 | 0.04 |
| GO_LEUKOCYTE_CHEMOTAXIS | 5.17 | 4.31 | 3.32 | 7.12 | 0.00 | 0.00 | 0.00 | 0.04 |
| GO_REGULATION_OF_INTERFERON_BETA_PRODUCTION | 6.06 | 5.24 | 3.54 | 7.11 | 0.00 | 0.00 | 0.55 | 0.00 |
| GO_COMPLEMENT_ACTIVATION | 4.63 | 1.08 | 0.61 | 7.10 | 0.00 | 0.00 | 0.00 | 0.30 |
| GO_CELLULAR_RESPONSE_TO_BIOTIC_STIMULUS | 6.75 | 5.45 | 8.38 | 7.06 | 0.00 | 0.00 | 0.00 | 0.02 |
| GO_POSITIVE_REGULATION_OF_INTERFERON_GAMMA_PRODUCTION | 6.83 | 4.88 | 2.90 | 7.05 | 0.00 | 0.00 | 0.00 | 0.15 |
| GO_ENDOPEPTIDASE_ACTIVITY | 6.14 | 4.04 | 0.49 | 6.96 | 0.00 | 0.31 | 0.10 | 0.02 |
| GO_POSITIVE_REGULATION_OF_IMMUNE_EFFECTOR_PROCESS | 6.96 | 4.95 | 3.09 | 6.95 | 0.00 | 0.00 | 0.00 | 0.14 |
| GO_ANTIGEN_RECEPTOR_MEDIATED_SIGNALING_PATHWAY | 10.38 | 4.17 | 1.72 | 6.86 | 0.00 | 0.06 | 0.01 | 0.23 |
| GO_VACUOLAR_MEMBRANE | 6.18 | 0.89 | 0.59 | 6.85 | 0.00 | 0.01 | 0.21 | 0.15 |
| GO_T_CELL_RECEPTOR_SIGNALING_PATHWAY | 11.12 | 3.95 | 1.36 | 6.84 | 0.00 | 0.07 | 0.01 | 0.30 |
| GO_CHEMOKINE_RECEPTOR_BINDING | 6.99 | 6.06 | 5.61 | 6.80 | 0.00 | 0.34 | 0.00 | 0.00 |
| GO_REGULATION_OF_LEUKOCYTE_MEDIATED_IMMUNITY | 6.51 | 6.18 | 5.93 | 6.76 | 0.00 | 0.00 | 0.00 | 0.01 |
| GO_DEFENSE_RESPONSE_TO_BACTERIUM | 6.20 | 5.14 | 2.12 | 6.76 | 0.00 | 0.16 | 0.00 | 0.24 |
| GO_NEGATIVE_REGULATION_OF_NATURAL_KILLER_CELL_MEDIATED_IMMUNITY | 6.30 | 5.76 | 3.48 | 6.67 | 0.00 | 0.00 | 0.00 | 0.00 |
| GO_REGULATION_OF_PEPTIDASE_ACTIVITY | 5.78 | 6.29 | 4.50 | 6.65 | 0.00 | 1.22 | 0.05 | 0.96 |
| GO_CELLULAR_RESPONSE_TO_OXYGEN_CONTAINING_COMPOUND | 3.49 | 2.89 | 9.14 | 6.62 | 0.28 | 1.08 | 0.02 | 0.92 |
| GO_REGULATION_OF_INTERFERON_ALPHA_PRODUCTION | 5.19 | 4.66 | 0.69 | 6.62 | 0.00 | 0.00 | 0.00 | 0.37 |
| GO_REGULATION_OF_ALPHA_BETA_T_CELL_PROLIFERATION | 6.72 | 6.08 | 1.70 | 6.62 | 0.00 | 0.00 | 0.00 | 0.00 |
| GO_ANTIGEN_PROCESSING_AND_PRESENTATION_OF_EXOGENOUS_PEPTIDE_ANTIGEN_VIA_MHC_CLASS_I | 12.19 | 10.53 | 5.90 | 6.60 | 0.00 | 0.00 | 0.00 | 0.12 |
| GO_REGULATION_OF_INTERLEUKIN_6_PRODUCTION | 3.18 | 2.62 | 2.94 | 6.57 | 0.00 | 0.27 | 0.00 | 0.23 |
| GO_NEGATIVE_REGULATION_OF_T_CELL_PROLIFERATION | 7.66 | 0.62 | 0.00 | 6.52 | 0.00 | 0.00 | 0.21 | 0.13 |
| GO_REGULATION_OF_INTERLEUKIN_1_PRODUCTION | 3.62 | 3.04 | 0.84 | 6.51 | 0.00 | 0.32 | 0.00 | 0.09 |
| GO_REGULATION_OF_SEQUENCE_SPECIFIC_DNA_BINDING_TRANSCRIPTION_FACTOR_ACTIVITY | 3.49 | 5.60 | 9.27 | 6.48 | 0.00 | 0.37 | 0.02 | 0.19 |
| GO_POSITIVE_REGULATION_OF_INTERLEUKIN_1_PRODUCTION | 3.66 | 2.24 | 0.00 | 6.47 | 0.00 | 0.00 | 0.00 | 0.18 |
| GO_NEGATIVE_REGULATION_OF_CELL_CELL_ADHESION | 7.16 | 0.96 | 0.74 | 6.43 | 0.00 | 0.77 | 0.03 | 0.01 |
| GO_INTRINSIC_COMPONENT_OF_ENDOPLASMIC_RETICULUM_MEMBRANE | 10.38 | 4.74 | 3.27 | 6.35 | 0.00 | 0.00 | 0.38 | 0.08 |
| GO_POSITIVE_REGULATION_OF_PHOSPHORUS_METABOLIC_PROCESS | 2.31 | 5.53 | 11.29 | 6.35 | 0.20 | 0.23 | 0.07 | 0.44 |
| GO_NEGATIVE_REGULATION_OF_IMMUNE_EFFECTOR_PROCESS | 7.47 | 5.42 | 3.84 | 6.31 | 0.00 | 0.00 | 0.06 | 0.11 |
| GO_ACTIVATION_OF_INNATE_IMMUNE_RESPONSE | 7.68 | 8.91 | 4.79 | 6.27 | 0.00 | 0.03 | 0.00 | 0.31 |
| GO_REGULATION_OF_LEUKOCYTE_DIFFERENTIATION | 2.43 | 4.40 | 8.09 | 6.24 | 0.00 | 2.17 | 0.01 | 0.01 |
| GO_NEGATIVE_REGULATION_OF_CELL_PROLIFERATION | 5.82 | 2.75 | 6.16 | 6.21 | 0.10 | 1.16 | 0.06 | 0.30 |
| GO_STAT_CASCADE | 5.05 | 3.40 | 3.61 | 6.21 | 0.00 | 0.37 | 0.00 | 0.00 |
| GO_NEGATIVE_REGULATION_OF_LEUKOCYTE_MEDIATED_IMMUNITY | 5.73 | 3.95 | 5.29 | 6.11 | 0.00 | 0.00 | 0.00 | 0.00 |
| GO_SINGLE_ORGANISM_CELL_ADHESION | 3.78 | 10.11 | 6.08 | 6.10 | 0.22 | 0.54 | 0.00 | 0.63 |
| GO_IMMUNE_RESPONSE_REGULATING_CELL_SURFACE_RECEPTOR_SIGNALING_PATHWAY | 8.12 | 3.48 | 2.10 | 6.02 | 0.30 | 0.37 | 0.06 | 1.99 |
| GO_MHC_CLASS_II_PROTEIN_COMPLEX_BINDING | 7.77 | 0.00 | 0.00 | 6.02 | 0.00 | 0.00 | 0.43 | 0.89 |
| GO_MHC_PROTEIN_COMPLEX_BINDING | 7.77 | 0.00 | 0.00 | 6.02 | 0.00 | 0.00 | 0.43 | 0.89 |
| GO_LEUKOCYTE_MIGRATION | 6.21 | 9.24 | 6.18 | 6.01 | 0.00 | 0.17 | 0.03 | 0.29 |
| GO_ANTIGEN_PROCESSING_AND_PRESENTATION_OF_PEPTIDE_OR_POLYSACCHARIDE_ANTIGEN_VIA_MHC_CLASS_II | 8.41 | 0.15 | 0.00 | 5.99 | 0.00 | 0.00 | 0.04 | 0.60 |
| GO_REGULATION_OF_INTRACELLULAR_SIGNAL_TRANSDUCTION | 3.79 | 9.53 | 17.00 | 5.95 | 0.01 | 0.19 | 0.03 | 0.21 |
| GO_BIOLOGICAL_ADHESION | 4.45 | 12.38 | 6.00 | 5.91 | 1.40 | 1.64 | 0.00 | 0.89 |
| GO_NEGATIVE_REGULATION_OF_RESPONSE_TO_STIMULUS | 6.59 | 11.06 | 9.95 | 5.89 | 0.01 | 0.37 | 0.04 | 0.06 |
| HALLMARK_KRAS_SIGNALING_UP | 1.78 | 6.77 | 7.76 | 5.85 | 1.08 | 3.25 | 0.03 | 0.43 |
| GO_CELL_CELL_ADHESION | 4.24 | 7.59 | 4.97 | 5.80 | 0.52 | 1.06 | 0.00 | 0.44 |
| GO_RESPONSE_TO_ORGANIC_CYCLIC_COMPOUND | 1.96 | 3.85 | 8.72 | 5.79 | 0.90 | 0.23 | 0.09 | 1.53 |
| GO_COATED_VESICLE_MEMBRANE | 7.67 | 3.48 | 1.43 | 5.73 | 0.00 | 0.00 | 0.39 | 2.79 |
| GO_REGULATION_OF_EXTRINSIC_APOPTOTIC_SIGNALING_PATHWAY | 1.96 | 4.48 | 10.09 | 5.72 | 0.00 | 0.43 | 0.12 | 0.51 |
| GO_POSITIVE_REGULATION_OF_I_KAPPAB_KINASE_NF_KAPPAB_SIGNALING | 6.70 | 5.26 | 2.47 | 5.69 | 0.00 | 0.16 | 0.36 | 0.40 |
| GO_REGULATION_OF_CELLULAR_COMPONENT_MOVEMENT | 5.04 | 7.59 | 8.36 | 5.66 | 1.00 | 0.81 | 0.15 | 0.60 |
| GO_PATTERN_RECOGNITION_RECEPTOR_SIGNALING_PATHWAY | 4.24 | 6.54 | 3.51 | 5.63 | 0.00 | 0.14 | 0.04 | 0.41 |
| GO_SIGNAL_TRANSDUCER_ACTIVITY | 6.90 | 6.41 | 2.33 | 5.55 | 0.88 | 1.83 | 0.08 | 0.12 |
| GO_POSITIVE_REGULATION_OF_BIOSYNTHETIC_PROCESS | 1.24 | 2.89 | 11.90 | 5.50 | 0.38 | 1.27 | 0.01 | 3.55 |
| GO_TAXIS | 3.70 | 5.71 | 7.34 | 5.47 | 0.62 | 3.20 | 0.51 | 0.97 |
| GO_CELLULAR_RESPONSE_TO_VIRUS | 7.05 | 5.01 | 2.45 | 5.31 | 0.00 | 0.00 | 0.00 | 0.00 |
| GO_REGULATION_OF_CELL_DEATH | 2.65 | 6.21 | 15.35 | 5.29 | 0.53 | 0.97 | 0.73 | 5.01 |
| GO_POSITIVE_REGULATION_OF_MOLECULAR_FUNCTION | 2.55 | 6.64 | 8.09 | 5.22 | 0.11 | 0.03 | 0.00 | 0.80 |
| GO_REGULATION_OF_PROTEIN_SECRETION | 7.13 | 3.30 | 2.00 | 5.20 | 0.00 | 0.17 | 0.00 | 0.43 |
| GO_REGULATION_OF_HEMOPOIESIS | 1.76 | 3.53 | 8.87 | 5.04 | 0.00 | 1.31 | 0.07 | 0.01 |
| GO_REGULATION_OF_INTERLEUKIN_10_SECRETION | 6.30 | 0.64 | 0.00 | 5.03 | 0.00 | 0.00 | 0.00 | 0.00 |
| GO_LEUKOCYTE_DIFFERENTIATION | 3.36 | 2.44 | 6.84 | 4.79 | 0.00 | 0.86 | 0.13 | 0.08 |
| GO_RESPONSE_TO_MECHANICAL_STIMULUS | 0.99 | 3.07 | 10.66 | 4.77 | 0.00 | 0.39 | 0.03 | 0.03 |
| GO_POSITIVE_REGULATION_OF_PROTEIN_MODIFICATION_PROCESS | 2.87 | 6.35 | 11.09 | 4.74 | 0.13 | 0.14 | 0.05 | 0.45 |
| HALLMARK_EPITHELIAL_MESENCHYMAL_TRANSITION | 2.05 | 5.81 | 6.34 | 4.74 | 1.63 | 1.52 | 0.10 | 5.26 |
| GO_RECEPTOR_ACTIVITY | 6.03 | 6.50 | 1.26 | 4.69 | 0.68 | 1.66 | 0.08 | 0.04 |
| GO_POSITIVE_REGULATION_OF_PROTEIN_METABOLIC_PROCESS | 2.84 | 6.23 | 13.42 | 4.57 | 0.16 | 0.26 | 0.03 | 2.51 |
| GO_REGULATION_OF_ERK1_AND_ERK2_CASCADE | 2.21 | 4.63 | 8.51 | 4.54 | 0.00 | 0.66 | 0.09 | 0.14 |
| GO_LOCOMOTION | 3.51 | 10.07 | 7.56 | 4.52 | 0.40 | 2.84 | 0.46 | 1.21 |
| GO_CLATHRIN_COATED_ENDOCYTIC_VESICLE_MEMBRANE | 6.29 | 0.20 | 0.00 | 4.40 | 0.00 | 0.00 | 1.14 | 0.12 |
| GO_TUMOR_NECROSIS_FACTOR_MEDIATED_SIGNALING_PATHWAY | 9.20 | 11.26 | 5.20 | 4.32 | 0.55 | 0.00 | 0.02 | 0.23 |
| GO_REGULATION_OF_PHOSPHORUS_METABOLIC_PROCESS | 1.74 | 5.39 | 8.64 | 4.23 | 0.16 | 0.14 | 0.19 | 0.37 |
| GO_POSITIVE_REGULATION_OF_LEUKOCYTE_DIFFERENTIATION | 1.01 | 3.41 | 6.22 | 4.21 | 0.00 | 1.43 | 0.00 | 0.02 |
| GO_POSITIVE_REGULATION_OF_HEMOPOIESIS | 1.06 | 3.68 | 6.80 | 4.11 | 0.00 | 1.05 | 0.02 | 0.01 |
| GO_INTRACELLULAR_SIGNAL_TRANSDUCTION | 2.60 | 8.52 | 10.04 | 4.09 | 0.06 | 0.05 | 0.00 | 0.23 |
| GO_REGULATION_OF_GRANULOCYTE_CHEMOTAXIS | 3.48 | 6.15 | 5.29 | 4.06 | 0.00 | 0.00 | 0.00 | 0.99 |
| GO_RESPONSE_TO_INTERFERON_ALPHA | 6.84 | 4.85 | 0.00 | 4.02 | 0.00 | 0.00 | 0.00 | 0.00 |
| GO_BLOOD_VESSEL_MORPHOGENESIS | 1.38 | 6.59 | 8.37 | 4.01 | 0.00 | 0.57 | 1.56 | 0.87 |
| GO_POSITIVE_REGULATION_OF_MAPK_CASCADE | 1.88 | 5.02 | 10.14 | 3.97 | 0.00 | 0.30 | 0.07 | 0.56 |
| GO_REGULATION_OF_MONONUCLEAR_CELL_MIGRATION | 1.46 | 6.08 | 4.29 | 3.95 | 0.00 | 0.00 | 0.00 | 1.00 |
| GO_CELLULAR_RESPONSE_TO_LIPID | 3.72 | 3.66 | 6.71 | 3.91 | 0.00 | 0.64 | 0.17 | 1.13 |
| GO_REGULATION_OF_MAPK_CASCADE | 1.24 | 5.22 | 10.69 | 3.89 | 0.00 | 0.60 | 0.05 | 0.18 |
| GO_POSITIVE_REGULATION_OF_ERK1_AND_ERK2_CASCADE | 2.22 | 3.99 | 6.24 | 3.87 | 0.00 | 0.69 | 0.26 | 0.45 |
| GO_INTRINSIC_COMPONENT_OF_ORGANELLE_MEMBRANE | 6.31 | 3.20 | 2.42 | 3.65 | 0.28 | 0.01 | 0.33 | 0.10 |
| GO_REGULATION_OF_PROTEIN_MODIFICATION_PROCESS | 2.37 | 6.08 | 8.67 | 3.60 | 0.11 | 0.12 | 0.07 | 0.49 |
| GO_CXCR_CHEMOKINE_RECEPTOR_BINDING | 6.23 | 3.09 | 2.70 | 3.58 | 0.00 | 0.65 | 0.00 | 0.00 |
| GO_REGULATION_OF_APOPTOTIC_SIGNALING_PATHWAY | 1.70 | 3.28 | 6.76 | 3.57 | 0.00 | 0.65 | 0.05 | 2.21 |
| GO_RESPONSE_TO_ALCOHOL | 1.05 | 1.07 | 6.31 | 3.57 | 0.00 | 0.27 | 0.08 | 1.21 |
| GO_POSITIVE_REGULATION_OF_PEPTIDYL_TYROSINE_PHOSPHORYLATION | 2.90 | 6.32 | 4.56 | 3.38 | 0.00 | 0.10 | 0.00 | 0.47 |
| GO_REGULATION_OF_CYTOKINE_BIOSYNTHETIC_PROCESS | 1.08 | 2.73 | 6.15 | 3.38 | 0.00 | 0.00 | 0.13 | 0.00 |
| GO_POSITIVE_REGULATION_OF_CATALYTIC_ACTIVITY | 1.47 | 3.85 | 6.57 | 3.33 | 0.06 | 0.04 | 0.01 | 0.53 |
| GO_NEGATIVE_REGULATION_OF_CELL_COMMUNICATION | 2.75 | 8.62 | 7.08 | 3.31 | 0.02 | 0.64 | 0.11 | 0.11 |
| GO_ENDOPLASMIC_RETICULUM_PART | 3.52 | 1.35 | 0.61 | 3.30 | 0.27 | 0.00 | 0.47 | 6.86 |
| GO_CELLULAR_RESPONSE_TO_EXTERNAL_STIMULUS | 2.22 | 1.48 | 8.95 | 3.24 | 0.00 | 0.19 | 0.15 | 0.04 |
| GO_POSITIVE_REGULATION_OF_CELL_DEATH | 1.48 | 0.90 | 6.64 | 3.21 | 0.70 | 0.88 | 0.86 | 1.63 |
| GO_CELL_MOTILITY | 3.68 | 8.12 | 7.58 | 3.07 | 0.08 | 1.16 | 0.19 | 1.19 |
| HALLMARK_UV_RESPONSE_UP | 0.76 | 2.33 | 11.28 | 3.07 | 0.45 | 0.05 | 0.73 | 0.55 |
| GO_VASCULATURE_DEVELOPMENT | 1.27 | 5.47 | 8.37 | 2.98 | 0.16 | 0.68 | 1.04 | 0.74 |
| GO_ANGIOGENESIS | 1.64 | 5.44 | 7.76 | 2.96 | 0.00 | 0.34 | 1.01 | 1.02 |
| GO_POSITIVE_REGULATION_OF_GENE_EXPRESSION | 0.72 | 2.15 | 11.03 | 2.87 | 0.39 | 1.14 | 0.05 | 1.81 |
| GO_I_KAPPAB_KINASE_NF_KAPPAB_SIGNALING | 0.45 | 6.40 | 6.53 | 2.78 | 0.00 | 0.00 | 0.09 | 0.40 |
| GO_ENDOPLASMIC_RETICULUM | 3.34 | 1.40 | 0.36 | 2.76 | 0.27 | 0.01 | 0.47 | 9.20 |
| GO_NEGATIVE_REGULATION_OF_TRANSPORT | 2.86 | 5.33 | 6.10 | 2.73 | 0.00 | 0.18 | 0.09 | 0.36 |
| GO_RNA_POLYMERASE_II_TRANSCRIPTION_FACTOR_ACTIVITY_SEQUENCE_SPECIFIC_DNA_BINDING | 1.04 | 3.13 | 8.28 | 2.73 | 0.44 | 3.32 | 0.81 | 0.31 |
| GO_REGULATION_OF_MULTICELLULAR_ORGANISMAL_DEVELOPMENT | 2.76 | 4.77 | 8.32 | 2.60 | 0.15 | 2.70 | 0.26 | 1.71 |
| GO_MEMBRANE_PROTEIN_COMPLEX | 4.70 | 1.93 | 1.14 | 2.57 | 0.44 | 0.05 | 0.37 | 7.77 |
| GO_PROTEIN_COMPLEX_BINDING | 1.24 | 1.07 | 0.07 | 2.53 | 0.05 | 0.01 | 0.86 | 8.93 |
| GO_POSITIVE_REGULATION_OF_TRANSCRIPTION_FROM_RNA_POLYMERASE_II_PROMOTER | 0.66 | 1.98 | 10.21 | 2.45 | 0.70 | 1.02 | 0.06 | 0.84 |
| GO_REGULATION_OF_SMOOTH_MUSCLE_CELL_PROLIFERATION | 0.60 | 2.18 | 7.29 | 2.43 | 0.00 | 0.89 | 0.05 | 0.42 |
| GO_POSITIVE_REGULATION_OF_DEVELOPMENTAL_PROCESS | 0.89 | 2.80 | 6.52 | 2.43 | 0.16 | 1.93 | 0.07 | 1.80 |
| GO_REGULATION_OF_TYROSINE_PHOSPHORYLATION_OF_STAT_PROTEIN | 2.01 | 4.07 | 6.82 | 2.42 | 0.00 | 0.00 | 0.00 | 0.34 |
| GO_NIK_NF_KAPPAB_SIGNALING | 4.09 | 6.32 | 5.11 | 2.27 | 0.00 | 0.00 | 0.00 | 0.38 |
| GO_NEGATIVE_REGULATION_OF_CELL_DEATH | 0.61 | 6.24 | 11.35 | 2.08 | 0.18 | 0.72 | 0.23 | 4.13 |
| GO_RESPONSE_TO_ABIOTIC_STIMULUS | 0.53 | 1.55 | 7.93 | 1.83 | 0.17 | 0.35 | 0.13 | 0.27 |
| GO_CELLULAR_RESPONSE_TO_EXTRACELLULAR_STIMULUS | 1.33 | 0.61 | 6.03 | 1.81 | 0.00 | 0.38 | 0.09 | 0.03 |
| GO_REGULATION_OF_CELL_DIFFERENTIATION | 0.49 | 3.08 | 8.25 | 1.71 | 0.08 | 3.11 | 0.41 | 1.55 |
| GO_REGULATION_OF_STAT_CASCADE | 1.10 | 2.97 | 6.53 | 1.57 | 0.00 | 0.16 | 0.05 | 0.25 |
| GO_REGULATION_OF_MACROPHAGE_DERIVED_FOAM_CELL_DIFFERENTIATION | 0.00 | 1.95 | 6.77 | 1.56 | 0.00 | 0.00 | 0.00 | 0.00 |
| GO_REGULATION_OF_TRANSCRIPTION_FROM_RNA_POLYMERASE_II_PROMOTER | 0.42 | 1.97 | 11.60 | 1.53 | 0.39 | 0.81 | 1.36 | 0.35 |
| GO_MACROMOLECULAR_COMPLEX_BINDING | 0.29 | 1.10 | 0.61 | 1.50 | 0.18 | 0.07 | 1.51 | 10.34 |
| GO_NEGATIVE_REGULATION_OF_GENE_EXPRESSION | 0.85 | 1.98 | 6.28 | 1.32 | 0.01 | 0.84 | 1.70 | 1.92 |
| GO_RESPONSE_TO_INORGANIC_SUBSTANCE | 0.12 | 1.00 | 8.85 | 1.30 | 0.17 | 0.04 | 0.07 | 0.56 |
| GO_CIRCULATORY_SYSTEM_DEVELOPMENT | 0.85 | 3.63 | 7.13 | 1.24 | 0.24 | 0.88 | 0.51 | 0.78 |
| GO_TRANSCRIPTION_FACTOR_ACTIVITY_RNA_POLYMERASE_II_CORE_PROMOTER_PROXIMAL_REGION_SEQUENCE_{—} | 0.76 | 1.46 | 6.53 | 1.15 | 0.00 | 5.27 | 0.41 | 0.69 |
| SPECIFIC_BINDING | | | | | | | | |
| GO_POSITIVE_REGULATION_OF_CELL_DIFFERENTIATION | 0.40 | 2.03 | 6.78 | 1.10 | 0.08 | 1.65 | 0.01 | 1.46 |
| GO_ANATOMICAL_STRUCTURE_FORMATION_INVOLVED_IN_MORPHOGENESIS | 0.06 | 3.20 | 6.37 | 1.09 | 0.19 | 1.19 | 0.55 | 1.42 |
| GO_RESPONSE_TO_ALKALOID | 0.57 | 2.08 | 7.10 | 0.97 | 0.00 | 0.14 | 0.04 | 0.65 |
| GO_CATABOLIC_PROCESS | 3.19 | 1.65 | 0.19 | 0.95 | 0.02 | 17.00 | 0.48 | 6.78 |
| GO_NUCLEIC_ACID_BINDING_TRANSCRIPTION_FACTOR_ACTIVITY | 0.51 | 1.34 | 8.70 | 0.85 | 0.58 | 1.55 | 1.66 | 0.01 |
| GO_CELLULAR_CATABOLIC_PROCESS | 3.26 | 1.25 | 0.42 | 0.82 | 0.06 | 17.00 | 0.03 | 7.22 |
| GO_TRANSCRIPTIONAL_ACTIVATOR_ACTIVITY_RNA_POLYMERASE_II_TRANSCRIPTION_REGULATORY_REGION_SEQUENCE_{—} | 0.20 | 1.26 | 7.62 | 0.78 | 0.87 | 4.82 | 0.93 | 1.58 |
| SPECIFIC_BINDING | | | | | | | | |
| GO_CELLULAR_RESPONSE_TO_HYDROGEN_PEROXIDE | 0.15 | 0.33 | 6.61 | 0.75 | 0.00 | 0.23 | 0.32 | 0.75 |
| GO_RESPONSE_TO_HYDROGEN_PEROXIDE | 0.05 | 0.11 | 7.12 | 0.68 | 0.00 | 0.10 | 0.26 | 0.68 |
| GO_ANCHORING_JUNCTION | 0.60 | 0.77 | 0.55 | 0.55 | 0.38 | 17.00 | 0.05 | 17.00 |
| GO_VIRAL_LIFE_CYCLE | 0.80 | 1.30 | 0.52 | 0.51 | 0.64 | 17.00 | 0.00 | 17.00 |
| GO_CELL_JUNCTION | 0.63 | 1.60 | 0.15 | 0.49 | 0.63 | 17.00 | 0.01 | 12.04 |
| GO_CELL_SUBSTRATE_JUNCTION | 0.22 | 1.36 | 0.76 | 0.34 | 0.45 | 17.00 | 0.06 | 17.00 |
| GO_REGULATION_OF_PROTEIN_LOCALIZATION_TO_CHROMOSOME_TELOMERIC_REGION | 0.52 | 0.00 | 0.00 | 0.32 | 0.00 | 0.00 | 0.43 | 7.41 |
| GO_MACROMOLECULAR_COMPLEX_ASSEMBLY | 0.32 | 0.37 | 0.28 | 0.29 | 0.49 | 0.44 | 0.01 | 7.99 |
| GO_MACROMOLECULE_CATABOLIC_PROCESS | 2.79 | 1.70 | 0.78 | 0.28 | 0.13 | 17.00 | 0.00 | 8.37 |
| GO_ORGANIC_CYCLIC_COMPOUND_CATABOLIC_PROCESS | 0.59 | 0.51 | 0.16 | 0.26 | 0.49 | 17.00 | 0.05 | 17.00 |
| GO_CYTOSOLIC_PART | 0.18 | 0.08 | 0.07 | 0.24 | 0.84 | 17.00 | 0.03 | 17.00 |
| GO_HYDROGEN_TRANSPORT | 0.18 | 0.26 | 0.09 | 0.24 | 0.00 | 0.00 | 0.10 | 7.15 |
| GO_POSTTRANSCRIPTIONAL_REGULATION_OF_GENE_EXPRESSION | 1.67 | 1.09 | 2.88 | 0.23 | 0.13 | 0.91 | 0.27 | 11.85 |
| GO_ENERGY_COUPLED_PROTON_TRANSPORT_DOWN_ELECTROCHEMICAL_GRADIENT | 0.40 | 0.00 | 0.00 | 0.22 | 0.00 | 0.00 | 0.31 | 7.10 |
| GO_HYDROGEN_ION_TRANSMEMBRANE_TRANSPORT | 0.08 | 0.17 | 0.13 | 0.20 | 0.00 | 0.00 | 0.04 | 7.85 |
| GO_TRANSCRIPTIONAL_ACTIVATOR_ACTIVITY_RNA_POLYMERASE_II_CORE_PROMOTER_PROXIMAL_REGION_SEQUENCE_{—} | 0.09 | 0.70 | 4.78 | 0.19 | 0.00 | 6.56 | 0.98 | 1.66 |
| SPECIFIC_BINDING | | | | | | | | |
| GO_PROTEIN_STABILIZATION | 0.33 | 0.02 | 0.00 | 0.17 | 0.00 | 0.00 | 0.63 | 6.99 |
| GO_TRANSLATION_FACTOR_ACTIVITY_RNA_BINDING | 0.06 | 0.00 | 0.36 | 0.15 | 0.00 | 0.76 | 0.03 | 9.61 |
| GO_ATP_BIOSYNTHETIC_PROCESS | 0.30 | 0.00 | 0.00 | 0.15 | 0.00 | 0.00 | 0.66 | 6.86 |
| GO_PIGMENT_GRANULE | 0.22 | 0.03 | 0.00 | 0.15 | 0.00 | 0.00 | 0.13 | 13.19 |
| GO_RIBONUCLEOSIDE_TRIPHOSPHATE_BIOSYNTHETIC_PROCESS | 0.20 | 0.00 | 0.00 | 0.08 | 0.00 | 0.00 | 0.87 | 7.09 |
| GO_NAD_METABOLIC_PROCESS | 0.00 | 0.45 | 0.00 | 0.08 | 0.00 | 0.29 | 3.63 | 6.12 |
| GO_REGULATION_OF_CELLULAR_AMIDE_METABOLIC_PROCESS | 0.04 | 0.17 | 2.23 | 0.05 | 0.19 | 1.54 | 0.16 | 12.66 |
| GO_PROTEIN_LOCALIZATION_TO_MEMBRANE | 0.01 | 0.05 | 0.22 | 0.05 | 1.08 | 17.00 | 0.16 | 17.00 |
| GO_INTRACELLULAR_PROTEIN_TRANSPORT | 0.02 | 0.02 | 0.09 | 0.05 | 0.72 | 17.00 | 0.10 | 17.00 |
| GO_SINGLE_ORGANISM_CELLULAR_LOCALIZATION | 0.00 | 0.00 | 0.12 | 0.05 | 1.07 | 17.00 | 0.16 | 17.00 |
| GO_NUCLEOBASE_CONTAINING_SMALL_MOLECULE_METABOLIC_PROCESS | 0.49 | 0.07 | 0.00 | 0.04 | 0.00 | 0.02 | 2.62 | 6.14 |
| GO_ESTABLISHMENT_OF_PROTEIN_LOCALIZATION | 0.00 | 0.00 | 0.00 | 0.03 | 0.73 | 17.00 | 0.00 | 17.00 |
| GO_PROTEIN_TARGETING | 0.03 | 0.07 | 0.22 | 0.03 | 1.46 | 17.00 | 0.11 | 17.00 |
| GO_GLYCOSYL_COMPOUND_METABOLIC_PROCESS | 0.10 | 0.03 | 0.00 | 0.02 | 0.00 | 0.00 | 3.87 | 7.59 |
| GO_PROTEIN_LOCALIZATION | 0.00 | 0.03 | 0.04 | 0.02 | 0.73 | 17.00 | 0.01 | 15.48 |
| GO_REGULATION_OF_TRANSLATIONAL_INITIATION | 0.00 | 0.17 | 1.35 | 0.02 | 0.00 | 0.43 | 0.16 | 8.71 |
| GO_PEPTIDE_METABOLIC_PROCESS | 0.04 | 0.01 | 0.01 | 0.01 | 0.28 | 17.00 | 0.12 | 17.00 |
| GO_ESTABLISHMENT_OF_PROTEIN_LOCALIZATION_TO_ORGANELLE | 0.02 | 0.11 | 0.68 | 0.01 | 1.59 | 17.00 | 0.06 | 17.00 |
| GO_ESTABLISHMENT_OF_PROTEIN_LOCALIZATION_TO_MEMBRANE | 0.04 | 0.08 | 0.24 | 0.01 | 0.72 | 17.00 | 0.25 | 17.00 |
| GO_SINGLE_ORGANISM_BIOSYNTHETIC_PROCESS | 0.21 | 0.19 | 0.01 | 0.01 | 0.00 | 0.00 | 6.15 | 1.41 |
| GO_UNFOLDED_PROTEIN_BINDING | 0.06 | 0.13 | 0.11 | 0.01 | 0.58 | 0.00 | 0.90 | 10.39 |
| GO_MITOCHONDRION | 0.03 | 0.00 | 0.01 | 0.01 | 0.00 | 0.00 | 3.81 | 6.32 |
| GO_NUCLEOLUS | 0.12 | 0.00 | 0.00 | 0.01 | 0.13 | 7.16 | 0.60 | 8.75 |
| GO_RNA_CATABOLIC_PROCESS | 0.05 | 0.19 | 0.28 | 0.01 | 0.76 | 17.00 | 0.04 | 17.00 |
| GO_ORGANONITROGEN_COMPOUND_METABOLIC_PROCESS | 0.15 | 0.22 | 0.09 | 0.01 | 0.02 | 17.00 | 0.80 | 17.00 |
| GO_ESTABLISHMENT_OF_LOCALIZATION_IN_CELL | 0.00 | 0.00 | 0.01 | 0.01 | 0.17 | 17.00 | 0.43 | 17.00 |
| GO_PROTEIN_TARGETING_TO_MEMBRANE | 0.01 | 0.00 | 0.00 | 0.01 | 1.04 | 17.00 | 0.03 | 17.00 |
| GO_NUCLEOSIDE_MONOPHOSPHATE_METABOLIC_PROCESS | 0.00 | 0.00 | 0.00 | 0.00 | 0.00 | 0.02 | 3.04 | 10.56 |
| GO_CYTOSOLIC_RIBOSOME | 0.00 | 0.00 | 0.00 | 0.00 | 1.26 | 17.00 | 0.00 | 17.00 |
| GO_CELLULAR_AMIDE_METABOLIC_PROCESS | 0.01 | 0.00 | 0.01 | 0.00 | 0.20 | 17.00 | 0.12 | 17.00 |
| GO_PURINE_CONTAINING_COMPOUND_METABOLIC_PROCESS | 0.30 | 0.03 | 0.00 | 0.00 | 0.00 | 0.02 | 2.19 | 7.96 |
| GO_MITOCHONDRIAL_ENVELOPE | 0.00 | 0.00 | 0.00 | 0.00 | 0.06 | 0.00 | 1.95 | 7.10 |
| GO_ENVELOPE | 0.00 | 0.01 | 0.00 | 0.00 | 0.22 | 0.00 | 1.92 | 6.13 |
| GO_CELLULAR_MACROMOLECULE_LOCALIZATION | 0.00 | 0.04 | 0.28 | 0.00 | 0.61 | 17.00 | 0.07 | 17.00 |
| GO_CELLULAR_MACROMOLECULAR_COMPLEX_ASSEMBLY | 0.04 | 0.00 | 0.01 | 0.00 | 1.24 | 1.30 | 0.00 | 10.21 |
| GO_MRNA_BINDING | 0.00 | 0.01 | 0.69 | 0.00 | 0.00 | 1.60 | 0.11 | 11.14 |
| GO_NUCLEOSIDE_TRIPHOSPHATE_METABOLIC_PROCESS | 0.09 | 0.00 | 0.00 | 0.00 | 0.00 | 0.00 | 1.91 | 11.03 |
| GO_PROTEIN_FOLDING | 0.03 | 0.01 | 0.00 | 0.00 | 0.00 | 0.08 | 0.46 | 11.03 |
| GO_MYELIN_SHEATH | 0.02 | 0.10 | 0.04 | 0.00 | 0.42 | 0.17 | 1.22 | 14.07 |
| GO_MULTI_ORGANISM_METABOLIC_PROCESS | 0.01 | 0.00 | 0.04 | 0.00 | 1.05 | 17.00 | 0.00 | 17.00 |
| GO_ORGANELLE_INNER_MEMBRANE | 0.00 | 0.00 | 0.00 | 0.00 | 0.00 | 0.01 | 1.30 | 7.51 |
| GO_PROTEIN_LOCALIZATION_TO_ORGANELLE | 0.00 | 0.02 | 0.48 | 0.00 | 1.61 | 17.00 | 0.10 | 17.00 |
| GO_RRNA_METABOLIC_PROCESS | 0.03 | 0.00 | 0.00 | 0.00 | 0.65 | 17.00 | 0.42 | 17.00 |
| GO_ORGANONITROGEN_COMPOUND_BIOSYNTHETIC_PROCESS | 0.04 | 0.03 | 0.02 | 0.00 | 0.10 | 17.00 | 0.91 | 17.00 |
| GO_MEMBRANE_ORGANIZATION | 0.00 | 0.01 | 0.04 | 0.00 | 0.69 | 17.00 | 0.31 | 17.00 |
| GO_STRUCTURAL_MOLECULE_ACTIVITY | 0.00 | 0.03 | 0.02 | 0.00 | 1.70 | 17.00 | 0.19 | 17.00 |
| GO_RIBONUCLEOPROTEIN_COMPLEX_SUBUNIT_ORGANIZATION | 0.03 | 0.01 | 0.02 | 0.00 | 0.86 | 6.94 | 0.00 | 11.60 |
| GO_RNA_BINDING | 0.22 | 0.01 | 0.00 | 0.00 | 1.24 | 17.00 | 0.06 | 17.00 |
| GO_STRUCTURAL_CONSTITUENT_OF_RIBOSOME | 0.00 | 0.00 | 0.00 | 0.00 | 0.83 | 17.00 | 0.83 | 17.00 |
| GO_AMIDE_BIOSYNTHETIC_PROCESS | 0.00 | 0.00 | 0.00 | 0.00 | 0.33 | 17.00 | 0.28 | 17.00 |
| GO_RIBONUCLEOPROTEIN_COMPLEX | 0.02 | 0.01 | 0.01 | 0.00 | 0.68 | 17.00 | 0.85 | 17.00 |
| GO_RIBOSOME | 0.00 | 0.00 | 0.01 | 0.00 | 1.38 | 17.00 | 1.09 | 17.00 |
| GO_GENERATION_OF_PRECURSOR_METABOLITES_AND_ENERGY | 0.00 | 0.01 | 0.04 | 0.00 | 0.00 | 0.04 | 3.12 | 9.14 |
| GO_RIBOSOME_BIOGENESIS | 0.00 | 0.00 | 0.00 | 0.00 | 0.54 | 17.00 | 0.33 | 17.00 |
| GO_POLY_A_RNA_BINDING | 0.01 | 0.00 | 0.00 | 0.00 | 1.07 | 17.00 | 0.04 | 17.00 |
| GO_MRNA_METABOLIC_PROCESS | 0.00 | 0.00 | 0.00 | 0.00 | 0.51 | 17.00 | 0.01 | 17.00 |
| GO_RIBONUCLEOPROTEIN_COMPLEX_BIOGENESIS | 0.00 | 0.00 | 0.00 | 0.00 | 0.35 | 17.00 | 0.11 | 17.00 |
| GO_NCRNA_PROCESSING | 0.00 | 0.00 | 0.00 | 0.00 | 0.42 | 17.00 | 0.75 | 14.81 |
| GO_NCRNA_METABOLIC_PROCESS | 0.00 | 0.00 | 0.00 | 0.00 | 0.28 | 17.00 | 1.02 | 11.58 |
| GO_RNA_PROCESSING | 0.00 | 0.00 | 0.00 | 0.00 | 0.89 | 17.00 | 0.12 | 17.00 |
| GO_POLYSOME | 0.00 | 0.16 | 0.31 | 0.00 | 0.90 | 2.42 | 0.00 | 6.51 |
| GO_RIBOSOMAL_SUBUNIT | 0.00 | 0.00 | 0.00 | 0.00 | 1.71 | 17.00 | 0.40 | 17.00 |
| GO_LARGE_RIBOSOMAL_SUBUNIT | 0.00 | 0.00 | 0.00 | 0.00 | 2.37 | 17.00 | 0.25 | 17.00 |
| GO_TRANSLATIONAL_INITIATION | 0.00 | 0.00 | 0.13 | 0.00 | 1.03 | 17.00 | 0.00 | 17.00 |
| GO_PROTEIN_LOCALIZATION_TO_ENDOPLASMIC_RETICULUM | 0.00 | 0.00 | 0.05 | 0.00 | 1.16 | 17.00 | 0.00 | 17.00 |
| GO_NUCLEAR_TRANSCRIBED_MRNA_CATABOLIC_PROCESS_NONSENSE_MEDIATED_DECAY | 0.00 | 0.01 | 0.00 | 0.00 | 1.17 | 17.00 | 0.00 | 17.00 |
| GO_ESTABLISHMENT_OF_PROTEIN_LOCALIZATION_TO_ENDOPLASMIC_RETICULUM | 0.00 | 0.00 | 0.00 | 0.00 | 1.27 | 17.00 | 0.00 | 17.00 |
| GO_CYTOSOLIC_LARGE_RIBOSOMAL_SUBUNIT | 0.00 | 0.00 | 0.00 | 0.00 | 1.75 | 17.00 | 0.00 | 17.00 |
| GO_CYTOPLASMIC_TRANSLATION | 0.00 | 0.00 | 0.00 | 0.00 | 0.86 | 14.45 | 0.00 | 17.00 |
| GO_CYTOSOLIC_SMALL_RIBOSOMAL_SUBUNIT | 0.00 | 0.00 | 0.00 | 0.00 | 0.00 | 17.00 | 0.00 | 12.03 |
| GO_MITOCHONDRIAL_PROTEIN_COMPLEX | 0.00 | 0.00 | 0.00 | 0.00 | 0.43 | 0.18 | 1.28 | 10.36 |
| GO_SMALL_RIBOSOMAL_SUBUNIT | 0.00 | 0.00 | 0.00 | 0.00 | 0.00 | 17.00 | 0.44 | 10.32 |
| GO_MITOCHONDRIAL_MEMBRANE_PART | 0.00 | 0.00 | 0.03 | 0.00 | 0.38 | 0.03 | 0.70 | 10.23 |
| GO_FORMATION_OF_TRANSLATION_PREINITIATION_COMPLEX | 0.00 | 0.00 | 0.00 | 0.00 | 0.00 | 0.53 | 0.00 | 10.10 |
| GO_INNER_MITOCHONDRIAL_MEMBRANE_PROTEIN_COMPLEX | 0.00 | 0.00 | 0.00 | 0.00 | 0.00 | 0.08 | 0.98 | 8.64 |
| GO_REGULATION_OF_TELOMERASE_RNA_LOCALIZATION_TO_CAJAL_BODY | 0.00 | 0.00 | 0.00 | 0.00 | 0.00 | 0.00 | 0.41 | 8.54 |
| GO_TRANSLATION_INITIATION_FACTOR_ACTIVITY | 0.00 | 0.00 | 0.24 | 0.00 | 0.00 | 0.25 | 0.00 | 8.11 |
| GO_RRNA_BINDING | 0.00 | 0.00 | 0.00 | 0.00 | 0.00 | 17.00 | 0.27 | 7.91 |
| GO_MITOCHONDRIAL_ATP_SYNTHESIS_COUPLED_PROTON_TRANSPORT | 0.00 | 0.00 | 0.00 | 0.00 | 0.00 | 0.00 | 0.37 | 7.89 |
| GO_RIBOSOME_ASSEMBLY | 0.00 | 0.00 | 0.00 | 0.00 | 1.83 | 12.90 | 0.00 | 7.59 |
| GO_RIBOSOMAL_LARGE_SUBUNIT_BIOGENESIS | 0.00 | 0.00 | 0.00 | 0.00 | 1.92 | 12.21 | 0.36 | 7.26 |
| GO_EPHRIN_RECEPTOR_SIGNALING_PATHWAY | 0.00 | 0.22 | 0.16 | 0.00 | 0.69 | 1.00 | 0.48 | 7.15 |
| GO_TRANSLATION_PREINITIATION_COMPLEX | 0.00 | 0.00 | 0.00 | 0.00 | 0.00 | 0.62 | 0.00 | 7.10 |
| GO_EUKARYOTIC_TRANSLATION_INITIATION_FACTOR_3_COMPLEX | 0.00 | 0.00 | 0.00 | 0.00 | 0.00 | 0.62 | 0.00 | 7.10 |
| GO_SPERM_EGG_RECOGNITION | 0.00 | 0.00 | 0.00 | 0.00 | 0.00 | 0.00 | 0.51 | 6.98 |
| GO_BINDING_OF_SPERM_TO_ZONA_PELLUCIDA | 0.00 | 0.00 | 0.00 | 0.00 | 0.00 | 0.00 | 0.51 | 6.98 |
| GO_REGULATION_OF_ESTABLISHMENT_OF_PROTEIN_LOCALIZATION_TO_CHROMOSOME | 0.00 | 0.00 | 0.00 | 0.00 | 0.00 | 0.00 | 0.51 | 6.98 |
| GO_PROTON_TRANSPORTING_ATP_SYNTHASE_COMPLEX | 0.00 | 0.00 | 0.00 | 0.00 | 0.00 | 0.00 | 0.83 | 6.88 |
| GO_CELL_CELL_RECOGNITION | 0.00 | 0.00 | 0.00 | 0.00 | 0.00 | 0.00 | 0.37 | 6.55 |
| GO_COPI_COATED_VESICLE | 0.00 | 0.00 | 0.00 | 0.00 | 0.00 | 0.00 | 0.00 | 6.32 |
| GO_NADH_METABOLIC_PROCESS | 0.00 | 0.26 | 0.00 | 0.00 | 0.00 | 0.00 | 4.02 | 6.28 |
| GO_CATALYTIC_STEP_2_SPLICEOSOME | 0.00 | 0.00 | 0.00 | 0.00 | 0.00 | 0.00 | 0.03 | 6.13 |
| GO_RIBOSOMAL_LARGE_SUBUNIT_ASSEMBLY | 0.00 | 0.00 | 0.00 | 0.00 | 2.57 | 10.40 | 0.00 | 5.70 |
| GO_RIBOSOMAL_SMALL_SUBUNIT_BIOGENESIS | 0.00 | 0.00 | 0.00 | 0.00 | 0.00 | 9.84 | 0.00 | 3.89 |
| GO_RIBOSOMAL_SMALL_SUBUNIT_ASSEMBLY | 0.00 | 0.00 | 0.00 | 0.00 | 0.00 | 6.48 | 0.00 | 2.21 |

### Example 6—Evidence of Antitumor Immune Activity Despite Low Immune Infiltration

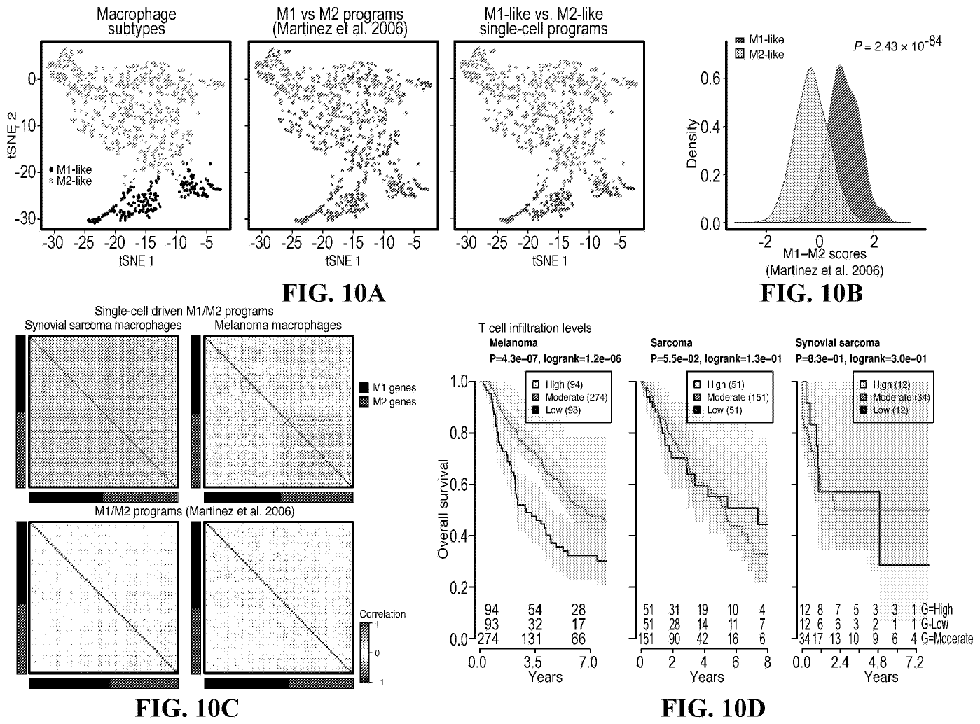

Having mapped the malignant cell states, Applicants turned to characterize immune cells within SyS tumors. Single-cell data revealed diverse cell states indicative of antitumor immunity (FIG. 9C, Table 12): analyzing macrophages Applicants observed M1-like and M2-like states, reminiscent of pro- and anti-inflammatory properties, respectively (FIGS. 9C, 10A-10C; Table 12). Applicants also observed various T cell subsets, including naïve, cytotoxic, exhausted, and regulatory T cells (FIG. 9C-9D).

TABLE 12
scRNA-Seq-based M1 and M2 signatures.

| M1 | | M2 | | M1 (top 50) | M2 (top 50) |
|---|---|---|---|---|---|
| ABRACL | LIMD2 | A2M | KCNMA1 | ALDH2 | A2M |
| ACAP1 | LIMS1 | ABCC5 | KCTD12 | ANPEP | AP1B1 |
| ACOT9 | LIPN | ABCD4 | KIAA1683 | ANXA2 | C1QA |
| ACSL5 | LOC100288069 | ABL2 | KIF1B | AQP9 | C1QB |
| ACTN1 | LOC100506801 | ACTR8 | KLF2 | BCL2A1 | C1QC |
| ACTN4 | LPCAT1 | ADAM9 | KLHL24 | C15orf48 | CCDC152 |
| ACTR3 | LPPR2 | ADAP2 | LAIR1 | CAPN2 | CCL3 |
| ADA | LPXN | ADORA3 | LAMB2 | CD300E | CD163L1 |
| ADAM19 | LSP1 | ADRB2 | LEPREL1 | CD44 | CD209 |
| ADAM8 | LST1 | AFF1 | LGALS3BP | CD48 | CD59 |
| ADD3 | LTA4H | AIG1 | LGMN | CD52 | CTSC |
| ADORA2A | LUZP6 | AKR1B1 | LHFPL2 | CD55 | CTSD |
| AGO2 | LYN | ALOX5AP | LILRB5 | CFP | DAB2 |
| AGPAT9 | LYPD3 | AMDHD2 | LIPA | CLEC12A | DNAJB1 |
| AGTPBP1 | LYST | ANKH | LMAN1 | CORO1A | EGR1 |
| AGTRAP | LYZ | ANKRD36 | | COTL1 | F13A1 |
| AHNAK | MAP2K1 | ANKRD36B | LOXL3 | CRIP1 | FOLR2 |
| AKAP2 | MAP2K3 | ANTXR1 | LPAR5 | CYTIP | FOS |
| ALDH2 | MAPK1IP1L | AP1B1 | LPAR6 | EMP3 | FRMD4A |
| ALOX5 | MAPKAPK3 | AP2A2 | LTC4S | EREG | FUCA1 |
| AMICA1 | MARCO | APOC1 | LYVE1 | FAM65B | GADD45B |
| AMPD2 | MBOAT7 | APOE | MAF | FCN1 | GPR34 |
| ANPEP | MCOLN2 | APPL2 | MAMDC2 | FGR | GYPC |
| ANXA1 | MGST1 | ARHGAP12 | ME1 | FLNA | HSPA1B |
| ANXA2 | MNDA | ARHGAP18 | MEF2C | G0S2 | IER2 |
| ANXA6 | MOB3A | ARHGAP21 | MERTK | GLIPR2 | IGF1 |
| AOAH | MPHOSPH6 | ARHGAP24 | MGAT4A | ITGAX | JUN |
| AP1S2 | MTHFS | ARMCX1 | MGAT5 | KYNU | LGMN |
| APOBEC3A | MTMR11 | ATF3 | MITF | LCP1 | LILRB5 |
| AQP9 | MTPN | ATF6 | MKNK1 | LGALS2 | MAF |
| AREG | MVP | ATP1B1 | MMD | LIMD2 | MAMDC2 |
| ARF5 | MX2 | ATP2C1 | MMP14 | LIPN | ME1 |
| ARFGAP3 | MXD1 | AXL | MMP2 | LSP1 | MERTK |
| ASGR2 | MYADM | BAG3 | MPC2 | LST1 | MRC1 |
| ATG3 | MYD88 | BAIAP2 | MRC1 | LYZ | MS4A4A |
| ATP2B1 | MYL12B | BCL2L1 | MRO | OLR1 | MS4A7 |
| B4GALT5 | MYO1G | BEX4 | MS4A4A | P2RX1 | MSR1 |
| BACH1 | NAAA | BLNK | MS4A7 | PLP2 | NRP1 |
| BASP1 | NAP1L1 | BMP2K | MSR1 | S100A10 | PLD3 |
| BCL11A | NAPSB | C1orf85 | MTMR9LP | S100A4 | PLTP |
| BCL2A1 | NBEAL2 | C1QA | MTSS1 | S100A6 | PLXDC1 |
| BCL3 | NBPF1O | C1QB | MTUS1 | S100A8 | RNASE1 |
| BID | NCF2 | C1QC | MVB12B | SERPINA1 | SEPP1 |
| BIRC3 | NDEL1 | C2 | MYLIP | SH3BGRL3 | SIGLEC1 |
| BST1 | NEDD9 | C20orf194 | MYO5A | TIMP1 | SLC40A1 |
| C11orf21 | NFAT5 | C3 | NAA20 | TKT | SLC7A8 |
| C15orf39 | NFKB1 | C5orf4 | NAIP | UPP1 | SLCO2B1 |
| C15orf48 | NOD2 | CADM1 | NASP | VCAN | STAB1 |
| C19orf38 | NOTCH2 | CARD11 | NCF4 | VDR | TMEM176B |
| C19orf59 | NOTCH2NL | CCDC152 | NCKAP5 | WARS | WLS |
| C1orf162 | NUP210 | CCL2 | NEK6 | | |
| C9orf72 | OGDH | CCL3 | NEU1 | | |
| CAMKK2 | OLR1 | CCL3L1 | NFATC2 | | |
| CAPN2 | OPTN | CCL3L3 | NFIA | | |
| CARD16 | OXSR1 | CCL4 | NGFRAP1 | | |
| CASP9 | P2RX1 | CCL4L1 | NISCH | | |
| CCDC69 | P2RY1 | CCL4L2 | NMRK1 | | |
| CCL20 | PARM1 | CCL8 | NPL | | |
| CCND3 | PCBP1 | CCND1 | NR4A2 | | |
| CCR2 | PDE4A | CD14 | NRP1 | | |
| CCR5 | PDE4D | CD163 | NRP2 | | |
| CCRN4L | PDLIM7 | CD163L1 | NUPR1 | | |
| CCT5 | PFKP | CD200R1 | NXF1 | | |
| CD101 | PGAM1 | CD209 | OLFML2B | | |
| CD1C | PGLS | CD276 | OLFML3 | | |
| CD1D | PIM3 | CD28 | P2RY13 | | |
| CD1E | PLAC8 | CD59 | P4HA1 | | |
| CD244 | PLP2 | CD81 | PCDH12 | | |
| CD300E | PNPLA8 | CD84 | PDGFA | | |
| CD37 | PPA1 | CD9 | PDGFB | | |
| CD38 | PPIF | CDKN2AIP | PDGFC | | |
| CD44 | PPP1CA | CH25H | PDIA4 | | |
| CD48 | PRELID1 | CHD7 | PDK4 | | |
| CD52 | PRKCB | CHID1 | PDPN | | |
| CD55 | PSEN1 | CITED2 | PEAK1 | | |
| CD58 | PSMA6 | CKS2 | PEBP1 | | |
| CD97 | PSMB8 | CLDN1 | PER3 | | |
| CDA | PSMB9 | CMKLR1 | PIK3IP1 | | |
| CDC42EP2 | PSME1 | CNRIP1 | PIK3R1 | | |
| CDCA4 | PSME2 | COLEC12 | PLA2G15 | | |
| CEACAM4 | PSTPIP2 | CPEB4 | PLAU | | |
| CECR1 | PTGER2 | CPED1 | PLD3 | | |
| CFP | PTGES | CPM | PLEKHG5 | | |
| CHST15 | PTP4A2 | CREB3L2 | PLTP | | |
| CKAP4 | QPCT | CREG1 | PLXDC1 | | |
| CLCF1 | RAB11FIP1 | CSGALNACT1 | PLXND1 | | |
| CLEC10A | RAB24 | CTSC | PMP22 | | |
| CLEC12A | RAB27A | CTSD | PRDM1 | | |
| CLEC4A | RAB3D | CTSL1 | PROS1 | | |
| CLEC4D | RAC2 | CXCL12 | RASGRP3 | | |
| CLEC4E | RAP1B | CXCL3 | RASSF4 | | |
| CLIP4 | RARA | CYB5R1 | RB1 | | |
| CNN2 | RASSF5 | CYBRD1 | RCAN1 | | |
| CORO1A | RHOF | CYFIP1 | RGL1 | | |
| COTL1 | RILPL2 | CYTL1 | RGPD5 | | |
| CPPED1 | RIPK2 | DAB2 | RGS1 | | |
| CRIP1 | RNF19B | DHRS3 | RGS10 | | |
| CRLF2 | RUNX3 | DHRS7 | RGS16 | | |
| CSF3R | S100A10 | DIP2C | RHOB | | |
| CSK | S100A12 | DNAJA4 | RNASE1 | | |
| CST7 | S100A4 | DNAJB1 | RND3 | | |
| CSTA | S100A6 | DNAJB4 | RNF15O | | |
| CYB5R3 | S100A8 | DOCK4 | SCARB1 | | |
| CYFIP2 | S100A9 | DPP7 | SCARB2 | | |
| CYP1B1 | SAMHD1 | DSC2 | SCD | | |
| CYP27A1 | SAMSN1 | DST | SDC3 | | |
| CYTIP | SDC4 | DTNA | SEPP1 | | |
| DAPP1 | SELL | DTNB | 11-Sep | | |
| DDX21 | SEMA6B | EBI3 | SERPING1 | | |
| DDX60L | 9-Sep | EGFL7 | SESN1 | | |
| DENND5A | SERPINA1 | EGR1 | SGK1 | | |
| DESI1 | SERPINB1 | EGR2 | SIGLEC1 | | |
| DIAPH1 | SERPINB2 | EGR3 | SIGLEC8 | | |
| DOCK5 | SERPINB8 | EIF4A2 | SLC16A10 | | |
| DYSF | SH2D3C | EMB | SLC18B1 | | |
| EAF1 | SH3BGRL3 | ENG | SLC1A3 | | |
| ECE1 | SH3BP2 | ENPP2 | SLC29A1 | | |
| EFHD2 | SHKBP1 | EPAS1 | SLC2A5 | | |
| EHD1 | SIDT2 | EPB41L2 | SLC35F6 | | |
| EIF4E2 | SIRPB1 | EPS15 | SLC36A1 | | |
| EIF6 | SLAMF7 | ERO1LB | SLC37A4 | | |
| ELF4 | SLC22A4 | ETV5 | SLC38A6 | | |
| EMP3 | SLC25A37 | F13A1 | SLC38A7 | | |
| EMR1 | SLC2A3 | FABP3 | SLC40A1 | | |
| EREG | SLC2A6 | FAM105A | SLC41A1 | | |
| EVI2B | SLC35E4 | FAM13A | SLC4A7 | | |
| FAM157B | SLC38A1 | FAM174B | SLC7A8 | | |
| FAM65B | SLC6A6 | FAM213A | SLC9A9 | | |
| FBP1 | SLC9A3R1 | FAM46A | SLCO2B1 | | |
| FCAR | SLCO4A1 | FARP1 | SMA5 | | |
| FCER1A | SNAI1 | FCGBP | SNHG12 | | |
| FCN1 | SNHG16 | FCGR1A | SNX6 | | |
| FFAR2 | SNN | FCGR1B | SORBS3 | | |
| FGR | SNX10 | FCGR1C | SPATS2L | | |
| FKBP1A | SNX20 | FCGR3A | SPIN1 | | |
| FLNA | SPATA13 | FCGR3B | SPP1 | | |
| FLT1 | SPN | FCHO2 | SPSB2 | | |
| FPR2 | SRC | FHIT | SPTLC3 | | |
| FYN | STAT6 | FMNL2 | SRGAP1 | | |
| G0S2 | STK10 | FMNL3 | ST3GAL6 | | |
| G6PD | STK17B | FOLR2 | ST6GAL1 | | |
| GALNT3 | STK38 | FOS | STAB1 | | |
| GBP5 | STX11 | FRMD4A | STMN1 | | |
| GCH1 | STXBP2 | FRMD4B | STOM | | |
| GK | SUB1 | FSCN1 | SWAP70 | | |
| GLIPR2 | SULF2 | FUCA1 | TBC1D9B | | |
| GMFG | SYAP1 | GAA | TCEAL3 | | |
| GPCPD1 | SYTL1 | GADD45B | TCF12 | | |
| GPR132 | TAGLN2 | GAL3ST4 | TCF4 | | |
| GPR35 | TBC1D7 | GAS6 | TEX14 | | |
| GPSM3 | TBC1D8 | GATM | TGFBR1 | | |
| GST01 | TCIRG1 | GGTA1P | TLR1 | | |
| GSTP1 | TES | GIMAP5 | TLR7 | | |
| GTPBP1 | TESC | GNPDA1 | TM6SF1 | | |
| H2AFY | TET2 | GOLIM4 | TMEM176A | | |
| H3F3A | TGM2 | GPNMB | TMEM176B | | |
| HCK | THBS1 | GPR155 | TMEM198B | | |
| HCST | TICAM1 | GPR34 | TMEM2 | | |
| HIGD2A | TIMP1 | GSN | TMEM37 | | |
| HK3 | TKT | GYPC | TMEM86A | | |
| HLA-F | TMEM120A | HADH | TNF | | |
| HMGA1 | TMEM71 | HERC2P2 | TNFRSF21 | | |
| ICAM2 | TNFAIP6 | HERPUD1 | TNS3 | | |
| ICAM3 | TNFRSF1B | HES1 | TP53I11 | | |
| IFITM1 | TNFSF14 | HEXA | TPCN1 | | |
| IL18R1 | TNIP1 | HGF | TREM2 | | |
| IL1R1 | TNIP2 | HIST2H2BF | TRPM2 | | |
| IL1R2 | TNIP3 | HLA-DOA | TSPAN15 | | |
| IL1RN | TP53BP2 | HMOX1 | TSPAN4 | | |
| IL2RG | TRA2A | HNMT | TTYH3 | | |
| IL3RA | TRAF1 | HPGDS | ULK3 | | |
| IMPDH1 | TRAF3IP3 | HRH1 | USP53 | | |
| IQSEC1 | TREM1 | HSD17B14 | VAT1 | | |
| IRAK3 | TRIM25 | HSPA1A | VSIG4 | | |
| IRG1 | TRMT6 | HSPA1B | WBP5 | | |
| ISG20 | TSPAN32 | HSPB1 | WDR91 | | |
| ITGA5 | TUBA1A | HSPH1 | WFS1 | | |
| ITGAL | TUBA4A | HTRA1 | WLS | | |
| ITGAX | TYMP | ICA1 | ZFP36L1 | | |
| ITGB2-AS1 | UBE2D1 | IDH1 | ZFP36L2 | | |
| KCNN4 | UBE2J1 | IER2 | ZNF812 | | |
| KCTD20 | UBXN11 | IER5 | LOC100505702 | | |
| KMO | UPP1 | IFI16 | | | |
| KYNU | UXT | IFITM10 | | | |
| LCP1 | VAMP5 | IGF1 | | | |
| LDLR | VCAN | IGFBP4 | | | |
| LDLRAD3 | VCL | IGSF21 | | | |
| LGALS1 | VDR | IL2RA | | | |
| LGALS2 | VNN2 | ING1 | | | |
| LGALS3 | VRK2 | ISCU | | | |
| LILRA1 | WARS | ISYNA1 | | | |
| LILRA2 | WDR1 | ITGAV | | | |
| LILRA3 | ZAK | ITGB5 | | | |
| LILRA5 | ZC3H12A | ITPR2 | | | |
| LILRA6 | ZDHHC20 | ITSN1 | | | |
| LILRB2 | ZFC3H1 | JKAMP | | | |
| LILRB3 | ZYX | JUN | | | |

### Evidence of Antitumor Immune Activity Despite Low Immune Infiltration

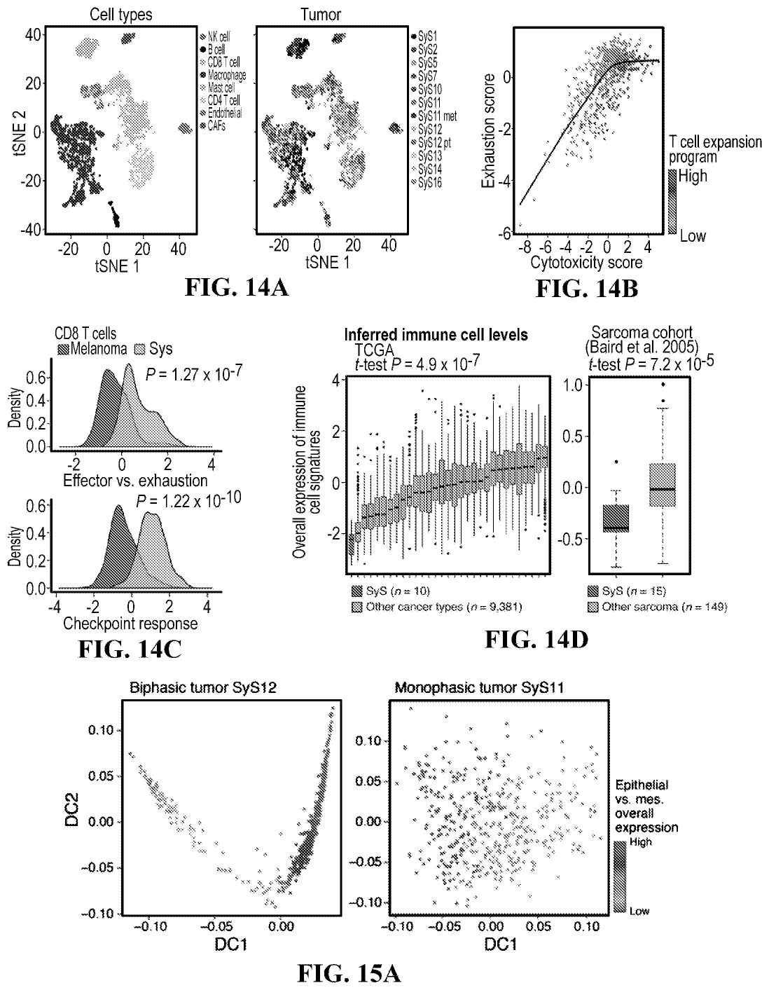

The lack of effective antitumor immunity in SyS may results from: either the inactivity of immune cells, limiting their recognition of or response to SyS malignant cells, or hampered immune cell infiltration and recruitment into the tumor parenchyma. To test the first possibility, Applicants examined CD8 T cell states (FIG. 14A, Table 1F), and found clear hallmarks of antitumor immunity and recognition. T cell subsets span naïve, cytotoxic, exhausted, and regulatory T cells (FIG. 14B; METHODS), with evidence of expansion based on TCR reconstruction (31) (showing 57 clones, all patient-specific, with shared clones between matched samples from the same patient). While cytotoxic and exhaustion markers were generally co-expressed in T cells (FIG. 14B, consistent with previous reports (29)), clonally expanded T cells had unique transcriptional features (Methods, Table 12), suggestive of an effector-like non-exhausted state (FIG. 14B, P<6.6*10⁻¹², mixed-effects). These expanded T cells might respond to SyS-specific CTAs, which were specifically expressed in large fractions of the malignant cell populations (FIG. 4A). Moreover, CD8 T cells in SyS have features suggesting they are even more active than those in melanoma tumors, where anti-tumor immunity is relatively pronounced. First, compared to CD8 T cells from melanoma (32), CD8 T cells in SyS tumors overexpressed a program characterizing T cells in melanoma tumors that were responsive to immune checkpoint blockade (33) (FIG. 14C bottom, P=1.22*10⁻¹⁰, mixed-effects). In addition, compared to melanoma CD8 T cells, the SyS CD8 T cells also overexpressed effector and cytotoxic gene modules (34, 35) (e.g., GZMB, CX3CR1, P=6.36*10⁻⁹, mixed-effects), and repressed exhaustion markers (P=6.36*10⁻³, mixed-effects), including LAYN (34), and multiple checkpoint genes (CTLA4, HAVCR2, LAGS, PDCD1, and TIGIT; P=7.69*10⁻⁷, mixed-effects, FIG. 14C, top).

Other immune cells in the tumor microenvironment also showed features of antitumor immunity. Macrophages span M1-like and M2-like states, suggestive of both pro- and anti-inflammatory properties, respectively (FIG. 10A-10C; Methods, Table 12), and expressed relatively high levels of TNF (P=1.13*10⁻⁷, mixed-effects, >4 fold more compared to melanoma macrophages). However, mastocytes show regulatory features, with 39% of them expressing PD-L1 (as opposed to only 2% PD-L1 expressing malignant cells).

Applicants next examined the alternative hypothesis that T cell abundance might be a limiting factor in SyS, despite these favorable T cell states. Applicants compared SyS to 30 other cancer and sarcoma types. SyS tumors showed extremely low levels of immune cells, which cannot be explained by variation in the mutational load (FIG. 14D; P=2.58*10⁻¹¹, mixed effects when conditioning on the tumor mutational load), and despite the malignant-cell specific expression of immunogenic CTAs (FIG. 3C). In addition, unlike melanoma (FIG. 10D, left), T cell levels were not correlated with prognosis in SyS (FIG. 10D, right), indicating that they may not cross the critical threshold to impact clinical outcomes. Only mastocytes had a moderate positive association with improved prognosis (P=0.012, Cox regression). These findings suggest that the lack of proper immune cell recruitment and infiltration is a key immune evasion mechanism in SyS, potentially mediated by the SyS cells.

Among CD8 T cells, TCR reconstruction (Stubbington et al. Nat Methods 13:329-332 (2016)) identified 57 clones, all patient-specific (with 6 shared clones between the primary and metastatic lesions of patient S11, and 7 shared clones between the pre- and post-treatment samples of patient S12). Clonally expanded T cells had unique features that Applicants characterized with an expansion program (Methods, Table 12). Interestingly, while cytotoxic and exhaustion markers were generally co-expressed (FIG. 9D, consistent with previous reports (Tirosh et al. Science 352:189-196 (2016)), the expansion program was particularly high in non-exhausted and highly cytotoxic T cells (FIG. 9D, P<6.6*10−12, mixed-effects). It was also associated with non-exhausted cytotoxic T cells in hepatocellular carcinoma (Puram et al. Cell 171:1611-1624.e24 (2017)) and melanoma (Jerby-Arnon et al. Cell 175:984-997.e24 (2018)) (P<4.89*10−19, mixed effects).

To further evaluate CD8 T cells in SyS, Applicants compared them to T cells from melanoma tumors (Jerby-Arnon et al. Cell 175:984-997.e24 (2018)) where anti-tumor immunity is relatively pronounced. In comparison to melanoma, CD8 T cells in SyS tumors overexpressed a program that was recently found to characterize T cells in tumors responsive to immune checkpoint blockade (Sade-Feldman et al. Cell 175:998-1013.e20 (2018)) (FIG. 9E). The SyS T cells also overexpressed effector and cytotoxic gene modules (Zheng et al. Cell 169:1342-1356.e16 (2017); Böttcher et al. Nat Comun 6:8306 (2015)) (e.g., GZMB, CX3CR1, P=6.36*10−9, mixed-effects), and repressed exhaustion markers (P=6.36*10−3, mixed-effects), including LAYN (Zheng et al. Cell 169:1342-1356.e16 (2017)), and multiple checkpoint genes (CTLA4, HAVCR2, LAG3, PDCD1, and TIGIT; P=7.69*10−7, mixed-effects, FIG. 9E). These findings suggest that T cells in SyS tumors have a cytotoxic potential, which might be unleashed by immune checkpoint blockade.

Further analyses demonstrated that despite these favorable T cell states, T cell abundance might be a limiting factor in SyS. Comparing SyS tumors to 30 other cancer and sarcoma types demonstrated that SyS tumors have extremely low levels of immune cells, beyond those expected by their relatively low mutational load (FIG. 9F; P=2.58*10−11, mixed effects when conditioning on the tumor mutational load). In addition, unlike melanoma (FIG. 10D), T cell levels were not correlated with prognosis in SyS (FIG. 10D), and only mastocytes had a moderate positive association with prognosis (P=0.012, Cox regression).

### Example 7—HDAC and CDK4/6 Inhibitors Synergistically Repress the Immune Resistant Features of Synovial Sarcoma Cells

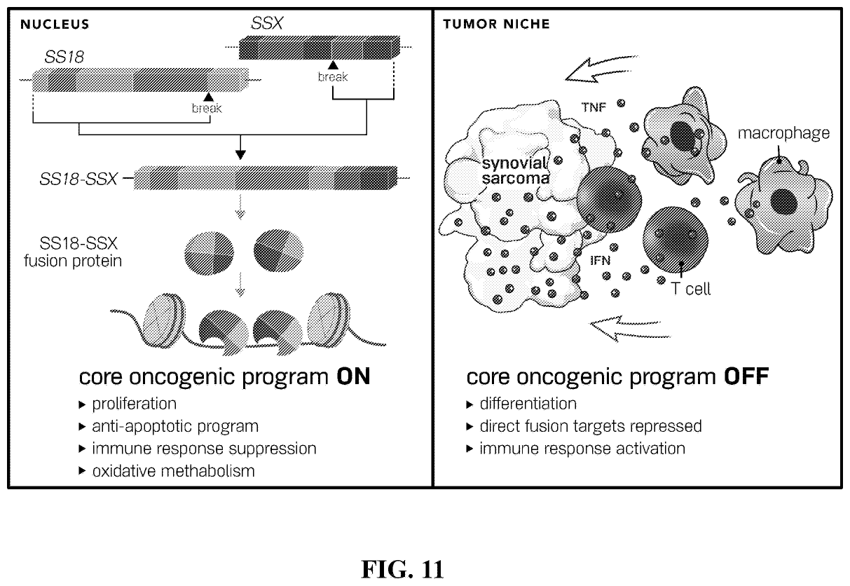

Given the aggressive features of the core oncogenic program, its association with poor clinical outcome and T cell exclusion, and its dependency on the oncoprotein expression, Applicants set to identify pharmacological interventions that could block the program, aiming to selectively target synovial sarcoma cells. Here Applicants describe: (1) the computational model that led to the selection of HDAC/CDK inhibitors, (2) the results of the ongoing experiments, hopefully confirming predictions (FIGS. 11A-11C).

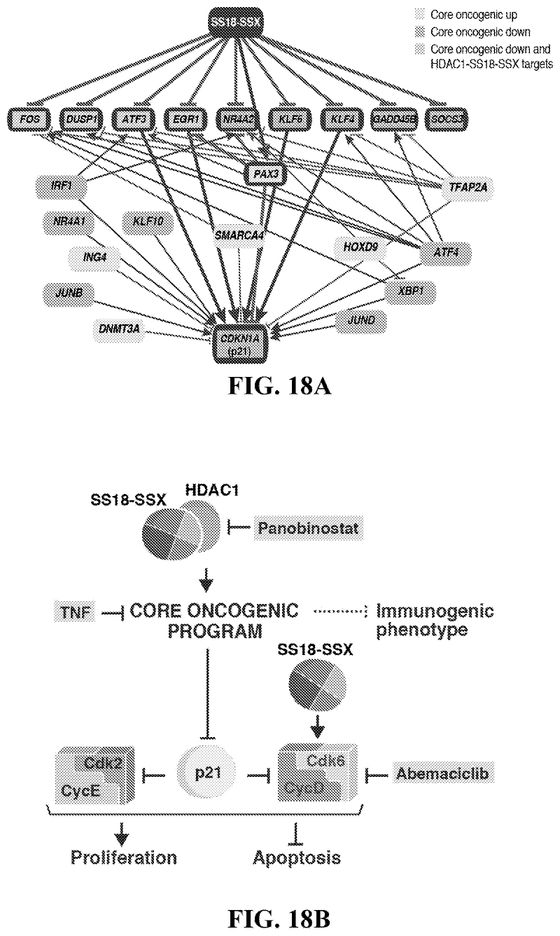

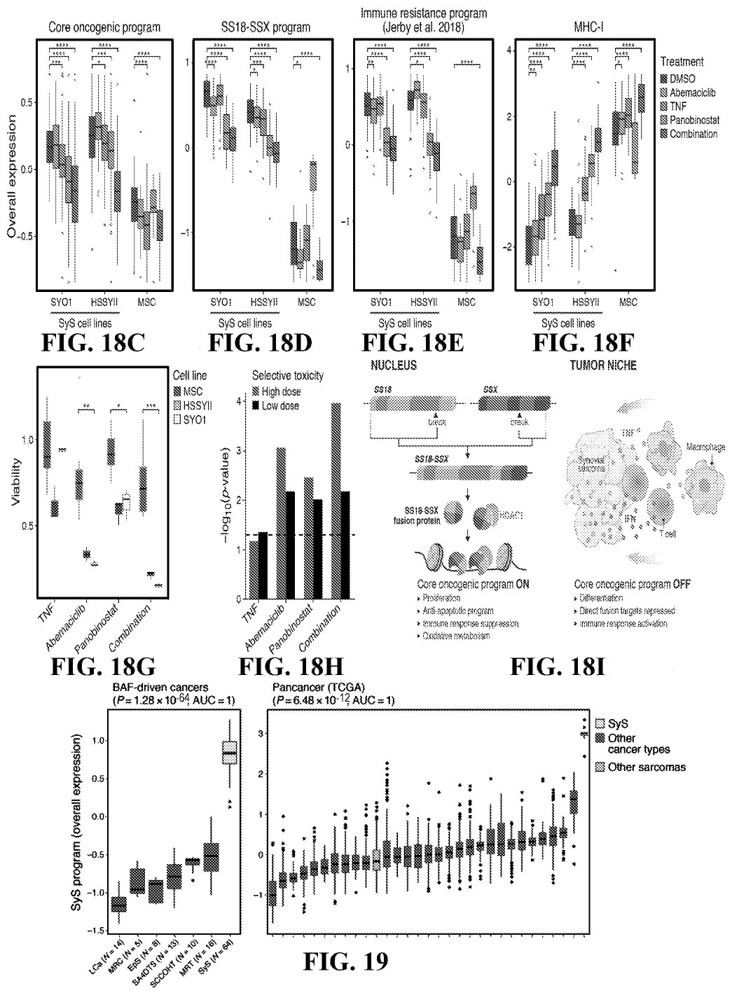

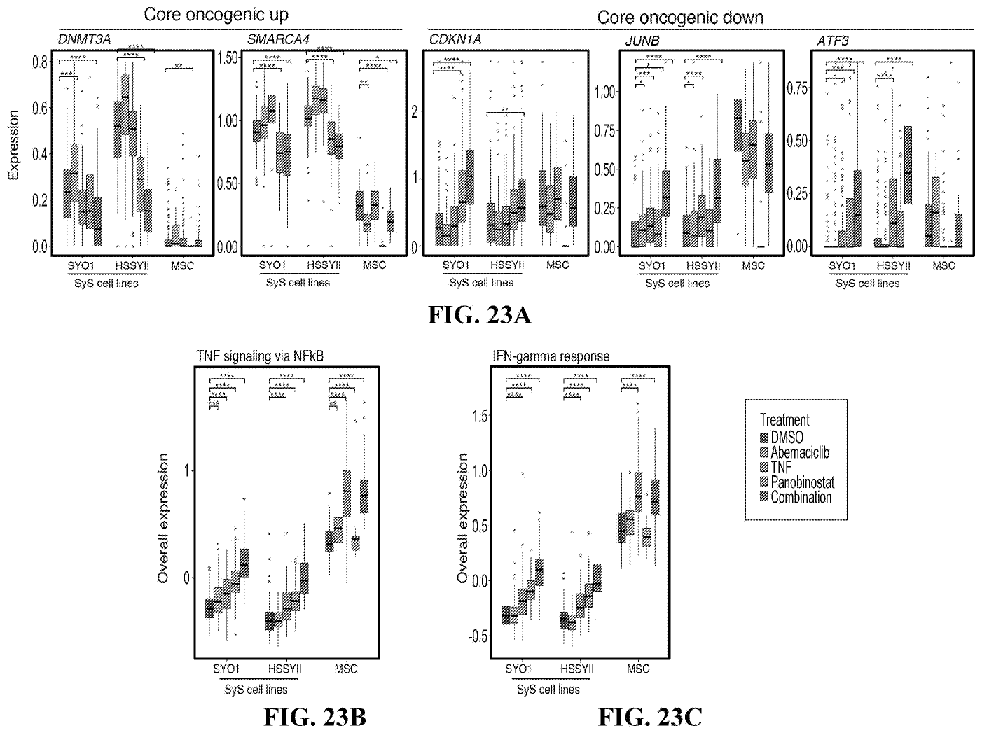

Applicants examined whether pharmacological agents could potentially repress the core oncogenic program and induce more immunogenic cell states in SyS cells. Computational modeling of the core oncogenic regulatory network (METHODS) highlighted the SSX-SS18-HDAC1 complex (20) as the program's master regulator (FIG. 18A), and the tumor suppressor CDKN1A (p21) as its most repressed target. The latter indicates that the core oncogenic program is regulating, rather than regulated by, cell cycle genes through the p21-CDK2/4/6 axis, potentially reinforcing the direct induction of cyclin D and CDK6 by SS18-SSX (FIG. 18B). According to this model (FIG. 18B), modulators of cell cycle (e.g., CDK4/6 inhibitors) and SS18-SSX (e.g., HDAC inhibitors) could synergistically target the immune resistance features of SyS cells, especially in the presence of tumor microenvironment cytokines as TNF. To test these predictions, Applicants treated SyS lines and primary mesenchymal stem cells (MSCs) with low doses of HDAC and CDK4/6 inhibitors, in order to avoid global toxicity-related effects, and examined their impact on the transcriptional state of the cells. As predicted, the HDAC inhibitor panobinostat markedly repressed the core oncogenic program (P=3.34*10⁻¹⁴, mixed-effects; FIG. 18C) and selectively induced CDKN1A in SyS cells (P=2.13*10⁻⁸) (FIG. 23A). Panobinostat also repressed the SS18-SSX program (P=5.32*10⁻⁷²; FIG. 18D), decreased the expression of cell cycle genes (P<1.78*10⁻²⁰), and induced an immunogenic phenotype (32) with enhanced antigen presentation and IFNγ responses (P<9.53*10⁻³¹; FIGS. 18E, 18F, FIG. 23B, 23CC). The CDK4/6 inhibitor abemaciclib repressed cell cycle gene expression (P=3.63*10⁻⁸), without impacting the core oncogenic program (P>0.1; FIG. 18C), supporting the notion that cell cycle regulation is down-stream of the core oncogenic program. Lastly, a low dose combination of panobinostat, abemaciclib and TNF synergistically repressed the core oncogenic program (P=1.72*10⁻³⁷, FIG. 18C, FIG. 23A) and multiple immune resistant features, while inducing antigen presentation, IFN responses, and induced-self antigens as MICA/B (P=3.12*10⁻⁷⁶; FIG. 18E, 18F, 23B, 23C). It also repressed MIF (Macrophage Migration Inhibitory Factor), a member of the core oncogenic and SS18-SSX programs, which has been previously shown to hamper T cell recruitment into the tumor (40). The effect of the drug combination on these programs and genes in viable SyS cells significantly exceeded the expected additive effect (P<0.01, mixed-effects interaction term, METHODS), and could potentially help both T cells (MHC-1) and NK cells (MICA/B) bind to and eliminate SyS cell. Consistent with the transcriptional changes, the drug combination displayed a significantly higher detrimental effect on the SyS cells compared to primary MSCs (P=5.7*10⁻¹³; FIG. 18F, 18G).

### Discussion

Here, Applicants describe mapping of malignant and immune cell states and interactions in human SyS tumors, through integrative analyses of clinical and functional data. By leveraging scRNA-Seq Applicants mapped cell states in human SyS tumors, revealing active antitumor immunity in this relatively cold tumor, alongside malignant cellular plasticity and immune excluding features, centered around a core oncogenic program—a yet unappreciated cell modality that captures intra- and inter-tumor heterogeneity and is associated with aggressive disease (FIG. 11).

This program is regulated by the tumor's primary genetic driver and may hamper proper immune recruitment and infiltration. Nonetheless, immune cells can impact the malignant cells through TNF and IFNγ secretion, counteracting the transcriptional alterations induced by the oncoprotein. Targeting the oncogenic program and its downstream effects with HDAC and CDK4/6 inhibitors induced cell autonomous immune responses, repressed immune resistant features, and was selectively detrimental to SyS cells, thus providing a basis for the development of specific therapeutic strategies, which are currently lacking.

The findings demonstrate that different cancer hallmarks are co-regulated in SyS. The associations between the different malignant programs identified (FIG. 4A) demonstrate the connection between stem-like properties, cellular proliferation and the core oncogenic program (FIGS. 4C, 4D). In accordance with this, the repression of SS18-SSX blocked the core oncogenic program, arrested cell cycle and triggered cellular differentiation, suggesting that these three cellular features are co-regulated by the oncoprotein. The core oncogenic program itself couples different aggressive cellular characteristics, and associates aerobic metabolism with the repression of immune responses.

The metabolic features of the core oncogenic program may also impact the tumor microenvironment. Supporting this notion, recent studies have shown that malignant cells use oxidative phosphorylation to create a hypoxic niche and promote T cell dysfunction (41). These metabolic features might reflect the conserved role of the SWI/SNF complex in regulating carbon metabolism and sucrose non-fermenting phenotypes in the yeast Saccharomyces cerevisiae (42). These connections might generalize to other cancer types, as mutations in the BAF complex have been recently shown to induce a targetable dependency on oxidative phosphorylation in lung cancer (43).

Despite the extremely cold phenotypes displayed by SyS (FIG. 14D), expanded effector T cells are present in SyS tumors (FIGS. 14B-C), potentially responding to the CTAs expressed specifically in the malignant cells, including NY-ESO-1 and PRAME (FIG. 3C). Consistently, vaccines triggering dendritic-cells to prime NY-ESO-1 specific T cells can lead to durable responses in SyS patients (7), further supporting the notion that SyS immune evasion operates primarily through impaired T cell or dendritic cell recruitment (44). The latter may also be mediated through Wnt/β-catenin signaling pathway, which has been previously shown to interfere with CD8 T cell recruitment to tumors by dendritic cells (44), and is indeed active in all the malignant SyS cells and directly induced by SS18-SSX (FIG. 22A, Tables 5, 8). The core oncogenic program itself includes several CTAs, linking between malignant immune evasion and testicular immune privileges.

The analyses demonstrate that SyS tumors manifest extremely cold phenotypes, despite the overexpression of several cancer-testis antigens (FIG. 3C) and antitumor T cell reactivity (FIGS. 9D-9E). Indicating that the malignant cells may promote this cold phenotype, the core oncogenic program overlaps a transcriptional program that was previously linked to T cell exclusion in melanoma (Jerby-Arnon et al. Cell 175:984-997.e24 (2018)). In addition, Applicants found that Wnt/β-catenin signaling, which has been shown to drive T cell exclusion in mouse models (Spranger et al. Nature 523:231-235 (2015)), is directly upregulated by S S18-SSX (FIG. 7E) and is activated in all the SyS cells in the tumor (Tables 4 and 5). Further studies are needed to examine the underlying mechanisms of the different cancer-immune associations mapped here. Such mechanisms might be relevant in other cancer types given the role of PBAF genes in determining immune checkpoint blockade responses in melanoma and renal cancer (Pan et al. Science 359:770-775 (2018); Miao et al. Science 359:801-806 (2018)).

The association between the core oncogenic program and T cell exclusion is observed in situ in the SyS samples from Applicants' single-cell cohort. Applicants measured in situ expression of 12 proteins across 4,310,120 cells in 9 samples using multiplexed immunofluorescence (t-CyCIF) (39) (FIGS. 4E,F; METHODS), and profiled the in situ expression of 1,412 genes in 24 spatially distinct areas in two samples using the GeoMx high plex RNA Assay (early version for Next-Generation Sequencing; METHODS). Both approaches showed that CD45⁺ immune cells were exceptionally low in SyS (<0.4%, compared to >8.7% in melanoma samples (32)). Moreover, the malignant cells in the more immune infiltrated areas show a marked decrease in the core oncogenic program (r=−0.53, P=6.9*10⁻³, Pearson correlation, and P<1*10⁻¹⁰, mixed effects; METHODS). This suggests that the status of the malignant cells and the composition of the tumor microenvironment might be interconnected in SyS.

The findings also demonstrate that immune resistance, metabolic processes, cell cycle and de-differentiation are tightly co-regulated in SyS. Thus, beyond the targeted cytotoxicity of the adoptive immune system, CD8 T cells and macrophages may alleviate some of the aggressive features of SyS cells through the secretion of TNF and IFNγ, also impacting malignant cells with repressed antigen presentation or unrecognized antigens.

While the core oncogenic program shares some similar features with a T cell exclusion program we recently identified in melanoma (Jerby-Arnon et al., 2018), there are also substantial distinctions between the two programs, and >90% do not overlap between the two, likely reflecting the dramatic differences in driving events, cell of origin and tissue environment of the two tumors. This emphasizes the importance of understanding immune evasion for each tumor context. In particular, unlike the melanoma program, the core oncogenic program highlights a metabolic shift and is strongly connected to the genetic driver. In SyS tumors (but not in melanoma) Applicants successfully decoupled, through computational inference, the intrinsic and extrinsic signals which modulate this transcriptional program, facilitating the reconstruction of multicellular circuits. This new approach revealed a bi-directional interaction between malignant and immune cells where CD8 T cells and macrophages can in turn repress the core oncogenic program through the secretion of TNF and IFNγ. Thus, beyond their direct cytotoxic activity, immune cells can alleviate some of the aggressive features of SyS cells through cytokine secretion, targeting also malignant cells with repressed antigen presentation or unrecognized epitopes.

The tight co-regulation of processes indicate targeted therapies may be able to sensitize the tumor to immune surveillance. Supporting this notion, Applicants demonstrate that the combined inhibition of HDAC and CDK4/6, two known repressors of SS18-SSX (45, 46) and cellular proliferation (47), respectively, trigger immunogenic cell states even at sub-cytotoxic doses. This combinatorial treatment is also selectively cytotoxic to SyS cells, consistent with previous reports where HDAC and CDK4/6 inhibitors were used separately to induce cell death in SyS (45, 47). The basal antitumor immune response reported, and the ability of T cells and macrophages to repress the core oncogenic and SS18-SSX programs support the potential of exploiting HDAC and CDK4/6 inhibitors together with immunotherapy.

The epithelial and mesenchymal programs defined here might also be relevant in other cancer settings, given the role of the epithelial to mesenchymal transition (EMT) in drug resistance and metastatic disease. Interestingly, Applicants found a strong connection between TNF and IFNγ responses and the epithelial program (FIG. 12A, P<8.49*10−6, hypergeometric test), suggesting that EMT may also promote immune evasion capabilities, as previously suggested (Datar et al. Clin Cancer Res 22:3422 (2016); Terry et al. Mol Oncol 11:824-846 (2017)).

The programs identified by Applicants are tightly linked to clinical outcomes. While additional prospective data are needed to further examine their predictive value, the results shown here demonstrate that the overall expression of the programs in bulk tumors could be used for patient stratification. Alternatively, specific genes within the programs could potentially be used as biomarkers. For example, ALDH1A1 is a stem-cell marker which is among the top genes in the core oncogenic program. Its protein levels have been previously shown to be predictive of poor prognosis and metastatic disease in SyS patients (Zhou et al. Oncol Rep 37:3351-3360 (2017)).

Taken together, this study comprehensively maps and interrogates cell states in SyS, along with their regulatory circuits and clinical implications. Applicants demonstrated that the SS18-SSX oncoprotein and the tumor microenvironment coordinately shape cell states in SyS, setting the basis for the development of more effective treatment strategies.

Applicants demonstrated that the SS18-SSX oncoprotein and the tumor microenvironment coordinately shape cell states in SyS, resulting in the establishment of an immune privileged environment (FIG. 18I). The possibility to selectively target the underlying mechanisms to reverse immune evasion offers a new perspective for the clinical management of SyS, and potentially other malignancies driven by similar genetic events.

### Materials and Methods

Human Tumor Specimen Collection and Dissociation

Patients at Massachusetts General Hospital and University Hospital of Lausanne were consented preoperatively in all cases according to their respective Institutional Review Boards (protocol numbers: CER-VD 260/15, DF/HCC 13-416). Fresh tumors were collected directly from the operating room at the time of surgery and presence of malignancy was confirmed by frozen section. Tumor tissues were mechanically and enzymatically dissociated using a human tumor dissociation kit (Miltenyi Biotec, Cat. No. 130-095-929), following the manufacturers recommendations. Clinical annotations are provided in Table 1.

Tissue Handling and Tumor Disaggregation

Resected tumors were transported in DMEM (ThermoFisher Scientific, Waltham, Mass.) on ice immediately after surgical procurement. Tumors were rinsed with PBS (Life Technologies, Carlsbad, Calif.). A small fragment was stored in RNA-Protect (Qiagen, Hilden, Germany) for bulk RNA and DNA isolation. Using scalpels, the remainder of the tumor was minced into tiny cubes <1 mm3 and transferred into a 50 ml conical tube (BD Falcon, Franklin Lakes, N.J.) containing 10 ml pre-warmed M199-media (ThermoFisher Scientific), 2 mg/ml collagenase P (Roche, Basel, Switzerland) and 10 U/μl DNase I (Roche). Tumor pieces were digested in this media for 10 minutes at 37° C., then vortexed for 10 seconds and pipetted up and down for 1 minute using pipettes of descending sizes (25 ml, 10 ml and 5 ml). As needed, this was repeated twice more until a single-cell suspension was obtained. This suspension was then filtered using a 70 μm nylon mesh (ThermoFisher Scientific) and residual cell clumps were discarded. The suspension was supplemented with 30 ml PBS (Life Technologies) with 2% fetal calf serum (FCS) (Gemini Bioproducts, West Sacramento, Calif.) and immediately placed on ice. After centrifuging at 580 g at 4° C. for 6 minutes, the supernatant was discarded and the cell pellet was re-suspended in PBS with 1% FCS and placed on ice prior to staining for FACS.

Fluorescence-Activated Cell Sorting (FACS)

Tumor cells were kept in Phosphate Buffered Saline with 1% bovine serum albumin (PBS/BSA) while staining. Cells were stained using calcein AM (Life Technologies) and TO-PRO-3 iodide (Life Technologies) to identify viable cells. For all tumors, Applicants used CD45-VioBlue (human antibody, clone REA747, Miltenyi Biotec) to identify immune cells and in few cases, Applicants also used CD3-PE to specifically identify lymphocytes (human antibody, clone BW264/56, Miltenyi Biotec). For all the samples, Applicants used unstained cells as control. Standard, strict forward scatter height versus area criteria were used to discriminate doublets and gate only single cells. Viable single cells were identified as calcein AM positive and TO-PRO-3 negative. Sorting was performed with the FACS Aria Fusion Special Order System (Becton Dickinson) using 488 nm (calcein AM, 530/30 filter), 640 nm (TO-PRO-3, 670/14 filter), 405 nm (CD45-VioBlue, 450/50 filter) and 561 nm (PE, 586/15 filter) lasers. Applicants sorted individual, viable, immune and non-immune single cells into 96-well plates containing TCL buffer (Qiagen) with 1% beta-mercaptoethanol. Plates were snap frozen on dry ice right after sorting and stored at −80° C. prior to whole transcriptome amplification, library preparation and sequencing.

Library Construction and Sequencing

For plate-based scRNA-seq, Whole transcriptome amplification was performed using the Smart-seq2 protocol (Picelli et al Nat Protoc 9:171-181 (2014)), with some modifications as previously described (Tirosh et al. Nature 539, 309-313 (2016); Venteicher et al. Science. 355 (2017), doi:10.1126/science.aai8478; Fisher et al. Genome Biol. 12, R1 (2011)). The Nextera XT Library Prep kit (Illumina) with custom barcode adapters (sequences available upon request) was used for library preparation. Libraries from 384 to 768 cells with unique barcodes were combined and sequenced using a NextSeq 500 sequencer (Illumina).

In addition to SMART-seq2, cells from three samples (SS12pT, SS13 and SS14) were also sequenced using droplet-based scRNA-Seq with the 10× genomics platform. The samples were partitioned for SMART-seq2 and 10× genomics after dissociation. For each tumor, approximately two thirds of the sample was used for SMART-seq2 and one third for droplet based scRNA-seq (10× genomics). Applicants sorted viable cells using MACS (Dead Cell Removal Kit, Miltenyi Biotec) and ran up to 2 channels per sample with a targeted number of cell recovery of 2,000 cells per channel. The samples were processed using the 10× Genomics Chromium 3′ Gene Expression Solution (version 2) based on manufacturer instructions and sequenced using a NextSeq 500 sequencer (Illumina).

Whole Exome Sequencing (WES)

DNA and RNA were extracted from fresh frozen tissue or Formalin-Fixed Paraffin-Embedded (FFPE) blocks for each patient (obtained according to their respective Institutional Review Board—approved protocols) using the AllPrep DNA/RNA extraction kit (Qiagen). Applicants used tumor tissue and matched normal muscle tissue from the same patient as reference. Library construction was performed as previously described (Fisher et al. Genome Biol. 12, R1 (2011)), with the following modifications: initial genomic DNA input into shearing was reduced from 3 μg to 20-250 ng in 50 μL of solution. For adapter ligation, Illumina paired end adapters were replaced with palindromic forked adapters, purchased from Integrated DNA Technologies, with unique dual-indexed molecular barcode sequences to facilitate downstream pooling. Kapa HyperPrep reagents in 96-reaction kit format were used for end repair/A-tailing, adapter ligation, and library enrichment PCR. In addition, during the post-enrichment SPRI cleanup, elution volume was reduced to 30 μL to maximize library concentration, and a vortexing step was added to maximize the amount of template eluted. After library construction, libraries were pooled into groups of up to 96 samples. Hybridization and capture were performed using the relevant components of Illumina's Nextera Exome Kit and following the manufacturer's suggested protocol, with the following exceptions: first, all libraries within a library construction plate were pooled prior to hybridization. Second, the Midi plate from Illumina's Nextera Exome Kit was replaced with a skirted PCR plate to facilitate automation. All hybridization and capture steps were automated on the Agilent Bravo liquid handling system. After post-capture enrichment, library pools were quantified using qPCR (automated assay on the Agilent Bravo), using a kit purchased from KAPA Biosystems with probes specific to the ends of the adapters. Based on qPCR quantification, libraries were normalized to 2 nM. Cluster amplification of DNA libraries was performed according to the manufacturer's protocol (Illumina) using exclusion amplification chemistry and flowcells. Flowcells were sequenced utilizing Sequencing-by-Synthesis chemistry. The flowcells are then analyzed using RTA v.2.7.3 or later. Each pool of whole exome libraries was sequenced on paired 76 cycle runs with two 8 cycle index reads across the number of lanes needed to meet coverage for all libraries in the pool.

### RNA In Situ Hybridization

Paraffin-embedded tissue sections from human tumors from Massachusetts General Hospital and and University Hospital of Lausanne were obtained according to their respective Institutional Review Board-approved protocols. Sections were mounted on glass slides and stored at −80° C. Slides were stained using the RNAscope 2.5 HD Duplex Detection Kit (Advanced Cell Technologies, Cat. No. 322430), as previously described (2, 3, 6): slides were baked for 1 hour at 60° C., deparaffinized and dehydrated with xylene and ethanol. The tissue was pretreated with RNAscope Hydrogen Peroxide (Cat. No. 322335) for 10 minutes at room temperature and RNAscope Target Retrieval Reagent (Cat. No. 322000) for 15 minutes at 98° C. RNAscope Protease Plus (Cat. No. 322331) was then applied to the tissue for 30 minutes at 40° C. Hybridization probes were prepared by diluting the C2 probe (red) 1:50 into the C1 probe (green). Advanced Cell Technologies RNAscope Target Probes used included Hs-EGR1 (Cat. No. 457671-C2) and Hs-IGF2 (Cat. No. 594361). Probes were added to the tissue and hybridized for 2 hours at 40° C. A series of 10 amplification steps was performed using instructions and reagents provided in the RNAscope 2.5 HD Duplex Detection Kit. Tissue was counterstained with Gill's hematoxylin for 25 seconds at room temperature followed by mounting with VectaMount mounting media (Vector Laboratories).

### In Situ Immunofluorescence Imaging

Formalin-fixed, paraffin-embedded (FFPE) tissue slides, 5 μm in thickness, were generated at the at the Massachusetts General Hospital from tissue blocks collected from patients under IRB-approved protocols (DF/HCC 13-416). Multiplexed, tissue cyclic immunofluorescence (t-CyCIF) was performed as described recently (5). For direct immunofluorescence, Applicants used the following antibodies (manufacturer, clone, dilution): c-Jun-Alexa-488 (Abcam, Clone E254, 1:200), CD45-PE (R&D, Clone 2D1, 1:150), p21-Alexa-647 (CST, Clone 12D1, 1:200), Hes1-Alexa-488 (Abcam, Clone EPR4226, 1:500), FoxP3-Alexa-570 (eBioscience, Clone 236A/E7, 1:150), NF-κB (Abcam, Clone E379, 1:200), E-Cadherin-Alexa-488 (CST, Clone 24E10, 1:400), pRB-Alexa-555 (CST, Clone D20B12, 1:300), COXIV-Alexa-647 (CST, Clone 3E11, 1:300), β-catenin-Alexa-488 (CST, Clone L54E2, 1:400), HSP90-PE (Abcam, polyclonal, lot #GR3201402-2, 1:500) and vimentin-Alexa-647 (CST, Clone D21H3, 1:200). Stained slides from each round oft-CyCIF were imaged with a CyteFinder slide scanning fluorescence microscope (RareCyte Inc. Seattle Wash.) using either a 10× (NA=0.3) or 40× long-working distance objective (NA=0.6). Imager5 software (RareCyte Inc.) was used to sequentially scan the region of interest in 4 fluorescence channels. Image processing, background subtraction, image registration, single-cell segmentation and quantification were performed as previously described (Lin et al. eLife. 7 (2018), doi:10.7554/eLife.31657).

### RNA Profiling In Situ Hybridization (ISH)

DNA oligo probes were designed to bind mRNA targets. From 5′ to 3′, they each comprised of a 35-50 nt target complementary sequence, a UV photocleavable linker, and a 66 nt indexing oligo sequence containing a unique molecular identifier (UMI), RNA ID sequence, and primer binding sites. Up to 10 RNA detection probes were designed per target mRNA. RNA detection probes were provided by Nanostring Technologies. To perform the ISH, 5 um FFPE tissue sections from two patients were mounted on positively charged histology slides. Sections were baked at 65 C for 45 minutes in a HybEZ II hybridization oven (Advanced Cell Diagnostics, INC.), Slides were deparaffinized using Citrsolv (Decon Labs, Inc., 1601) rehydrated in an ethanol gradient, and washed in 1× phosphate-buffered saline pH 7.4 (PBS: Invitrogen, AM9625). Slides were incubated for 15 minutes in 1× Tris-EDTA pH 9.0 buffer (Sigma Aldrich, SRE0063) at 100 C with low pressure in a TintoRetriever Pressure cooker (bioSB 7008). Slides were washed then incubated in 1 ug/mL proteinase K (Thermo Fisher Scientific, Inc., AM2546) in PBS for 15 minutes at 37° C. and washed again in PBS. Tissues were then fixed in 10% neutral-buffered formalin (Thermo Fisher Scientific, 15740) for 5 minutes, incubated in NBF stop buffer (0.1M Tris Base, 0.1M Glycine, Sigma) for 5 minutes twice, then washed for 5 minutes in PBS. Tissues were then incubated overnight at 37° C. with GeoMx™ RNA detection probes in Buffer R (Nanostring Technologies) using a Hyb EZ II hybridization oven (Advanced cell Diagnostics, Inc). During incubation, slides were covered with HybriSlip Hybridization Covers (Grace BioLabs, 714022). Following incubation, HybriSlip covers were gently removed and 25-minute stringent washes were performed twice in 50% formamide and 2×SSC at 37° C. Tissues were washed for 5 minutes in 2×SSC then blocked in Buffer W (Nanostring Technologies) for 30 minutes at room temperature in a humidity chamber. 500 nM Syto13 and antibodies targeting PanCK and CD45 (Nanostring technologies) in Buffer W were applied to each section for 1 hour at room temperature. Slides were washed twice in fresh 2×SSC then loaded on the GeoMx™ Digital Spatial Profiler (DSP) (7). In brief, entire slides were imaged at 20× magnification and 12 circular regions of interest (ROI) with 200-300 μm diameter were selected per sample. The DSP then exposed ROIs to 385 nm light (UV) releasing the indexing oligos and collecting them with a microcapillary. Indexing oligos were then deposited in a 96-well plate for subsequent processing. The indexing oligos were dried down overnight and resuspended in 10 μL of DEPC-treated water.

Sequencing libraries were generated by PCR from the photo-released indexing oligos and ROI-specific Illumina adapter sequences and unique i5 and i7 sample indices were added. Each PCR reaction used 4 μL of indexing oligos, 1 μL of indexing PCR primers, 2 μL of Nanostring 5×PCR Master Mix, and 3 μL PCR-grade water. Thermocycling conditions were 37° C. for 30 min, 50° C. for 10 min, 95° C. for 3 min; 18 cycles of 95° C. for 15 sec, 65° C. for 1 min, 68° C. for 30 sec; and 68° C. 5 min. PCR reactions were pooled and purified twice using AMPure XP beads (Beckman Coulter, A63881) according to manufacturer's protocol. Pooled libraries were sequenced at 2×75 base pairs and with the single-index workflow on an Illumina NextSeq to generate 458M raw reads.

### Primary Cell Cultures and Cell Lines

Human primary synovial sarcoma (SyS) spherogenic cultures (SScul1, SScul2 and SScul3) were derived from patients undergoing surgery at Massachusetts General Hospital and University Hospital of Lausanne according to their respective Institutional Review Board-approved protocols. Directly after dissociation (as above), the dissociated bulk tumor cells were put in culture and were grown as spheres using ultra-low attachment cell culture flasks in IMDM 80% (Gibco, Cat. No. 1244053), KnockOut Serum Replacement 20% (Gibco, Cat. No. 10828028), Recombinant Human EGF Protein 10 ng/mL (R&D systems, Cat. No. 236-EG-200), Recombinant Human FGF basic, 145 aa (TC Grade) Protein long/mL (R&D systems, Cat. No. 4114-TC-01M) and Penicillin-Streptomycin (Gibco, Cat. No. 15140122). Cells were expanded by mechanical and enzymatic dissociation every week using TrypLE Express Enzyme (ThermoFisher, Cat. No. 12605010).

The SyS cell lines used in the SS18-SSX KD experiments, and the functional drug assays include: Aska, a generous gift from Kazuyuki Itoh, Norifumi Naka, and Satoshi Takenaka (Osaka University, Japan), and SYO1, a generous gift from Akira Kawai (National Cancer Center Hospital, Japan), and HS-SY-II (purchased from RIKEN Bio Resource Center, 3-1-1 Koyadai, Tsukuba, Ibaraki 305-0074, Japan). All three cell lines were cultured using standard protocols in DMEM medium (Gibco) supplemented with 10-20% fetal bovine serum, 1% Glutamax (Gibco), 1% Sodium Pyruvate (Gibco) and 1% Penicillin-Streptomycin (Gibco) and grown in a humidified incubator at 37° C. with 5% CO₂.

Human primary pediatric Mesenchymal Stem Cells (MSCs) were isolated from healthy donors undergoing corrective surgery in agreement with the Institutional Review Board-approved protocol of the University Hospital of Lausanne (Protocol number 2017-0100). Samples were deidentified prior to culture and analysis. Cells were expanded in 90% IMDM (Gibco, Cat. No. 1244053) containing 10% Fetal Bovine Serum (Gibco), 1% Penicillin-Streptomycin (Gibco) and long/mL Platelet-Derived Growth Factor BB (PDGF-BB, PeproTech) as previously described.

### SS18-SSX Knockdown in Aska and SYO1 Cell Lines

The SyS cell lines Aska and SYO1 were cultured using standard protocols in DMEM medium (Gibco) supplemented with 10-20% fetal bovine serum, 1% Glutamax (Gibco), 1% Sodium Pyruvate (Gibco) and 1% Penicillin-Streptomycin (Gibco) and grown in a humidified incubator at 37° C. with 5% CO2. Cells expressing a pLKO.1 vector with a scrambled shRNA hairpin control (5′-CCTAAGGTTAAGTCGCCCTCGCTCGAGCGAGGGCGACTTAAC CTTAGG-3′) (SEQ ID NO: 5) or a shSSX hairpin targeting SSX of the SS18-SSX fusion (5′-CAGTCACTGACAGTTAATAAA-3′) (SEQ ID NO: 6) were prepared by lentiviral infection. In brief, lentivirus was prepared by transfection of HEK293T cells with gene delivery vector and the packaging vectors pspax2 and pMD2.G, filtration of media followed by ultracentrifugation, and then resuspension of viral pellet in PBS. Aska and SYO1 cells were infected with lentivirus for 48 hours and then underwent 5 days of selection with puromycin (2 μg/mL) prior to collection for single cell RNA-seq analysis.

### In Vitro IFN/TNF Experiment

Cells were dissociated 12 hours before adding the drugs at the concentrations indicated directly to the growing media and cells were collected at different time point (ranging from 4 hours to 4 days) for SMART-seq2. Viability was determined by CellTiter-Glo Luminescent Cell Viability Assay (Promega) after 5 to 7 days of treatment. TNF-alpha (Miltenyi Biotec, Human TNF-α, Cat. No. 130-094-014) IFN-gamma (R&D systems, Recombinant Human IFN-gamma Protein, Cat. No. 285-IF-100) were suspended in deionized sterile-filtered water.

### In Vitro Drug Assay and Cell Proliferation Measurements

For the functional drug assay, 200,000 SYO-1 cells and HSSYII cells, and 100,000 MSCs were seeded in 60×15 mm plates (Falcon). Cells were stimulated for five days with the following compounds: 100 or 200 nM Abemaciclib (Selleckchem, U.S.A.), 15 or 30 ng/ml TNF (Miltenyi Biotech, Germany) or a combination of the two. Compounds were refreshed at days three and four, and the solvent (DMSO) was used as control. At day 4, 12.5 or 25 nM Panobinostat (Selleckchem, U.S.A.) was added to the cultures, and the cells were harvested 24 hours later for proliferation scoring. To assessment cellular proliferation, cells were detached with trypsin, washed in PBS, and re-suspended in 1 ml of complete medium. After diluting 1:2 with Trypan blue (Invitrogen) viable cells were counted using the Automated Cell Counter Countess II FL (Thermo Fisher Scientific). Each experimental condition was measured in triplicate.

### Computational Analysis Methods

scRNA-Seq Pre-Processing and Gene Expression Quantification

BAM files were converted to merged, demultiplexed FASTQ files. The paired-end reads obtained with SMART-Seq2 were mapped to the UCSC hg19 human transcriptome using Bowtie (9), and transcript-per-million (TPM) values were calculated with RSEM v1.2.8 in paired-end mode (10). The paired-end reads obtained with droplet scRNA-Seq (10× Genomics) were mapped to the UCSC hg19 human transcriptome using STAR (11), and gene counts/TPM values were obtained using CellRanger (cellranger-2.1.0, 10× Genomics).

For bulk RNA-Seq data, expression levels were quantified as E=log 2(TPM+1). For scRNA-seq data, expression levels were quantified as E=log 2(TPMi,j/10+1). TPM values were divided by 10 because the complexity of the single-cell libraries is estimated to be within the order of 100,000 transcripts. The 10-1 factoring prevents counting each transcript ˜10 times and overestimating the differences between positive and zero TPM values. The average expression of a gene i across a population of N cells, denoted here as P, was defined as

$${E}_{i , p} = {log}_{2} ( 1 + \frac{{Σ}_{j ∈ P} T P {M}_{i , j}}{N} )$$

For each cell, Applicants quantified the number of genes with at least one mapped read, and the average expression level of a curated list of housekeeping genes (Tirosh et al. Science. 352, 189-196 (2016)). Applicants excluded all cells with either fewer than 1,700 detected genes or an average housekeeping expression (E, as defined above) below 3 (Table 2B). For the remaining cells, Applicants calculated the average expression of each gene (Ep), and excluded genes with an average expression below 4, which defined a different set of genes in different analyses depending on the subset of cells included. In cases where Applicants analyzed different cell subsets together, genes were removed only if they had an average Ep below 4 in each of the different cell subsets included in the analysis. Different cell types and malignant cells from different tumors were considered as different cell subsets in this regard.

### WES Data Pre-Processing

BAM file was produced with the Picard pipeline (sourceforge.net/), which aligns the tumor and normal sequences to the hg19 human genome build using Illumina sequencing reads. The BAM was uploaded into the Firehose pipeline (broadinstitute.org/cancer/cga/Firehose). Quality control modules within Firehose were applied to all sequencing data for comparison of the origin for tumor and normal genotypes and to assess fingerprinting concordance. Cross-contamination of samples was estimated using ContEst (13).

### Somatic Alteration Assessment

MuTect (14) was applied to identify somatic single-nucleotide variants. Indelocator (broadinstitute.org/cancer/cga/indelocator), Strelka (15), and MuTect2 (broadinstitute.org/gatk/documentation/tooldocs/current/org_broadinstitute_gatk_tools_walkers_cancer_m2_MuTect2) were applied to identify small insertions or deletions. A voting scheme was used with inferred indels requiring a call by at least 2 out of 3 algorithms.

Artifacts introduced by DNA oxidation during sequencing were computationally removed using a filter-based method (16). In the analysis of primary tumors that are formalin-fixed, paraffin-embedded samples (FFPE) Applicants further applied a filter to remove FFPE-related artifacts (17). Reads around mutated sites were realigned with Novoalign (www.novocraft.com/products/novoalign/) to filter out false positive that are due to regions of low reliability in the reads alignment. At the last step, Applicants filtered mutations that are present in a comprehensive WES panel of 8,334 normal samples (using the Agilent technology for WES capture) aiming to filter either germline sites or recurrent artifactual sites. Applicants further used a smaller WES panel of 355 normal samples that are based on Illumina technology for WES capture, and another panel of 140 normal samples sequenced without Applicants' cohort (18) to further capture possible batch-specific artifacts. Annotation of identified variants was done using Oncotator (19) (broadinstitute.org/cancer/cga/oncotator).

### Copy Number and Copy Ratio Analysis

To infer somatic copy number from WES, Applicants used ReCapSeg (on gatk forums available at broadinstitute.org/categories/recapseg-documentation), calculating proportional coverage for each target region (i.e., reads in the target/total reads) followed by segment normalization using the median coverage in a panel of normal samples. The resulting copy ratios were segmented using the circular binary segmentation algorithm (20). To infer allele-specific copy ratios, Applicants mapped all germline heterozygous sites in the germline normal sample using GATK Haplotype Caller (21) and then evaluated the read counts at the germline heterozygous sites in order to assess the copy profile of each homologous chromosome. The allele-specific copy profiles were segmented to produce allele specific copy ratios.

### Gene Sets Overall Expression

Applicants used the following scheme to compute the overall expression (OE) of a gene set, namely, a signature. The OE metric filters technical variation and highlights biologically meaningful patterns. The procedure is based on the notion that the measured expression of a specific gene is correlated with its true expression (signal), but also contains a technical (noise) component. The latter may be due to various stochastic processes in the capture and amplification of the gene's transcripts, sample quality, as well as variation in sequencing depth. OE of a gene signature is computed in a way that accounts for the variation in the signal-to-noise ratio across genes and cells.

Given a gene signature and a gene expression matrix E (as defined above), Applicants first binned the genes into 50 expression bins according to their average expression across the cells or samples. The average expression of a gene across a set of cells within a sample is Ei,p (see: scRNA-seq pre-processing and gene expression quantification) and the average expression of a gene across a set of N tumor samples was defined as:

$${𝔼}_{j} [ {E}_{ij} ] = {Σ}_{j} \frac{{E}_{ij}}{N} .$$

Given a gene signature S that consists of K genes, with kb genes in bin b, Applicants sample random S-compatible signatures for normalization. A random signature is S-compatible with signature S if it consists of overall K genes, such that in each bin b it has exactly kb genes. The OE of signature Sin cell or sample j is then defined as:

$$O {E}_{j} = \frac{{Σ}_{i ∈ S} {C}_{ij}}{{𝔼}_{\overline{S}} [ {Σ}_{i ∈ \overline{S}} {C}_{ij} ]}$$

where \tilde{S} is a random S-compatible signature, and Cij is the centered expression of gene i in cell or sample j, defined as Cij=Eij−E[Eij]. Because the computation is based on the centered gene expression matrix C, genes that generally have a higher expression compared to other genes will not skew or dominate the signal. Applicants found that 100 random S-compatible signatures are sufficient to yield a robust estimate of the expected value [symbol] _{\tilde{S}}[Σ_{i∈\tilde{S}}Cᵢⱼ]. The distribution of the OE values was normal or a mixture of normal distributions, facilitating subsequent analyses.

The term transcriptional program (e.g., the core oncogenic program) is used to denote cell states defined by a pair of signatures, such that one (S-up) is overexpressed and the other (S-down) is underexpressed. The OE of a program is then the OE of S-up minus the OE of S-down.

In cases where the OE of a given signature has a bimodal distribution across the cell population, it can be used to naturally separate the cells into two subsets. To this end, Applicants applied the Expectation Maximization (EM) algorithm for mixtures of normal distributions to define the two underlying normal distributions. Applicants then assigned cells to the two subsets, depending on the distribution (high or low) that they were assigned to. Applicants use the term a transcriptional program (e.g., the core oncogenic program) to characterize cell states which are defined by a pair of signatures, such that one (S-up) is overexpressed and the other (S-down) is underexpressed. Applicants define the OE of the program as the OE of S-up minus the OE of S-down.

### Cell Type Assignments

Cell type assignments were performed on the basis of genetic and transcriptional features, according to the four analyses described below.

(1) Fusion detection. Fusion detection was performed with STAR-Fusion (Haas et al. bioRxiv (2017), doi:10.1101/120295), to detect any transcript that indicates the fusion of two genes.

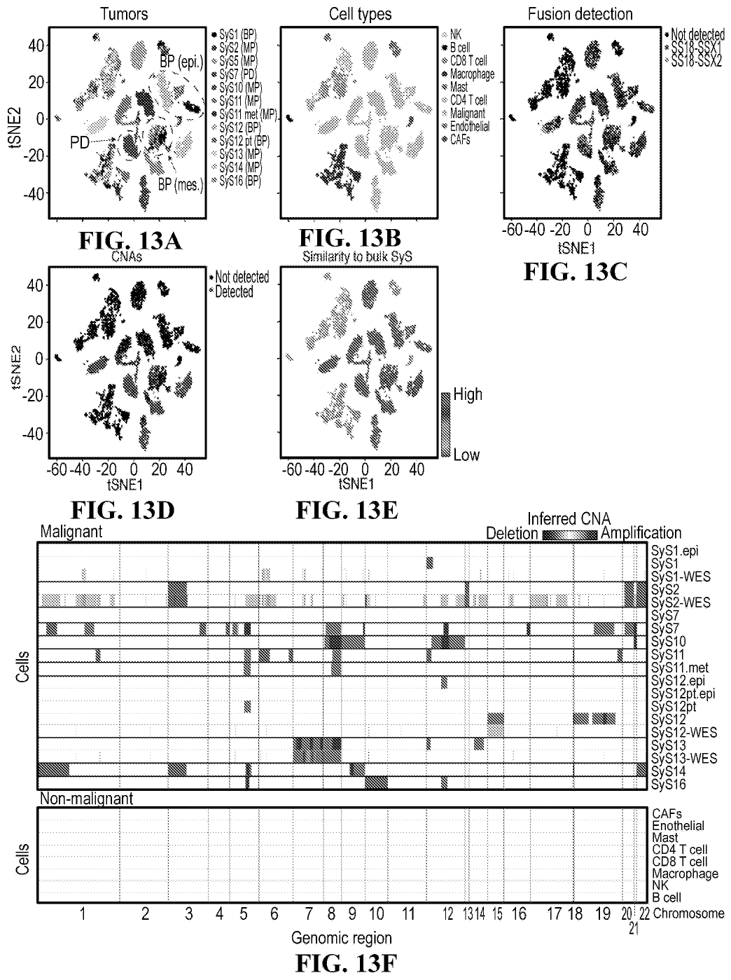

(2) Copy Number Alterations (CNA) inference. To infer CNAs from the scRNA-seq data Applicants used the approach described in (Tirosh et al. Science. 352, 189-196 (2016)) as implemented in the R code provided in github.com/broadinstitute/inferCNA with the default parameters. To identify malignant cells based on CNA patterns, Applicants defined the overall CAN level of a given cell as the sum of the absolute CNA estimates across all genomic windows. Within each tumor, Applicants identified CD45− cells with the highest overall CNA level (top 10%), and considered their average CNA profile as the CAN profile of the pertaining tumor. For each cell Applicants then computed a CNA-R-score, that is, the Spearman correlation coefficient obtained when comparing its CNA profile to the CNA profile of its tumor. Cells with a high CNAV-R-score (greater than the 25% of the CD45− cell population) were considered as malignant according to the CNA criterion. As certain tumors/malignant cells have a stable genome, Applicants did not use the CNA criterion to identify non-malignant cells. Large-scale CNAs were visualized (FIG. 13F) using a Bayesian approach, as described in github.com/broadinstitute/infercnv/wiki/infercnv-i6-HMM-type.

(3) Differential similarity to bulk tumors. Applicants compared the scRNA-Seq profiles to those of bulk sarcoma tumors (Abeshouse et al. Cell. 171, 950-965.e28 (2017)). RNA-Seq of bulk sarcoma tumors was downloaded from TCGA (xena.ucsc.edu). For each cell in Applicants' scRNA-Seq cohort Applicants: (1) computed the spearman correlation between its expression profile and the expression profiles of the bulk sarcoma tumors, and (2) examined if the rs coefficients obtained when comparing the cell to SyS tumors were higher compared to those obtained when comparing the cell to non-SyS sarcoma tumors, using a one-sided Wilcoxon ranksum test. Cells with a ranksum p-value <0.05 were considered as potentially malignant, and as potentially non-malignant otherwise.

(4) Transcription-based clustering. Applicants clustered the cells by applying a shared nearest neighbor (SNN) modularity optimization algorithm (Waltman et al. Eur Phys J B. 86 (2013), doi:10.1140/epjb/e2013-40829-0), as implemented in the Seurat R package. First Principle Component Analysis (PCA) was preformed and k-nearest neighbors were calculated to construct the SNN graph. The latter was used to identify clusters that optimize the modularity function. Next, Applicants assigned clusters to cell types. Clusters where the majority of cells had the SS18-SSX1/2 fusion were considered malignant clusters. Non-malignant clusters were assigned to cell types by computing the overall expression of well-established cell type markers across the non-malignant cells (Tables 4 and 5). The OE of each of these cell type signature had a bimodal distribution across the cell population. Applying Expectation Maximization (EM) algorithm for mixtures of normal distributions, Applicants defined the two underlying normal distributions, and assigned cells to cell types. Each non-malignant cluster was enriched with cells of a particular cell type, and was assigned to the pertaining cell type.

Applicants used these four converging criteria to assign the cells to nine cell subss: malignant cells, epithelial cells, CAFs, CD8 and CD4 T cells, B cells, NK cells, macrophages, and mastocytes. More specifically, a cell was classified as malignant if it was CD45- and classified as malignant according to analyses (3) and (4) above. A cell was classified as non-malignant if it was classified as non-malignant according to analyses (1), (3)-(4) above. Non-malignant cells were then further assigned to cell types based on their cluster assignment. Cells with inconsistent assignments were removed from further analyses. Lastly, within malignant cells Applicants identified epithelial cells by clustering each of the biphasic tumors into two clusters.

Cell type assignments were preformed separately for the Smart-Seq2 cohort and the 10× Genomics (Zheng et al. Nat. Commun. 8, 14049 (2017)) cohort, such that fusion detection was used only in the former, where full length transcripts were sequenced.

### Malignant Epithelial and Mesenchymal Differentiation Programs

First, Applicants performed intra-tumor analyses to identify differentially expressed genes when comparing the epithelial malignant cells to the mesenchymal malignant cells. Applicants performed this analysis for each of the three biphasic tumor samples (S1, and S12 pre- and post-treatment). The fourth biphasic tumor (S16) was not included in this analysis as its sample did not include epithelial malignant cells. Genes that were overexpressed in the epithelial cells compared to the mesenchymal cells in all three samples were defined as epithelial genes, and likewise for mesenchymal genes. When using these signatures in the analysis of bulk gene expression profiles Applicants removed genes that were included in the non-malignant cell type signatures.

Using these signatures Applicants defined: (1) the epithelial vs. mesenchymal differentiation score as the OE of the epithelial signature minus the OE of the mesenchymal signature, and (2) the differentiation score as the OE of the epithelial signature plus the OE of the mesenchymal signature.

### Cell Type Signatures

Cell type signatures were generated based on pairwise comparisons between identified cell subtypes: malignant cells, epithelial cells, CAFs, CD8 and CD4 T cells, B cells, NK cells, macrophages, and mastocytes. For each pair of cell subtypes Applicants identified differentially expressed genes using the likelihood-ratio test (26), as implemented in the Seurat package (satijalab.org/seurat). Genes were considered as cell type specific if they were overexpressed in a particular cell subtype compared to all other cell subtypes (log-fold change >0.25 and p-value <0.05, following Bonferroni correction). Applicants defined a general T cell signature for both CD4 and CD8 cells by identifying genes that were overexpressed in both CD4 and CD8 compared to all other (non T) cells. A more permissive version of this generic T cell signature includes genes which were overexpressed in CD4 or CD8 T cells compared to all other (non T) cells.

### Inferring Tumor Composition

Tumor composition was assessed based on the Overall Expression of the different cell type specific signatures Applicants identified from the scRNA-seq data (Table 5). For example, the CD8 T cell signature was used to infer the level of CD8 T cells in the tumor, and likewise for other cell types. To estimate tumor purity Applicants used the malignant SyS signature identified here (Table 5), which consists of genes that are exclusively expressed by malignant SyS cells compared to non-malignant cells in SyS tumors.

To evaluate the performance of this approach, Applicants simulated 200 bulk RNA-Seq profiles. For each simulated bulk RNA-Seq profile we: (1) randomly chose one of the tumors in the cohort; (2) sampled 100 cells from different cell types profiled in this tumor—these cells include a mix of immune, stroma and malignant cells, at a randomly chosen composition; (3) summed the scRNA-Seq profiles of this randomly chosen population (P) of 100 cells, such that the bulk expression of

gene i across this population was defined as

$${E}_{i , P} = {log}_{2} ( 1 + \frac{{Σ}_{j ∈ P} T P {M}_{i , j}}{100} )$$

Applicants also used cell type signatures Applicants previously derived from melanoma scRNA-Seq data (22) to predict the tumor composition of the simulated SyS bulk RNA-Seq profiles, and vice versa. Applicants then compared the predictions to the known cell type composition. The predicted composition was highly correlated with the known composition (r>0.9, P<1*10⁻³⁰, Spearman correlation) for all cell types.

### Multilevel Mixed-Effects Models

To examine the association between two cell features, denoted here as x and y, across different patients or experiments Applicants used multilevel mixed-effects regression models (random intercepts models). The models include patient/experiment-specific intercepts to control for the dependency between the scRNA-seq profiles of cells that were obtained from the same patient/experiment. The models also control for data quality by providing the number of reads (log-transformed) that were detected in each cell as a covariate. To compute the association between features x and y Applicants provided x as another covariate and used y as the dependent variable. The models were implemented using the lme4 and lmerTest R packages (CRAN.R-project.org/package=lme4, CRAN.R-project.org/package=lmerTest).

For example, to test if malignant cycling cells were more frequent in treatment naïve samples, Applicants used a logistic mixed-effects model as described above. The dependent variable y was the cycling status of the malignant cells. The independent covariate x was a binary variable denoting if the sample was obtained before or after treatment. Only malignant cells were included in this model.

### T Cell Receptor (TCR) Reconstruction and T Cell Expansion Program

TCR reconstruction was performed using TraCeR (27), with the Python package in github.com/Teichlab/tracer. To characterize the transcriptional state of clonally expanded T cells, Applicants first identified the clonality level of the T cells in Applicants' cohort. T cell that were obtained from tumors with a larger number of T cells with reconstructed TCRs were more likely to be

defined as expanded. To control for this confounder Applicants performed the following down-sampling procedure. First, Applicants removed T cells without a reconstructed alpha or beta TCR chain, and samples with less than 20 T cells with a reconstructed TCR. Next, Applicants computed the probability that a given cell will be a part of a clone when subsampling 20 T cells from each tumor. T cells with a high probability to be a part of a clone (above the median) were considered expanded, and non-expanded otherwise. To identify the genes differentially expressed in expanded CD8 T cells Applicants used mixed-effects models with a binary covariate, denoting if the cell was classified as expanded or not.

### CD8 T Cell Analyses

The analysis of T cell exhaustion vs. T cell cytotoxicity was performed as previously described (12), with the exhaustion signature provided in (12). First, Applicants computed the cytotoxicity and exhaustion scores of each CD8 T cell. Next, to control for the association between the expression of exhaustion and cytotoxicity markers, Applicants estimated the relationship between the cytotoxicity and exhaustion scores using locally-weighted polynomial regression (LOWESS, black line in FIG. 2B). Based on these values Applicants classified the CD8 T cells into four groups: Cells with a low cytotoxicity score (below the 25^{th} percentile) were classified as naïve or memory-like cells, while the others were considered effector or exhausted if their cytotoxicity scores were significantly higher or lower than expected given their exhaustion scores, respectively. According to this classification, Applicants examined if the clonal expansion program was higher in the effector-like cells. In addition, Applicants compared the SyS CD8 T cells to CD8 T cells from human melanoma tumors (22) using mixed-effects models with a sample-level covariate denoting if the sample was obtained from a SyS or melanoma tumor.

### Malignant Epithelial and Mesenchymal Differentiation Programs

The epithelial and mesenchymal signatures were obtained through intra-tumor differential expression analysis, using the likelihood-ratio test for single cell gene expression (26), as implemented in the Seurat package (satijalab.org/seurat). Applicants compared the mesenchymal to epithelial cells in each of the three biphasic tumor samples (SyS1, SyS12 and SyS12pt). The tumor SyS16 was not included in this analysis (although it was annotated as partially biphasic according to its histology), because its scRNA-Seq sample did not include any epithelial malignant cells. Genes that were up-regulated in the epithelial cells compared to the mesenchymal cells in all three samples were defined as epithelial genes, and likewise for mesenchymal genes. When using the epithelial and mesenchymal signatures in the analysis of bulk gene expression Applicants removed from these signatures those genes that are also part of non-malignant cell type signatures.

Using these signatures Applicants defined: (1) the epithelial vs. mesenchymal differentiation score as the OE of the epithelial signature minus the OE of the mesenchymal signature, and (2) the differentiation score as the OE of the epithelial signature plus the OE of the mesenchymal signature. An alternative way to define the differentiation score of a particular cell is first to assign it to the epithelial or mesenchymal subset, and then use only the pertaining signature to estimate its differentiation level. However, this approach will not distinguish between poorly-differentiated mesenchymal cells, and mesenchymal cells which have begun to transition to an epithelial state. Hence, Applicants used the inclusive definition of differentiation.

Based on the genes in the epithelial and mesenchymal signatures Applicants then generated diffusion maps (28) for each one of the tumors in the cohort, using the density R package (bioconductor.org/packages/release/bioc/html/destiny) with the default parameters.

### Identifying Co-Regulated Gene Modules

To identify co-regulated gene modules that capture intra-tumor heterogeneity Applicants analyzed each tumor separately. To identify patterns that explain the cell-cell variation both in epithelial and in mesenchymal malignant cells, Applicants further divided the biphasic samples (SyS1, SyS12, and SyS12pt) to their epithelial and mesenchymal compartments. Applicants used PAGODA (29) as implemented in github.com/hms-dbmi/scde to filter technical variation and identify co-regulated gene modules in each sample. To identify genes that were repeatedly co-regulated Applicants then constructed a gene-gene co-regulation graph. In this graph, an edge between two genes denotes that the two genes appeared together in the same gene module in at least five samples. Next, Applicants identified dense clusters in the graph using the Newman-Girvan (30) community clustering as previously implemented (31). Applicants filtered out small gene clusters (<20 genes). Lastly, for each gene cluster Applicants identified the opposing gene module by identifying genes that were negatively correlated with its Overall Expression (OE) across the malignant cells. Correlation was computed using partial Spearman correlation, when controlling for the number of genes and (log-transformed) reads detected per cells, and correcting for multiple hypotheses testing using the Benjamini-Hochberg procedure (32).

For comparison Applicants applied another complementary approach, LIGER (33), which identifies repeating gene modules in the malignant cells using integrative non-negative matrix factorization (NMF) (34). Integrative NMF learns a low-dimensional space, where cells are defined by one set of dataset-specific factors (denoted as Vᵢ), and another set of shared factors (denoted as W). Each factor, or metagene, represents a distinct pattern of gene co-regulation. To find these metagenesit

solves the following optimization problem

argmin_{H}_{ᵢ}_{,V}_{ᵢ}_{,W≥0}Σᵢ∥Eᵢ−Hᵢ(W+Vᵢ)∥_{F}²+λΣᵢ∥HᵢVᵢ∥_{F}²

Where Eᵢ denotes the expression matrix (log-transformed TPM) of the malignant cells in sample i, Vᵢ denotes sample-specific metagenes and W denotes the shared metagenes across all samples. For this analysis, each biphasic tumor was again split to two “samples”, of epithelial and mesenchymal cells. Applicants used the top 100 genes of each metagene in Was the iNMF signatures, and then computed the overall expression of these signatures in the malignant cells. The resulting signatures and their expression across the malignant cells matched the signatures identified with the PCA-based approach, and specifically the core-oncogenic program was re-discovered (FIG. 21A).

### Quantifying RNA Velocity

Estimates of RNA velocity were computed using the Velocyto toolkit (velocyto.org/). Applicants applied Velocyto with the default parameters, using a gene-relative model. To explore the potential transitions between the epithelial and mesenchymal cell states and avoid confounders, Applicants used only the genes from these differentiation programs (Table 6) for the analysis.

### Predicting Patient Prognosis

To test if a given program predicts metastasis free-survival or overall survival, Applicants first computed the OE of the program in each tumor based on the bulk gene expression data. Next, Applicants used a Cox regression model with censored data to compute the significance of the association between the expression values and survival. To visualize the predictions of a specific signature in a Kaplan Meier (KM) plot, Applicants stratified the patients into three groups according to the program expression: high or low expression correspond to the top or bottom 20% of the population, respectively, and intermediate otherwise. Applicants used a log-rank test to examine if there was a significant difference between the survival rates of the three patient groups.

### Analysis of In Situ Immunofluorescence Imaging

Immune cells were detected based on the protein level of CD45 (>7.5 log-transformed). Malignant cells were identified based on histological morphology, and high protein levels of Hes1. High protein expression was detected by applying the EM algorithm for mixtures of normal distributions. The core oncogenic program score was computed only in the malignant cells based the combined expression of its repressed protein markers: Hsp90, p21, NFkB, and cJun (minus sum of centered log-transformed values). Each image—corresponding to a specific sample in the scRNA-Seq cohort—was divided to frames of 100 cells. The average expression of the core oncogenic program in the malignant cells and the fraction of immune cells in each frame was computed. Using these frame-level values Applicants examined the association between the expression of the core oncogenic program in the malignant cells and the fraction of the immune cells, using a mixed-effects model, with a sample-level intercept (see Multilevel mixed-effects models). The mixed-effect model accounts for the nested structure of the data (frames are associated with samples), and ensures the pattern repeatedly appears across different samples.

### Analysis of In Situ RNA Profiling

FASTQ files from multiple lanes were merged to generate single files for processing and insure proper removal of PCR duplicates later in the pipeline. Illumina adapter sequences were trimmed using Trim Galore (version 0.4.5) with a minimum base pair overlap stringency of four bases and a base quality threshold of 20. Paired end reads were stitched using Paired-End reAd mergeR (PEAR, version 0.9.10) specifying a minimum stitched read length of 24 bp and a maximum stitched read length of 28 bp. The 14 bp UMI sequence was extracted from the stitched FASTQ files from the 5′ end of the sequence reads using umi tools (version 0.5.3). The FASTQ files with extracted UMIs were then aligned to a genome containing the 12 bp reference sequence tags using bowtie2 (version 2.3.4.1) in end-to-end mode with a seed length of four. Using a custom python

function, the generated SAM files were split into multiple SAM files based on the tag to which they aligned to limit memory usage when removing PCR duplicates. The split SAM files were converted to bam files, sorted, and index using samtools (version 1.9) with the import, sort, and index options respectively. PCR duplicates were removed from the sorted and indexed bam files using the dedup command from umi tools with an edit distance threshold of three. An edit distance threshold of three was used. Using custom python functions, the SAM files with PCR duplicates removed were merged for each sample and used to generate digital counts of the tags.

Outlier counts were removed before generating a consensus count for each target. Outlier tags were identified as those with counts 90% below the mean of the probe group in at least 20% of the ROIs analyzed and removed them from the analysis. Subsequently, Applicants removed tags from the analysis if they were flagged as outliers in at least 20% of the AOIs analyzed. This was done using the Rosner Test if there were at least 10 probes for the target (k=0.2*Number of Probes, alpha=0.01), or the Grubbs test if there were less than 10 probes for the target. Probes flagged as outliers in less than 20% of the ROIs analyzed were only removed from the analysis for the ROIs in which they were flagged. Count reported for each target transcript were calculated as the geometric mean of the remaining probes.

The counts for each target transcript were then normalized to the count of the house keeper genes (C1orf43, GPI, OAZ1, POLR2A, PSMB2, RAB7A, SDHA, SNRPD3, TBC1D10B, TPM4, TUBB,

UBB). The geometric mean of the house keeper gene counts was calculated for each ROI. These geometric means were then divided by the geometric mean of the geometric mean of the house keeper genes to generate a normalization factor for each ROI. The counts of the transcripts in each AOI were than multiplied by the associated normalization factor.

The normalized in situ RNA measures were used to compute: (1) the T cell levels as described in the Inferring tumor composition section; (2) the overall expression of the malignant programs in each of the regions of interest (ROI), as described in the Gene sets Overall Expression section;
and (3) the differentiation scores, as described in the Malignant epithelial and mesenchymal differentiation programs section.

### Identifying SS18-SSX Targets

The fusion program consists of genes that were differentially expressed in the Aska or SYO1 cells with the SS18-SSX shRNA (shSSX) compared to those with control shRNA (shCt) after 3 or 7 days post-infection. Gene that were previously reported (35, 36) to be bound by the SS18-SSX oncoprotein in at least two SyS cell lines were defined as direct SS18-SSX targets, and were used to stratify the SS18-SSX program to direct and indirect targets.

### Mapping Cancer-Immune Interactions

The association between the core oncogenic program in the malignant cells and the expression of different ligands/cytokines in the immune cells was examined using the multilevel mixed-effects regression model described above, using the scRNA-Seq data collected from SyS tumors. The dependent variable y was the OE of the core oncogenic program and the covariate x was the average expression of a certain ligand/cytokine in a specific type of immune cells (e.g., macrophages) that were profiled from the same tumor. The model also corrected for inter-patient dependencies using the patient-specific intercepts and for cell complexity (log(number of reads)). Applicants restricted the analysis to ligands/cytokines that can physically bind to proteins expressed by the malignant cells (37). The immune cells were either macrophages or CD8 T cells, as other immune cell types were not sufficiently represented in the data.

Applicants used a similar approach to further stratify the program to its TNF/IFN-dependent and independent components. Applicants repeated the same analysis described above, using each one of the genes in the core oncogenic program as the dependent variable. Genes which were associated with both TNF and IFN (P<0.05, following Bonferroni correction) were considered as TNF/IFN dependent, and genes which were not associated with both cytokines (P>0.05) were considered as TNF/IFN-independent.

### TNF and IFNγ Impact on SyS Cell Cultures

SyS cell cultures were treated with TNF and IFNγ, separately and in combination (see In vitro IFN/TNF experiment section), and profiled with scRNA-Seq. Given this data, differentially expressed genes and gene sets were identified using mixed-effects regression models (Multilevel mixed-effects models section), with experiment-specific intercepts. The dependent variable was the expression of a gene or the OE of a gene set. The model included three treatment covariates: only TNF, only IFN, and a combination of TNF and IFN. Another binary covariate denoted the duration of the treatment (1 for <24 h duration and 0 otherwise). The model corrected for differences between the different SyS cultures and experiments, and identified patterns that repeatedly appeared across the different experiments. The effect-size and significance of the combination covariate denotes the effect of the combination, and not the synergy between the two cytokines.

To examine if the combined treatment with TNF and IFNγ had synergistic effects, Applicants used only the control cells and the cells treated for 4 days with one or two of the cytokines. This model also included 3 binary treatment covariates (TNF, IFN, and the combination), but this time cells that were treated with the combination were positive for all three treatment covariates. The effect-size and significance of the combination covariate hence denotes the synergistic effect of the combination.

### Reconstructing Regulatory Networks

To reconstruct the gene regulatory network controlling the core oncogenic program Applicants assembled a database of transcription factor (TF) to target mapping based on four sources: JASPAR (38), HTRIdb (39), MSigDB (40, 41), and TRRUST (42), and augmented it with the direct SS18-SSX targets identified here (Table 8) and TF-target pairs Applicants identified in a cis-regulatory motif analysis of the core oncogenic program. Specifically, for the cis-regulatory analysis, Applicants used RcisTarget (43) (a R/Bioconductor implementation of icisTarget (44) and iRegulon (45)) to identify cis-regulatory elements significantly overrepresented in a window of 500 bp around the transcription start site of the core oncogenic genes (normalized enrichment score >3.0) along with their cognate TFs.

Applicants pruned the resulting network to include only core oncogenic program genes (and SS18-SSX) (i.e., all TFs and targets aside from SS18-SSX are program genes). An edge in the network between a TF and its target denotes that: (1) the TF is regulating the target according to at least one of the sources described above, and (2) there is an association between their expression levels in the scRNA-Seq data of SyS tumors. Edges are weighted 1 and −1 to reflect positive and negative associations. Applicants used pageRank (46) (with the R implementation as provided in igraph (igraph.org/r/)) as a measure of TF and target importance in the network. To compute TF importance Applicants first flipped the direction of the edges in the network, going from target to TFs. Consistent with the network weights, targets from the up- or down-regulated side of the network were considered induced or repressed, respectively. Likewise, TFs from the up- or down-regulated side of the network were considered activators and repressors, respectively.

### Selectivity and Synergy in Drug Experiments

To evaluate the impact of each drug on the expression of a certain program or gene in a specific cell lines (SYO1, HSSYII, or MSCs), Applicants used a regression model with four binary treatment covariates: abemaciclib, TNF, panobinostat, and the combination of all three drugs. As in the case of TNF/IFN analysis, to examine the synergy of the combination, the cells treated with the combination were positive for all four treatment covariates. The model also included the number of reads detected in each cell (log-transformed) to control for technical variation. When examining the impact on the two SyS cell lines together, Applicants used a mixed-effects model with a cell line specific intercept, to control for cell line specific baseline states. Drug selectivity was examined by using a mixed-effects model that accounts for all three cell lines and has another covariate to denote if the treated cells were SyS or not.

### Data Availability

Processed scRNA-seq data and interactive plots generated for this study are provided through the Single Cell Portal available at broadinstitute.org/single_cell/study/synovial-sarcoma. The processed scRNA-seq data is provided via the Gene Expression Omnibus (GEO), accession number GSE131309 (available at National Library of Medicine of the NCBI; nih.gov/geo/query/acc.cgi?acc=GSE131309); access currently requires a secure token avcjkioijjylryp. Raw scRNA-Seq data will be deposited in DUOS (duos is available at broadinstitute.org/#/home).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

### REFERENCES

1. T. O. Nielsen, N. M. Poulin, M. Ladanyi, Synovial sarcoma: recent discoveries as a roadmap to new avenues for therapy. Cancer Discov. 5, 124-134 (2015).
2. C. Kadoch, G. R. Crabtree, Reversible disruption of mSWI/SNF (BAF) complexes by the SS18-SSX oncogenic fusion in synovial sarcoma. Cell. 153, 71-85 (2013).
3. M. Ayyoub et al., CD4+ T Cell Responses to SSX-4 in Melanoma Patients. J. Immunol. 174, 5092 (2005).
4. M. Ayyoub et al., Tumor-reactive, SSX-2-specific CD8⁺ T Cells Are Selectively Expanded during Immune Responses to Antigen-expressing Tumors in Melanoma Patients. Cancer Res. 63, 5601 (2003).
5. H. A. Smith, D. G. McNeel, The SSX Family of Cancer-Testis Antigens as Target Proteins for Tumor Therapy. Clin. Dev. Immunol. 2010, 18 (2010).
6. H. A. Smith, D. G. McNeel, Vaccines targeting the cancer-testis antigen SSX-2 elicit HLA-A2 epitope-specific cytolytic T cells. J. Immunother. Hagerstown Md. 1997. 34, 569-580 (2011).
7. M. J. McBride et al., The SS18-SSX Fusion Oncoprotein Hijacks BAF Complex Targeting and Function to Drive Synovial Sarcoma. Cancer Cell (2018), doi:10.1016/j.ccell.2018.05.002.
8. A. Banito et al., The SS18-SSX Oncoprotein Hijacks KDM2B-PRC1.1 to Drive Synovial Sarcoma. Cancer Cell. 33, 527-541.e8 (2018).
9. L. Su et al., Deconstruction of the SS18-SSX fusion oncoprotein complex: insights into disease etiology and therapeutics. Cancer Cell. 21, 333-347 (2012).
10. R. Nakayama et al., Gene expression profiling of synovial sarcoma: distinct signature of poorly differentiated type. Am. J Surg. Pathol. 34, 1599-1607 (2010).
11. P. Lagarde et al., Chromosome instability accounts for reverse metastatic outcomes of pediatric and adult synovial sarcomas. J. Clin. Oncol. Off. J. Am. Soc. Clin. Oncol. 31, 608-615 (2013).
12. S. Picelli et al., Full-length RNA-seq from single cells using Smart-seq2. Nat. Protoc. 9, 171-181 (2014).
13. G. X. Y. Zheng et al., Massively parallel digital transcriptional profiling of single cells. Nat. Commun. 8, 14049 (2017).
14. B. Haas et al., STAR-Fusion: Fast and Accurate Fusion Transcript Detection from RNA-Seq. bioRxiv (2017), doi:10.1101/120295.
15. A. P. Patel et al., Single-cell RNA-seq highlights intratumoral heterogeneity in primary glioblastoma. Science. 344, 1396-1401 (2014).
16. Comprehensive and Integrated Genomic Characterization of Adult Soft Tissue Sarcomas. Cell. 171, 950-965.e28 (2017).
17. S. V. Puram et al., Single-Cell Transcriptomic Analysis of Primary and Metastatic Tumor Ecosystems in Head and Neck Cancer. Cell. 171, 1611-1624.e24 (2017).
18. I. Tirosh et al., Dissecting the multicellular ecosystem of metastatic melanoma by single-cell RNA-seq. Science. 352, 189-196 (2016).
19. A. S. Venteicher et al., Decoupling genetics, lineages, and microenvironment in IDH-mutant gliomas by single-cell RNA-seq. Science. 355 (2017), doi:10.1126/science.aai8478.
20. A. Tsherniak et al., Defining a Cancer Dependency Map. Cell. 170, 564-576.e16 (2017).
21. J. H. Taube et al., Core epithelial-to-mesenchymal transition interactome gene-expression signature is associated with claudin-low and metaplastic breast cancer subtypes. Proc. Natl. Acad. Sci. U.S.A. 107, 15449-15454 (2010).
22. C. J. Gröger, M. Grubinger, T. Waldhör, K. Vierlinger, W. Mikulits, Meta-Analysis of Gene Expression Signatures Defining the Epithelial to Mesenchymal Transition during Cancer Progression. PLOS ONE. 7, e51136 (2012).
23. J. Fan et al., Characterizing transcriptional heterogeneity through pathway and gene set overdispersion analysis. Nat. Methods. 13, 241-244 (2016).
24. L. Jerby-Arnon et al., A Cancer Cell Program Promotes T Cell Exclusion and Resistance to Checkpoint Blockade. Cell. 175, 984-997.e24 (2018).
25. J.-R. Lin et al., Highly multiplexed immunofluorescence imaging of human tissues and tumors using t-CyCIF and conventional optical microscopes. eLife. 7, e31657 (2018).
26. K. Baird et al., Gene expression profiling of human sarcomas: insights into sarcoma biology. Cancer Res. 65, 9226-9235 (2005).
27. Y. Sun et al., IGF2 is critical for tumorigenesis by synovial sarcoma oncoprotein SYT-SSX1. Oncogene. 25, 1042-1052 (2006).
28. A. Subramanian et al., A Next Generation Connectivity Map: L1000 Platform and the First 1,000,000 Profiles. Cell. 171, 1437-1452.e17 (2017).
29. M. J. T. Stubbington et al., T cell fate and clonality inference from single-cell transcriptomes. Nat. Methods. 13, 329-332 (2016).
30. M. Sade-Feldman et al., Defining T Cell States Associated with Response to Checkpoint Immunotherapy in Melanoma. Cell. 175, 998-1013.e20 (2018).
31. C. Zheng et al., Landscape of Infiltrating T Cells in Liver Cancer Revealed by Single-Cell Sequencing. Cell. 169, 1342-1356.e16 (2017).
32. J. P. Böttcher et al., Functional classification of memory CD8+ T cells by CX3CR1 expression. Nat. Commun. 6, 8306 (2015).
33. N. E. Scharping, A. V. Menk, R. D. Whetstone, X. Zeng, G. M. Delgoffe, Efficacy of PD-1 Blockade Is Potentiated by Metformin-Induced Reduction of Tumor Hypoxia. Cancer Immunol. Res. 5, 9-16 (2017).
34. N. E. Scharping, A. V. Menk, R. D. Whetstone, X. Zeng, G. M. Delgoffe, Efficacy of PD-1 Blockade Is Potentiated by Metformin-Induced Reduction of Tumor Hypoxia. Cancer Immunol. Res. 5, 9-16 (2017).
35. S. Spranger, R. Bao, T. F. Gajewski, Melanoma-intrinsic β-catenin signalling prevents anti-tumour immunity. Nature. 523, 231-235 (2015).
36. D. Pan et al., A major chromatin regulator determines resistance of tumor cells to T cell-mediated killing. Science. 359, 770-775 (2018).
37. D. Miao et al., Genomic correlates of response to immune checkpoint therapies in clear cell renal cell carcinoma. Science. 359, 801-806 (2018).
38. I. Datar, K. A. Schalper, Epithelial-Mesenchymal Transition and Immune Evasion during Lung Cancer Progression: The Chicken or the Egg? Clin. Cancer Res. 22, 3422 (2016).
39. S. Terry et al., New insights into the role of EMT in tumor immune escape. Mol. Oncol. 11, 824-846 (2017).
40. Y. Zhou et al., Evaluation of expression of cancer stem cell markers and fusion gene in synovial sarcoma: Insights into histogenesis and pathogenesis. Oncol. Rep. 37, 3351-3360 (2017).
41. A. Butler, P. Hoffman, P. Smibert, E. Papalexi, R. Satija, Integrating single-cell transcriptomic data across different conditions, technologies, and species. Nat. Biotechnol. 36, 411 (2018).

What is claimed is:

1. A method of detecting an expression signature in synovial sarcoma (Sys) tumor comprising detecting in tumor cells obtained from a subject the expression or activity of a malignant cell gene signature comprising one or more biomarkers selected from the group consisting of
a) epithelial malignant signature as defined in Table 1E;
b) mesenchymal malignant cell signature as defined in Table 1D;
c) cell cycle signature as defined in Table 1C;
d) core oncogenic signature as defined in Table 1A.1;
e) a fusion signature as defined in Table 8; or
f) a combination thereof

2. The method of claim 1, wherein detection of the cell cycle signature indicates an increased risk of metastatic disease compared to a sample not expressing the cell cycle signature.

3. The method of claim 2, wherein the one or more biomarkers comprise cyclin D2 (CND2), CDK6, or both CND2 and CDK6.

4. The method of claim 1, wherein detection of the core oncogenic signature indicates an increased risk of metastatic disease compared to a sample not expressing the core oncogenic signature.

5. The method of claim 1, wherein absence of the core oncogenic signature indicates higher progression free survival.

6. A method of diagnosing a subject with synovial sarcoma, comprising detecting one or more signatures of claim 1.

7. A method of diagnosing a subject with increased risk of metastatic disease, comprising detecting one or more signatures of claim 1.

8. A method of treating SyS in a subject in need thereof comprising administering inhibitor of HDAC, CDK4/6, or a combination thereof to selectively target synovial sarcoma cells.

9. The method of claim 7, further comprising administration with immune checkpoint inhibitors.

10. A method of monitoring a cancer in a subject in need thereof comprising detecting the expression or activity of one or more expression signatures of claim 1 in tumor samples obtained from the subject for at least two time points.

11. The method of claim 10, wherein at least one sample obtained before treatment.

12. The method of claim 10, wherein the tumor sample obtained after treatment.

13. A method of treatment comprising targeting one or more genes or polypeptides of one or expression signatures of claim 1.

14. A method of treatment for Synovial Sarcoma comprising treatment with TNF and IFN-gamma, the treatment providing a synergistic effect.

15. A method of treatment comprising administration of a modulator of one or more genes of cell cycle signature as defined in Table 1C, a SS18-SSX signature as defined in Table 8, or a combination thereof.

16. The method of treatment of claim 15, wherein a combination of a modulator of cell cycle signature and SS18-SSX signature are administered and provide a synergistic effect.

17. An isolated CD8+ T cell characterized by expression of one or more biomarkers of an expression signature as defined in Table 1F.

18. An isolated or engineered CD8+ T cell characterized by increased expression of TNF alpha and/or interferon gamma.

19. A method of treating a subject with SyS comprising administration of the isolated or engineered CD8+ T cell of claim 17 or 18 to a subject in need thereof.

20. A method of treating Synovial Sarcoma (Sys) in a subject comprising:
i) detecting the expression or activity of a malignant cell gene signature is a sample from a subject, the signature comprising one or more biomarkers selected from the group consisting of:
a) epithelial malignant signature as defined in Table 1E;
b) mesenchymal malignant cell signature as defined in Table 1D;
c) cell cycle signature as defined in Table 1C;
d) core oncogenic signature as defined in Table 1A.1;
e) a fusion signature as defined in Table 8; or
f) a combination thereof; and
ii) administering an effective amount of a modulating agent of the signature.

21. The method of claim 20, wherein the modulating agent is inhibitor of HDAC, CDK4/6, or a combination thereof, to selectively target synovial sarcoma cells.


SEQUENCE LISTING (23 sequences)

SEQ ID NO: 1 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 2 - protein, 9 aa, Homo sapiens
IEVMYPPPY

SEQ ID NO: 3 - protein, 288 aa, Artificial Sequence
  Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 4 - protein, 183 aa, Homo sapiens
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 5 - DNA, 48 bp, Artificial Sequence
  Synthetic
cctaaggttaagtcgccctcgctcgagcgagggcgacttaaccttagg

SEQ ID NO: 6 - DNA, 21 bp, Artificial Sequence
  Synthetic
cagtcactgacagttaataaa

SEQ ID NO: 7 - protein, 7 aa, Simian Virus 40
PKKKRKV

SEQ ID NO: 8 - protein, 10 aa, Simian Virus 40
PKKKRKVEAS

SEQ ID NO: 9 - protein, 16 aa, Artificial Sequence
  Synthetic
KRPAATKKAGQAKKKK

SEQ ID NO: 10 - protein, 9 aa, Artificial Sequence
  Synthetic
PAAKRVKLD

SEQ ID NO: 11 - protein, 11 aa, Artificial Sequence
  Synthetic
RQRRNELKRSP

SEQ ID NO: 12 - protein, 38 aa, Artificial Sequence
  Synthetic
NQSSNFGPMKGGNFGGRSSGPYGGGGQYFAKPRNQGGY

SEQ ID NO: 13 - protein, 42 aa, Artificial Sequence
  Synthetic
RMRIZFKNKGKDTAELRRRRVEVSVELRKAKKDEQILKRRNV

SEQ ID NO: 14 - protein, 8 aa, Homo sapiens
VSRKRPRP

SEQ ID NO: 15 - protein, 8 aa, Homo sapiens
PPKKARED

SEQ ID NO: 16 - protein, 8 aa, Homo sapiens
PQPKKKPL

SEQ ID NO: 17 - protein, 12 aa, Mus sp.
SALIKKKKKMAP

SEQ ID NO: 18 - protein, 5 aa, Influenza virus NS1
DRLRR

SEQ ID NO: 19 - protein, 7 aa, Influenza virus NS1
PKQKKRK

SEQ ID NO: 20 - protein, 10 aa, Hepatitis virus
RKLKKKIKKL

SEQ ID NO: 21 - protein, 10 aa, Mus sp.
REKKKFLKRR

SEQ ID NO: 22 - protein, 20 aa, Homo sapiens
KRKGDEVDGVDEVAKKKSKK

SEQ ID NO: 23 - protein, 17 aa, Homo sapiens
RKCLQAGMNLEARKTKK